(19) United States
(12) Patent Application Publication
(10) Pub. No.: US 2015/0307520 A1
(43) Pub. Date: Oct. 29, 2015

(54) NOVEL HETEROARYL AND HETEROCYCLE COMPOUNDS, COMPOSITIONS AND METHODS THEREOF

(71) Applicant: HUTCHISON MEDIPHARMA LIMITED, Shanghai (CN)

(72) Inventors: Wei-Guo SU, Shanghai (CN); Guangxiu DAI, Shanghai (CN); Kun XIAO, Shanghai (CN); Hong JIA, Shanghai (CN); Zhulin ZHANG, Shanghai (CN); Jennifer Diane VENABLE, Shanghai, CA (US); Scott Damian BEMBENEK, San Diego, CA (US); Wenying CHAI, San Diego, CA (US)

(21) Appl. No.: 14/417,694

(22) PCT Filed: Jul. 26, 2013
(86) PCT No.: PCT/CN2013/080195

§ 371 Date: Jan. 27, 2015

(30) Foreign Application Priority Data
Jul. 27, 2012 (CN) PCT/CN2012/079290
Mar. 15, 2013 (CN) PCT/CN2013/072686

(51) Int. Cl. C07D 519/00; A61K 45/06; A61K 31/506; A61K 31/5377; A61K 31/52; A61K 31/519; A61K 31/53; C07D 487/04

(52) U.S. Cl. CPC C07D 519/00; A61K 31/53; A61K 45/06; C07D 487/04; A61K 31/5377; A61K 31/52; A61K 31/519; A61K 31/506

(57) ABSTRACT

Disclosed are novel heteroaryl and heterocycle compounds of formula I-1, I-2 or I-3 and pharmaceutical compositions comprising them, uses and methods thereof for inhibiting the activity of PI₃K and for treating inflammatory and autoimmune diseases and cancer.

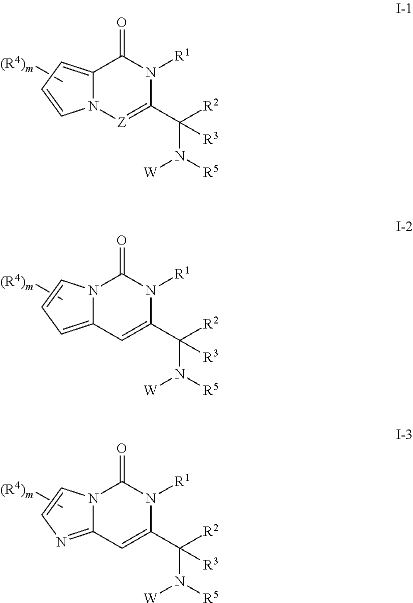

### FIELD OF THE INVENTION

This invention relates generally to the field of medicine and, more specifically, to novel heteroaryl and heterocycle compounds and pharmaceutical compositions comprising them, uses and methods thereof for inhibiting the activity of PI₃K and for treating inflammatory and autoimmune diseases and cancer.

### BACKGROUND OF THE INVENTION

Phosphoinositide 3-kinases (PI 3-kinases or PI₃Ks) are a family of enzymes involved in cellular functions such as cell growth, proliferation, differentiation, motility, survival and intracellular trafficking. After exposure of cells to various biological stimuli, PI₃Ks primarily phosphorylate phosphatidylinositol-4,5-bisphosphate (PtdIns(4,5)P2, PIP2) at the 3′-OH position of the inositol ring to generate phosphatidylinositol-3,4,5-trisphosphate (PtdIns(3,4,5)P3, PIP3) which has an important role as second messengers by working as a docking platform for lipid-binding domains, such as the pleckstrin homology (PH) domains of various cellular proteins. These include kinases (such as 3-phosphoinositide-dependent protein kinase 1 (PDK1) and protein kinase B (PKB)/Akt) that trigger downstream kinase cascades, and guanine-nucleotide exchange factors (such as Vav and P-Rex) that control the activity of small GTPases (T Rückle, M. K. et al. Nature Reviews Drug Discovery, 2006, 5, 903-9018).

Based on sequence homology and lipid substrate specificity, the PI₃K family is divided into three classes: I, II, and III. The most studied and the focus of this invention, the class I PI₃Ks, are heterodimeric proteins, each containing a smaller regulatory domain and a larger 110 kDa catalytic domain which occur in four isoforms differentiated as p110α, p110β, p110γ and p110δ (T. J. Sundstrom. et al Org. Biomol. Chem., 2009, 7, 840-850). Among them, p110α, p110β and p110δ together, termed as the class IA PI₃K, bind to p85 regulatory subunit and are primarily activated by protein tyrosine kinase-coupled receptors (RTK) and/or Ras proteins, whereas PI₃Kγ as the sole class IB member, binds to one of two noncatalytic subunits, p101 or p87, is activated by G-protein coupled receptors (GPCRs) through direct interaction with G-protein βγ dimers and Ras proteins, which are widely implicated in various aspects of immune function and regulation.

All four class I catalytic PI₃K isoforms show a characteristic expression pattern in vivo. p110α and p110β are ubiquitously expressed, while p110γ and p110δ are found predominantly in leukocytes, endothelial cells and smooth muscle cells (T. J. Sundstrom. et al Org. Biomol. Chem., 2009, 7, 840-850). Deletion of the class IA isoform p110α or β induces embryonic lethality (E9.5-E10) (Bi L, Okabe I. et al. J Biol Chem, 1999, 274: 10963-8; Bi L, Okabe I. et al. Mamm Genome. 2002, 13, 169-72) p110γ-deficient mice develop and reproduce normally, although they have suboptimal immune responses because of defects in T-cell activation as well as in neutrophil and macrophage migration. The loss of p110δ mice are also viable and fertile but exhibit significant defects in T, B cell activation (A Ghigo. et al. BioEssays 2010, 32: 185-196).

Dysregulation and overactivation of the PI₃K/AKT pathway has been firmly established in cancer cells. In principle, modulating PI₃K and thus controlling PIP3 levels should regulate AKT activity and ultimately suppress tumor growth. The expression of PI₃Kδ is generally restricted to hematopoietic cell types. The p110δ isoform is constitutively activated in B cell tumors. Genetic and pharmacologic approaches that specifically inactivate the p110δ isoform have demonstrated its important role for the treatment of B cell malignancy (B. J. Lannutti. et al. Blood. 2011, 117, 591-594). Previous studies have shown that CAL-101, a potent and selective p110 inhibitor, has broad antitumor activity against cancer cells of hematologic origin. (Lannutti B. J. Am Soc Hematol. 2008; 112. Abstract 16; Flinn I. W. et al. J. Clin. Oncol. 2009; 27(A3543))

In addition to cancer, PI₃K has also been suggested as a target for inflammatory and autoimmune disorders. The isoforms p110δ and p110γ are mainly expressed in cells of the immune system and contributes to innate and adaptive immunity. p110δ and p110γ regulate diverse immune cell function. For example, inhibition of p110δ leads to suppression of B-cell activation and function, suppression of T-lymphocyte proliferation, T-cell trafficking, and Th1-Th2 differentiation and Treg function. Inhibition of both p110δ and p110γ results in inhibition of neutrophil (leukocyte) chemotaxis, inhibition of mast cell activation, intact macrophage phagocytosis and endothelium activation. Inhibition of p110γ could activate microglial (C. Rommel. et al. Current Topics in Microbiology and Immunology, 2010, 1, 346, 279-299). So isoform-specific p110δ or p110γ inhibitors are expected to have therapeutic effects on these diseases without interfering with general PI₃K signaling critical to the normal function of other cellular systems. p110δ and p110γ supporting the hypothesis that p110γ alone, p110δ alone, or dual-blockade of both, all present a unique therapeutic opportunity in that pharmacological inhibition, but the two PI₃K isoforms simultaneously may yield more superior clinical results in the treatment of a variety of complex immune-mediated inflammatory diseases. In the case of RA, Phosphoinositide 3-kinases (PI₃Ks), most notably PI₃Kδ and PI₃Kγ, have crucial and specific roles at all stages of disease progression: in antigen signalling in B and T cells, and in signalling downstream of FcRs, cytokine receptors and chemokine receptors in mast cells, macrophages, neutrophils and synoviocytes (C. Rommel. et al. Nature Reviews Immunology, 2007, 7, 191-201). Although the pathogenesis of RA is not yet completely understood, chemokines and other chemoattractants have been detected in the inflamed joint and are responsible for the recruitment of leukocytes into the joints. Amongst these, neutrophils constitute the most abundant population and are capable of inducing inflammatory response and tissue damage (T Riickle, M. K. et al. Nature Reviews Drug Discovery, 2006, 5, 903-9018). Blockade of hematopoietic PI₃Kγ and/or PI₃Kδ can potently suppresses neutrophil chemotaxis and, in turn, the progression of joint inflammation and cartilage erosion.

Novel compounds are disclosed which in some instances are inhibitors of PI₃Ks kinase activity including p110δ, p110γ, p110α, and p110β. These compounds therefore have potential therapeutic benefit in the treatment of a variety of diseases associated with inappropriate p110δ, p110γ, p110α, and p110β activity, such as cancer, inflammatory, allergic and autoimmune diseases and leukemia etc, in particular systemic lupus erythematosus (SLE) and rheumatoid arthritis (RA), allergic disorders, respiratory diseases like asthma and chronic obstructive pulmonary disease (COPD), multiple sclerosis, all pathologic conditions whose onset and/or progression is driven by an inflammatory insult, such as myocardial infarction and cancer.

### SUMMARY OF THE INVENTION

The present invention provides a compound of formula I-1, I-2 or I-3:

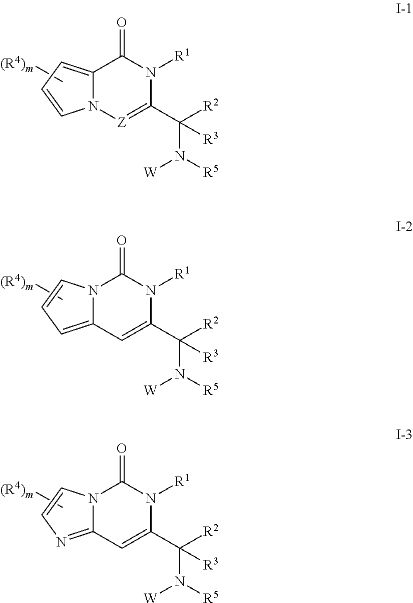

and/or its solvates, racemic mixture, enantiomers, diasteromers, tautomers, or mixtures of any ratio or pharmaceutically acceptable salts thereof, wherein all substituents are as defined in the detailed description.

Also provided is a pharmaceutical composition, comprising at least one compound of formula I-1, I-2 or I-3 and/or at least one pharmaceutically acceptable salt thereof and at least one pharmaceutically acceptable carrier.

Also provided is a method of inhibiting the activity of PI₃K kinase, comprising contacting the kinase with an effective amount of at least one compound of formula I-1, I-2 or I-3 and/or at least one pharmaceutically acceptable salt thereof.

Also provided is a method of treating a disease responsive to inhibition of PI₃K in a subject, comprising administering a therapeutically effective amount of at least one compound of formula I-1, I-2 or I-3 and/or at least one pharmaceutically acceptable salt thereof.

Also provided is at least one compound and/or at least one pharmaceutically acceptable salt described herein for use in the treatment of diseases responsive to inhibition of PI₃K.

Also provided is a use of at least one compound and/or at least one pharmaceutically acceptable salt described herein in the manufacture of a medicament for use in the treatment of diseases responsive to inhibition of PI₃K.

The subject described herein can be human.

### DETAILED DESCRIPTION OF THE INVENTION

Provided is at least one compound of formula I-1, I-2 or I-3:

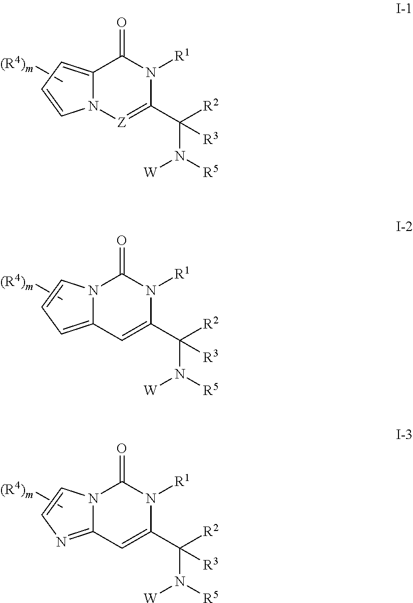

and/or its solvates, racemic mixture, enantiomers, diasteromers, tautomers, or mixtures of any ratio, or pharmaceutically acceptable salts thereof, wherein
Z═N or CH;
R¹ is selected from, optionally substituted C₁₋₆ alkyl, optionally substituted C₃₋₆ cycloalkyl, —(CR′R″)ₙ-heterocycle, and —(CR′R″)ₙ-aryl, —(CR′R″)ₙ-heteroaryl, wherein heterocycle, aryl and heteroaryl independently are 5-6 membered monocyclic ring, which are optionally substituted with one or more groups selected from hydrogen, halo, optionally substituted C₁₋₆ alkyl, optionally substituted C₁₋₆ alkoxyl, —CN, —CF₃, and —SO₂R′;
R² and R³ are each independently selected from hydrogen, and optionally substituted C₁₋₄ alkyl;
R⁴ is selected from hydrogen, halo, —CN, optionally substituted C₁₋₆ alkyl, optionally substituted C₃₋₆ cycloalkyl, optionally substituted C₂₋₆ alkenyl, optionally substituted C₂₋₆ alkynyl, —C(O)NR′R″, and optionally substituted 5-6 membered monocyclic heteroaryl;
R⁵ is selected from hydrogen and optionally substituted C₁₋₄ alkyl;
or R³, R⁵ and the atoms they are attached to form an optionally substituted 4-6 membered mono- or bicyclic saturated or partially unsaturated heterocyclic ring;
R′ and R″ are each independently selected from hydrogen, halo, optionally substituted C₁₋₆ alkyl, optionally substituted C₃₋₆ cycloalkyl, and optionally substituted 4-6 membered monocyclic heterocycle;
or R′, R″ and the nitrogen or carbon atom they are both attached to form an optionally substituted 3-7 membered heterocycle;
each of m and n is 0, 1, 2, or 3;
each of p is 1 or 2;
W is a heteroaryl, which is optionally substituted with one or more groups selected from halo, —CN, —CF₃, —NO₂, —OR′, —NR′R″, —NR′COR″, —(CR′R″)ₙ—C(O)R′, —(CR′R″)ₙ—C(═N—OR′)—R″, —(CR′R″)ₙ—C(O)NR′R″, —(CR′R″)ₙ—S(O)ₚR′, —(CR′R″)ₙ—SR′, optionally substituted C₁₋₆ alkyl, optionally substituted C₂₋₆ alkenyl, optionally substituted C₂₋₆ alkynyl, optionally substituted C₁₋₆ alkoxy, optionally substituted 5-6 membered monocyclic heterocycle and optionally substituted 5-6 membered monocyclic heteroaryl;
provided that for formula I-1, when Z═N, R³, R⁵ and the atoms they are attached to must form an optionally substituted 4-6 membered mono- or bicyclic saturated or partially unsaturated heterocyclic ring, with the provision that when R³, R⁵ and the atoms they are attached to form an optionally substituted 5 membered mono- or bicyclic saturated or partially unsaturated heterocyclic ring, R⁴ is not hydrogen, —CN, or aminomethyl;

wherein each optionally substituted group above for which the substituent(s) is (are) not specifically designated, can be unsubstituted or independently substituted with one or more, such as one, two, or three, substituents independently chosen from halo, —OH, —CN, —CF₃, —SO₂R′, —NR′R″, alkyl, alkenyl, alkynyl, alkoxy, cycloalkyl, heterocycle, aryl, and heteroaryl, in which alkoxy, cycloalkyl, heterocycle, aryl and heteroaryl can be further optionally substituted with one or more groups selected from halo, —OH, —CN, —CF₃, —SO₂R′, —NR′R″, alkyl, alkenyl, alkynyl, alkoxy, cycloalkyl, heterocycle, aryl, and heteroaryl.

In some embodiments, the each optionally substituted group can be unsubstituted or independently substituted with one or more, such as one, two, or three, substituents independently chosen from halogen, —OH, —CN, —CF₃, —SO₂R′, —NR′R″, C₁-C₁₀ alkyl (preferably C₁-C₆ alkyl, more preferably C₁-C₄ alkyl), C₂-C₁₀ alkenyl (preferably C₂-C₆ alkenyl, more preferably C₂-C₄ alkenyl), C₂-C₁₀ alkynyl (preferably C₂-C₆ alkynyl, more preferably C₂-C₄ alkynyl), C₁-C₁₀ alkoxy (preferably C₂-C₆ alkoxy, more preferably C₂-C₄ alkoxy), C₃-C₁₂ cycloalkyl, 3-12 membered heterocycle, aryl and heteroaryl, in which alkoxy, cycloalkyl, heterocycle, aryl and heteroaryl can be further optionally substituted with one or more groups selected from halo, —OH, —CN, —CF₃, —SO₂R′, —NR′R″, alkyl, alkenyl, alkynyl, alkoxy, cycloalkyl, heterocycle, aryl, and heteroaryl.

In some embodiments, the each optionally substituted group can be unsubstituted or independently substituted with one or more, such as one, two, or three, substituents independently chosen from halogen, —OH, —CN, —CF₃, —SO₂CH₃, —N(C₁-C₄ alkyl) (C₁-C₄ alkyl), C₁-C₄ alkyl, C₁-C₄ alkoxy, C₃-C₆ cycloalkyl, morpholinyl, phenyl and pyrimidinyl, in which morpholinyl, phenyl and pyrimidinyl can be further optionally substituted with one or more groups selected from halo, —OH, —CN, —CF₃, and C₁-C₄ alkyl.

In some embodiments, optionally substituted alkyl can be unsubstituted or independently substituted with one or more substituents independently chosen from: halogen, —OH, —CN, —CF₃, C₁-C₄ alkoxy, C₃-C₆ cycloalkyl, 4-6 membered heterocycle, 5-6 membered aryl, 5-6 membered heteroaryl, —N(C₁-C₄ alkyl) (C₁-C₄ alkyl), and SO₂R′; wherein R′ is selected from C₁₋₆ alkyl and C₃₋₆ cycloalkyl.

In some embodiments, optionally substituted alkenyl can be unsubstituted or independently substituted with one or more substituents independently chosen from: C₁-C₄ alkoxy and C₁-C₄ alkyl.

In some embodiments, optionally substituted alkynyl can be unsubstituted or independently substituted with one or more substituents independently chosen from: —OH, C₁-C₄ alkoxy and C₁-C₄ alkyl.

In some embodiments, optionally substituted cycloalkyl can be unsubstituted or independently substituted with one or more substituents independently chosen from: halogen, —OH, —CN, —CF₃, C₁-C₄ alkoxy, and C₁-C₄ alkyl.

In some embodiments, optionally substituted heteroaryl can be unsubstituted or independently substituted with one or more substituents independently chosen from: halogen, —CN, —CF₃, —NO₂, —OR′, —NR′R″, —NR′COR″, —COR′, —CONR′R″, —SO₂R′, —SR′, and —C(═NOR′)—R″, C₁-C₄ alkyl, C₃-C₆ alkenyl, C₂-C₆ alkynyl, C₃-C₆ cycloalkyl, C₁-C₄ alkoxy, 4-6 membered heterocycle, and 5-6 membered heteroaryl; wherein
R′ and R″ are each independently selected from hydrogen, C₁₋₆ alkyl, C₃₋₆ cycloalkyl, and C₁₋₆ haloalkyl;
or R′, R″ and the nitrogen or carbon atom they are both attached to form an optionally substituted 3-7 membered heterocycle.

In some embodiments, optionally substituted aryl can be unsubstituted or independently substituted with one or more substituents independently chosen from: halogen, —CN, C₁-C₄ alkoxy, C₁-C₄ alkyl, and SO₂R′; wherein R′ is selected from C₁₋₆ alkyl and C₃₋₆ cycloalkyl.

In some embodiments, optionally substituted heterocycl can be unsubstituted or independently substituted with one or more substituents independently chosen from: halogen, —OH, —CN, —CF₃, —SO₂R′, oxo, C₁-C₄ alkyl, and C₁-C₄ alkoxy; wherein C₁-C₄ alkoxy is optionally substituted by C₁-C₄ alkoxy, R′ is selected from C₁₋₆ alkyl and C₃₋₆ cycloalkyl.

In some embodiments, provided is at least one compound of formula I-1,
Z═N,
R¹ is selected from, optionally substituted C₁₋₆ alkyl, optionally substituted C₃₋₆ cycloalkyl, —(CR′R″)ₙ-heterocycle, —(CR′R″)ₙ-aryl, and —(CR′R″)ₙ-heteroaryl, wherein heterocycle, aryl and heteroaryl independently are 5-6 membered monocyclic ring, which are optionally substituted with one or more groups selected from halo, optionally substituted C₁₋₆ alkyl, optionally substituted C₁₋₆ alkoxyl, —CN, —CF₃, and —SO₂R′;
R² is selected from hydrogen and optionally substituted C₁₋₄ alkyl;
R³, R⁵ and the atoms they are attached to form an optionally substituted 4-6 membered mono- or bicyclic saturated or partially unsaturated heterocyclic ring;
R⁴ is selected from halo, C₁₋₆ alkyl, optionally substituted C₃₋₆ cycloalkyl, optionally substituted C₂₋₆ alkenyl, optionally substituted C₂₋₆ alkynyl, —C(O)NR′R″, and optionally substituted 5-6 membered monocyclic heteroaryl, wherein C₁-C₆ alkyl is optionally substituted with one or more groups selected from C₁-C₄ alkoxyl, —OH, and halo;
R′ and R″ are each independently selected from hydrogen, halo, optionally substituted C₁₋₆ alkyl, optionally substituted C₃₋₆ cycloalkyl, and optionally substituted 5-6 membered monocyclic heterocycle; or R′, R″ and the nitrogen or carbon atom they are both attached to form an optionally substituted 3-7 membered heterocycle;
each of m and n is 0, 1, 2, or 3;
each of p is 1 or 2;
W is a heteroaryl, which is optionally substituted with one or more groups selected from halo, —CN, —CF₃, —NO₂, —OR′, —NR′R″, —NR′COR″, —(CR′R″)ₙ—C(O)R′, —(CR′R″)ₙ—C(═N—OR′)—R″, —(CR′R″)ₙ—C(O)NR′R″, —(CR′R″)ₙ—S(O)ₚR′, —(CR′R″)ₙ—SR′, optionally substituted C₁₋₆ alkyl, optionally substituted C₂₋₆ alkenyl, optionally substituted C₂₋₆ alkynyl, optionally substituted C₁₋₆ alkoxy, optionally substituted 5-6 membered monocyclic heterocycle, and optionally substituted 5-6 membered monocyclic heteroaryl.

In some embodiments, provided is at least one compound of formula I-1, wherein Z═N, R³, R⁵ and the atoms they are attached to form an heterocyclic ring, which is optionally substituted

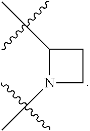 [SMILES: *C1CCN1*]

In some embodiments, provided is at least one compound of formula I-1, wherein Z═N, R³, R⁵ and the atoms they are attached to form an heterocyclic ring, which is optionally substituted

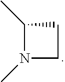

In some embodiments, provided is at least one compound of formula I-1, wherein Z═N, R³, R⁵ and the atoms they are attached to form an optionally substituted 5 membered saturated or partially unsaturated monocyclic heterocyclic ring, which contains one or more, preferably one or two heteroatoms selected from N, O, and S; R¹, R², R⁴, and W are as defined herein.

In some embodiments, the said 5 membered monocyclic saturated or partially unsaturated heterocyclic ring, which is formed by R³, R⁵ and the atoms they are attached to, is selected from

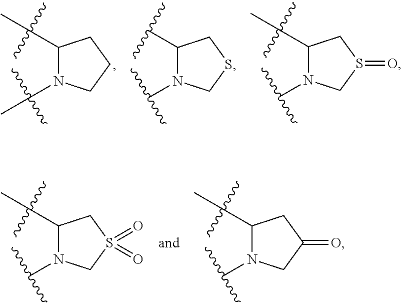 [SMILES: *C1CCCN1* ; *C1CSCN1* ; *C1CS(=O)CN1* ; *C1CS(=O)(=O)CN1* ; "and" ; *C1CC(=O)CN1*]

each of which is optionally substituted.

In some embodiments, the said 5 membered monocyclic saturated or partially unsaturated heterocyclic ring, which is formed by R³, R⁵ and the atoms they are attached to, is

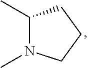

which is optionally substituted.

In some embodiments, provided is at least one compound of formula I-1, wherein Z═N, R³, R⁵ and the atoms they are attached to form an optionally substituted 6 membered mono- or bicyclic saturated or partially unsaturated heterocyclic ring, which contains one or more, preferably one or two heteroatoms selected from N, O, and S; R¹, R², R⁴, and W are as defined herein.

In some embodiments, the said 6 membered mono- or bicyclic saturated heterocyclic ring, which is formed by R³, R⁵ and the atoms they are attached to, is

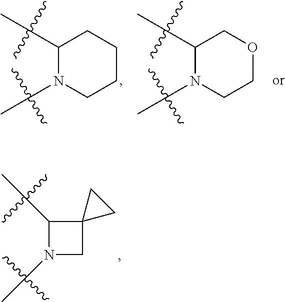 [SMILES: *C1CCCCN1* ; *C1COCCN1* ; *C1N(*)CC12CC2]

each of which is optionally substituted.

In some embodiments, the said 6 membered mono- or bicyclic saturated heterocyclic ring, which is formed by R³, R⁵ and the atoms they are attached to, is

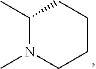

which is optionally substituted.

In some embodiments, provided is at least one compound of formula I-1, Z═N, the said heterocyclic ring, which is formed by R³, R⁵ and the atoms they are attached to, can be optionally substituted with one or more groups selected from halo, —OH, —CN, oxo, —SO₂R^{a}, —OR^{a} and optionally substituted C₁₋₆ alkyl; wherein R^{a} is C₁₋₆ alkyl, which is optional substituted with C₁-C₆ alkoxy.

In some embodiments, provided is at least one compound of formula I-1, Z═N, the said heterocyclic ring, which is formed by R³, R⁵ and the atoms they are attached to, can be optionally substituted with one or more groups selected from oxo, —SO₂R^{a}, and —OR^{a}; or can be optionally substituted with one or more groups selected from methyl, ethyl, n-propyl, i-propyl, n-butyl, i-butyl, t-butyl, each of which is optionally substituted;
R^{a} is selected from methyl, ethyl, n-propyl, i-propyl, n-butyl, i-butyl, and t-butyl, each of which is optionally substituted with C₁₋₄ alkoxyl.

In some embodiments, provided is at least one compound of formula I-1, Z═N, R³ and R⁵ are as defined above; R² is hydrogen.

In some embodiments, provided is at least one compound of formula I-1, Z═N, R³ and R⁵ are as defined above; R⁴ is selected from halo, C₁₋₆ alkyl, C₃-C₆ cycloalkyl, C₂-C₆ alkenyl, C₂-C₆ alkynyl, —C(O)NR′R″, wherein C₁-C₆ alkyl is optionally substituted with one or more groups selected from: C₁-C₄ alkoxyl, —OH, and halo.

In some embodiments, provided is at least one compound of formula I-1, Z═N, R³ and R⁵ are as defined above; R⁴ is selected from halo, —CF₃, and C₁₋₄ alkyl.

In some embodiments, provided is at least one compound of formula I-1, Z═N, R³ and R⁵ are defined as above; R⁴ is F, Cl or Br.

In some embodiments, m is 1.

In some embodiments, the said formula I-1 is

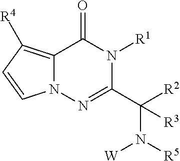

wherein R¹, R², R³, R⁴, R⁵ and Ware as defined herein.

In some embodiments, provided is at least one compound of formula I-1, I-2 or I-3, wherein Z═CH; R³, R⁵ and the atoms they are attached to form an optionally substituted 4-6 membered mono- or bi-cyclic saturated or partially unsaturated heterocyclic ring, which contains one or more, preferably one or two heteroatoms selected from N, O, and S; R¹, R², R⁴, and W are as defined herein.

In some embodiments, provided is at least one compound of formula I-1, I-2 or I-3, Z═CH; R³, R⁵ and the atoms they are attached to form an optionally substituted heterocycle selected from:

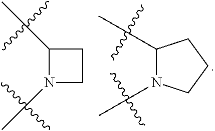 [SMILES: *C1CCN1* ; *C1CCCN1*]

In some embodiments, provided is at least one compound of formula I-1, I-2 or I-3, Z═CH; the said heterocyclic ring, which is formed by R³, R⁵ and the atoms they are attached to, can be optionally substituted with one or more groups selected from halo, —OH, —CN, oxo, —SO₂R^{a}, —OR^{a} and optionally substituted C₁₋₆ alkyl; wherein R^{a} is C₁₋₆ alkyl, which is optional substituted with C₁-C₆ alkoxy.

In some embodiments, provided is at least one compound of formula I-1, I-2 or I-3, Z═CH; the said heterocyclic ring, which is formed by R³, R⁵ and the atoms they are attached to, can be optionally substituted with one or more groups selected from oxo, —SO₂R^{a} and —OR^{a} and optionally substituted C₁₋₄ alkyl; wherein R^{a} is C₁₋₄ alkyl, which is optionally substituted with C₁₋₄ alkoxyl.

In some embodiments, provided is at least one compound of formula I-1, I-2 or I-3, Z═CH; R³ and R⁵ are as defined above; R² is hydrogen.

In some embodiments, provided is at least one compound of formula I-1, I-2 or I-3, Z═CH; R² and R³ are each independently H, methyl or ethyl.

In some embodiments, provided is at least one compound of formula I-1, I-2 or I-3, Z═CH; R⁵ is hydrogen.

In some embodiments, provided is at least one compound of formula I-1, I-2 or I-3, Z═CH; R¹, R², R³, R⁵, and W are as defined above; R⁴ is selected from hydrogen, halo, optionally substituted C₁-C₆ alkyl, and optionally substituted 5-6 membered monocyclic heteroaryl.

In some embodiments, provided is at least one compound of formula I-1, I-2 or I-3, Z═CH; R¹, R², R³, R⁵, and W are as defined above; R⁴ is selected from hydrogen, halo, C₁-C₄ alkyl and 5-6 membered monocyclic heteroaryl, wherein 5-6 membered monocyclic heteroaryl is optionally substituted with C₁₋₄ alkyl.

In some embodiments, m is 0, 1 or 2.

In some embodiments, m is 1.

In some embodiments, the said formula I-1, I-2 and I-3 are II-1, II-2 and II-3 respectively.

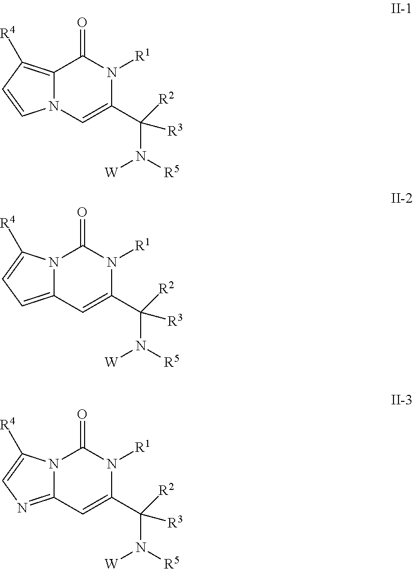

wherein R¹, R², R³, R⁴, R⁵, and W are as defined herein.

In some embodiments, R¹ is selected from, C₁-C₆ alkyl, C₃-C₆ cycloalkyl, —(CR′R″)ₙ-morpholinyl, —(CR′R″)ₙ-phenyl, —(CR′R″)ₙ-pyridinyl, or —(CR′R″)ₙ-pyrimidinyl, in which each of alkyl, morpholinyl, phenyl, pyridinyl and pyrimidinyl independently are optionally substituted with one or more groups selected from halo, C₁-C₄ alkyl, C₁-C₄ alkoxyl, —CN, —CF₃, and —SO₂R′. n, R′ and R″ are as defined herein.

In some embodiments, R¹ is (CR′R″)ₙ-aryl, n is 0 and said aryl can be optionally substituted with one or more groups selected from halo, —CN, C₁-C₄ alkoxyl and —SO₂R′. n. R′ and R″ are as defined herein. In some embodiments, R¹ is C₁₋₄ alkyl, which is optionally substituted with one or more groups selected from halo, —OH, —NR′R″, —CN, —CF₃, —SO₂R′, C₃-C₆ cycloalkyl, 5-6 membered heteroaryl and 5-6 membered heterocycle.

In some embodiments, R¹ is selected from C₃-C₆ cycloalkyl, phenyl, pyridyl, and pyrimidinyl, each of which is optionally substituted with one or more groups selected from halo, C₁₋₄ alkyl, —CN, —CF₃ and —SO₂R′; R′ and R″ are each independently hydrogen or C₁-C₄ alkyl.

In some embodiments, R¹ is (CR′R″)ₙ-phenyl, n is 0 and said phenyl can be optionally substituted with one or more groups selected from halo, —CN, C₁-C₄ alkoxyl, and —SO₂R′.

In some embodiments, R¹ is phenyl optionally substituted with one or more halo.

In some embodiments, R′ and R″ are each independently selected from hydrogen, C₁₋₆ alkyl, C₃₋₆ cycloalkyl and 4-6 membered heterocycle. In some embodiments, R′ and R″ are each independently selected from hydrogen, halo, —CN, —OH, and —CF₃.

In some embodiments, n is 0, 1 or 2.

In some embodiments, W is selected from IV-1 to IV-22,

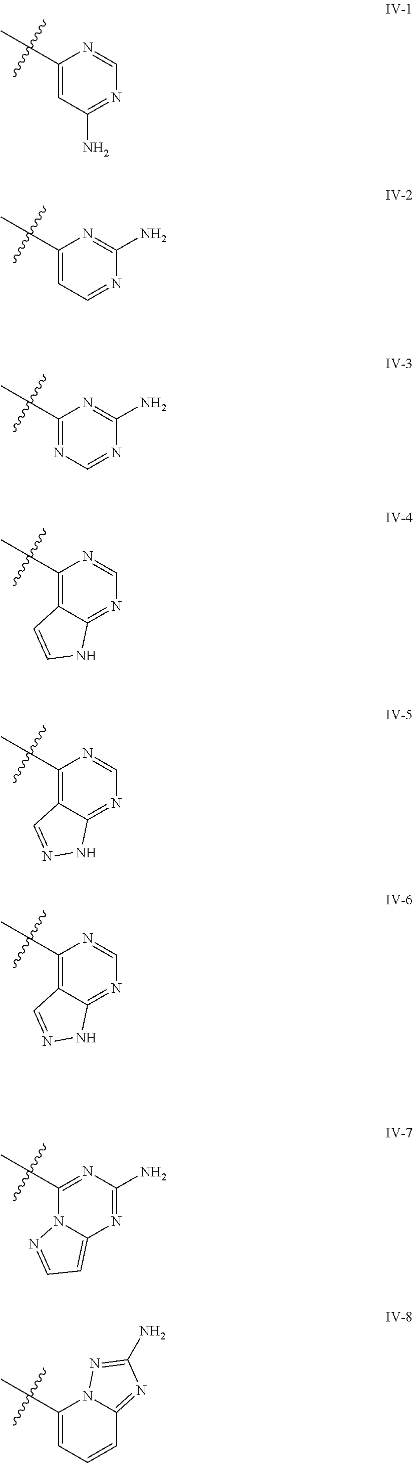

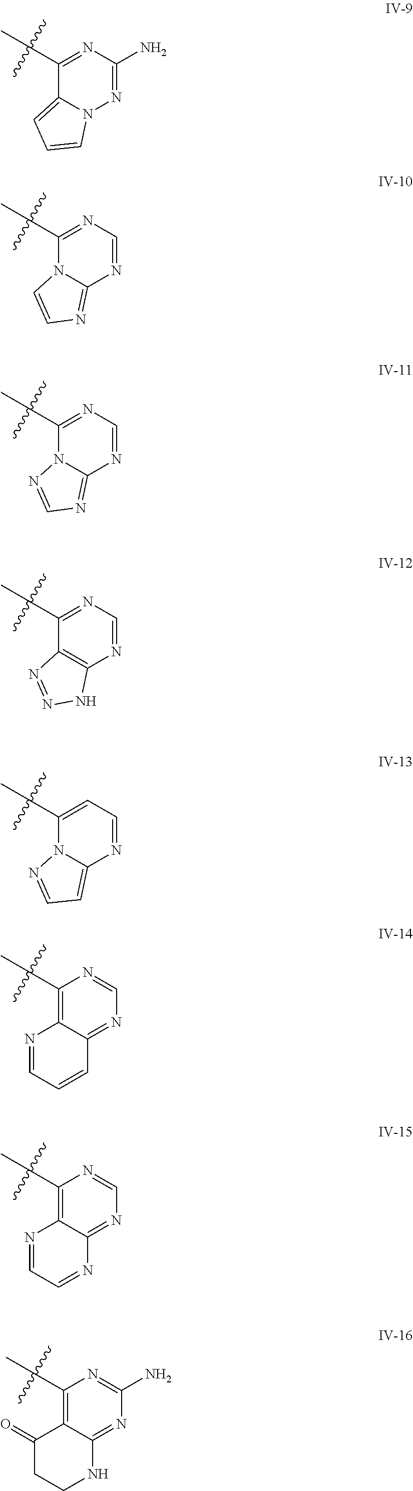

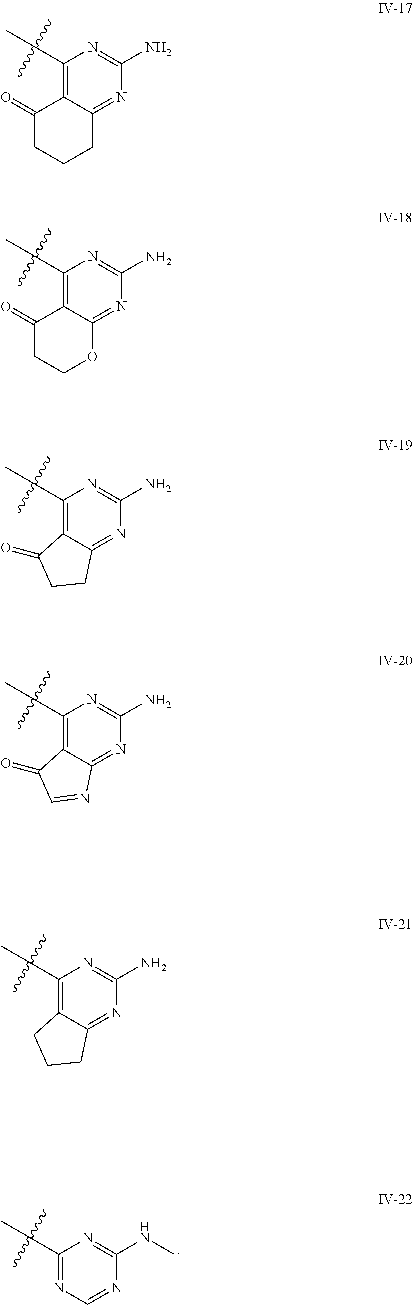

In some embodiments, W is selected from IV-1 to IV-22, which is optionally substituted with one or more groups selected from halo, —CN, —CF₃, —NO₂, —OR′, —NR′R—C(O)NR′R″, —NR′COR″, —C(O)R′, —C(═N—OR′)—R″, —S(O)ₚR′, —SR′, C₁₋₆ alkyl, C₂₋₆ alkenyl, C₂₋₆alkynyl, C₁₋₆alkoxy, 5-6 membered monocyclic heterocycle and 5-6 membered monocyclic heteroaryl; wherein alkyl, alkenyl, alkynyl, heterocycle and heteroaryl is optionally substituted with one or more groups selected from —OH, —CN, C₁₋₄alkoxy, C₁₋₄ alkyl, and —NR′R″;

R′ and R″ are each independently hydrogen, C₁₋₄ alkyl, C₃₋₆ cycloalkyl or 4-6 membered heterocycle; wherein alkyl is optionally substituted with one or more groups selected from —OH, halo and C₁₋₄alkoxy.

In some embodiments, W is IV-2, which is substituted with one or more groups selected from —CN, —NH₂, C₁-C₆ alkyl and —C(O)R′; R′ is C₁-C₆ alkyl optionally substituted with one or more halo, or R′ is C₃₋₆ cyclcoalkyl optionally substituted with one or more halo.

In some embodiments, W is IV-2, which is substituted with —C(O)R′; R′ is C₁-C₄ alkyl optionally substituted with one or more halo.

In some embodiments, W is IV-2, which is substituted with —C(O)CF₃.

In some embodiments, W is IV-2, which is substituted with —C(O)R′; R′ is C₁-C₄ alkyl.

In some embodiments, W is IV-4, which is substituted with one or more groups selected from —CN, halo and —C(O)R′.

In some embodiments, W is IV-4, which is substituted with —CN.

In some embodiments, W is selected from IV-1 to IV-22, which is optionally substituted with halo, —CN, —CF₃, —NH₂, —S(O)CH₃, —C(O)CH₃, —C(O)NH₂, —C(O)NHCH₃, —C(O)N(CH₃)₂, —NHCOCH₃, ethenyl, —CH≡CCH₂OH, morpholinyl, 1H-pyrazolyl, pyridyl, pyrimidyl, wherein pyridyl and pyrimidyl can be optionally substituted with methyl, halo, —NH₂ or methoxyl.

In some embodiments, m is 0, 1, or 2.

In some embodiments, Z═N.

In some embodiments, Z═CH.

In some embodiments, provided is at least one compound of formula I-1, I-2 or I-3, Z═CH. R² and R³ are each independently H, methyl and ethyl; and R⁵ is hydrogen.

In some embodiments, provided is at least one compound of formula I-1, wherein Z═N; R¹ is selected from 5-6 membered monocyclic aryl and heteroaryl, which are optionally substituted with one or more groups selected from halo and C₁₋₆ alkyl; R², R³, R⁴, R⁵, and W are as defined herein.

In some embodiments, provided is at least one compound of formula I-1, wherein Z═N; R¹ is phenyl or pyridyl, which are optionally substituted with one or more groups selected from halo and C₁₋₆ alkyl; R², R³, R⁴, R⁵, and W are as defined herein.

In some embodiments, provided is at least one compound of formula I-1, wherein Z═N; R³, R⁵ and the atoms they are attached to form an heterocyclic ring, which is optionally substituted

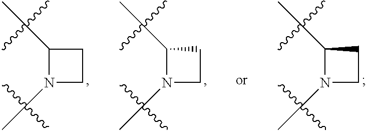 [SMILES: *C1CCN1* ; *[C@@H]1CCN1* ; *[C@H]1CCN1*]

R¹, R², R⁴, and W are as defined above.

In some embodiments, provided is at least one compound of formula I-1, wherein Z═N; R³, R⁵ and the atoms they are attached to form

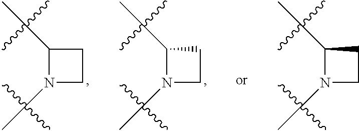 [SMILES: *C1CCN1* ; *[C@@H]1CCN1* ; *[C@H]1CCN1*]

which is optionally substituted with one or more groups selected from C₁₋₆ alkyl and C₁-C₄ alkoxy; R¹, R², R⁴, and W are as defined above.

In some embodiments, provided is at least one compound of formula I-1, wherein Z═N; R³, R⁵ and the atoms they are attached to form

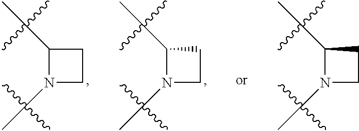 [SMILES: *C1CCN1* ; *[C@@H]1CCN1* ; *[C@H]1CCN1*]

which is optionally substituted with one or more groups selected from methyl and ethyl; R¹, R², R⁴, and W are as defined above.

In some embodiments, provided is at least one compound of formula I-1, wherein Z═N; R⁴ is selected from halo, —CN, C₁₋₆ alkyl, C₁-C₆ haloalkyl, and C₂-C₆ alkynyl; R¹, R², R³, R⁵, and W are as defined herein. In some embodiments, said C₁-C₆ haloalkyl is —CF₃.

In some embodiments, provided is at least one compound of formula I-1, wherein Z═N; R¹, R², R³, R⁴, and R⁵ are as defined herein; W is selected from the formula of IV-2, IV-3, IV-4, IV-6, and IV-16, each of which is optionally substituted with one or more groups selected from halo, —CN, —NR′R″, C₁₋₆ alkyl, and —C(O)R′, wherein R′ and R″ are each independently selected from hydrogen, C₁₋₆ alkyl, and C₁-C₆ haloalkyl.

In some embodiments, provided is at least one compound of formula I-1, wherein Z═N; R¹, R², R³, R⁴, and R⁵ are as defined herein; W is selected from the formula of IV-2, IV-3, IV-4, IV-6, and IV-16, each of which is optionally substituted with one or more groups selected from halo, —CN, —NH₂, —CH₃, —C(O)CH₃, and —C(O)CHF₂.

Also provided is at least one compound selected from compounds 1 to 521 and/or at least one its solvates, racemic mixture, enantiomers, diasteromers, tautomers, or mixtures of any ratio, or pharmaceutically acceptable salt thereof.

Also provided is a composition comprising at least one compound of formula I-1, I-2 or I-3, and/or at least one pharmaceutically acceptable salt described herein, and at least one pharmaceutically acceptable carrier.

Also provided is a method of inhibiting the activity of PI₃K kinase comprising contacting the kinase with an effective amount of at least one compound of formula I-1, I-2 or I-3 and/or its solvates, racemic mixture, enantiomers, diasteromers, tautomers, or mixtures of any ratio, or pharmaceutically acceptable salts thereof described herein to the subject in need thereof.

Also provided is a method of treating a disease responsive to inhibition of PI₃K comprising administering to a subject in need thereof a therapeutically effective amount of at least one compound of formula I-1, I-2 or I-3 and/or its solvates, racemic mixture, enantiomers, diasteromers, tautomers, or mixtures of any ratio, or pharmaceutically acceptable salts thereof described herein.

Also provided is at least one compound of formula I-1, I-2 or I-3 and/or its solvates, racemic mixture, enantiomers, diasteromers, tautomers, or mixtures of any ratio, or pharmaceutically acceptable salts thereof described herein for use in the treatment of diseases responsive to inhibition of PI₃K.

Also provided is a use of at least one compound of formula I-1, I-2 or I-3 and/or its solvates, racemic mixture, enantiomers, diasteromers, tautomers, or mixtures of any ratio, or pharmaceutically acceptable salts thereof described herein in the manufacture of a medicament for treating diseases responsive to inhibition of PI₃K.

In some embodiments, the disease responsive to inhibition of PI₃K described above is immune-based disease or cancer.

In some embodiments, the said immune-based disease is rheumatoid arthritis, COPD, multiple sclerosis, asthma, glomerulonephritis, lupus, or inflammation related to any of the aforementioned; the said cancer is lymphoma or acute myeloid leukemia, multiple myeloma and chronic lymphocytic leukemia.

In some embodiments, the said compound described herein can be administered in combination with another kinase inhibitor that inhibits a kinase activity other than a PI₃K kinase.

### DEFINITIONS

As used in the present specification, the following words, phrases and symbols are generally intended to have the meanings as set forth below, except to the extent that the context in which they are used indicates otherwise. The following abbreviations and terms have the indicated meanings throughout:

A dash (“-”) that is not between two letters or symbols is used to indicate a point of attachment for a substituent. For example, —CONH₂ is attached through the carbon atom.

The term “alkyl” herein refers to a C₁₋₁₀ straight or branched hydrocarbon. Preferably “alkyl” refers to a straight or branched hydrocarbon, containing 1-6 carbon atoms. More preferably “alkyl” refers to a straight or branched hydrocarbon, containing 1-4 carbon atoms. Examples of alkyl groups include, but are not limited to, methyl, ethyl, n-propyl, i-propyl, n-butyl, i-butyl, and t-butyl. “Hydroxylalkyl” refers to the alkyl which is substituted with OH. “Haloalkyl” refers to the alkyl which is substituted with halogen. “Alkoxylalkyl” refers to the alkyl which is substituted with alkoxy. “Aminoalkyl” refers to the alkyl which is substituted with NR^{a}R^{b}, R^{a} and R^{b} can be hydrogen, alkyl, alkenyl, alkynyl, cycloalkyl, heterocyclo, aryl, heteroaryl.

By “alkoxy” is meant a straight or branched alkyl group of the indicated number of carbon atoms attached through an oxygen bridge. Alkoxy groups will usually have from 1 to 10 carbon atoms attached through the oxygen bridge. Preferably “alkoxy” refers to a straight or branched alkoxy, wherein the alkyl portion contains 1-6 carbon atoms. More preferably “alkoxy” refers to a straight or branched alkoxy, wherein the alkyl portion contains 1-4 carbon atoms. Examples of alkyl groups include, but not limited to, methoxy, ethoxy, propoxy, i-propoxy, n-butoxy, s-butoxy, t-butoxy, pentoxy, 2-pentyloxy, i-pentoxy, neopentoxy, hexoxy, 2-hexoxy, 3-hexoxy, 3-methylpentoxy, and the like.

The term “alkenyl” herein refers to a C₂₋₁₀ straight or branched hydrocarbon, containing one or more C═C double bonds. Preferably “alkenyl” refers to a C₂₋₆ straight or branched hydrocarbon, containing one or more C═C double bonds. More preferably “alkenyl” refers to a C₂₋₄ straight or branched hydrocarbon, containing one or more C═C double bonds. Examples of alkenyl groups include, but are not limited to, vinyl, 1-propenyl, and 1-butenyl.

The term “alkynyl” herein refers to a C₂₋₁₀ straight or branched hydrocarbon, containing one or more C≡C triple bonds. Preferably “alkynyl” refers to a C₂₋₆ straight or branched hydrocarbon, containing one or more CC triple bonds. More preferably “alkynyl” refers to a C₂₋₄ straight or branched hydrocarbon, containing one or more C≡C triple bonds. Examples of alkynyl groups include, but are not limited to, ethynyl, 1-propynyl, and 1-butynyl.

The term “cycloalkyl” refers to a saturated and partially unsaturated monocyclic or bicyclic hydrocarbon group having 3 to 12 carbons. The ring may be saturated or have one or more double bonds (i.e. partially unsaturated), but not fully conjugated. Examples of bicycle cycloalkyl groups include, but are not limited to octahydropentalene, decahydronaphthalene, bicyclo[3.2.0]heptane, octahydro-1H-indene. Examples of single cycle cycloalkyl groups include, but are not limited to, cyclopropyl, cyclobutyl, cyclopentyl, cyclopentenyl, cyclohexyl, cyclohexenyl, cycloheptyl, and cyclooctyl.

Cycloalkyl also includes 3- to 12-membered monocyclic or bicyclic carbocyclic ring fused with a 5- or 6-membered aromatic ring, and the point of the attachment is on the cycloalkyl ring.

“Aryl” encompasses: 5- and 6-membered C₅₋₆ carbocyclic aromatic rings, for example, benzene; 8- to 12-membered bicyclic ring systems wherein at least one ring is carbocyclic and aromatic, for example, naphthalene; and 11- to 14-membered tricyclic ring systems wherein at least one ring is carbocyclic and aromatic, for example, fluorene.

For bi- or tricyclic rings, wherein one or two carbocyclic aromatic rings are fused with other rings (such as carbocyclic, heterocyclic or heterocyclic aromatic ring), the resulting ring system is aryl, provided that the point of attachment is at the carbocyclic aromatic ring.

For example, aryl includes 5- and 6-membered C₅₋₆ carbocyclic aromatic rings fused to a 5- to 7-membered non-aromatic carbocyclic or heterocyclic ring containing one or more heteroatoms selected from N, O, and S, or a 3- to 12-membered cycloalkyl, provided that the point of the attachment is on the carbocyclic aromatic rings.

Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below.

The term “halo” includes fluoro, chloro, bromo, and iodo, and the term “halogen” includes fluorine, chlorine, bromine, and iodine.

The term “heteroaryl” refers to
5- to 8-membered aromatic, monocyclic rings containing one or more, for example, from 1 to 4, or, in some embodiments, from 1 to 3, or, in some embodiments, from 1 to 2, heteroatoms selected from N, O, and S, with the remaining ring atoms being carbon; In some embodiments monocyclic “heteroaryl” refers to 5- to 6-member aromatic containing one or more heteroatoms selected from N, O, and S, with the remaining ring atoms being carbon;
8- to 12-membered bicyclic rings containing one or more, for example, from 1 to 6, or, in some embodiments, from 1 to 5, or, in some embodiments, from 1 to 4, or, in some other embodiments, from 1 to 3, heteroatoms selected from N, O, and S, with the remaining ring atoms being carbon and wherein at least one heteroatom is present in an aromatic ring; In some embodiments “heteroaryl” refer to 9- to 10-member bicyclic aromatic rings containing one or more heteroatoms selected from N, O, and S, with the remaining ring atoms being carbon and wherein at least one heteroatom is present in an aromatic ring; and
11- to 14-membered tricyclic rings containing one or more, for example, from 1 to 6, or in some embodiments, from 1 to 5, or, in some embodiments, from 1 to 4, or, in some embodiments, from 1 to 3, heteroatoms selected from N, O, and S, with the remaining ring atoms being carbon and wherein at least one heteroatom is present in an aromatic ring.

For bi- or tricyclic rings, wherein one or two heterocyclic aromatic rings are fused with other rings (such as carbocyclic, heterocyclic or carbocyclic aromatic ring), the resulting ring system is heteroaryl, provided that the point of attachment is at the heteroaromatic ring.

For example, heteroaryl includes 5- to 6-membered heterocyclic aromatic ring fused to a 5- to 7-membered heterocyclic ring containing one or more heteroatoms selected from N, O, and S, or a 5- to 7-membered cycloalkyl ring, provided that the point of the attachment is on the heterocyclic aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of heteroaryl groups include, but are not limited to, pyridyl, pyrazinyl, pyridazinyl, pyrimidinyl, triazinyl, pyrrolyl, pyrazolyl, imidazolinyl, isoxazolyl, oxazolyl, thiazolyl, thiadiazolyl, tetrazolyl, thienyl, benzothienyl, furyl, benzofuryl, benzoimidazolinyl, indazolyl, indolyl, triazolyl, quinolinyl, quinoxalinyl, pyrido[3,2-d]pyrimidinyl, quinazolinyl, naphthyridinyl, benzothiazolyl, benzoxazolyl, purinyl, pyrrolopyridinyl, pyrrolopyrimidinyl, imidazolopyridinyl, imidazolopyrimidinyl, imidazolotriazinyl, triazolopyridinyl, triazolopyrimidinyl and triazolotriazinyl.

Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl as defined above.

Substituted heteroaryl also includes ring systems substituted with one or more oxide substituents, such as pyridinyl N-oxides.

The terms “heterocycle” refers to 3- to 12-membered monocyclic, bicyclic and tricyclic rings containing one or more, for example, from 1 to 5, or, in some embodiments, from 1 to 4, heteroatoms selected from N, O, and S, with the remaining ring atoms being carbon; The rings may be saturated or partially unsaturated (i.e. have one or more double bonds), but not fully conjugated. In some embodiments “heterocycle” refers to 4-6 membered monocyclic rings containing one or more heteroatoms selected from N, O, and S, with the remaining ring atoms being carbon.

Heterocycle also includes 5- to 7-membered heterocyclic ring containing one or more heteroatoms selected from N, O, and S fused with a 5- or 6-membered carbocyclic aromatic ring or a 5- or 6-membered heterocyclic aromatic ring, and the point of the attachment is on the cycloalkyl ring. The point of the attachment may be on a carbon or heteroatom in the heterocyclic ring. The heterocycle can be substituted by oxo.

Heterocycle also refers to an aliphatic spirocyclic ring containing one or more heteroatoms selected from N, O, and S, provided that the point of attachment is at the heterocyclic ring.

Suitable heterocycles include, but not limited to, azetidinyl, pyrrolidinyl, imidazolidinyl, pyrazolidinyl, piperidinyl, morpholinyl, piperazinyl, tetrahydropyranyl, tetrahydrofuranyl, tetrahydrothiophenyl, tetrahydrothiopyranyl, oxazolidinyl, thiazolidinyl and thiomorpholinyl.

By “optional” or “optionally” is meant that the subsequently described event or circumstance may or may not occur, and that the description includes instances where the event or circumstance occurs and instances in which it does not. For example, “optionally substituted alkyl” encompasses both “unsubstituted alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

The term “substituted”, as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

Compounds described herein include, but are not limited to, their optical isomers, racemates, and other mixtures thereof. In those situations, the single enantiomers or diastereomers, i.e., optically active forms, can be obtained by asymmetric synthesis or by resolution of the racemates or mixtures of diastereomers. Resolution of the racemates or mixtures of diastereomers can be accomplished, for example, by conventional methods such as crystallization in the presence of a resolving agent, or chromatography, using, for example a chiral high-pressure liquid chromatography (HPLC) column. In addition, such compounds include R- and S-forms of compounds with chiral centers. Such compounds also include crystal forms including polymorphs and clathrates. Similarly, the term “salt” is intended to include all isomers, racemates, other mixtures, R- and S-forms, tautomeric forms and crystal forms of the salt of the compound.

The invention includes also pharmaceutically acceptable salts of the compounds represented by Formula I-1, I-2 or I-3, preferably of those described below and of the specific compounds exemplified herein, and methods using such salts.

A “pharmaceutically acceptable salt” is intended to mean a salt of a free acid or base of a compound represented by Formula I-1, I-2 or I-3 that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002.

Preferred pharmaceutically acceptable salts are those that are pharmacologically effective and suitable for contact with the tissues of patients without undue toxicity, irritation, or allergic response. A compound of Formula I-1, I-2 or I-3 may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methyl benzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula I-1, I-2 or I-3 contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula I-1, I-2 or I-3 is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

A “solvate,” such as a “hydrate,” is formed by the interaction of a solvent and a compound. The term “compound” is intended to include solvates, including hydrates, of compounds. Similarly, “salts” includes solvates, such as hydrates, of salts. Suitable solvates are pharmaceutically acceptable solvates, such as hydrates, including monohydrates and hemi-hydrates.

As used herein the terms “group”, “radical” or “fragment” are synonymous and are intended to indicate functional groups or fragments of molecules attachable to a bond or other fragments of molecules.

The term “active agent” is used to indicate a chemical substance which has biological activity. In some embodiments, an “active agent” is a chemical substance having pharmaceutical utility.

The terms “treating” or “treatment” or “alleviation” refers to administering at least on compounds/or at least one pharmaceutically acceptable salt described herein to a subject to slow down (lessen) an undesired physiological change or disorder, such as the development or spread of inflammation or cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of disease state, and remission (whether partial or total), whether detectable or undetectable. “Treatment” can also mean prolonging survival as compared to expected survival if not receiving treatment. Those in need of treatment include those with the condition or disorder.)

The term “effective amount” means an amount or dose of a PI₃K-inhibiting agent sufficient to generally bring about a therapeutic benefit in patients in need of treatment for a disease, disorder, or condition mediated by PI₃K activity. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.0001 to about 200 mg of active agent per kg of subject's body weight per day, preferably about 0.001 to 100 mg/kg/day, or about 0.01 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 5 g/day. Once improvement of the patient's disease, disorder, or condition has occurred, the dose may be adjusted for maintenance treatment. For example, the dosage or the frequency of administration, or both, may be reduced as a function of the symptoms, to a level at which the desired therapeutic effect is maintained. Of course, if symptoms have been alleviated to an appropriate level, treatment may cease. Patients may, however, require intermittent treatment on a long-term basis upon any recurrence of symptoms.

The term “inhibition” indicates a decrease in the baseline activity of a biological activity or process. “Inhibition of PI₃K activity” refers to a decrease in the activity of PI₃K as a direct or indirect response to the presence of at least one at least one compound and/or at least one pharmaceutically acceptable salt described herein, relative to the activity of PI₃K in the absence of the at least one compound and/or the at least one pharmaceutically acceptable salt thereof. The decrease in activity may be due to the direct interaction of the at least one compound and/or at least one pharmaceutically acceptable salt described herein with PI₃K, or due to the interaction of the at least one compound and/or at least one pharmaceutically acceptable salt described herein, with one or more other factors that in turn affect PI₃K activity. For example, the presence of at least one compound and/or at least one pharmaceutically acceptable salt described herein, may decrease PI₃K activity by directly binding to the PI₃K, by causing (directly or indirectly) another factor to decrease PI₃K activity, or by (directly or indirectly) decreasing the amount of PI₃K present in the cell or organism.

In addition, the active agents of the invention may be used in combination with additional active ingredients in the treatment of the above conditions. The additional active ingredients may be coadministered separately with an active agent of Formula I-1, I-2 or I-3 or included with such an agent in a pharmaceutical composition according to the invention. In an exemplary embodiment, additional active ingredients are those that are known or discovered to be effective in the treatment of conditions, disorders, or diseases mediated by PI₃K activity, such as another PI₃K modulator or a compound active against another target associated with the particular condition, disorder, or disease. The combination may serve to increase efficacy (e.g., by including in the combination a compound potentiating the potency or effectiveness of an active agent according to the invention), decrease one or more side effects, or decrease the required dose of the active agent according to the invention.

The active agents of the invention are used, alone or in combination with one or more additional active ingredients, to formulate pharmaceutical compositions of the invention. A pharmaceutical composition of the invention comprises: (a) an effective amount of at least one active agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 5 mg to 5 g daily, or from about 50 mg to 5 g daily, in single or divided doses. For example, a total daily dosage of about 5 mg to 5 g daily may be accomplished by dosing once, twice, three, or four times per day.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

The compounds described herein, and/or the pharmaceutically acceptable salts thereof, can be synthesized from commercially available starting materials by methods well known in the art. The following schemes illustrate methods for most of compound preparation. In each of the schemes, R¹, R², R³, R⁴, R⁵ and W are as defined herein.

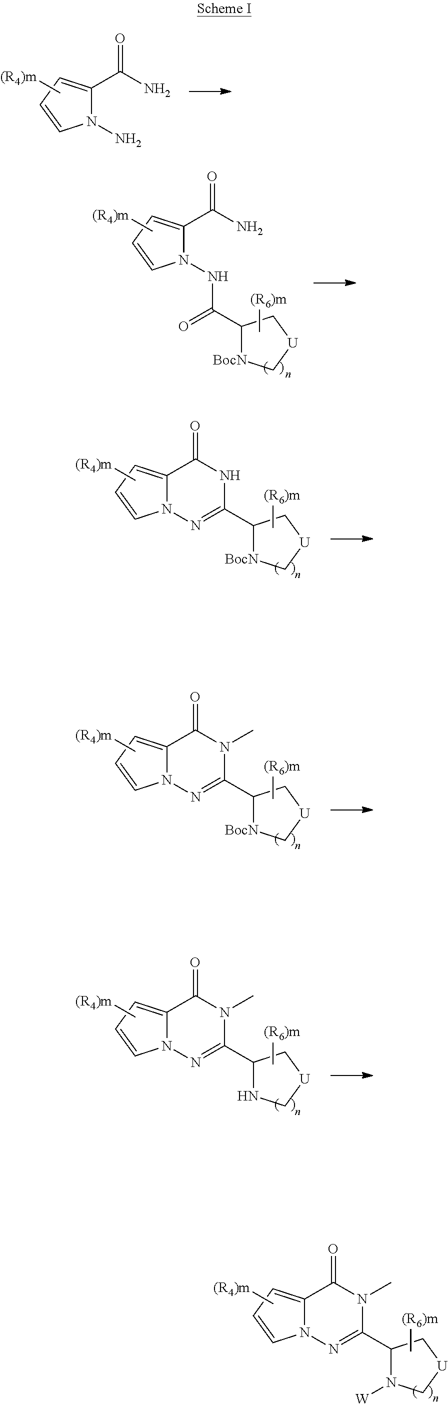

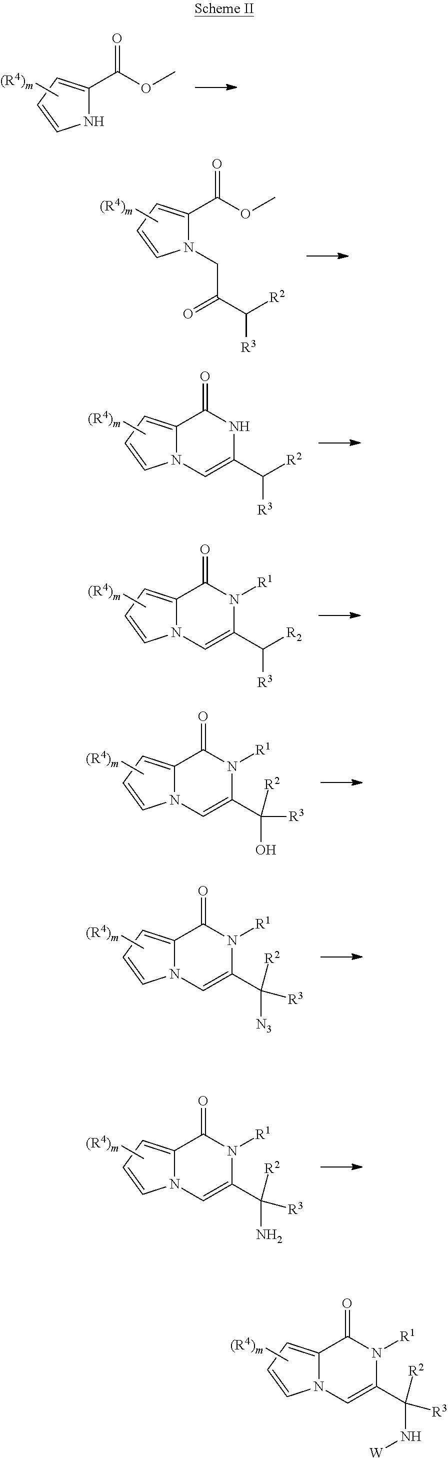

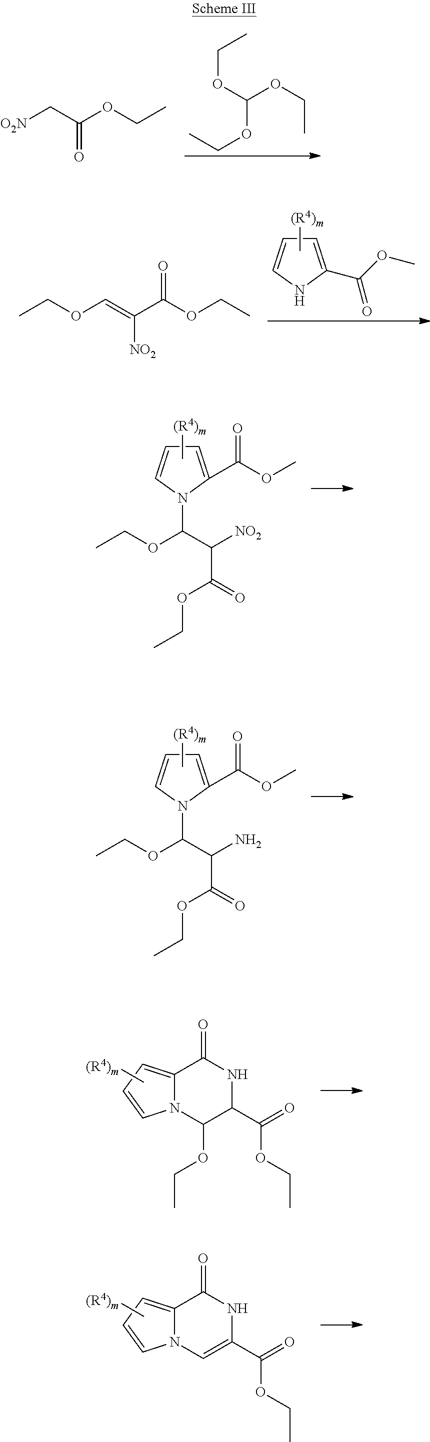

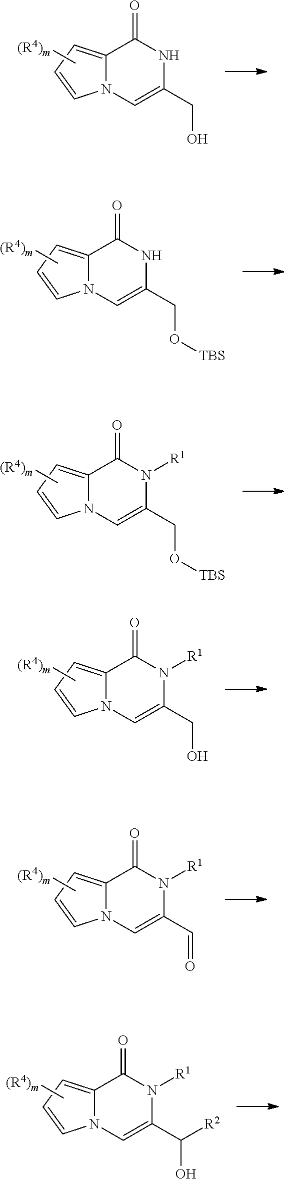

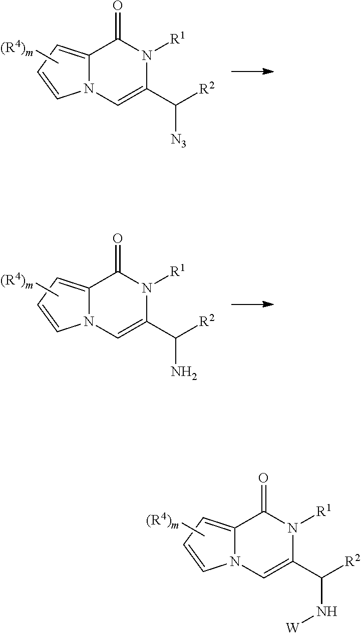

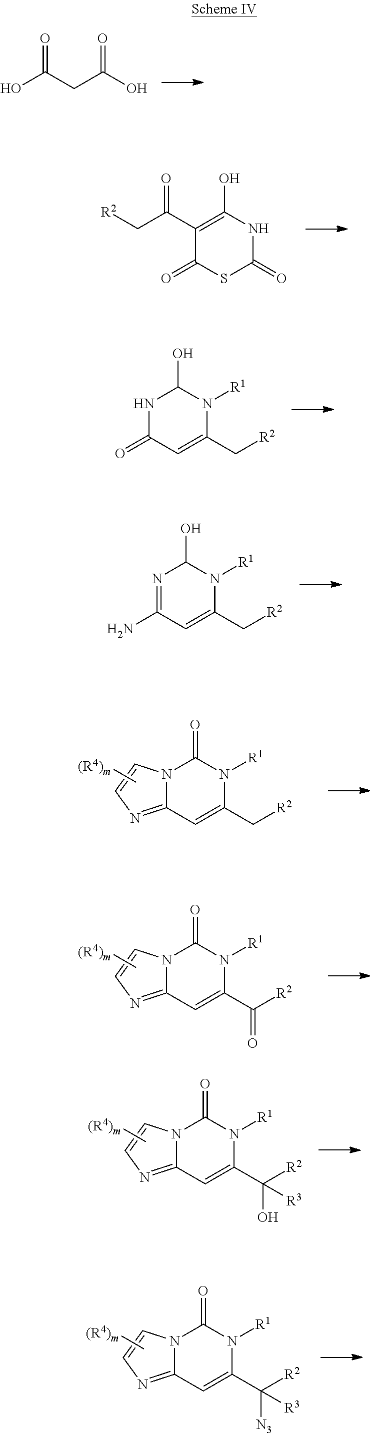

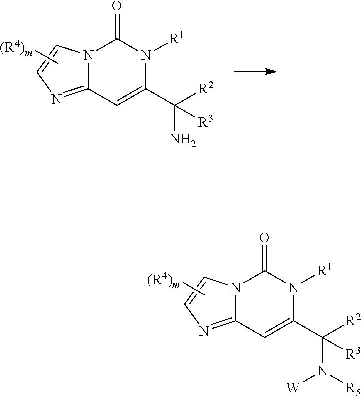

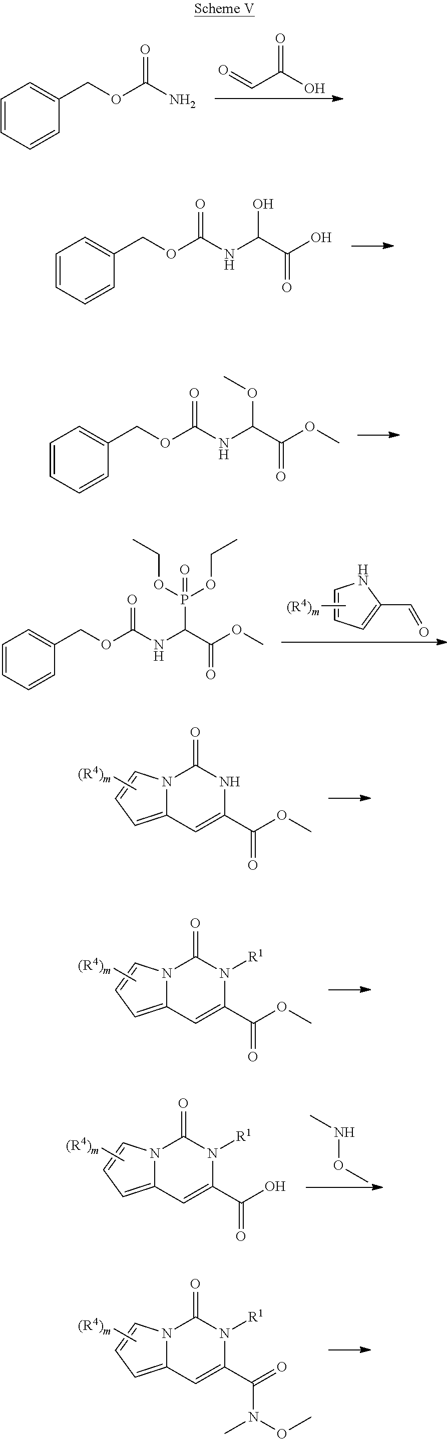

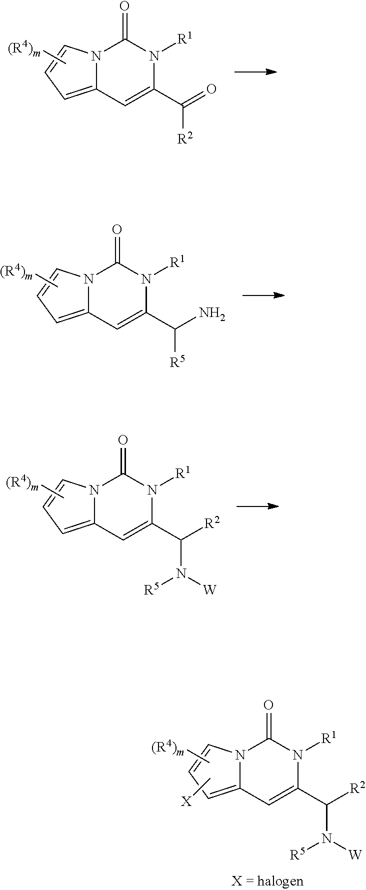

The compounds thus obtained can be further modified at their peripheral positions to provide the desired compounds. Synthetic chemistry transformations are described, for example, in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

### EXAMPLES

The examples below are intended to be purely exemplary and should not be considered to be limiting in any way. Efforts have been made to ensure accuracy with respect to numbers used (for example, amounts, temperature, etc.) but some experimental errors and deviations should be accounted for. Unless indicated otherwise, parts are parts by weight, temperature is in degrees Centigrade, and pressure is at or near atmospheric. All MS data were checked by agilent 6120 or agilent 1100. All NMR data were generated using a Varian 400-MR machine. All reagents, except intermediates, used in this invention are commercially available. All compound names except the reagents were generated by Chemdraw 10.0.

In the following examples, the abbreviations below are used:
4AMS 4A Molecular sieves
aq. aqueous solution
ADP Adenosine diphosphate
ATP Adenosine triphospahte
n-BuOH n-butanol
BOP benzotriazol-1-yloxytris(dimethylamino)-phosphonium hexafluorophosphate
CHAPS 3-[(3-Cholamidopropyl)dimethylammonio]propanesulfonate
conc. concentrated
DAST diethylaminosulfur trifluoride
dba dibenzylideneacetone
DBU 1,8-diazabicyclo[5.4.0]undec-7-ene
DCM dichloromethane
DHP 3,4-dihydro-2H-pyran
DIEA N,N-diisopropylethylamine
DIBAL-H Diisobutylaluminum hydride
DMA N,N-dimethylacetamide
DMF N,N-dimethylformamide
DPPA diphenylphosphoryl azide
dppf 1,1′-bis(diphenylphosphino)ferrocene
DTT DL-Dithiothreitol
Eaton's reagent 7.7 wt % phosphorus pentoxide solution in methanesulfonic acid
EDC 1-(3-dimethylaminopropyl)-3-ethylcarbodiimide hydrochloride
EGTA Glycol-bis-(2-aminoethylether)-N,N,N′,N′-tetraacetic acid
EtOAc ethyl acetate
g gram(s)
h hour(s)
HATU 2-(1H-7-azabenzotriazol-1-yl)-1,1,3,3-tetramethyl uronium hexafluorophosphate methanaminium
HBTU 2-(1H-Benzotriazole-1-yl)-1,1,3,3-Tetramethyluronium hexafluorophosphate
HEPES 4-(2-Hydroxyethyl)-1-piperazineethanesulfonic acid
m-CPBA 3-chloroperoxybenzoic acid
MeOH methanol
mg milligram(s)
min minute(s)
mL milliliter(s)
NC S N-chlorosuccinimide
PE petroleum ether
PyBrOP Bromo-tris-pyrrolidinophosphoniumhexafluorophosphate
PCC Pyridinium Chlorochromate
r.t. room temperature
Selectfluor 1-chloromethyl-4-fluoro-1,4-diazoniabicyclo[2.2.2]octane bis(tetrafluoroborate)
SEM 2-(trimethylsilyl)ethoxymethyl
TBAF tetrabutylammonium fluoride
TBSCl t-butylchlorodimethylsilane
TEA triethylamine
TFA trifluoroacetic acid
THF tetrahydrofuran
THP tetrahydropyran
TLC thin-layer chromatography
TMS trimethylsilyl
TsOH p-toluenesulfonic acid
TsCl p-toluenesulfonic chloride
Xantphos 4,5-Bis(diphenylphosphino)-9,9-dimethyxanthene

### Intermediate 1

### Methyl 3-chloro-1H-pyrrole-2-carboxylate

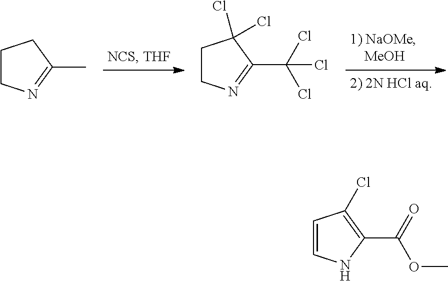

At 55-60° C. with vigorous stirring to a mixture of NCS (107 g, 800 mmol) in THF (250 mL) in a 2 L flask was added 5-methyl-3,4-dihydro-2H-pyrrole (83 g, 1000 mmol) in one-portion. After addition, the reaction spontaneously heated to reflux for about 5 min, then reacted at 60-70° C. for another 1.5 hours. After cooled to r.t., hexane (300 mL) and water (300 mL) were added to the mixture. The organic layer was separated, collected and concentrated. The residue was used in the next step without further purification. To a mixture of the crude 4,4-dichloro-5-(trichloromethyl)-3,4-dihydro-2H-pyrrole (240 g, 941 mmol) in MeOH (2 L) in an ice-bath was added a solution of NaOMe (324 g, 6 mol) in MeOH (1.5 L) drop-wise over an hour. After addition, the mixture was stirred at r.t. for another one hour. Then 2N HCl aq. was added to adjust its pH to 2 and the resulting was stirred at room temperature for 15 minutes. The mixture was concentrated and diluted with EtOAc (2.5 L) and water (2 L). The organic layer was separated, concentrated and purified by column chromatography eluting with EtOAc/PE and then crystallize upon standing. Methyl 3-chloro-1H-pyrrole-2-carboxylate was obtained as an orange solid (91.3 g, yield: 61%). MS (m/z): 160.1 (M+H)⁺. ¹H NMR (400 MHz, DMSO-d₆) δ 12.05 (s, 1H), 6.98 (m, 1H), 6.21 (t, J=2.6 Hz, 1H), 3.75 (s, 3H).

### Intermediate 2

### Ethyl 3-bromo-1H-pyrrole-2-carboxylate

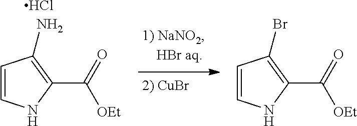 [SMILES: Cl ; CCOC(=O)c1[nH]ccc1N ; CCOC(=O)c1[nH]ccc1Br]

To a solution of ethyl 3-amino-1H-pyrrole-2-carboxylate hydrochloride (953 mg, 5.0 mmol) in 48% HBr aq. (3 mL, 26.0 mmol) and water (20 mL) was added NaNO₂ (966 mg, 14.0 mmol) in water (3 mL) at −5° C. The resulting mixture was then stirred at −5° C. for another 30 minutes. CuBr (2.01 g, 14.0 mmol, fine powder) was added portion-wise at this temperature, and the mixture was stirred at r.t. for 30 minutes and refluxed for 2 hours. The reaction mixture was then extracted with EtOAc. The organic layer was separated, concentrated and purified by flash column chromatography, eluting with EtOAc/PE to afford ethyl 3-bromo-1H-pyrrole-2-carboxylate as a yellow solid (562 mg, yield: 52%). MS (m/z): 218.0, 220.0 (M+H)⁺. ¹H NMR (400 MHz, DMSO-d₆) δ 9.22 (s, 1H), 6.86 (t, J=2.8 Hz, 1H), 6.34 (t, J=2.8 Hz, 1H), 4.36 (q, J=7.1 Hz, 2H), 1.39 (t, J=7.1 Hz, 3H).

### Intermediate 3

### 1-Amino-3-chloro-1H-pyrrole-2-carboxamide

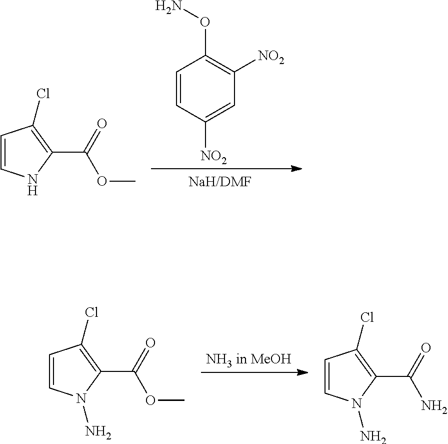

To a mixture of 60% NaH (12 g, 0.3 mol) in DMF (100 mL) at 0° C. was added methyl 3-chloro-1H-pyrrole-2-carboxylate (32 g, 0.2 mol) in DMF (100 mL) dropwise over one hour. After stirred at 0° C. for another 2.5 hours, to the light brown mixture was added a solution of O-(2,4-dinitrophenyl)hydroxylamine (48 g, 0.24 mol) in DMF (100 mL) slowly over 30 minutes. The reaction was stirred at 0° C. for 2.5 hours and warmed to room temperature overnight. The mixture was quenched by Na₂S₂O₃ aq. and extracted with EtOAc and washed with 10% LiCl aq. The organic layer was separated, concentrated and purified by flash column chromatography eluting with MeOH/water to give methyl 1-amino-3-chloro-1H-pyrrole-2-carboxylate as a yellow solid (30 g, yield: 86%). MS (m/z): 174.9 (M+H)⁺.

A mixture of methyl 1-amino-3-chloro-1H-pyrrole-2-carboxylate (30 g, 0.172 mol) in 7N NH₃/MeOH (300 mL) was allowed to heat to 130° C. in a sealed tube overnight. After concentrated, the residue was purified by flash column chromatography over silica gel eluting with EtOAc/PE to give 1-amino-3-chloro-1H-pyrrole-2-carboxamide as a white solid (16 g, yield: 58%). MS (m/z): 160.1 (M+H)⁺.

### Intermediate 4

### 1-amino-3-bromo-1H-pyrrole-2-carboxamide

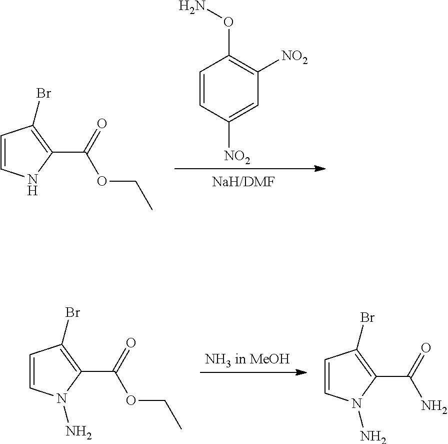

To a solution of 60% NaH (2.88 g, 72 mmol) in dry DMF (90 mL) was drop-wise added a solution of ethyl 3-bromo-1H-pyrrole-2-carboxylate (13.08 g, 60 mmol) in dry DMF (30 mL) at 0-5° C. over 30 min, then the reaction was stirred at 0-5° C. for 30 min. Subsequently, O-(2,4-dinitrophenyl)hydroxylamine (14.34 g, 72 mmol) in dry DMF (30 mL) was added drop-wise and the reaction was stirred at r.t. for another 16 hours. The mixture was poured into water and extracted with EtOAc. The combined layers were washed with brine, concentrated and purified by flash column chromatography eluting with PE/EA to afford ethyl 1-amino-3-bromo-1H-pyrrole-2-carboxylate as a yellow oil (12.5 g, yield: 89%). MS (m/z): 233.0, 235.0 (M+H)⁺.

A mixture of ethyl 1-amino-3-bromo-1H-pyrrole-2-carboxylate (12.5 g, 53.6 mol) in 7N NH₃/MeOH (80 mL) was heat at 130° C. overnight in a sealed tube. After concentration, the residue was purified by flash column chromatography eluting with MeOH/H₂O, and further purified by flash column chromatography over silica gel eluting with EtOAc/PE to give 1-amino-3-bromo-1H-pyrrole-2-carboxamide as a yellow solid (6.0 g, yield: 55%). MS (m/z): 203.9, 205.9 (M+H)⁺. ¹H NMR (400 MHz, DMSO-d₆) δ 7.71 (s, 1H), 7.47 (s, 1H), 6.89 (d, J=2.9 Hz, 1H), 6.47 (s, 2H), 6.09 (d, J=2.9 Hz, 1H).

### Intermediate 5

### 1-amino-3-cyclopropyl-1H-pyrrole-2-Carboxamide

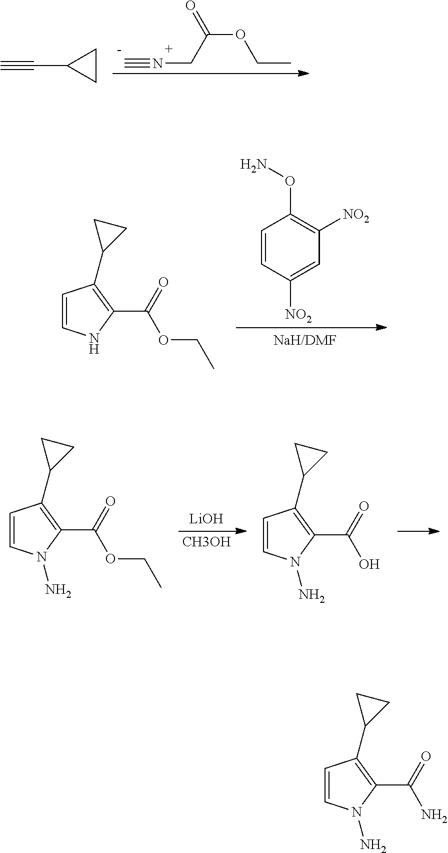

To a solution of CuBr (7.25 g, 50 mmol) and Cs₂CO₃ (16.25 g, 50 mmol) in DMF (150 mL) was added cyclopropylacetylene (3.3 g, 50 mmol) at r.t. under N₂. The reaction was stirred at 120° C. for 15 min, then ethyl isocyanoacetate (11.4 g, 100 mmol) in DMF (20 mL) was added drop-wise and the reaction was stirred at 120° C. for 2 h. The mixture was concentrated and purified by flash column chromatography to give ethyl 3-cyclopropyl-1H-pyrrole-2-carboxylate as a white solid (4.0 g, yield: 49.9%). MS (m/z): 180.1 (M+H)⁺.

To a mixture of NaH (210 mg, 60%, 5.25 mmol) in DMF (10 mL) was added ethyl 3-cyclopropyl-1H-pyrrole-2-carboxylate (626 mg, 3.5 mol) in DMF (8 mL) dropwise at 0° C., the reaction was stirred at 0° C. for 1 h, then O-(2,4-dinitrophenyl)hydroxylamine (836 mg, 4.2 mmol) in DMF (5 mL) was added dropwise, the reaction was continued at 0° C. for 2 h. The mixture was poured into water and extracted with EtOAc. The organic layers were washed with brine, dried over Na₂SO₄, concentrated and purified by flash column chromatography to give ethyl 1-amino-3-cyclopropyl-1H-pyrrole-2-carboxylate as a yellow solid (679 mg). MS (m/z): 195.1 (M+H)⁺.

Ethyl 1-amino-3-cyclopropyl-1H-pyrrole-2-carboxylate (679 mg, 3.5 mmol) was dissolved in MeOH (5 mL), 5 mL of aq. LiOH solution (1 N) was added. The reaction was stirred at reflux for 1 h. The mixture was concentrated, the resulting aqueous mixture was adjusted to pH˜7.0 using 1 N HCl, then extracted with EtOAc, the organic layer was dried over Na₂SO₄, concentrated to give the crude product 1-amino-3-cyclopropyl-1H-pyrrole-2-carboxylic acid (581 mg) which was used in the next step without further purification.

The mixture of 1-amino-3-cyclopropyl-1H-pyrrole-2-carboxylic acid (581 mg, about 3.5 mmol), NH₄Cl (1855 mg, 35 mmol), HATU (1330 mg, 3.5 mmol) and DIPEA (2 mL, 11.5 mmol) in DMF (4 mL) was stirred at r.t. overnight. The reaction mixture was poured into water, extracted with EtOAc, dried over Na₂SO₄, concentrated and purified by flash column chromatography to give the title product (166 mg, yield: 28%) as a white solid. MS (m/z): 166.1 (M+H)⁺.

### Intermediate 6 and 7

### 1-amino-3-(methoxymethyl)-1H-pyrrole-2-carboxamide and 2-ethyl 3-methyl 1-amino-1H-pyrrole-2,3-dicarboxylate

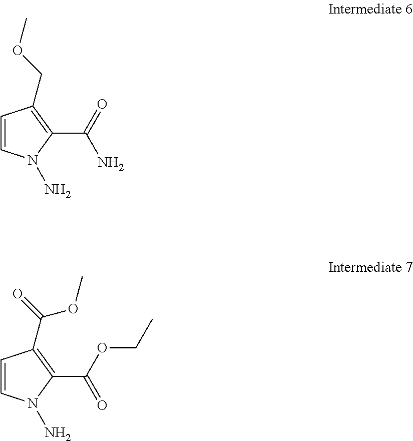

These intermediates were prepared according to the procedure of Intermediate 5 using the corresponding reagents and intermediates under appropriate conditions that will be recognized by one skilled in the art.

### Intermediate 8

### 4-chloro-3-(methylthio)-1H-pyrazolo[3,4-d]pyrimidine

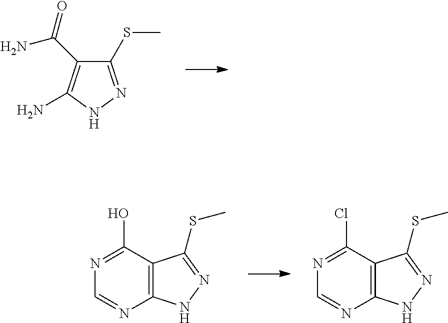

The mixture of 5-amino-3-(methylthio)-1H-pyrazole-4-carboxamide (516 mg, 3 mmol) and formamide (1 mL) was stirred at 180° C. for 1 h. The reaction was cooled to r.t., and added water. The precipitate was collected and recrystallized from MeOH to give 3-(methylthio)-1H-pyrazolo[3,4-d]pyrimidin-4-ol as a white solid. Yield: 99%. MS (m/z): 182.9 (M+1)⁺.

The mixture of 3-(methylthio)-1H-pyrazolo[3,4-d]pyrimidin-4-ol (540 mg, 3 mmol) and POCl₃ (3 mL) was stirred at reflux for 4 h. The reaction was concentrated, and added ice-cold water, the resulting precipitate was filtered and washed with water to give the desired product as a yellow solid, which was used for the next step without further purification. MS (m/z): 200.8 (M+1)⁺.

### Intermediate 9

### 2-amino-4-chloro-7,8-dihydropyrido[2,3-d]pyrimidin-5(6H)-one

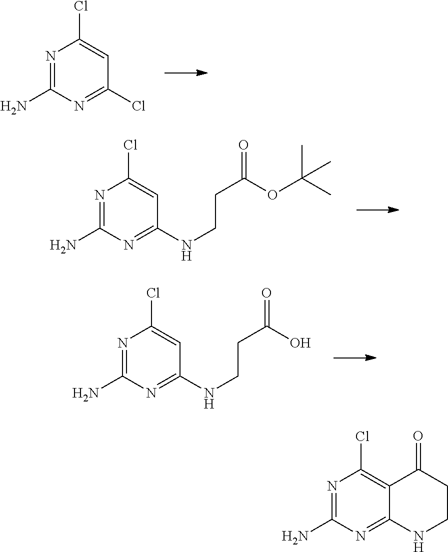

To a solution of 4,6-dichloropyrimidin-2-amine (5.4 g, 33 mmol) and tert-butyl 3-aminopropanoate hydrochloride (6.0 g, 33 mmol) in DMF (3 mL) was added Et₃N (5 mL). The reaction was stirred at 60° C. overnight. The mixture was poured into water, extracted with EtOAc, the organic layers were washed with brine, dried over Na₂SO₄, and concentrated to give tert-butyl 3-((2-amino-6-chloropyrimidin-4-yl)amino) propanoate as a white solid, which was used for the next step without further purification. MS (m/z): 273.0 (M+1)⁺.

The mixture of tert-butyl 3-((2-amino-6-chloropyrimidin-4-yl)amino)propanoate (6.0 g, 22 mmol) and TFA (20 mL) was stirred at r.t. for 1 h, then concentrated, and adjusted to pH=3-4 with 1N NaOH solution. The precipitate was filtered and washed with water to give 3-((2-amino-6-chloropyrimidin-4-yl)amino)propanoic acid as a white solid, which was used for the next step without further purification. Yield: 61%. MS (m/z): 217.0 (M+1)⁺.

The mixture of 3-((2-amino-6-chloropyrimidin-4-yl)amino)propanoic acid (2.9 g, 13.4 mmol) and Eaton's reagent (30 mL) was stirred at 75° C. for 3 h. The reaction mixture was poured into iced NH₄OH, extracted with EtOAc, the organic layers were washed with brine, dried over Na₂SO₄, concentrated to give the desired title compound as a yellow solid, which was used for the next step without further purification. MS (m/z): 199.0 (M+1)⁺.

### Intermediate 10

### (2S)-3-methyl-1-picolinoylazetidine-2-carboxylic acid

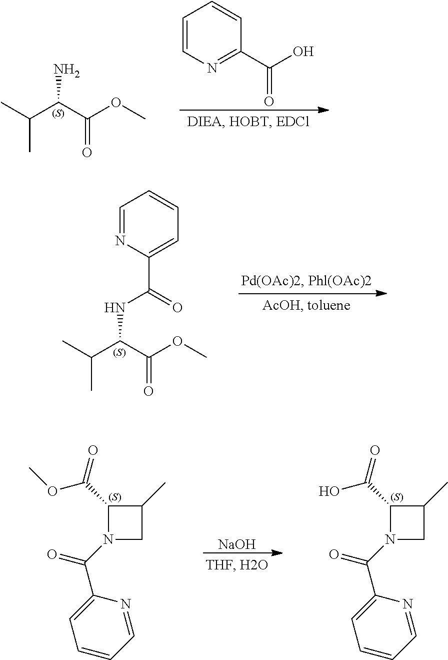

To a solution of (S)-methyl 2-amino-3-methylbutanoate (6.0 g, 35.9 mmol) in DCM (150 mL) were added HOBT (5.34 g, 39.5 mmol), EDCI.HCl (7.55 g, 39.5 mmol) and picolinic acid (4.86 g, 39.5 mmol) followed with DIEA (14 g, 108 mmol). The reaction was stirred at r.t. overnight. The mixture was concentrated and purified by flash chromatography to afford (S)-methyl 3-methyl-2-(picolinamido)butanoate as a colorless oil. Yield: 52.3%. MS (m/z): 237.0 (M+1)⁺.

To a solution of (S)-methyl 3-methyl-2-(picolinamido)butanoate (1.5 g, 6.36 mmol) in toluene (15 mL) were added Pd(OAc)₂ (36 mg, 0.16 mmol), PhI(OAc)₂ (5.12 g, 15.9 mmol) and AcOH (71163 mg, 12.72 mmol) under N₂, the mixture was bubbled with N₂ for 5 min. The reaction was stirred at 110° C. for 24 h in a sealed tube. After cooling to the r.t., the reaction was concentrated and purified by flash chromatography to afford (2S)-methyl 3-methyl-1-picolinoylazetidine-2-carboxylate as a yellow oil. Yield: 57%. MS (m/z): 234.9 (M+1)⁺.

To a solution of (2S)-methyl 3-methyl-1-picolinoylazetidine-2-carboxylate (1.3 g, 5.56 mmol) in THF (7 mL) was added a solution of NaOH (267 mg, 6.67 mmol) in H₂O (7 mL) at r.t. The reaction was stirred at r.t for 2 h, then adjusted to pH=6 with aq. HCl solution (1N). The mixture was concentrated and purified by flash chromatography to afford the title compound as a white solid. MS (m/z): 221.1 (M+1)⁺.

### Intermediate 11

### 1-(4-chloro-2-(methylthio)pyrimidin-5-yl)propan-1-one

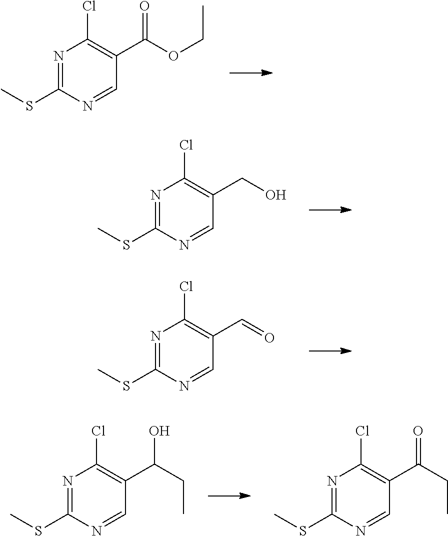

To a solution of ethyl 4-chloro-2-(methylthio)pyrimidine-5-carboxylate (2.32 g, 10 mmol) in THF (60 mL) was added DIBAL-H (1N in hexane, 30 mL) dropwise at 0° C., the reaction was stirred at 0° C. for 30 min, then H₂O was added followed by 2N HCl solution (45 mL). The mixture was extracted with EtOAc, the organic layers were washed with brine, dried over Na₂SO₄, concentrated to give (4-chloro-2-(methylthio)pyrimidin-5-yl)methanol as a yellow solid, which was used for the next step without further purification. Yield: 60%, MS (m/z): 190.9 (M+1)⁺.

To a solution of (4-chloro-2-(methylthio)pyrimidin-5-yl)methanol (1.14 g, 6 mmol) in DCM (200 mL) was added MnO₂ (8.7 g, 100 mmol), the reaction was stirred at r.t. overnight, then filtered, the filtrate was concentrated to give 4-chloro-2-(methylthio)pyrimidine-5-carbaldehyde as a yellow solid, which was used for the next step without purification. Yield: 72.7%, MS (m/z): 188.9 (M+1)⁺.

To a solution of 4-chloro-2-(methylthio)pyrimidine-5-carbaldehyde (376 mg, 2 mmol) in THF (5 mL) was added EtMgBr (3.0 M in hexane, 0.7 mL) dropwise at −78° C. The reaction was stirred at −78° C. for 30 min, then 1N HCl (2 mL) was added. The mixture was extracted with EtOAc, the organic layers were washed with brine, dried over Na₂SO₄, and concentrated to give 1-(4-chloro-2-(methylthio)pyrimidin-5-yl)propan-1-ol as a colorless oil, which was used for the next step without purification. MS (m/z): 219.0 (M+1)⁺.

To a solution of 1-(4-chloro-2-(methylthio)pyrimidin-5-yl)propan-1-ol (436 mg, 2 mmol) in DCM (10 mL) was added PCC (537 mg, 2.5 mmol), the mixture was stirred at r.t. under N₂ for 2 h, then filtered, the filtrate was concentrated to give 1-(4-chloro-2-(methylthio)pyrimidin-5-yl)propan-1-one as a yellow oil, which was used for next step without purification. MS (m/z): 217.0 (M+1)⁺.

### Intermediates 12 and 13

### 1-(4-chloro-2-(methylthio)pyrimidin-5-yl)-2,2,2-trifluoroethanone and (4-chloro-2-(methylthio)pyrimidin-5-yl)(cyclopropyl)methanone

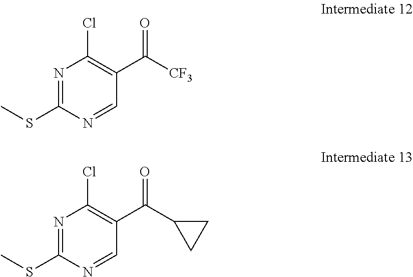

Intermediate 12 and Intermediate 13 were prepared according to the procedures described in Intermediate 11 using the corresponding reagents and intermediates.

Intermediate 12: MS (m/z): 256.8 (M+1)⁺.

Intermediate 13: MS (m/z): 229.0 (M+1)⁺.

### Example 1

### Compound 1

### (S)-4-(2-(4-oxo-3-phenyl-3,4-dihydropyrrolo[2,1-f][1,2,4]triazin-2-yl)pyrrolidin-1-yl)-7H-pyrrolo[2,3-d]pyrimidine-5-carbonitrile

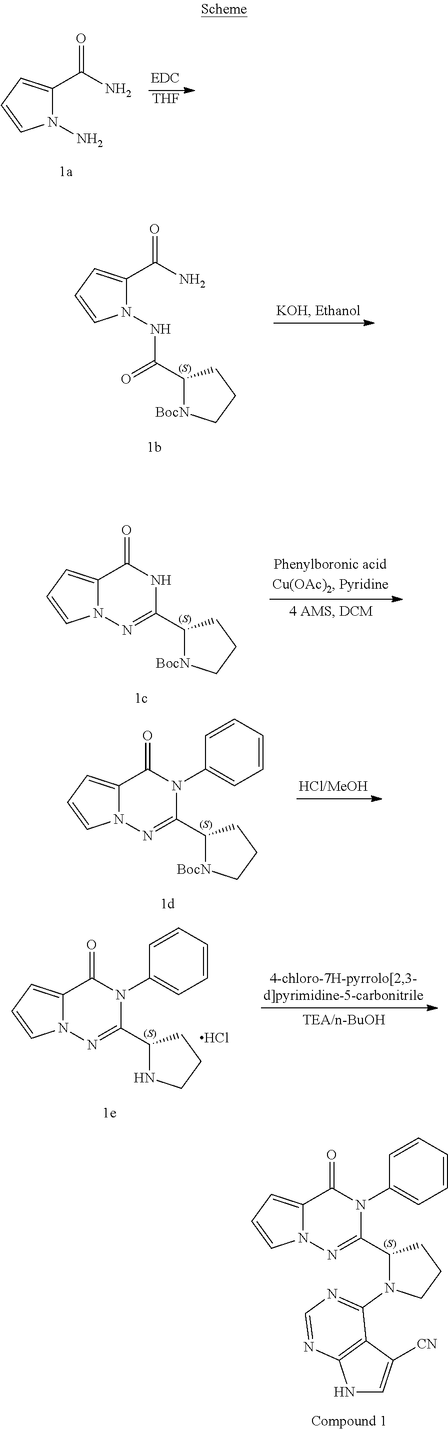

### Step 1-1 (S)-tert-butyl 2-(2-carbamoyl-1H-pyrrol-1-ylcarbamoyl)pyrrolidine-1-carboxylate (1b)

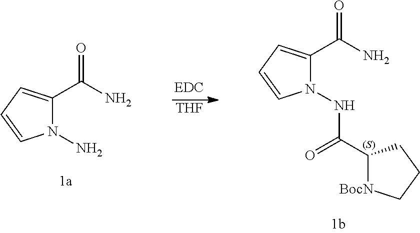

To a solution of 1a (3.0 g, 24.0 mmol) and (S)-1-(tert-butoxycarbonyl) pyrrolidine-2-carboxylic acid (7.1 g, 28.8 mmol) in THF (150 mL) was added EDC (5.52 g, 28.8 mmol). The reaction mixture was stirred at room temperature for 3.5 hours, then the mixture was diluted in water and extracted with EtOAc three times. The combined organic layers were separated, dried over anhydrous Na₂SO₄, filtered and concentrated to afford 1b as a white solid (4.6 g, yield: 60%). MS (m/z): 322.7 (M+H)⁺. It was used in the next step without further purification

### Step 1-2 (S)-tert-butyl 2-(4-oxo-3,4-dihydropyrrolo[2,1-f][1,2,4]triazin-2-yl) pyrrolidine-1-carboxylate (1c)

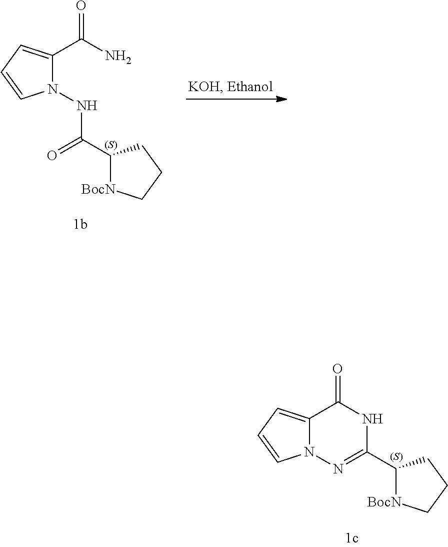

Ethanol (50 ml) was added to 1b (3.1 g, 9.6 mmol), then to the mixture was added a solution of KOH (2.88 g, 49.6 mmol) in water (50 mL). The reaction mixture was heated to 100° C. for 3 days. After cooling to room temperature, the reaction mixture was diluted in water and adjusted to pH=3-4 with 1N HCl aq. A precipitate was filtered off and dried to afford 1c as a white solid (1.7 g, yield: 58%). MS (m/z): 304.7 (M+H)⁺

### Step 1-3 (S)-tert-butyl 2-(4-oxo-3-phenyl-3,4-dihydropyrrolo[2,1-f][1,2,4]triazin-2-yl) pyrrolidine-1-carboxylate (1d)

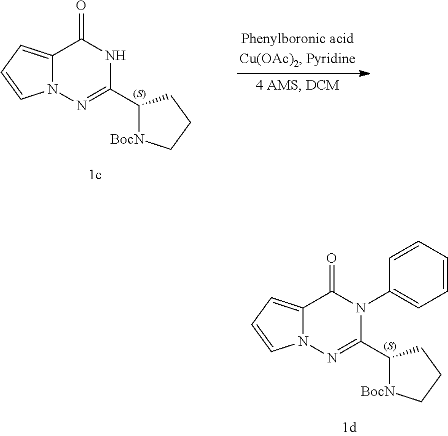

A mixture of 1c (604 mg, 2.0 mmol), phenylboronic acid (0.49 g, 4.0 mmol), 4AMS (2 g), Cu(OAc)₂ (0.73 g, 4.0 mmol) and Pyridine (0.8 mL, 10.0 mmol) in dry DCM (30 mL) was stirred for 18 hours at room temperature under dry air atmosphere. The mixture was concentrated in vacuo and purified by flash column chromatography eluting with MeOH/water to get 1d as a white solid (150 mg, yield: 20%). MS (m/z): 380.7 (M+H)⁺

### Step 1-4 (S)-3-phenyl-2-(pyrrolidin-2-yl)pyrrolo[2,1-f][1,2,4]triazin-4(3H)-one hydrochloride (1e)

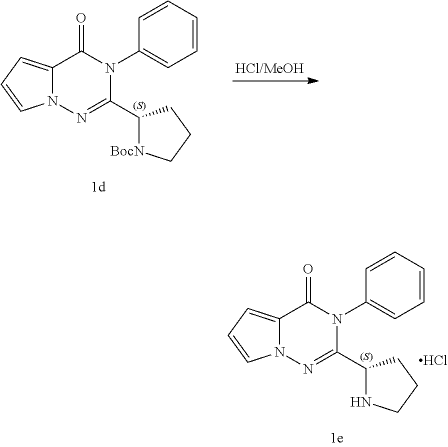

A solution of 1d (150 mg, 0.395 mmol) in 6N HCl/MeOH (20 mL) was stirred for 2.5 hours at room temperature, then concentrated under reduced pressure to afford 1e as a yellow oil which was used directly in next step without further purification.

### Step 1-5 (S)-4-(2-(4-oxo-3-phenyl-3,4-dihydropyrrolo[2,1-f][1,2,4]triazin-2-yl) pyrrolidin-1-yl)-7H-pyrrolo[2,3-d]pyrimidine-5-carbonitrile (Compound 1)

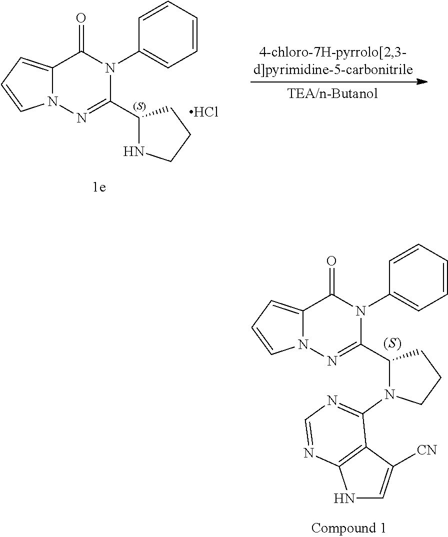

A mixture of 1e (30 mg, 0.095 mmol), 4-chloro-7H-pyrrolo[2,3-d]pyrimidine-5-carbonitrile (22 mg, 0.128 mmol) and TEA (0.05 ml, 0.360 mmol) in n-BuOH (3 mL) was stirred at reflux for 1.5 h. The reaction mixture was concentrated and purified by flash column chromatography eluting with MeOH/DCM to afford Compound 1 as a white solid (29 mg, yield: 64%). MS (m/z): 422.6 (M+H)⁺. ¹H NMR (400 MHz, DMSO-d₆) δ: 12.81 (s, 1H), 8.27-8.26 (m, 2H), 7.72-7.68 (m, 1H), 7.64-7.41 (m, 5H), 6.88 (dd, J=4.3, 1.7 Hz, 1H), 6.47 (dd, J=4.3, 2.7 Hz, 1H), 4.72-4.65 (m, 1H), 4.12-4.06 (m, 1H), 3.96-3.89 (m, 1H), 2.35-2.15 (m, 2H) 2.06-1.83 (m, 2H).

The following Compounds were prepared according to the procedure of Compound 1 using the corresponding reagents and intermediates under appropriate conditions that will be recognized by one skilled in the art:

| Compd. | | LC/MS | |
|---|---|---|---|
| No. | Structure | (M + H)⁺ | NMR |
| 6 | 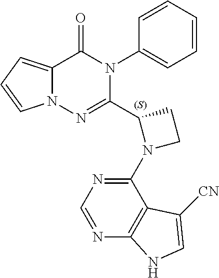 | 408.6 | ¹H NMR (400 MHz, DMSO-d₆) δ: 8.24 (m, 2H), 7.58 (m, 3H), 7.50 (m, 2H), 7.43 (m, 1H), 6.93 (m, 1H), 6.53 (m, 1H), 5.10 (m, 1H), 4.35 (m, 1H), 4.14 (m, 1H), 2.63 (m, 1H), 2.06 (m, 1H). |
| 7 | 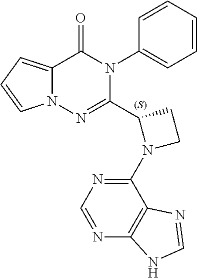 | 384.7 | ¹H NMR (400 MHz, DMSO-d₆) δ: 8.18 (s, 1H), 8.07 (m, 1H), 7.57-7.52 (m, 5H), 7.42 (m, 1H), 6.92 (m, 1H), 6.51 (m, 1H), 5.13 (m, 1H), 4.10 (m, 2H), 2.63 (m, 1H), 2.21 (m, 1H). |
| 8 | 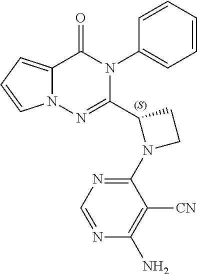 | 384.7 | ¹H NMR (400 MHz, CDCl₃) δ: 8.12 (s, 1H), 7.55 (m, 4H), 7.39 (s, 1H), 7.13 (m, 2H), 6.56 (m, 1H), 5.43 (s, 2H), 5.15 (m, 1H), 4.46 (m, 1H), 4.19 (m, 1H), 2.39 (m, 2H). |
| 25 | 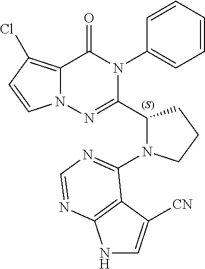 | 456.8 | ¹H NMR (400 MHz, DMSO-d₆) δ: 8.29 (s, 1H), 8.26 (s, 1H), 7.78-7.72 (m, 1H), 7.63-7.47 (m, 5H), 6.55 (d, J = 3.0 Hz, 1H), 4.68-4.60 (m, 1H), 4.12-4.04 (m, 1H), 3.96-3.88 (m, 1H), 2.36-2.16 (m, 2H), 2.03-1.86 (m, 2H). |
| 26 | 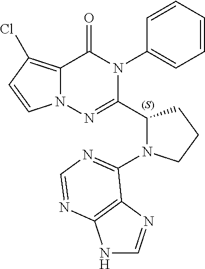 | 432.7 | ¹H NMR (400 MHz, DMSO-d₆) δ: 12.95 (s, 1H), 8.65-8.05 (m, 3H), 7.72-7.40 (m, 5H), 6.57-6.50 (m, 1H), 5.34-5.26 (m, 0.5H), 4.67-4.59 (m, 0.5H), 4.33-4.25 (m, 0.5H), 4.11-4.03 (m, 0.5H), 3.89-3.83 (m, 0.5H), 3.62-3.58 (m, 0.5H), 2.35-2.15 (m, 2H), 1.98-1.81 (m, 2H). |
| 27 | 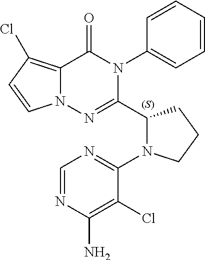 | 441.8 | ¹H NMR (400 MHz, DMSO-d₆) δ: 7.86 (s, 1H), 7.66-7.42 (m, 6H), 6.64-6.54 (m, 3H), 4.53-4.43 (m, 1H), 4.08-3.98 (m, 1H), 3.88-3.80 (m, 1H), 2.11-1.99 (m, 2H), 1.84-1.74 (m, 2H). |
| 28 | 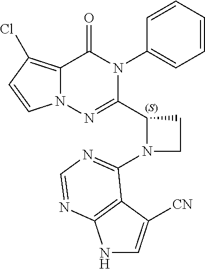 | 442.8 | ¹H NMR (400 MHz, DMSO-d₆) δ: 8.24-8.23 (m, 2H), 7.70-7.41 (m, 6H), 6.61 (s, 1H), 5.13-5.05 (m, 1H), 4.38-4.28 (m, 1H), 4.15-4.09 (m, 1H), 2.66-2.58 (m, 1H), 2.10-1.98 (m, 1H). |
| 29 | 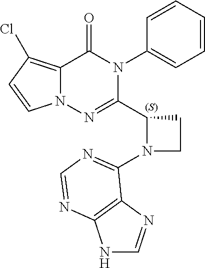 | 418.7 | ¹H NMR (400 MHz, DMSO-d₆) δ: 12.93 (s, 1H), 8.18 (s, 1H), 8.09 (s, 1H), 7.72-7.41 (m, 6H), 6.59 (s, 1H), 5.18-5.04 (m, 1H), 4.19-4.03 (m, 2H), 2.68-2.60 (m, 1H), 2.24-2.16 (m, 1H). |
| 30 | 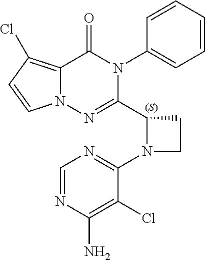 | 427.7 | ¹H NMR (400 MHz, DMSO-d₆) δ: 7.81 (s, 1H), 7.70-7.62 (m, 1H), 7.7-7.30 (m, 5H), 6.75-6.51 (m, 3H), 4.91-4.81 (m, 1H), 4.20-4.10 (m, 1H), 4.00-3.90 (m, 1H), 2.46-2.38 (m, 1H), 2.01-1.89 (m, 1H). |
| 31 | 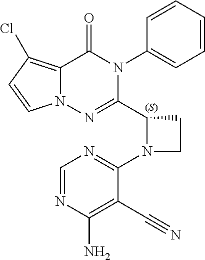 | 418.7 | ¹H NMR (400 MHz, DMSO-d₆) δ: 8.91-6.98 (m, 9H), 6.80-6.48 (m, 1H), 5.03-4.80 (m, 1H), 4.08-3.90 (m, 2H), 2.47-2.37 (m, 1H), 2.10-1.90 (m, 1H). |
| 32 | 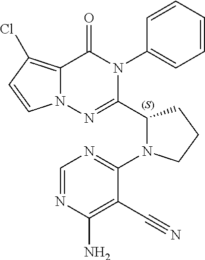 | 432.9 | ¹H NMR (400 MHz, DMSO-d₆) δ: 8.03 (s, 1H), 7.63-7.47 (m, 6H), 7.21 (s, 2H), 6.61-6.55 (m, 1H), 4.61-4.53 (m, 1H), 4.02-3.94 (m 1H), 3.82-3.74 (m 1H), 2.24-2.03 (m, 2H), 1.99-1.71 (m, 2H). |
| 33 | 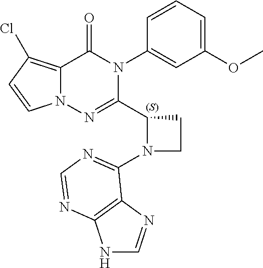 | 448.7 | ¹H NMR (400 MHz, CDCl₃) δ 8.36 (s, 1H), 7.95-7.68 (br, 1H), 7.51-6.60 (m, 5H), 6.45-6.20 (m, 1H), 5.50-5.20 (m, 1H), 4.61-4.16 (m, 2H), 2.75-2.25 (m, 2H). |
| 34 | 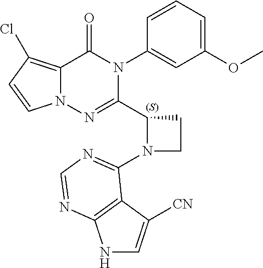 | 472.7 | ¹H NMR (400 MHz, DMSO-d₆) δ: 12.78 (s, 1H), 8.30-8.18 (m, 2H), 7.65-7.57 (m, 1H), 7.52-7.38 (m, 1H), 7.26-6.93 (m, 3H), 6.61 (s, 1H), 5.18-5.02 (m, 1H), 4.48-4.18 (m, 1H), 4.14-4.08 (m, 1H), 3.78 (s, 1.5H), 3.74 (s, 1.5H), 2.72-2.56 (m, 1H), 2.15-2.07 (m, 1H). |
| 35 | 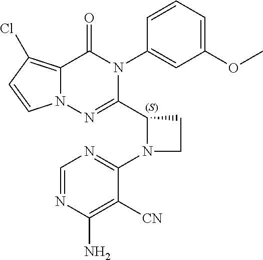 | 448.8 | ¹H NMR (400 MHz, DMSO-d₆) δ 7.98 (s, 1H), 7.71 (d, J = 2.7 Hz, 1H), 7.59-7.21 (m, 3H), 7.18-6.90 (m, 3H), 6.64 (d, J = 2.6 Hz, 1H), 5.15-4.95 (br, 1H), 4.13-3.93 (m, 2H), 3.76 (s, 1.5H), 3.73 (s, 1.5H), 2.65-2.50 (m, 1H), 2.15-2.03 (m, 1H). |
| 36 | 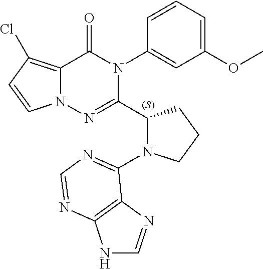 | 462.7 | ¹H NMR (400 MHz, CD₃OD) δ: 8.24 (s, 1H), 8.10-7.91 (m, 1H), 7.55-7.38 (m, 1H), 7.41-7.15 (m, 2H), 7.14-6.96 (m, 2H), 6.50-6.35 (m, 1H), 5.68-5.60 (m, 0.5H), 5.38-5.20 (m, 0.5H), 4.41-4.33 (m, 0.5H), 4.20-4.12 (m, 0.5H), 4.03-3.95 (m, 0.5H), 3.91-3.80 (m, 3H), 3.82-3.74 (m, 0.5H), 2.48-1.98 (m, 4H). |
| 37 | 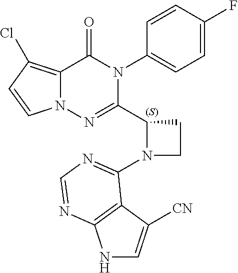 | 460.7 | ¹H NMR (400 MHz, DMSO-d₆) δ: 8.24-8.23 (m, 2H), 7.69-7.59 (m, 2H), 7.58-7.29 (m, 3H), 6.66-6.56 (m, 1H), 5.24-5.00 (m, 1H), 4.36-4.26 (m, 1H), 4.16-4.08 (m, 1H), 2.67-2.57 (m, 1H), 2.15-2.03 (m, 1H). |
| 38 | 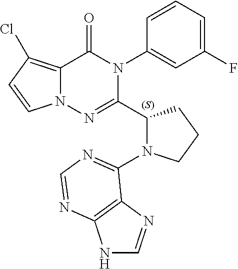 | 450.8 | ¹H NMR (400 MHz, DMSO-d₆) δ: 12.96 (s, 1H), 8.24-8.20 (m, 1H), 8.10 (s, 1H), 7.66-7.39 (m, 5H), 6.60-6.52 (m, 1H), 5.36-5.30 (m, 0.5H), 4.68-4.62 (m, 0.5H), 4.35-4.29 (m, 0.5H), 4.12-4.06 (m, 0.5H), 3.92-3.86 (m, 0.5H), 3.73-3.67 (m, 0.5H), 2.28-2.22 (m, 1H), 2.05-1.86 (m, 3H). |
| 39 | 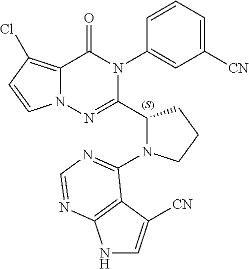 | 481.6 | ¹H NMR (400 MHz, CDCl₃) δ: 8.22 (s, 1H), 7.77 (s, 1H), 7.67 (s, 1H), 7.48 (d, J = 7.5 Hz, 1H), 7.37 (t, J = 7.6 Hz, 1H), 7.32-7.26 (m, 2H), 6.75 (d, J = 2.0 Hz, 1H), 5.46-5.38 (m, 1H), 4.07-3.99 (m, 1H), 3.90-3.80 (m, 1H), 2.40-2.18 (m, 2H), 2.11-2.03 (m, 2H). |
| 40 | 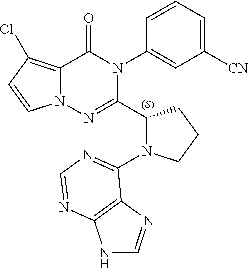 | 457.7 | ¹H NMR (400 MHz, CDCl₃) δ: 8.59-8.09 (m, 1H), 7.98 (s, 1H), 7.86-7.55 (m, 2H), 7.52-7.32 (m, 3H), 6.74 (s, 1H), 5.41-5.29 (m, 1H), 4.35-3.76 (m, 2H), 2.49-2.25 (m, 2H), 2.08-1.98 (m, 2H). |
| 41 | 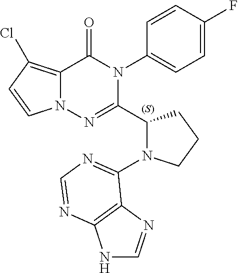 | 450.7 | ¹H NMR (400 MHz, DMSO-d₆) δ: 13.14-12.79 (m, 1H), 8.24-8.08 (m, 2H), 7.91-7.29 (m, 5H), 6.63-6.45 (m, 1H), 5.34-5.22 (m, 0.5H), 4.66-4.58 (m, 0.5H), 4.41-4.25 (m, 0.5H), 4.15-4.01 (m, 0.5H), 3.91-3.83 (m, 0.5H), 3.70-3.62 (m, 0.5H), 2.30-2.16 (m, 1H), 2.06-1.78 (m, 3H). |
| 42 | 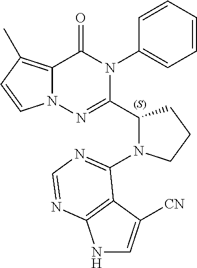 | 436.7 | ¹H NMR (400 MHz, DMSO-d₆) δ: 8.31-8.21 (m, 2H), 7.75-7.69 (m, 1H), 7.62-7.48 (m, 4H), 7.33 (d, J = 2.5 Hz, 1H), 6.28 (s, 1H), 4.69-4.61 (m, 1H), 4.11-4.03 (m, 1H), 3.96-3.88 (m, 1H), 2.34 (s, 3H), 2.32-2.24 (m, 1H), 2.20-2.12 (m, 1H), 2.00-1.93 (m, 2H). |
| 43 | 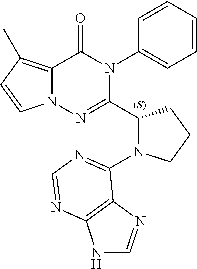 | 412.7 | ¹H NMR (400 MHz, DMSO-d₆) δ: 8.29-8.08 (m, 2H), 7.73-7.47 (m, 5H), 7.31-7.23 (m, 1H), 6.31-6.20 (m, 1H), 5.38-5.28 (m, 0.5H), 4.68-4.58 (m, 0.5H), 4.34-4.24 (m, 0.5H), 4.13-4.03 (m, 0.5H), 3.89-3.83 (m, 0.5H), 3.69-3.63 (m, 0.5H), 2.37-2.29 (m, 3H), 2.19-1.83 (m, 4H). |
| 44 | 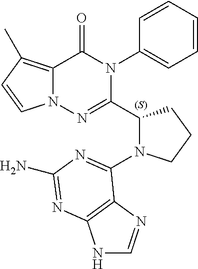 | 427.7 | ¹H NMR (400 MHz, DMSO-d₆) δ: 8.19 (s, 1H), 7.73-7.49 (m, 4H), 7.34-7.28 (m, 1H), 6.31-6.23 (m, 1H), 5.72-5.56 (m, 2H), 5.33-5.23 (m, 0.5H), 4.69-4.59 (m, 0.5H), 4.27-4.17 (m, 0.5H), 4.02-3.94 (m, 0.5H), 3.79-3.73 (m, 0.5H), 3.64-3.58 (m, 0.5H), 2.35 (s, 1.5H), 2.32 (s, 1.5H), 2.26-1.67 (m, 4H). |
| 45 | 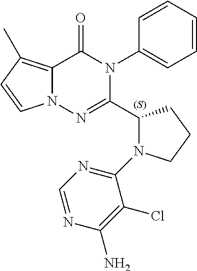 | 421.8 | ¹H NMR (400 MHz, DMSO-d₆) δ: 7.85 (s, 1H), 7.63-7.42 (m, 5H), 7.40-7.30 (m, 1H), 6.76-6.52 (br, 2H), 6.35-6.25 (m, 1H), 4.56-4.44 (m, 1H), 4.08-3.98 (m, 1H), 3.87-3.77 (m, 1H), 2.33 (s, 3H), 2.13-1.95 (m, 2H), 1.78-1.70 (m, 2H). |
| 46 | 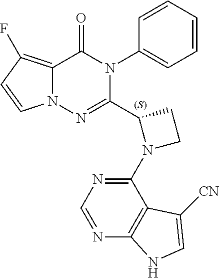 | 426.9 | ¹H NMR (400 MHz, CD₃OD) δ 8.22 (s, 1H), 7.93 (s, 1H), 7.76-7.68 (m, 1H), 7.64-7.51 (m, 3H), 7.40-7.29 (m, 1H), 7.30-7.17 (m, 1H), 6.28 (d, J = 3.2 Hz, 1H), 5.34-5.24 (m, 1H), 4.63-4.57 (m, 1H), 4.29-4.19 (m, 1H), 2.63-2.53 (m, 1H), 2.25-2.15 (m, 1H). |
| 47 | 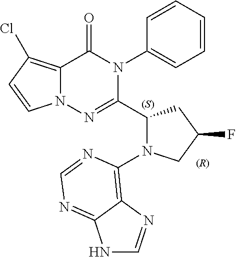 | 451.1 | ¹H NMR (400 MHz, DMSO-d₆) δ 8.22-8.10 (m, 2H), 7.70-7.35 (m, 6H), 6.53-6.47 (m, 1H), 5.54-4.85 (m, 2H), 4.52-4.44 (m, 1H), 4.03-3.66 (m, 1H), 2.27-1.93 (m, 2H). |
| 48 | 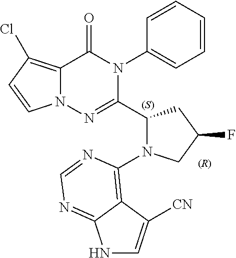 | 475 | ¹H NMR (400 MHz, DMSO-d₆) δ 8.39 (s, 1H), 8.33 (s, 1H), 7.80 (d, J = 7.8 Hz, 1H), 7.64-7.54 (m, 5H), 6.60 (d, J = 3.0 Hz, 1H), 5.67-5.53 (m, 1H), 4.77-4.73 (m, 1H), 4.39-4.27 (m, 1H), 4.19-4.10 (m, 1H), 2.59-2.29 (m, 2H). |
| 49 | 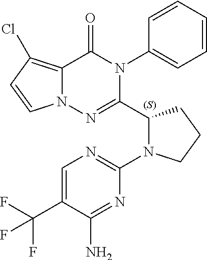 | 476.1 | ¹H NMR (400 MHz, DMSO-d₆) δ 8.22-8.18 (m, 1H), 8.06-7.71 (m, 1H), 7.68-7.61 (m, 5H), 6.91-6.81 (m, 2H), 6.67 (s, 1H), 4.59-4.51 (m, 1H), 3.81-3.73 (m, 1H), 3.59-3.51 (m, 1H), 2.22-2.07 (m, 2H), 1.93-1.81 (m, 2H). |
| 50 | 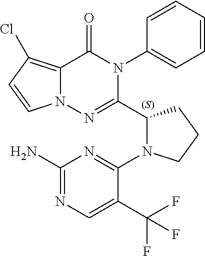 | 476.1 | ¹H NMR (400 MHz, DMSO-d₆) δ 8.17 (s, 1H), 7.92-7.84 (m, 1H), 7.75-7.48 (m, 5H), 6.83-6.59 (m, 3H), 4.70-4.62 (m, 1H), 3.72-3.62 (m, 1H), 3.58-3.48 (m, 1H), 2.17-1.97 (m, 2H), 1.89-1.79 (m, 1H), 1.73-1.63 (m, 1H). |
| 51 | 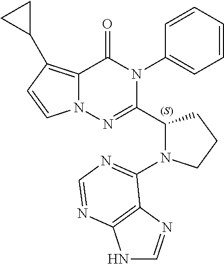 | 439.2 | ¹H NMR (400 MHz, CD₃OD) δ 8.25 (s, 1H), 8.03 (s, 1H), 7.68-7.53 (m, 5H), 7.21 (s, 1H), 6.10 (d, J = 2.4 Hz, 1H), 4.38-4.04 (m, 2H), 3.43 (br, 1H), 2.64-2.59 (m, 1H), 2.36-2.25 (m, 2H), 2.11-2.03 (m, 2H), 1.02-1.00 (m, 2H), 0.71-0.70 (m, 2H). |
| 52 | 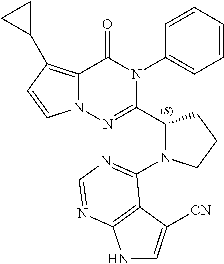 | 463.2 | ¹H NMR (400 MHz, DMSO-d₆) δ 8.55 (s, 1H), 8.51 (s, 1H), 8.01 (d, J = 7.6 Hz, 1H), 7.88-7.77 (m, 5H), 7.57 (d, J = 2.4 Hz, 1H), 6.31 (d, J = 2.8 Hz, 1H), 4.95-4.92 (m, 1H), 4.37-4.32 (m, 1H), 4.22-4.16 (m, 1H), 2.57-2.54 (m, 1H), 2.47-2.41 (m, 1H), 2.29-2.19 (m, 3H), 1.15-1.13 (m, 2H), 0.84 (m, 2H) |
| 53 | 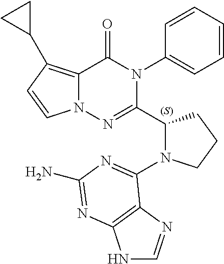 | 454.2 | ¹H NMR (400 MHz, CD₃OD) δ 7.76-7.63 (m, 5H), 7.53-7.52 (m, 1H), 7.26 (d, J = 2.4 Hz, 1H), 6.11 (d, J = 2.8 Hz, 1H), 4.37 (br, 1H), 4.11 (br, 1H), 3.44 (br, 1H), 2.67-2.62 (m, 1H), 2.33 (br, 1H), 2.20-2.17 (m, 1H), 2.08-2.06 (m, 1H), 2.00-1.90 (m, 1H), 1.02-1.00 (m, 2H), 0.71 (m, 2H). |
| 54 | 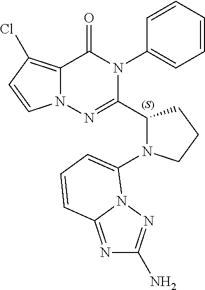 | 446.9 | ¹H NMR (400 MHz, CD₃OD) δ 7.75-7.65 (m, 2H), 7.62-7.52 (m, 2H), 7.48-7.35 (m, 2H), 7.15 (d, J = 2.7 Hz, 1H), 6.72 (d, J = 8.5 Hz, 1H), 6.43 (d, J = 2.7 Hz, 1H), 6.23 (d, J = 7.9 Hz, 1H), 5.73-5.67 (m, 1H), 3.85-3.77 (m, 1H), 3.59-3.51 (m, 1H), 2.20-2.08 (m, 2H), 1.98-1.90 (m, 2H). |
| 55 | 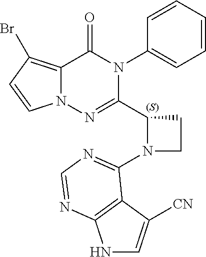 | 486.8 | ¹H NMR (400 MHz, DMSO-d₆) δ 8.42 (s, 2H), 7.85-7.60 (m, 6H), 6.86 (d, J = 2.9 Hz, 1H), 5.32-5.20 (br, 1H), 4.55-4.45 (m, 1H), 4.36-4.26 (m, 1H), 2.84-2.78 (m, 1H), 2.27-2.17 (m, 1H). |
| 56 | 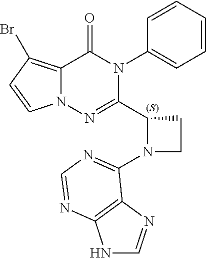 | 464.6 | ¹H NMR (400 MHz, DMSO-d₆) δ 8.21 (s, 1H), 8.13 (s, 1H), 7.68-7.40 (m, 6H), 6.70 (s, 1H), 5.22-5.08 (m, 1H), 4.25-4.08 (m, 2H), 2.73-2.63 (m, 1H), 2.28-2.18 (m, 1H). |
| 57 | 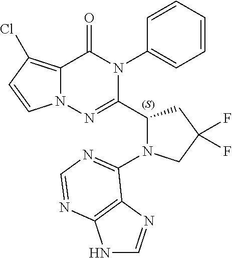 | 468.8 | ¹H NMR (400 MHz, DMSO-d₆) δ 8.35-8.29 (m, 1H), 8.23-8.10 (m, 1H), 7.86-7.42 (m, 6H), 6.62-6.52 (m, 1H), 5.66-5.56 (m, 0.5H), 4.94-4.82 (br, 1H), 4.62-4.52 (br, 0.5H), 4.41-4.31 (br, 0.5H), 4.21-4.11 (br, 0.5H), 3.03-2.91 (m, 2H). |
| 58 | 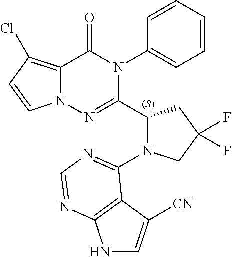 | 492.9 | ¹H NMR (400 MHz, DMSO-d₆) δ 8.28-8.24 (m, 1H), 8.12-8.05 (m, 1H), 7.83-7.75 (m, 1H), 7.68-7.54 (m, 5H), 6.66-6.60 (m, 1H), 4.96-4.81 (m, 1H), 4.64-4.36 (m, 2H), 3.03-2.83 (m, 2H). |
| 141 | 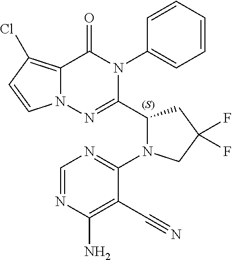 | 469 | ¹H NMR (400 MHz, DMSO-d₆) δ: 8.14 (s, 1H), 7.64-7.47 (m, 8H), 6.64 (d, J = 3.0 Hz, 1H), 4.92-4.84 (m, 1H), 4.45-4.29 (m, 2H), 2.93-2.81 (m, 1H), 2.47-2.41 (m, 1H). |
| 142 | 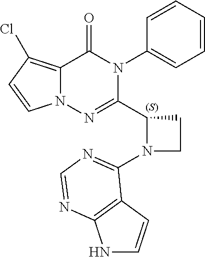 | 418.5 | ¹H NMR (400 MHz, DMSO-d₆) δ: 11.79 (s, 1H), 8.20 (s, 1H), 7.76-7.56 (m, 6H), 7.24 (s, 1H), 6.74 (s, 1H), 6.41 (s, 1H), 5.05-4.99 (br, 1H), 4.22-4.10 (m, 2H), 2.78-2.72 (m, 1H), 2.26-2.16 (m, 1H). |
| 143 | 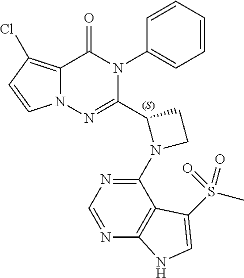 | 496. | ¹H NMR (400 MHz, DMSO-d₆) δ: 12.86 (s, 1H), 8.32 (s, 1H), 8.00 (s, 1H), 7.75-7.31 (m, 6H), 6.60 (d, J = 3.0 Hz, 1H), 5.33-5.10 (m, 1H), 4.69-4.59 (m, 1H), 4.02-3.81 (m, 1H), 3.41 (s, 3H), 2.58-2.48 ( m, 1H), 1.89-1.79 (m, 1H). |
| 144 | 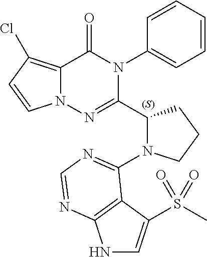 | 510. | ¹H NMR (400 MHz, DMSO-d₆) δ: 8.26 (s, 1H), 7.99 (s, 1H), 7.72-7.53 (m, 6H), 7.39 (d, J = 2.9 Hz, 1H), 6.58 (d, J = 2.9 Hz, 1H), 4.83 (t, J = 7.0 Hz, 1H), 4.31-4.21 (m, 1H), 3.65-3.61 (m, 1H), 3.44 (s, 3H), 2.13-2.03 (m, 2H), 1.93-1.89 (m, 1H), 1.72-1.68 (m, 1H). |
| 145 | 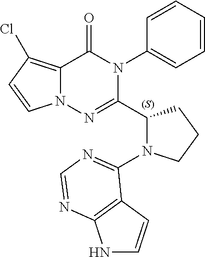 | 432. | ¹H NMR (400 MHz, DMSO-d₆) δ: 8.14 (s, 1H), 7.75 (d, J = 8.1 Hz, 1H), 7.65-7.51 (m, 6H), 7.15 (d, J = 3.4 Hz, 1H), 6.61 (d, J = 3.6 Hz, 1H), 6.59 (d, J = 2.9 Hz, 1H), 4.66 (d, J = 7.3 Hz, 1H), 4.13-4.05 (m, 1H), 3.87-3.79 (m, 1H), 2.30-2.19 (m, 2H), 2.05-2.01 (m, 1H), 1.90-1.84 (m, 1H). |
| 146 | 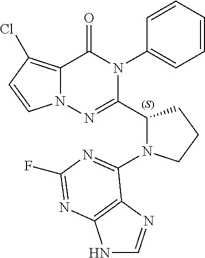 | 451.0 | ¹H NMR (400 MHz, DMSO-d₆) δ: 8.22-8.12 (m, 2H), 7.64-7.49 (m, 6H), 6.61-6.55 (m, 1H), 5.34-4.60 (m, 1H), 4.33-4.10 (m, 1H), 3.84-3.65 (m, 1H), 2.29-2.23 (m, 1H), 2.01-1.89 (m, 3H). |
| 147 | 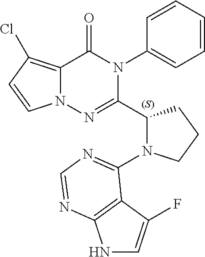 | 450.1 | ¹H NMR (400 MHz, DMSO-d₆) δ: 8.17 (s, 1H), 7.76 (d, J = 8.0 Hz, 1H), 7.61-7.53 (m, 6H), 7.17 (s, 1H), 6.58 (d, J = 2.9 Hz, 1H), 4.70-4.64 (m, 1H), 3.92-3.86 (m, 1H), 3.74-3.68 (m, 1H), 2.29-2.19 (m, 2H), 2.02-1.98 (m, 1H), 1.95-1.89 (m, 1H). |
| 148 | 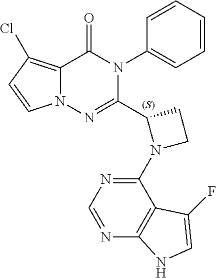 | 436.1 | ¹H NMR (400 MHz, DMSO-d₆) δ: 11.62 (s, 1H), 8.19 (s, 1H), 7.73-7.50 (m, 6H), 7.23-7.13 (m, 1H), 6.75-6.65 (m, 1H), 5.06-4.98 (m, 1H), 4.23-4.15 (m, 1H), 4.12-4.04 (m, 1H), 2.75-2.67 (m, 1H), 2.25-2.16 (m, 1H). |
| 149 | 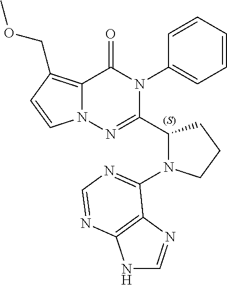 | 442.8 | ¹H NMR (400 MHz, CD₃OD) δ 8.28 (d, J = 7.1 Hz, 0.5H), 8.20 (s, 1H), 8.01(s, 0.5H), 7.98(s, 0.5H), 7.24(s, 0.5H), 7.16(s, 0.5H), 7.77-7.41 (m, 5H), 6.49(s, 0.5H), 6.45(s, 0.5H), 5.58(d, J = 2.4 Hz, 0.5H), 4.99-4.96 (m, 0.5H), 4.59 (s, 2H), 4.44-4.33 (m, 0.5H), 4.21-4.10 (m, 0.5H), 4.04-3.94 (m, 0.5H), 3.80-3.72 (m, 0.5H), 3.31 (s, 3H), 2.35-1.93 (m, 4H). |
| 150 | 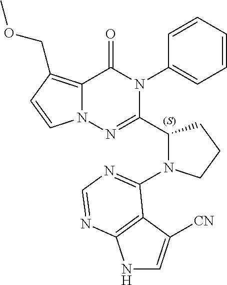 | 466.8 | ¹H NMR (400 MHz, CD₃OD) δ 8.24 (s, 1H), 7.97 (s, 1H), 7.77 (d, J = 7.9 Hz, 1H), 7.65-7.53 (m, 4H), 7.43 (d, J = 7.3 Hz, 1H), 7.29 (d, J = 2.7 Hz, 1H), 6.52 (d, J = 2.6 Hz, 1H), 4.93-4.92 (m, 1H), 4.51 (s, 2H), 4.29-4.25 (m, 1H), 4.09-4.05 (m, 1H), 3.35 (s, 3H), 2.47-2.40 (m, 1H), 2.19-2.18 (m, 1H), 2.11-2.04 (m, 2H). |
| 151 | 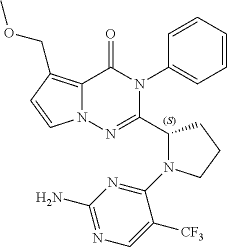 | 485.8 | ¹H NMR (400 MHz, CD₃OD) δ 8.15 (s, 1H), 7.76 (d, J = 7.5 Hz, 1H), 7.66-7.56 (m, 3H), 7.43 (d, J = 7.1 Hz, 1H), 7.31 (d, J = 2.3 Hz, 1H), 6.56 (d, J = 2.0 Hz, 1H), 4.95-4.93 (m, 1H), 4.53 (s, 2H), 3.86-3.82 (m, 1H), 3.72-3.67 (m, 1H), 3.37 (s, 3H), 2.26-2.17(m, 1H), 2.07-2.02 (m, 1H), 1.93-1.84 (m, 2H). |
| 152 | 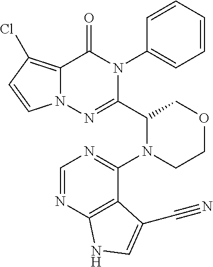 | 473.0 | ¹H NMR (400 MHz, CD₃OD) δ 8.14 (s, 1H), 8.00(s, 1H), 7.59 (d, J = 8.0 Hz, 1H), 7.52(d, J = 3.2 Hz, 1H), 7.48-7.41 (m, 2H), 7.29-7.26(m, 1H), 7.16-7.12(m, 1H), 6.59(d, J = 2.8 Hz, 1H), 5.07-5.05 (m, 1H), 4.29-4.24 (m, 2H), 4.01-3.97 (m, 2H), 3.84-3.79 (m, 1H), 3.72-3.68(m, 1H). |
| 153 | 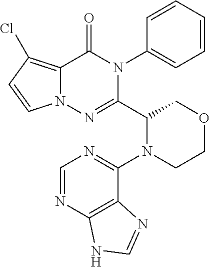 | 449.6 | ¹H NMR (400 MHz, CD₃OD) δ 8.11 (s, 1H), 7.87(s, 1H), 7.72-7.70 (m, 1H), 7.51-7.47(m, 1H), 7.44-7.37(m, 4H), 6.54(d, J = 2.8 Hz, 1H), 5.01-4.92 (m, 1H), 4.30-4.19 (m, 2H), 4.07-4.03 (m, 1H), 3.69-3.63 (m, 3H). |
| 186¹ | 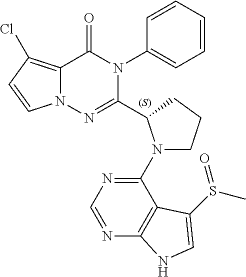 | 494.1 | ¹H NMR (400 MHz, DMSO-d₆) δ: 12.39 (s, 1H), 8.23 (s, 1H), 8.22(s, 1H), 7.88-7.30 (m, 7H), 6.56 (d, J = 3.0 Hz, 1H), 6.55 (d, J = 3.0 Hz, 1H), 4.76-4.60 (m, 1H), 4.15-3.63 (m, 2H), 2.90 (s, 1.5H), 2.85 (s, 1.5H), 2.31-2.15 (m, 1H), 2.01-1.69 (m, 2H). |
| 187² | 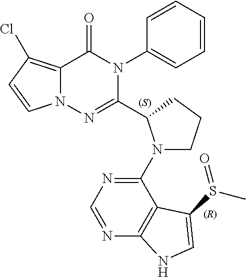 | 494.1 | ¹H NMR (400 MHz, DMSO-d₆) δ: 8.24(s, 1H), 7.76 (d, J = 8.0 Hz, 1H), 7.67-7.54 (m, 5H), 7.39 (d, J = 2.8 Hz, 1H), 6.60 (d, J = 2.8 Hz, 1H), 4.78 (t, J = 7.1 Hz, 1H), 3.87-3.79(m, 2H), 2.93(s, 3H), 2.15-2.07 (m, 2H), 2.00-1.94 (m, 1H), 1.85-1.73(m, 1H). |
| 188 | 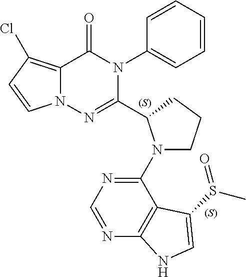 | 494.1 | ¹H NMR (400 MHz, DMSO-d₆) δ: 8.23(s, 1H), 7.85(s, 1H), 7.77 (d, J = 8.0 Hz, 1H), 7.64-7.53 (m, 4H), 7.49 (d, J = 2.8 Hz, 1H), 6.58 (d, J = 2.8 Hz, 1H), 4.68-4.65 (m, 1H), 4.25-4.18(m, 1H), 3.69-3.63(m, 1H), 2.88(s, 3H), 2.29-2.18 (m, 2H), 1.97-1.88 (m, 2H). |

¹compound was purified by flash column chromatography
²and ³compounds were purified by preparative TLC

| Compd. | | LC/MS | |
|---|---|---|---|
| No. | Structure | (M + H)⁺ | NMR |
| 190 | 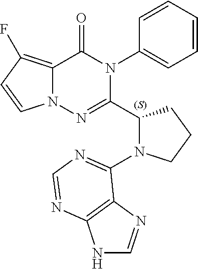 | 416.8 | ¹H NMR (400 MHz, CD₃OD) 8.24 (s, 1H), 8.03 (s, 1H), 7.84-7.41 (m, 5H), 7.15-7.09 (m, 1H), 6.30-6.15 (m, 1H), 5.65-5.50 (m, 0.5H), 4.91-4.85 (m, 0.5H), 4.42-4.37 (m, 0.5H), 4.23-4.13 (m, 0.5H), 4.05-3.95 (m, 0.5H), 3.85-3.78 (m, 0.5H), 2.37-1.97 (m, 4H). |
| 191 | 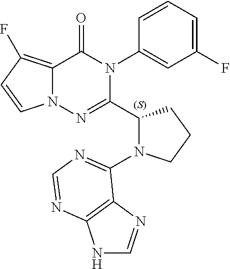 | 434.8 | ¹H NMR (400 MHz, CD₃OD) δ 8.18 (s, 1H), 7.97 (s, 1H), 7.65-7.45 (m, 2H), 7.34-7.20 (m, 2H), 7.10-7.03 (m, 1H), 6.23-6.10 (m, 1H), 5.58-5.48(m, 0.5H), 4.87-4.78 (m, 0.5H), 4.35-4.28 (m, 0.5H), 4.17-4.07 (m, 0.5H), 3.99-3.89 (m, 0.5H), 3.80-3.70 (m, 0.5H), 2.30-1.94 (m, 4H). |
| 192 | 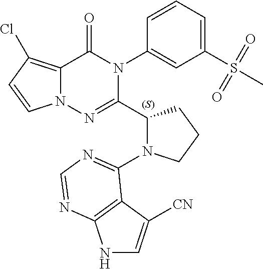 | 535.1 | ¹H NMR (400 MHz, DMSO-d₆) δ: 12.87 (s, 1H), 8.46-8.23 (m, 3H), 8.16-8.11 (m, 1H), 7.98-7.88 (m, 2H), 7.60-7.57 (m, 1H), 6.65-6.59 (m, 1H), 4.72-4.51 (m, 1H), 4.23-4.07 (m, 1H), 3.97-3.91(m, 1H), 3.32-3.28 (m, 3H), 2.43-2.21 (m, 2H), 2.13-1.96 (m, 2H). |
| 193 | 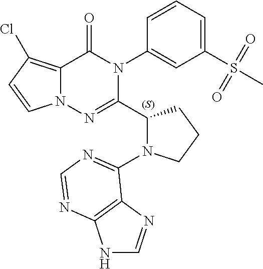 | 511.0 | ¹H NMR (400 MHz, DMSO-d₆) δ: 12.95 (s, 1H), 9.03-7.75 (m, 6H), 7.54-7.45 (m, 1H), 6.57-6.54 (m, 1H), 5.35-5.13 (m, 0.5H), 4.53-4.31 (m, 0.5H), 4.05-3.65 (m, 2H), 3.25-3.20(m, 3H), 2.38-1.84 (m, 4H). |
| 194 | 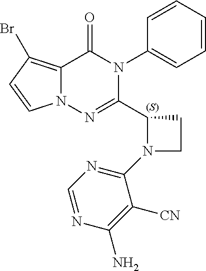 | 465.2 | ¹H NMR (400 MHz, DMSO-d₆) δ: 8.08 (s, 1H), 7.82 (d, J = 2.9 Hz, 1H), 7.74-7.60 (m, 3H), 7.53 (d, J = 7.2 Hz, 2H), 7.34 (br, 2H), 6.81 (d, J = 2.9 Hz, 1H), 5.09 (s, 1H), 4.16 (s, 2H), 2.72-2.60 (m, 1H), 2.25-2.08 (m, 1H). |
| 195 | 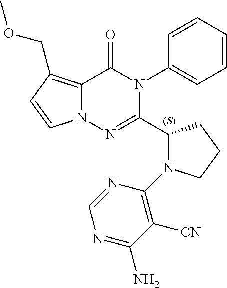 | 443.3 | ¹H NMR (400 MHz, DMSO-d₆) δ 8.02 (s, 1H), 7.61-7.49 (m, 6H), 7.22 (brs, 2H), 6.50 (d, J = 2.8 Hz, 1H), 4.60 (s, 3H), 4.00-3.94 (m, 1H), 3.81-3.75 (m, 1H), 3.340(brs, 1H), 3.22 (s, 2H), 2.19-2.07 (m, 2H), 1.97-1.90 (m, 1H), 1.83-1.73 (m, 1H). |
| 196 | 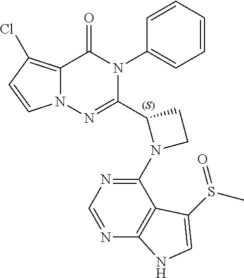 | 480.1 | ¹H NMR (400 MHz, DMSO-d₆) δ: 12.51 (br, 1H), 8.23 (d, J = 1.9 Hz, 1H), 8.04-7.35 (m, 7H), 6.69-6.53 (m, 1H), 5.15-4.98 (m, 1H), 4.50-4.28 (m, 1H), 3.97-3.90 (m, 1H), 2.89 (d, J = 4.2 Hz, 3H), 2.62-2.55 (m, 1H), 2.04-1.84 (m, 1H). |
| 197 | 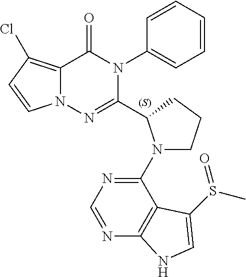 | 494.1 | ¹H NMR (400 MHz, DMSO-d₆) δ: 12.39 (s, 1H), 8.23 (d, J = 4.1 Hz, 1H), 7.85-7.31 (m, 7H), 6.56-6.53 (m, 1H), 4.75-4.67 (m, 1H), 4.13-3.64 (m, 2H), 2.90 (s, 1.5H), 2.85 (s, 1.5H), 2.23-1.71 (m, 4H). |
| 198 | 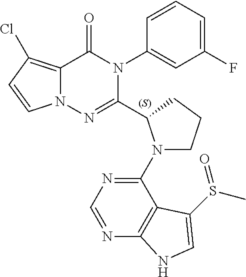 | 512.2 | ¹H NMR (400 MHz, DMSO-d₆) δ: 12.41 (br, 1H), 8.25-8.22 (m, 1H), 7.91-7.32 (m, 6H), 6.60-6.55 (m, 1H), 4.87-4.52 (m, 1H), 4.23-3.61 (m, 2H), 2.90 (s, 1.5H), 2.85 (s, 1.5H), 2.24-1.78 (m, 4H). |
| 199 | 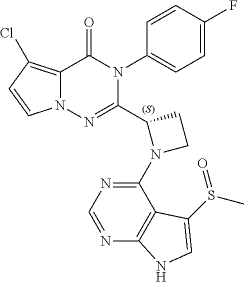 | 498.1 | ¹H NMR (400 MHz, DMSO-d₆) δ: 12.48 (br, 1H), 8.21 (d, J = 2.0 Hz, 1H), 8.05-7.30 (m, 6H), 6.74-6.51 (m, 1H), 5.10-5.03 (m, 1H), 4.52-4.25 (m, 1H), 3.96-3.93 (m, 1H), 2.88 (d, J = 6.3 Hz, 3H), 2.68-2.54 (m, 1H), 2.14-1.93 (m, 1H). |
| 200 | 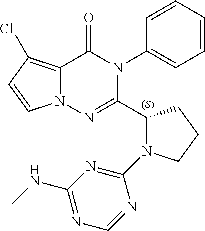 | 423.2 | ¹H NMR (400 MHz, CD3OD) δ 8.08-7.78 (m, 1H), 7.67-7.37 (m, 5H), 7.35 (s, 1H), 6.51-6.48 (m, 1H), 4.68-4.58 (m, 1H), 3.81-3.73 (m, 1H), 3.60-3.53 (m, 1H), 2.85 (s, 3H), 2.20-2.10 (m, 2H), 2.00-1.87 (m, 2H). |
| 201 | 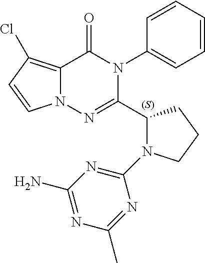 | 423.1 | ¹H NMR (400 MHz, DMSO-d6) δ 7.91 (s, 0.5H), 7.84-7.81 (m, 0.5H), 7.61-7.46 (m, 5H), 6.73 (s, 1H), 6.59 (d, J = 3.2 Hz, 0.5H), 6.58 (d, J = 2.8 Hz, 0.5H), 4.58-4.57(m, 0.5H), 4.51-4.49(m, 0.5H), 3.77-3.60 (m, 1H), 3.45-3.38 (m, 1H), 2.14 1.92 (m, 5H), 1.83-1.70 (m, 2H). |
| 202 | 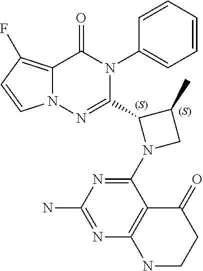 | 461.2 | ¹H NMR (400 MHz, CD3OD) δ 7.74-7.52 (m, 4H), 7.42-7.07 (m, 2H), 6.31 (d, J = 2.5 Hz, 1H), 5.44-5.22 (m, 1H), 4.48-4.26 (m, 1H), 3.55-3.35 (m, 2H), 3.25-3.04 (m, 1H), 2.64-2.42 (m, 2H), 2.37-2.18 (m, 1H), 0.85-0.44 (m, 3H). |
| 203 | 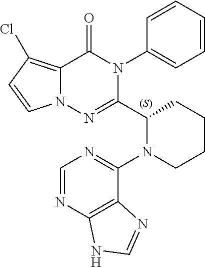 | 447.1 | ¹H NMR (400 MHz, CD3OD) δ 8.03 (s, 1H), 7.92 (s, 1H), 7.39 (d, J = 7.2 Hz, 1H), 7.36 (d, J = 3.2 Hz, 2H), 7.26 (s, 2H), 7.11-7.04 (m, 2H), 6.45 (d, J = 2.8 Hz, 1H), 5.25 (br, 1H), 4.48 (br, 1H), 3.60 (br, 1H), 2.12-2.03 (m, 2H), 1.74-1.40 (m, 4H) |
| 204 | 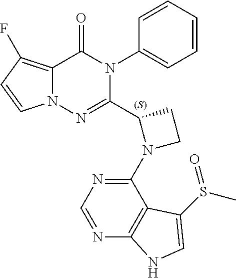 | 464.1 | ¹H NMR (400 MHz, CD₃OD) δ 8.24 (d, J = 2.6 Hz, 1H), 7.93 (s, 1H), 7.78 (s, 1H), 7.66-7.62 (m, 1H), 7.60-7.52 (m, 2H), 7.39-7.33 (m, 1H), 7.21 (dd, J = 7.5, 4.3 Hz, 1H), 6.30 (dd, J = 3.2, 2.0 Hz, 1H), 5.28-5.22 (m, 1H), 4.79-4.68 (m, 1H), 4.11-4.04 (m, 1H), 3.01 (s, 3H), 2.61-2.51 (m, 1H), 2.20-2.07 (m, 1H). |
| 205 | 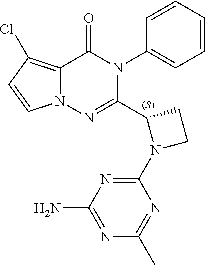 | 409.1 | ¹H NMR (400 MHz, DMSO-d6) δ 7.72 (d, J = 2.9 Hz, 1H), 7.60-7.36 (m, 5H), 6.79 (br, 2H), 6.65 (d, J = 3.0 Hz, 1H), 4.61 (s, 1H), 3.83-3.74 (m, 2H), 2.45-2.40 (m, 1H), 2.10-1.97 (m, 1H), 2.05 (s, 3H). |
| 206 | 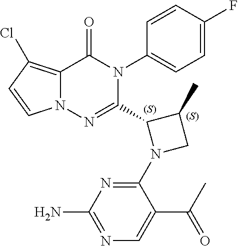 | 468.1 | ¹H NMR (400 MHz, CDCl₃) δ 8.37 (s, 1H), 7.67 (s, 1H), 7.31 (t, J = 8.4 Hz, 1H), 7.21 (dd, J = 8.2, 2.3 Hz, 1H), 7.18-7.12 (m, 1H), 7.10 (s, 1H), 6.44 (d, J = 2.0 Hz, 1H), 5.47 (s, 2H), 5.09 (br, 1H), 4.50-4.24 (m, 4H), 3.58-3.34 (m, 1H), 2.40 (br, 1H), 2.22 (s, 3H), 0.80 (d, J = 6.7 Hz, 3H). |
| 207 | 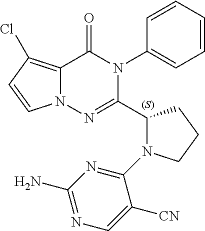 | 433.1 | ¹H NMR (400 MHz, DMSO-d₆) δ 8.18 (s, 1H), 7.85 (d, J = 7.7 Hz, 1H), 7.60-7.46 (m, 5H), 6.97 (br, 2H), 6.59 (d, J = 3.0 Hz, 1H), 4.57-4.56 (m, 1H), 3.93 (br, 1H), 3.77-3.73 (m, 1H), 2.07-2.04 (m, 2H), 1.89 (br, 1H), 1.70-1.60 (m, 1H). |
| 208 | 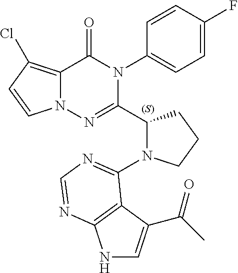 | 492.5 | ¹H NMR (400 MHz, DMSO-d₆) δ: 8.16 (s, 1H), 8.03 (s, 1H), 7.79-7.75 (m, 1H), 7.67-7.61 (m, 1H), 7.45-7.36 (m, 3H), 6.57 (d, J = 3.0 Hz, 1H), 4.50-4.44 (m, 1H), 3.86-3.82 (m, 1H), 3.68-3.64 (m, 1H), 2.48 (s, 3H), 2.30-1.94 (m, 3H), 1.67-1.59 (m, 1H). |
| 209 | 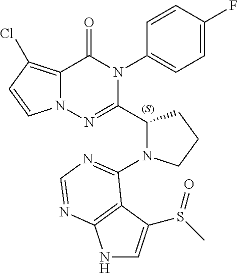 | 512.6 | ¹H NMR (400 MHz, DMSO-d₆) δ: 12.43 (s, 1H), 8.25 (s, 1H), 7.94-7.57 (m, 3H), 7.53-7.31 (m, 3H), 6.58-4.55 (m, 1H), 4.75-4.62 (m, 1H), 4.19-4.12 (m, 0.5H), 3.85-3.80 (m, 0.5H), 3.70-3.51 (m, 1H), 290 (s, 1.5H), 2.86 (s, 1.5H), 2.33-2.04 (m, 2H), 2.01-1.73 (m, 2H). |
| 210 | 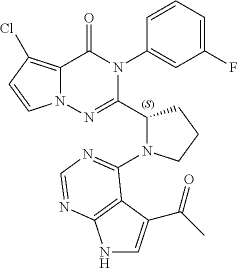 | 492.6 | ¹H NMR (400 MHz, DMSO-d₆) δ: 12.28 (br, 1H), 8.17 (s, 1H), 8.04 (s, 1H), 7.66-7.31 (m, 5H), 6.58 (s, 1H), 4.59-4.38 (m, 1H), 3.94-3.62 (m, 2H), 2.48 (s, 3H), 2.15-1.89 (m, 3H), 1.67-1.64 (m, 1H). |
| 211 | 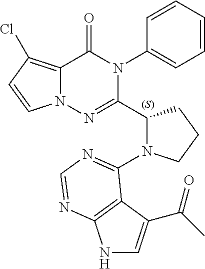 | 474.1 | ¹H NMR (400 MHz, DMSO-d₆) δ: 12.29 (br, 1H), 8.16 (s, 1H), 8.03 (s, 1H), 7.80-7.35 (m, 6H), 6.57 (s, 1H), 4.47-4.44 (m, 1H), 3.81-3.64 (m, 2H), 2.48 (s, 3H), 2.11-1.93 (m, 3H), 1.65-1.56 (m, 1H). |
| 212 | 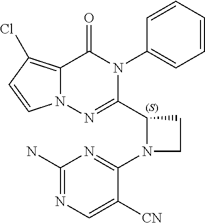 | 419.1 | 1H NMR (400 MHz, DMSO-d₆) δ 8.15 (s, 1H), 7.74 (s, 1H), 7.58-7.52 (m, 4H),, 7.45-7.38 (m, 1H), 7.14 (br, 2H), 6.66 (d, J = 3.0 Hz, 1H), 5.04-4.80 (m, 1H), 4.10-4.00 (m, 2H), 2.58-2.51 (m, 1H), 2.09-2.00 (m, 1H). |
| 213 | 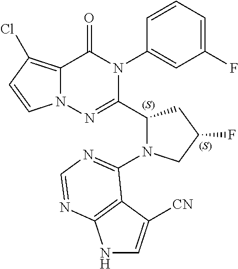 | 493.0 | 1H NMR (400 MHz, DMSO-d₆) δ 12.93 (brs, 1H), 8.39-8.35 (m, 2H), 7.71-7.53 (m, 4H), 7.45-7.41 (m, 1H), 6.60 (dd, J = 3.0, 1.2 Hz, 1H), 5.63-5.89(m, 0.5H), 5.48-5.44 (m, 0.5H), 4.93 (dd, J = 9.7, 3.2 Hz, 0.5H), 4.86(dd, J = 9.7, 3.2 Hz, 0.5H), 4.44-4.25 (m, 2H), 2.67-2.58 (m, 1H), 2.33-2.23 (m, 1H). |
| 214 | 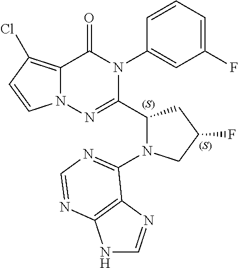 | 469.0 | 1H NMR (400 MHz, DMSO-d₆) δ 13.06 (brs, 1H), 8.29-8.18 (m, 2H), 8.06-7.40 (m, 5H), 6.59-6.55 (m, 1H), 5.51-5.29 (m, 2H), 4.86-4.30(m, 1H), 4.14-3.97 (m, 1H), 2.68-2.55 (m, 1H), 2.37-2.22 (m, 1H). |
| 215 | 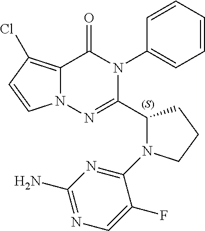 | 426.2 | ¹H NMR (400 MHz, DMSO-d₆) δ 7.72 (d, J = 6.0 Hz, 1H), 7.63-7.49 (m, 6H), 6.62 (d, J = 3.0 Hz, 1H), 5.96 (s, 2H), 4.56-4.55 (m, 1H), 3.86-3.81 (m, 1H), 3.63-3.56 (m, 1H), 2.16-1.99 (m, 2H), 1.87-1.71 (m, 2H). |
| 216 | 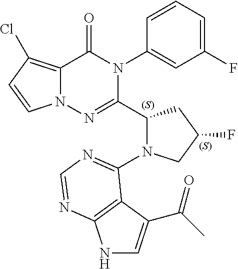 | 510.1 | ¹H NMR (400 MHz, DMSO-d₆) δ 8.25 (s, 1H), 8.17 (d, J = 5.4 Hz, 1H), 7.67-7.35 (m, 5H), 6.63 (d, J = 3.0 Hz, 1H), 5.32-4.06 (m, 1H), 4.82-4.70 (m, 1H), 4.22-4.06 (m, 1H), 4.03-3.87 (m, 1H), 2.55-2.51 (m, 3H), 2.43-2.26 (m, 2H). |
| 218 | 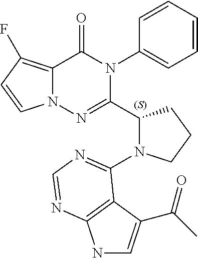 | 458.1 | ¹H NMR (400 MHz, CDCl₃) δ 11.60 (s, 1H), 8.23 (s, 1H), 7.76 (d, J = 7.9 Hz, 1H), 7.64 (s, 1H), 7.62-7.46 (m, 3H), 7.30-7.26 (m, 1H), 7.08-7.00 (m, 1H), 6.16 (d, J = 3.1 Hz, 1H), 4.81 (t, J = 6.8 Hz, 1H), 4.04-3.92 (m, 1H), 3.88-3.72 (m, 1H), 2.59 (s, 3H), 2.18-2.09 (m, 1H), 2.08-1.93 (m, 3H). |
| 219 | 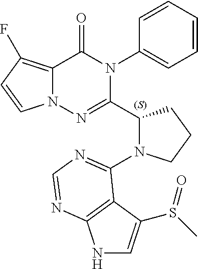 | 478.1 | ¹H NMR (400 MHz, DMSO-d₆) δ 12.33 (brs, 1H), 8.26 (s, 0.5H), 8.25(s, 0.5H), 7.87(s, 0.5H), 7.77-7.72 (m, 1H), 7.66-7.52 (m, 4.5H), 7.38-7.36(m, 0.5H), 7.27-7.25(m, 0.5H), 6.42 (d, J = 3.2 Hz, 0.5H), 6.40 (d, J = 3.2 Hz, 0.5H), 4.81-4.77(m, 0.5H), 4.68-4.65 (m, 0.5H), 4.19-4.14 (m, 0.5H), 3.84-3.80 (m, 1H), 3.71-3.65 (m, 0.5H), 2.93 (s, 1.5H), 2.88(s, 1.5H), 2.33-1.764 (m, 4H). |
| 220 | 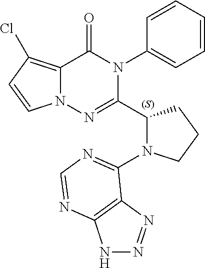 | 434.1 | ¹H NMR (400 MHz, CD₃OD) δ 8.64 (d, J = 8.0 Hz, 0.5H), 8.14 (s, 1H), 7.82 (d, J = 7.2 Hz, 0.5H), 7.66-7.62 (m, 1H), 7.58-7.53 (m, 2H), 7.47-7.43 (m, 1H), 7.27 (s, 0.5H), 7.162 (s, 0.5H), 6.43 (s, 0.5H), 6.38 (s, 0.5H), 5.72(br, 0.5 Hz), 4.71(br, 0.5 Hz), 4.48-4.424 (m, 0.5H), 4.27-4.22 (m, 0.5H), 4.02-3.96 (m, 0.5H), 3.82-3.75 (m, 0.5H), 2.41-2.23 (m, 0.5H), 2.29-2.24 (m, 0.5H), 2.21-2.15 (m, 1H), 2.10-1.95 (m, 2H). |
| 221 | 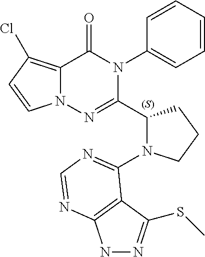 | 479.1 | ¹H NMR (400 MHz, DMSO-d₆) δ 8.21 (s, 1H), 7.78 (d, J = 8.4 Hz, 1H), 7.63-7.52 (m, 5H), 6.59 (d, J = 3.2 Hz, 1H), 4.67-4.64 (m, 1H), 4.23-4.18(m, 1H), 4.03-3.97 (m, 1H), 2.59 (s, 3H), 2.33-2.15 (m, 2H), 2.03-1.89 (m, 2H). |
| 222 | 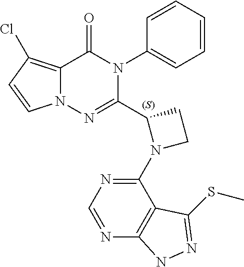 | 465.0 | ¹H NMR (400 MHz, DMSO-d₆) δ 8.18 (s, 1H), 7.63-7.48 (m, 6H), 6.64 (d, J = 2.8 Hz, 1H), 5.08 (br, 1H), 4.49 (br, 1H), 4.15-4.09 (m, 1H), 2.68-2.61 (m, 1H), 2.55 (s, 3H), 2.14-2.07 (m, 1H). |
| 223 | 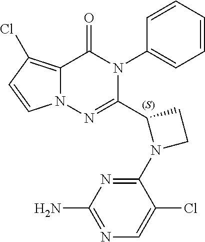 | 428.0 | ¹H NMR (400 MHz, DMSO-d₆) δ 7.71 (d, J = 2.8 Hz, 1H), 7.70 (s, 1H), 7.62-7.49 (m, 4H), 7.39-7.37 (m, 1H), 6.64 (d, J = 3.0 Hz, 1H), 6.28 (s, 2H), 4.81-4.77 (m, 1H), 4.18-4.12 (m, 1H), 4.02-3.96 (m, 1H), 2.46-2.39 (m, 1H), 2.01-1.95 (m, 1H). |
| 224 | 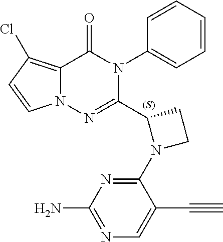 | 418.0 | ¹H NMR (400 MHz, CDCl₃) δ 8.00 (s, 1H), 7.60-7.47 (m, 3H), 7.40 (d, J = 7.5 Hz, 1H), 7.30 (d, J = 2.9 Hz, 1H), 7.15-7.10 (m, 1H), 6.49 (d, J = 3.0 Hz, 1H), 5.13-5.03 (m, 1H), 4.85 (s, 2H), 4.39-4.34 (m, 1H), 4.16-4.07 (m, 1H), 3.14 (s, 1H), 2.38-2.18 (m, 2H). |
| 225 | 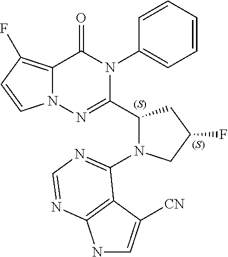 | 459.0 | ¹H NMR (400 MHz, DMSO-d₆) δ 8.33 (s, 1H), 8.31 (s, 1H), 7.69 (d, J = 8.7 Hz, 1H), 7.64-7.46 (m, 4H), 7.41-7.39 (m, 1H), 6.37 (d, J = 3.2 Hz, 1H), 5.55(br, 0.5H), 5.42(br, 0.5H), 4.87 (dd, J = 9.6, 3.0 Hz, 1H), 4.42-4.22 (m, 2H), 2.60-2.50 (m, 1H), 2.32-2.12 (m, 1H). |
| 226 | 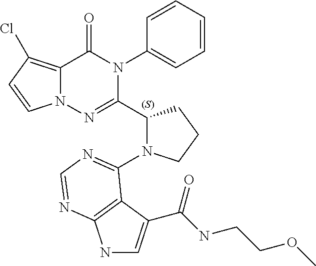 | 533.2 | ¹H NMR (400 MHz, DMSO-d₆) δ 8.28 (t, J = 5.5 Hz, 1H), 8.15 (s, 1H), 7.73 (d, J = 8.0 Hz, 1H), 7.64-7.49 (m, 4H), 7.46 (d, J = 2.9 Hz, 1H), 7.31 (s, 1H), 6.56 (d, J = 2.8 Hz, 1H), 4.53 (dd, J = 7.6, 4.1 Hz, 1H), 3.97-3.86 (m, 1H), 3.77-3.68 (m, 1H), 3.44-3.38 (m, 4H), 3.23 (s, 3H), 2.17-2.04 (m, 2H), 1.93-1.82 (m, 1H), 1.79-1.68 (m, 1H). |
| 227 | 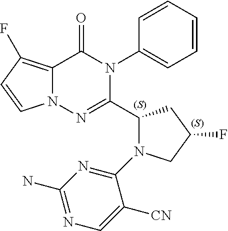 | 435.0 | ¹H NMR (400 MHz, DMSO-d₆) δ 8.23 (s, 1H), 7.84 (d, J = 7.8 Hz, 1H), 7.64-7.41 (m, 5H), 7.15 (s, 2H), 6.42 (d, J = 3.1 Hz, 1H), 5.38 (br, 0.5H), 5.26(br, 0.5H), 4.83 (br, 1H), 4.34-3.97 (m, 2H), 2.40-2.28 (m, 1H), 2.08-1.90 (m, 1H). |
| 228 | 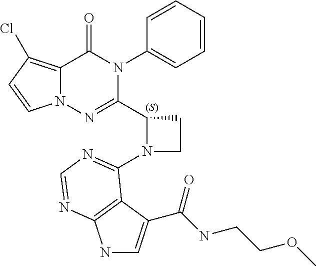 | 519.1 | ¹H NMR (400 MHz, DMSO-d₆) δ 8.17-8.11 (m, 2H), 7.62-7.55 (m, 3H), 7.54-7.48 (m, 2H), 7.47-7.41 (m, 2H), 6.62 (d, J = 3.0 Hz, 1H), 5.05 (t, J = 7.8 Hz, 1H), 4.22-4.21 (m, 1H), 3.75-3.74 (m, 1H), 3.43-3.36 (m, 4H), 3.19 (s, 3H), 2.07-1.67 (m, 2H). |
| 229 | 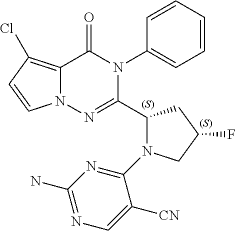 | 451.0 | ¹H NMR (400 MHz, DMSO-d₆) δ 8.23 (s, 1H), 7.84 (d, J = 7.8 Hz, 1H), 7.66-7.45 (m, 5H), 7.10 (s, 2H), 6.59 (d, J = 3.0 Hz, 1H), 5.39-5.26(m, 1H), 4.83 (br, 1H), 4.22-3.99 (m, 2H), 2.42-2.29 (m, 1H), 2.10-1.93 (m, 1H). |
| 230 | 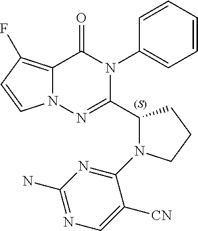 | 417.1 | ¹H NMR (400 MHz, DMSO-d₆) δ 8.18 (s, 1H), 7.85 (d, J = 7.7 Hz, 1H), 7.62-7.40 (m, 5H), 6.96 (s, 2H), 6.41 (d, J = 3.2 Hz, 1H), 4.60 (br, 1H), 3.94 (br, 1H), 3.76-3.74 (m, 1H), 2.15-1.99 (m, 2H), 1.96-1.82 (m, 1H), 1.70-1.60 (m, 1H). |
| 231 | 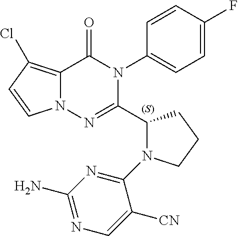 | 451.0 | ¹H NMR (400 MHz, DMSO-d₆) δ 8.19 (s, 1H), 7.93-7.89 (m, 1H), 7.61-7.58 (m, 2H), 7.40-7.35 (m, 2H), 7.05 (brs, 2H), 6.59 (d, J = 3.0 Hz, 1H), 4.57 (d, J = 7.4 Hz, 1H), 3.95 (brs, 1H), 3.78-3.72 (m, 1H), 2.07-2.00(m, 2H), 1.98-1.92 (m, 1H), 1.71-1.68 (m, 1H). |
| 232 | 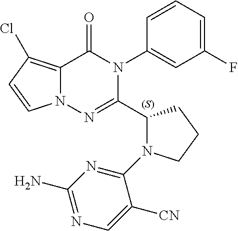 | 451.0 | ¹H NMR (400 MHz, DMSO-d₆) δ 8.21 (d, J = 2.0 Hz, 1H), 7.92 (d, J = 9.6 Hz, 0.5H), 7.78 (d, J = 7.6 Hz, 0.5H), 7.67-7.6274 (m, 1H), 7.60-7.55 (m, 2H), 7.46-7.38 (m, 2H), 7.04 (s, 1H), 6.64-6.63 (m, 1H), 4.64-4.53 (m, 1H), 4.01-3.92 (m, 1H), 3.80-3.74 (m, 1H), 2.16-2.06 (m, 2H), 1.99-1.90 (m, 1H), 1.78-1.68 (m, 1H). |
| 233 | 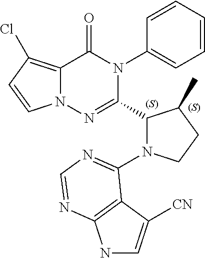 | 471.0 | ¹H NMR (400 MHz, DMSO-d₆) δ 8.21 (s, 1H), 8.16 (s, 1H), 7.76-7.74 (m, 1H), 7.63- 7.51 (m, 5H), 6.56 (d, J = 3.2 Hz, 1H), 4.37 (d, J = 0.8 Hz, 1H), 4.12-4.08 (m, 1H), 4.02- 3.96 (m, 1H), 2.64-2.59 (m, 1H), 2.54-2.50 (m, 1H), 1.81-1.75 (m, 1H), 0.62 (d, J = 6.8 Hz, 3H). |
| 234 | 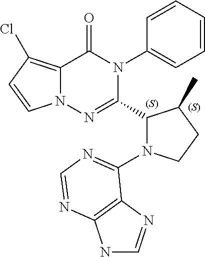 | 447.1 | ¹H NMR (400 MHz, DMSO-d₆) δ 8.13 (s, 1H), 7.98 (s, 1H), 7.62-7.49 (m, 6H), 6.55 (s, 1H), 5.07 (br, 0.5H), 4.36 (br, 0.5H), 3.92 (br, 1H), 3.69 (br, 1H), 2.62-2.58 (m, 1H), 2.17 (br, 1H), 1.60 (br, 1H), 0.38 (br, 3H). |
| 235 | 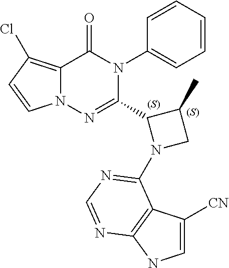 | 457.1 | ¹H NMR (400 MHz, CD₃OD) δ 8.04 (s, 1H), 7.86 (d, J = 6.9 Hz, 1H), 7.83 (s, 1H), 7.65-7.59 (m, 1H), 7.59-7.54 (m, 2H), 7.44-7.37 (m, 1H), 7.34 (d, J = 3.0 Hz, 1H), 6.49 (d, J = 3.1 Hz, 1H), 4.91-4.89 (m, 1H), 4.71-4.65 (m, 1H), 3.75 (dd, J = 8.2, 4.4 Hz, 1H), 2.78-2.60 (m, 1H), 0.54 (d, J = 6.8 Hz, 3H). |
| 236 | 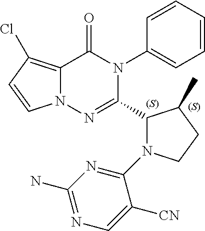 | 446.9 | ¹H NMR (400 MHz, DMSO-d₆) δ 8.22 (s, 1H), 7.91 (d, J = 2.4 Hz, 1H), 7.63 (d, J = 2.8 Hz, 1H), 7.60-7.59 (m, 3H), 7.56-7.52 (m, 1H), 7.11 (br, 2H), 6.63 (d, J = 2.8 Hz, 1H), 4.328 (br, 1H), 4.024 (br, 1H), 3.875 (br, 1H), 2.277 (br, 1H), 2.026-1.988 (m, 0.5H), 1.683 (br, 1H), 1.453-1.386 (m, 0.5H), 0.420 (d, J = 6.8 Hz, 3H). |
| 237 | 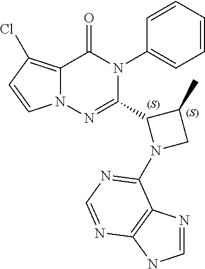 | 433.1 | ¹H NMR (400 MHz, DMSO-d₆) δ 8.18 (s, 1H), 8.10 (s, 1H), 7.75-7.47 (m, 6H), 6.59 (d, J = 2.4 Hz, 1H), 4.90-4.60 (m, 1H), 4.48-4.24 (m, 1H), 3.70-3.60 (m, 1H), 2.96-2.84 (m, 1H), 0.71 (d, J = 6.4 Hz, 3H). |
| 238 | 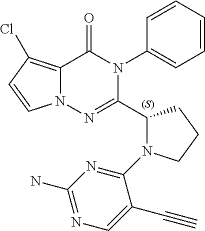 | 432.1 | ¹H NMR (400 MHz, CD₃OD) δ 7.90 (s, 1H), 7.76 (d, J = 7.8 Hz, 1H), 7.65-7.51 (m, 3H), 7.44-7.39 (m, 1H), 7.38 (d, J = 3.0 Hz, 1H), 6.49 (d, J = 3.0 Hz, 1H), 4.64-4.52 (m, 1H), 4.35-4.20 (m, 1H), 4.14-4.06 (m, 1H), 3.56 (s, 1H), 2.22-2.10 (m, 1H), 2.05-2.01 (m, 1H), 1.96-1.86 (m, 1H), 1.84-1.72 (m, 1H). |
| 239 | 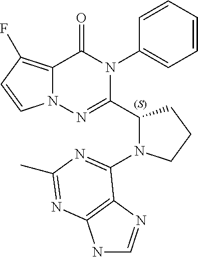 | 430.9 | ¹H NMR (400 MHz, CD₃OD) δ 8.30-8.12 (m, 0.5H), 7.96-7.93 (m, 1H), 7.77-7.75 (m, 0.5H), 7.58-7.45 (m, 4H), 7.17-7.09 (m, 1H), 6.25-6.20 (m, 1H), 5.53(br, 0.3H), 4.90 (br, 0.8H), 4.37 (br, 0.6H), 4.12 (br, 0.6H), 4.00 (br, 0.3H), 3.82 (br, 0.3H), 3.50 (s, 3H), 2.35 (br, 0.5H), 2.19 (br, 1H), 2.06 (br, 1H), 1.95 (br, 1.5H). |
| 240 | 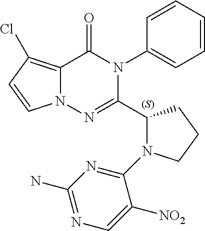 | 452.9 | ¹H NMR (400 MHz, DMSO-d₆) δ 8.59 (s, 1H), 7.87 (s, 1H), 7.54-7.49 (m, 5H), 7.25 (br, 2H), 6.59 (d, J = 2.8 Hz, 1H), 4.56 (br, 1H), 3.13-2.96 (m, 1H), 2.06-1.92 (m, 3H), 1.87-1.76 (m, 1H), 1.71-1.63 (m, 1H). |
| 241 | 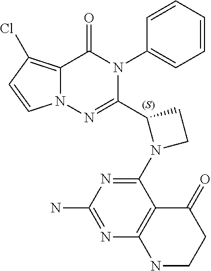 | 463.2 | ¹H NMR (400 MHz, CD₃OD) δ 7.62-7.30 (m, 7H), 6.52 (d, J = 3.0, 1H), 5.59 (br, 0.5H), 5.02 (br, 1H), 4.63 (br, 0.5H), 4.28 (br, 0.5H), 3.90-3.84 (m, 0.5H), 3.61-3.51 (m, 2H), 2.48-1.98 (m, 4H). |
| 242 | 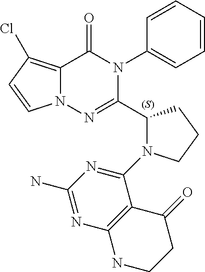 | 476.8 | ¹H NMR (400 MHz, DMSO-d₆) δ 7.88 (br, 1H), 7.57-7.50 (m, 5H), 7.37 (br, 1H), 6.60 (d, J = 3.0, 1H), 6.26 (br, 2H), 4.49 (br, 1H), 3.62 (br, 1H), 3.25-3.24 (m, 3H), 3.17- 3.16 (m, 1H), 2.37-2.25 (m, 2H), 2.03-1.94 (m, 2H), 1.84-1.77 (m, 1H), 1.63 (br, 1H). |
| 244 | 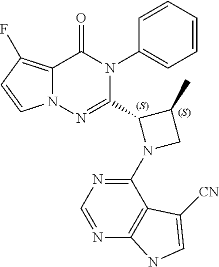 | 441.1 | ¹H NMR (400 MHz, CD₃OD) δ 8.24 (s, 1H), 7.94 (s, 1H), 7.81 (d, J = 7.4 Hz, 1H), 7.66-7.56 (m, 3H), 7.47-7.38 (m, 1H), 7.17 (br, 1H), 6.29 (d, J = 3.2 Hz, 1H), 4.98 (br, 1H), 4.68 (br, 1H), 3.84-3.81 (m, 1H), 2.79 (br, 1H), 0.64 (d, J = 6.7 Hz, 3H). |
| 245 | 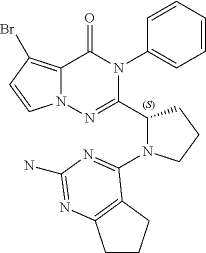 | 492.3 | ¹H NMR (400 MHz, DMSO-d₆) δ 7.82 (d, J = 8.0, 1H), 7.59-7.47 (m, 5H), 6.64 (d, J = 2.9, 1H), 5.65 (s, 2H), 4.52-4.49 (m, 1H), 3.90- 3.85 (m, 1H), 3.68-3.61 (m, 1H), 3.03-2.90 (m, 2H), 2.53-2.50 (m, 2H), 2.09-1.97 (m, 2H), 1.87-1.79 (m, 3H), 1.67-1.56 (m, 1H). |
| 246 | 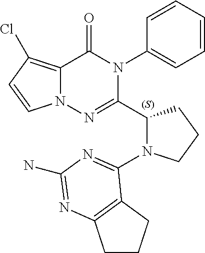 | 448.2 | ¹H NMR (400 MHz, DMSO-d₆) δ 7.82 (d, J = 8.0, 1H), 7.59-7.47 (m, 5H), 6.58 (d, J = 3.0, 1H), 5.65 (s, 2H), 4.52-4.49 (m, 1H), 3.90- 3.85 (m, 1H), 3.02-2.90 (m, 1H), 3.04-2.90 (m, 2H), 2.54-2.50 (m, 2H), 2.09-1.99 (m, 2H), 1.87-1.79 (m, 3H), 1.64-1.60 (m, 1H). |
| 247 | 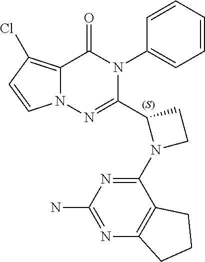 | 433.9 | ¹H NMR (400 MHz, DMSO-d₆) δ 7.73 (d, J = 2.7, 1H), 7.62-7.51 (m, 4H), 7.39 (br, 1H), 6.65 (d, J = 2.8, 1H), 5.82 (s, 2H), 4.69-4.66 (m, 1H), 3.94-3.83 (m, 2H), 2.71-2.45 (m, 4H), 2.07-1.70 (m, 4H). |
| 248 | 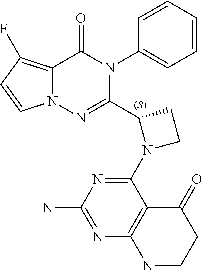 | 447.2 | ¹H NMR (400 MHz, DMSO-d₆) δ 7.75-7.60 (m, 5H), 7.43 (br, 1H), 6.52 (s, 1H), 4.82 (br, 1H), 4.49 (br, 1H), 3.75-3.70 (m, 1H), 3.37-3.34 (m, 2H), 2.41-2.38 (m, 3H), 1.87 (br, 1H). |
| 249 | 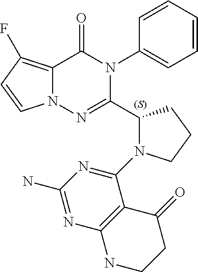 | 460.9 | ¹H NMR (400 MHz, DMSO-d₆) δ 8.49 (br, 0.4H), 7.83 (br, 0.6H), 7.53-7.47(m, 4H), 7.36-7.32 (m, 2H), 6.39 (d, J = 3.2, 1H), 6.20 (s, 2H), 4.49 (br, 1H), 3.62 (br, 1H), 3.23 (br, 3H), 2.30-2.22 (m, 2H), 1.97 (br, 2H), 1.77-1.76 (m, 1H), 1.61 (br, 1H). |
| 250 | 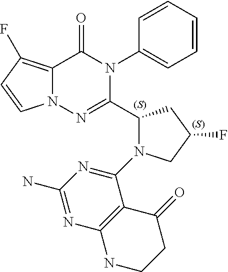 | 479 | ¹H NMR (400 MHz, DMSO-d₆) δ 7.56-7.48 (m, 5H), 7.41 (br, 1H), 7.38-7.36 (m, 1H), 6.41 (d, J = 3.2, 1H), 6.34 (s, 2H), 5.24 (br, 0.5H), 5.10 (br, 0.5H), 4.81 (br, 1H), 4.08-4.02 (m, 2H), 2.40-2.24 (m, 4H), 2.12-1.97 (m, 2H). |
| 261 | 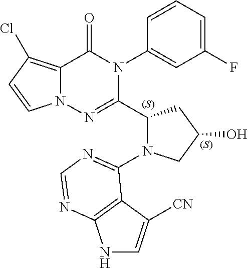 | 491.3 | ¹H NMR (400 MHz, CD₃OD) δ 8.29 (s, 1H), 7.99 (s, 1H), 7.71-7.54 (m, 2H), 7.40-7.25 (m, 3H), 6.54-6.40 (m, 1H), 4.95-3.88 (m, 1H), 4.48-4.41(m, 1H), 4.39-4.32 (m, 1H), 4.13-4.07 (m, 1H), 2.42-2.32 (m, 1H), 2.24-2.11 (m, 1H). |
| 262 | 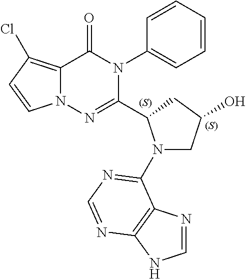 | 449.2 | ¹H NMR (400 MHz, DMSO-d₆) δ: 8.24-8.07 (m, 2H), 7.76-7.35 (m, 6H), 6.60-6.44 (m, 1H), 5.45-5.35 (br, 0.5H), 4.81-4.71 (br, 0.5H), 4.58-4.47 (br, 0.5H), 4.28-4.09 (br, 2H), 3.76-3.72 (br, 0.5H), 3.52-3.48 (br, 1H), 2.20-1.98 (m, 2H). |
| 430 | 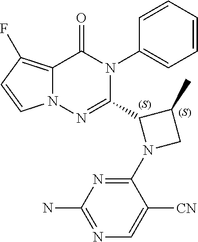 | 416.8 | ¹H NMR (400 MHz, DMSO-d₆) δ 8.15 (s, 1H), 7.58-7.19 (m, 6H), 7.09 (s, 2H), 6.48 (d, J = 3.0, 1H), 4.71 (br, 1H), 4.21 (br, 1H), 3.52 (br, 1H), 2.75 (br, 1H), 0.61 (br, 3H). |
| 431 | 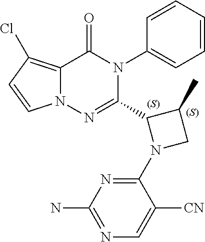 | 433.2 | ¹H NMR (400 MHz, CD₃OD) δ 8.09 (s, 1H), 7.61-7.57 (m, 4H), 7.40 (br, 1H), 7.38-7.35 (m, 1H), 6.55-6.53(m, 1H), 4.82 (s, 1H), 4.38 (br, 1H), 3.64 (br, 1H), 2.68 (br, 1H), 0.71 (d, J = 6.6, 3H). |
| 432 | 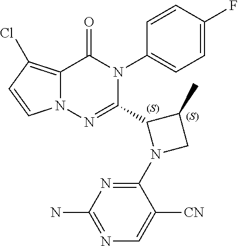 | 450.8 | ¹H NMR (400 MHz, DMSO-d₆) δ 8.14 (s, 1H), 7.69-7.40 (m, 5H), 7.08 (s, 2H), 6.64 (d, J = 3.0, 1H), 4.74 (br, 1H), 4.29 (br, 1H), 3.58 (br, 1H), 2.79 (br, 1H), 0.71 (br, 3H). |
| 433 | 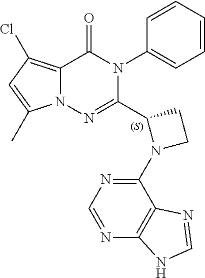 | 433.4 | ¹H NMR (400 MHz, CD₃OD) δ 8.25 (br, 1H), 7.97 (s, 1H), 7.70 (d, J = 7.6 Hz, 1H), 7.63-7.53 (m, 3H), 7.36-7.30 (m, 1H), 6.91-6.90 (m, 1H), 5.41-5.26 (m, 1H), 4.56-4.44 (m, 1H), 4.31-4.17 (m, 1H), 2.61-2.52 (m, 1H), 2.47-2.37 (m, 1H), 2.06 (s, 3H). |
| 461 | 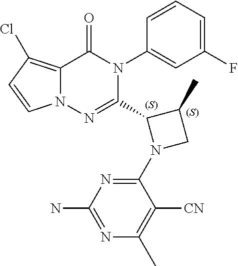 | 465.1 | ¹H NMR (400 MHz, CDCl₃) δ 7.60-7.48 (m, 1H), 7.39 (s, 1H), 7.30-7.26 (m, 1H), 7.25-7.21 (m, 1H), 6.97 (dd, J = 24.5, 8.1, 1H), 6.49 (d, J = 2.9, 1H), 5.06 (s, 2H), 4.89-4.68 (m, 1H), 4.64-4.29 (m, 1H), 3.89- 3.55 (m, 1H), 2.72-2.46 (m, 1H), 2.37 (s, 3H), 0.87 (d, J = 6.6, 3H). |
| 462 | 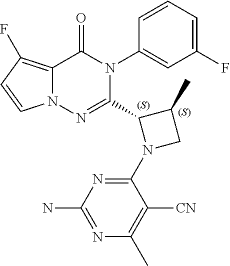 | 449.2 | ¹H NMR (400 MHz, CD3OD) δ 7.68-7.55 (m, 1H), 7.48 (d, J = 7.9, 1H), 7.42-7.29 (m, 2H), 7.27-7.19 (m, 1H), 6.34 (d, J = 3.2, 1H), 4.88-4.79 (m, 1H), 4.50-4.35 (m, 1H), 3.88-3.50 (m, 1H), 2.90-2.57 (m, 1H), 2.30 (s, 3H), 0.81 (dd, J = 6.8, 2.6, 3H). |
| 463 | 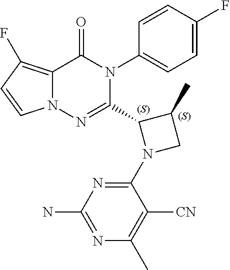 | 449.2 | ¹H NMR (400 MHz, CD3OD) δ 7.72-7.61 (m, 1H), 7.43-7.38 (m, 1H), 7.37-7.28 (m, 3H), 6.33 (d, J = 3.2, 1H), 4.88-4.79 (m, 1H), 4.49-4.35 (m, 1H), 3.78-3.57 (m, 1H), 2.84-2.57 (m, 1H), 2.30 (s, 3H), 0.80 (d, J = 6.8, 3H). |
| 464 | 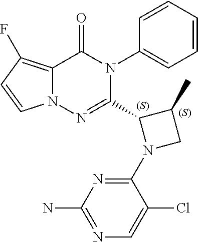 | 425.9 | ¹H NMR (400 MHz, DMSO-d6) δ 7.68 (s, 1H), 7.61- 7.51 (m, 5H), 7.45-7.43 (m, 1H), 6.45 (d, J = 3.2, 1H), 6.25 (s, 2H), 4.49 (br, 1H), 4.36-4.31(m, 1H), 3.58 (br, 1H), 2.75-2.66 (m, 1H), 0.68 (d, J = 6.8, 3H). |
| 465 | 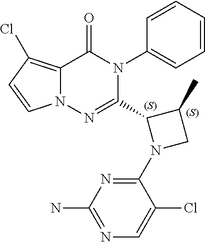 | 441.8 | ¹H NMR (400 MHz, CD3OD) δ 7.57-7.43 (m, 5H), 7.29 (d, J = 3.0, 1H), 7.28-7.29 (m, 1H), 6.45-6.43 (m, 1H), 4.64-4.62 (m, 1H), 4.45-4.41 (m, 1H), 3.61- 3.57 (m, 1H), 2.62-2.53 (m, 1H), 0.71 (d, J = 6.9, 3H). |
| 466⁴ | 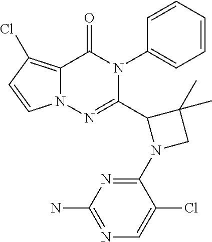 [SMILES: CC1(C)CN(c2nc(N)ncc2Cl)C1c1nn2ccc(Cl)c2c(=O)n1-c1ccccc1] | 455.8 | ¹H NMR (400 MHz, CD3OD) δ 7.63-7.45 (m, 5H), 7.37 (d, J = 2.4, 1H), 7.27 (d, J = 6.4, 1H), 6.46 (d, J = 3.0, 1H), 4.81 (br, 1H), 4.00 (br, 1H), 3.75 (br, 1H), 1.15 (s, 3H), 0.72 (s, 3H). |
| 467 | 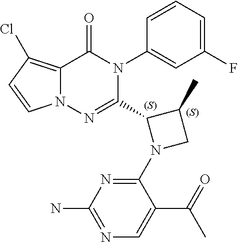 | 467.8 | ¹H NMR (400 MHz, CD3OD) δ 8.33 (s, 1H), 7.61- 7.49 (m, 1H), 7.47-7.44 (m, 1H), 7.29-7.22 (m, 2H), 7.16-7.10 (m, 1H), 6.43 (d, J = 3.0, 1H), 4.84 (br, 1H), 4.31 (br, 1H), 3.30 (br, 1H), 2.50 (br, 1H), 2.17 (s, 3H), 0.69 (d, J = 6.8, 3H). |
| 468 | 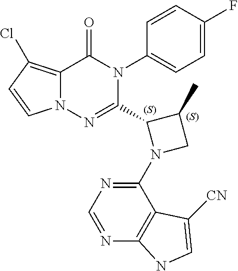 | 474.8 | ¹H NMR (400 MHz, CD3OD) δ 8.15 (s, 1H), 7.83 (s, 1H), 7.78-7.73 (m, 1H), 7.39-7.33 (m, 1H), 7.30- 7.17 (m, 3H), 6.39 (d, J = 3.0, 1H), 4.89-4.87 (m, 1H), 4.65-4.57 (m, 1H), 3.78-3.74 (m, 1H), 2.78- 2.71 (m, 1H), 0.67 (d, J = 6.8, 3H). |
| 469 | 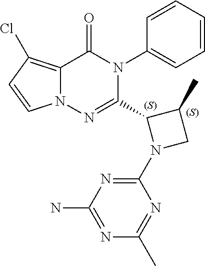 | 422.9 | ¹H NMR (400 MHz, CD3OD) δ 7.54-7.46 (m, 4H), 7.32 (d, J = 2.8, 1H), 7.33-7.26 (m, 1H), 6.46-6.44 (m, 1H), 4.42 (br, 1H), 4.17-4.12 (m, 1H), 3.42-3.38 (m, 1H), 2.58 (br, 1H), 2.12 (s, 3H), 0.63 (d, J = 6.4, 3H). |
| 470 | 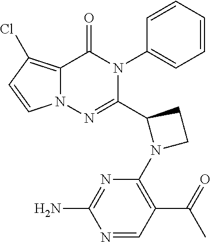 [SMILES: CC(=O)c1cnc(N)nc1N1CC[C@@H]1c1nn2ccc(Cl)c2c(=O)n1-c1ccccc1] | 436 | ¹H NMR (400 MHz, CD3OD) δ 8.34 (s, 1H), 7.58- 7.54 (m, 2H), 7.51-7.45 (m, 2H), 7.28 (d, J = 2.7 Hz, 1H), 7.24-7.21 (m, 1H), 6.45 (d, J = 3.0 Hz, 1H), 5.15 (brs, 1H), 4.25-4.19 (m, 1H), 3.69 (brs, 1H), 2.32- 2.24 (m, 1H), 2.19 (s, 3H), 2.08-1.98 (m, 1H). |
| 471⁴ | 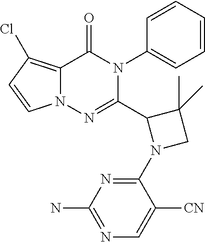 [SMILES: CC1(C)CN(c2nc(N)ncc2C#N)C1c1nn2ccc(Cl)c2c(=O)n1-c1ccccc1] | 447.2 | ¹H NMR (400 MHz, CD₃OD) δ 8.10 (s, 1H), 7.75 (s, 1H), 7.63-7.54 (m, 3H), 7.47 (s, 1H), 7.38 (d, J = 6.4, 1H), 6.56 (dd, J = 3.0, 1.7, 1H), 5.34-4.84 (m, 1H), 4.25-3.60 (m, 2H), 1.23 (s, 3H), 0.76 (s, 3H). |
| 491 | 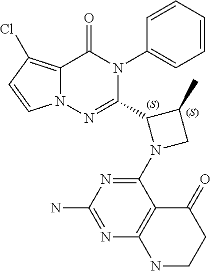 | 477.8 | ¹H NMR (400 MHz, CD₃OD) δ 8.61-8.58 (m, 1H), 8.02-7.98 (m, 1H), 7.64 (d, J = 7.6, 1H), 7.53-7.51 (m, 1H), 7.32 (br, 1H), 6.45 (d, J = 2.8, 1H), 4.73 (br, 2H), 4.50 (br, 1H), 3.25 (br, 2H), 2.59 (br, 1H), 2.39 (br, 1H), 2.25 (br, 1H), 0.75 (br, 3H). |
| 492 | 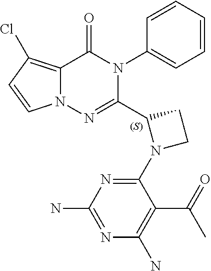 | 450.8 | ¹H NMR (400 MHz, CD₃OD) δ 7.61-7.59 (m, 2H), 7.55-7.52 (m, 2H), 7.45-7.43 (m, 1H), 7.33-7.30 (m, 1H), 6.55-6.53 (m, 1H), 5.18-5.13 (m, 1H), 4.21- 4.15 (m, 1H), 3.62-3.50 (m, 1H), 2.49 (s, 3H), 2.46- 2.39 (m, 1H), 2.05-1.95 (m, 1H). |
| 493 | 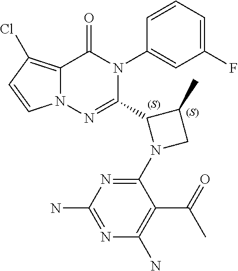 | 483.1 | ¹H NMR (400 MHz, DMSO-d₆) δ 7.74-7.26 (m, 7H), 6.66-6.65 (m, 1H), 6.33 (s, 2H), 6.30 (s, 1H), 4.65 (d, J = 5.2, 0.5H), 4.58 (d, J = 5.2, 0.5H), 4.22-4.16 (m, 1H), 3.06-3.00 (m, 1H), 2.74 (br, 1H), 2.35 (s, 1.5H), 2.34 (s, 1.5H), 0.58 (d, J = 6.8, 1.5H), 0.54 (d, J = 6.4, 1.5H). |
| 494 | 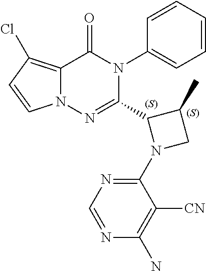 | 433 | ¹H NMR (400 MHz, CD₃OD) δ 7.90 (s, 1H), 7.54- 7.50 (m, 4H), 7.33 (br, 1H), 7.30 (br, 1H), 6.46 (d, J = 3.0, 1H), 4.75 (br, 1H), 4.39 (br, 1H), 3.61 (br, 1H), 2.62 (br, 1H), 0.63 (d, J = 6.8, 3H). |
| 495 | 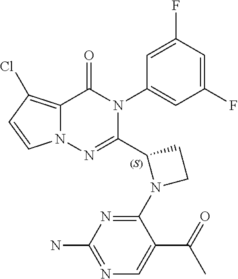 | 472.1 | ¹H NMR (400 MHz, CD₃OD) δ 8.43 (s, 1H), 7.41-7.36 (m, 2H), 7.26-7.19 (m, 1H), 7.09- 7.06 (m, 1H), 6.54-6.52 (m, 1H), 5.25 (br, 1H), 4.35-4.28 (m, 1H), 3.78 (br, 1H), 2.43- 2.18 (m, 5H). |
| 496 | 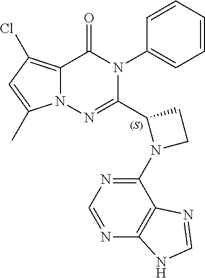 | 433.4 | ¹H NMR (400 MHz, CD₃OD) δ 8.25 (bs, 1H), 7.97 (s, 1H), 7.70 (d, J = 7.6 Hz, 1H), 7.63-7.53 (m, 3H), 7.36-7.30 (m, 1H), 6.91-6.90 (m, 1H), 5.41-5.26 (m, 1H), 4.56-4.44 (m, 1H), 4.31-4.17 (m, 1H), 2.61-2.52 (m, 1H), 2.47-2.37 (m, 1H), 2.06 (s, 3H). |
| 509 | 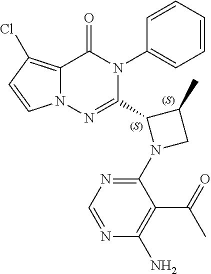 | 450.1 | 1H NMR (400 MHz, CD₃OD) δ 7.96 (s, 1H), 7.65-7.54 (m, 4H), 7.42-7.38 (m, 2H), 6.55 (d, J = 2.8, 1H), 4.84 (d, J = 4.4, 1H), 4.43 (t, J = 8.0, 1H), 3.19 (dd, J = 8.0, 4.8, 1H), 2.76-2.69 (m, 1H), 2.49 (s, 3H), 0.55 (d, J = 6.8, 3H). |
| 518 | 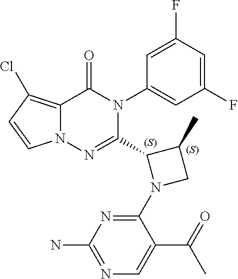 | 486.1 | 1H NMR (400 MHz, DMSO-d6) δ 8.44 (s, 1H), 7.64-7.40 (m, 4H), 6.84 (s, 2H), 6.64 (d, J = 2.8, 1H), 4.45-4.28 (m, 2H), 3.12 (br, 1H), 2.72 (br, 1H), 2.26 (s, 3H), 0.77 (d, J = 6.6, 3H). |

⁴prepared from (S)-methyl 3,3-dimethylazetidine-2-carboxylate

### Example 2

### Compound 59

### (S)-4-(2-(5-chloro-3-(2,2-difluoroethyl)-4-oxo-3,4-dihydropyrrolo[2,1-f][1,2,4]triazin-2-yl)azetidin-1-yl)-7H-pyrrolo[2,3-d]pyrimidine-5-carbonitrile

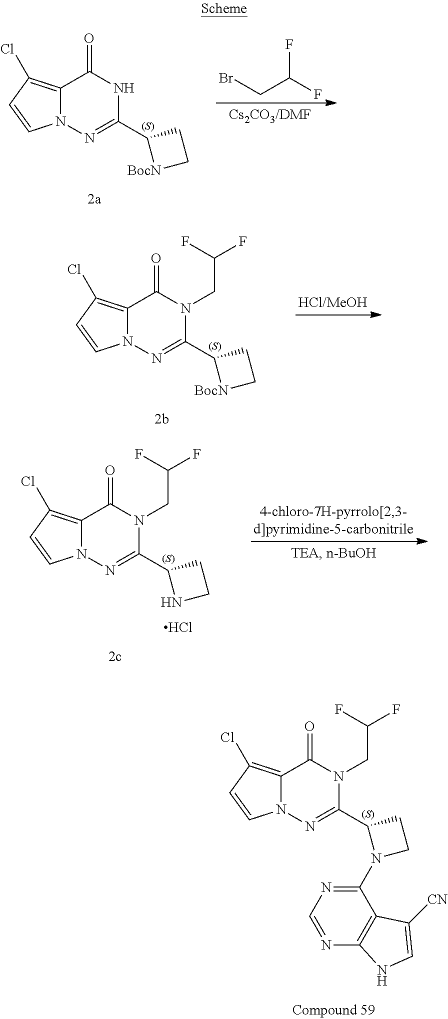

### Step 2-1 (S)-tert-butyl 2-(5-chloro-3-(2,2-difluoroethyl)-4-oxo-3,4-dihydropyrrolo[2,1-f][1,2,4]triazin-2-yl)azetidine-1-carboxylate (2b)

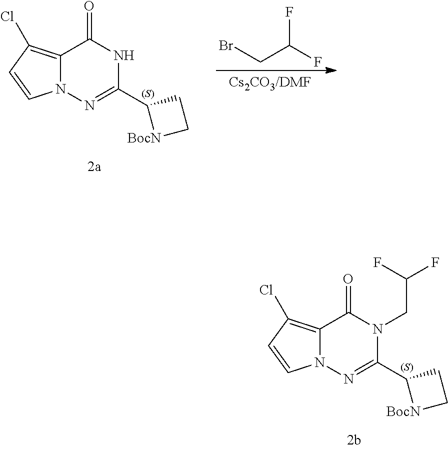

To a mixture of 2a (740 mg, 2.28 mmol) (2a was prepared according to the procedure of Example 1 using 1-amino-3-chloro-1H-pyrrole-2-carboxamide and (S)-azetidine-2-carboxylic acid instead of 1a and (S)-1-(tert-butoxycarbonyl) pyrrolidine-2-carboxylic acid) and Cs₂CO₃ (1.6 g, 4.92 mmol) in DMF (7 mL) was added 2-bromo-1,1-difluoroethane (0.4 mL, 5.02 mmol). The reaction was heated to 50° C. for one hour and 120° C. for another 1.5 hours. Then the mixture was diluted with water and extracted with EtOAc three times. The combined organic layers were washed with brine, dried over MgSO₄, filtered and concentrated to give the crude product which was further purified by flash column chromatography eluting with EtOAc/PE. 230 mg of 2b was obtained (yield: 26%) and 110 mg of 2a were recovered. MS (m/z): 289.0 (M-Boc+H)⁺.

### Step 2-2 (S)-2-(azetidin-2-yl)-5-chloro-3-(2,2-difluoroethyl)pyrrolo[2,1-f][1,2,4]triazin-4(3H)-one hydrochloride (2c)

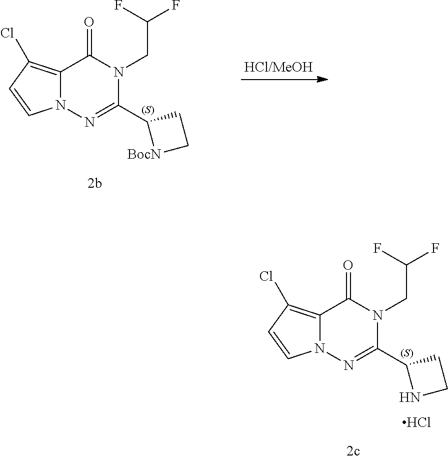

To a mixture of 2b (230 mg, 0.59 mmol) in MeOH (2 mL) was added conc. HCl aq. (2 mL), then the reaction was stirred at room temperature for about 3 hours. After concentration, 2c was obtained as a pale yellow solid which was used in the next step without further purification. MS (m/z): 289.0 (M+H)⁺.

### Step 2-3 (S)-4-(2-(5-chloro-3-(2,2-difluoroethyl)-4-oxo-3,4-dihydropyrrolo[2,1-f][1,2,4]triazin-2-yl)azetidin-1-yl)-7H-pyrrolo[2,3-d]pyrimidine-5-carbonitrile (59)

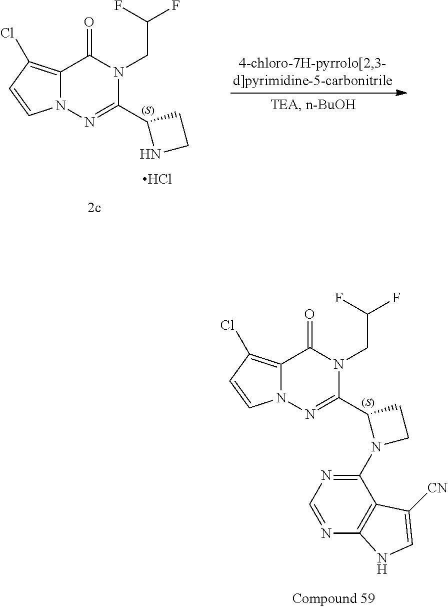

A mixture of 2c (0.59 mmol), 4-chloro-7H-pyrrolo[2,3-d]pyrimidine-5-carbonitrile (105 mg, 0.59 mmol) and TEA (0.41 mL, 2.95 mmol) in n-BuOH (9 mL) was heated at 130° C. for 2 hours. After concentration, the residue was washed with water and dried, then purified by preparative TLC and Compound 59 as a pale yellow solid was obtained (160 mg, yield: 63%). MS (m/z): 431.1 (M+H)⁺. ¹H NMR (400 MHz, DMSO-d₆) δ: 12.94 (s, 1H), 8.32 (m, 2H), 7.67 (s, 1H), 6.67 (s, 1H), 6.45 (t, J=55.2 Hz, 1H), 5.92-5.82 (m, 1H), 4.80-4.54 (m, 2H), 4.52-4.26 (m, 2H), 3.06-2.96 (m, 1H), 2.78-2.66 (m, 1H)

The following Compounds were prepared according to the procedure of Compound 59 using the corresponding reagents and intermediates under appropriate conditions that will be recognized by one skilled in the art:

| Compd. | | LC/MS | |
|---|---|---|---|
| No. | Structure | (M + H)⁺ | NMR |
| 60 | 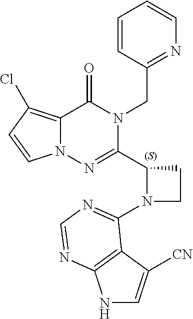 | 457.9 | ¹H NMR (400 MHz, DMSO-d₆) δ: 8.51-8.41 (m, 1H), 8.25-8.14 (m, 2H), 7.83-7.73 (m, 1H), 7.62-7.62 (m, 1H), 7.50-7.40 (m, 1H), 7.31-7.20 (m, 1H), 6.84 (s, 1H), 6.64-6.56 (m, 1H), 5.76-5.64 (m, 1H), 5.45-5.31 (m, 1H), 5.31-5.25 (m, 1H), 4.52-4.46 (m, 1H), 4.30-4.24 (m, 1H), 2.58-2.52 (m, 1H), 2.03-1.88 (m, 1H). |
| 61 | 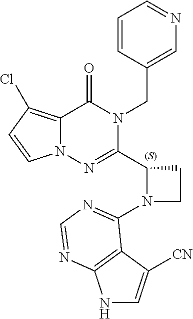 | 457.9 | ¹H NMR (400 MHz, DMSO-d₆) δ: 11.67 (s, 1H), 8.67-8.57 (m, 1H), 8.52-8.40 (m, 1H), 8.25-8.11 (m, 2H), 7.83-7.71 (m, 1H), 7.64-7.54 (m, 1H), 7.44-7.32 (m, 1H), 6.66-6.54 (m, 1H), 5.74-5.62 (m, 1H), 5.43-5.33 (m, 1H), 5.10-5.00 (m, 1H), 4.56-4.46 (m, 1H), 4.34-4.26 (m, 1H), 2.74-2.62 (m, 1H), 1.99-1.87 (m, 1H). |
| 62 | 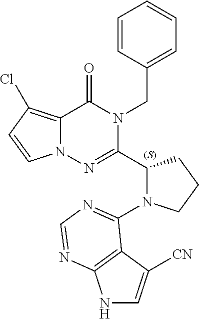 | 470.7 | ¹H NMR (400 MHz, DMSO-d₆) δ: 12.84 (s, 1H), 8.26 (s, 1H), 8.01 (s, 1H), 7.52-7.33 (m, 5H), 7.26 (dd, J = 17.6, 10.1 Hz, 1H), 6.58-6.45 (m, 1H), 5.47 (d, J = 16.6 Hz, 1H), 5.40-5.34 (m, 1H), 5.28 (d, J = 16.6 Hz, 1H), 4.19-4.07 (m, 1H), 4.00 (q, J =7.3 Hz, 1H), 2.33-2.20 (m, 1H), 2.20-2.03 (m, 2H), 2.03-1.91 (m, 1H). |
| 63 | 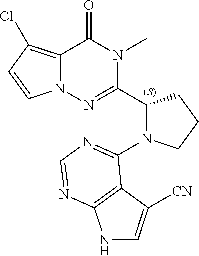 | 394.6 | ¹H NMR (400 MHz, DMSO-d₆) δ: 12.85 (s, 1H), 8.28 (s, 1H), 8.11 (s, 1H), 7.39 (d, J = 2.9 Hz, 1H), 6.48 (d, J = 2.9 Hz, 1H), 5.55 (dd, J = 7.8, 3.0 Hz, 1H), 4.18-4.02 (m, 2H), 3.58 (s, 3H), 2.44-2.36 (m, 1H), 2.33-2.11 (m, 3H). |
| 64 | 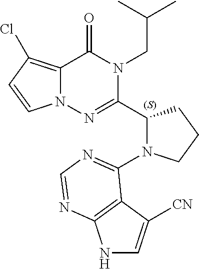 | 436.8 | ¹H NMR (400 MHz, DMSO-d₆) δ: 8.28 (s, 1H), 8.03 (s, 1H), 7.38 (d, J = 2.9 Hz, 1H), 6.48 (d, J = 2.9 Hz, 1H), 5.44 (d, J = 5.6 Hz, 1H), 4.22 (dd, J = 14.2, 8.4 Hz, 1H), 4.17-4.10 (m, 1H), 4.04 (d, J = 9.0 Hz, 1H), 3.65 (d, J = 6.9 Hz, 1H), 2.44-2.36 (m, 1H), 2.35-2.23 (m, 1H), 2.22-2.03 (m, 2H), 2.01-1.81 (m, 1H), 1.02 (d, J = 6.7 Hz, 3H), 0.94 (d, J = 6.6 Hz, 3H). |
| 65 | 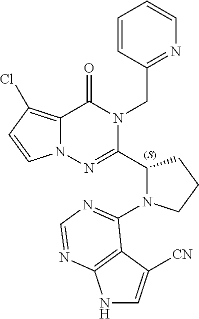 | 472.1 | ¹H NMR (400 MHz, DMSO-d₆) δ: 8.51 (s, 1H), 8.26 (s, 1H), 7.98-7.82 (m, 2H), 7.53-7.48 (m, 2H), 7.36-7.24 (br, 1H), 6.55 (s, 1H), 5.72-5.30 (m, 3H), 4.15-3.95 (m, 2H), 2.28-2.07 (m, 4H). |
| 66 | 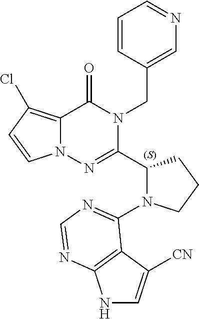 | 472.2 | ¹H NMR (400 MHz, DMSO-d₆) δ: 12.85 (s, 1H), 8.78-8.22 (m, 1H), 8.48 (s, 1H), 8.28 (s, 1H), 8.07-7.85 (br, 2H), 7.57-7.35 (br, 2H), 6.54 (s, 1H), 5.60-5.15 (m, 3H), 4.18-4.00 (m, 2H), 2.28-2.08 (m, 4H). |
| 67 | 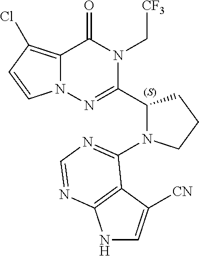 | 463.1 | ¹H NMR (400 MHz, DMSO-d₆) δ: 8.16 (s, 1H), 8.03 (s, 1H), 7.53 (s, 1H), 6.58 (s, 1H), 5.39 (s, 2H), 5.22-5.12 (m, 1H), 4.15-4.05 (m, 2H), 2.42-2.32 (m, 2H), 2.19-2.09 (m, 2H). |
| 68 | 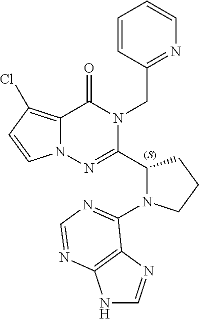 | 448.2 | ¹H NMR (400 MHz, DMSO-d₆) δ: 8.55-8.50 (m, 1H), 8.15-7.90 (m, 2H), 7.88-7.84 (m, 1H), 7.59-7.57 (m, 1H), 7.47-7.45 (m, 1H), 7.35-7.32 (m, 1H), 6.56-6.55 (m, 1H), 5.71-5.67 (m, 1H), 5.62-5.52 (m, 1H), 4.16-4.04 (m, 2H), 2.10-1.97 (m, 4H). |
| 69 | 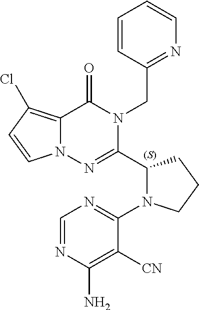 | 448.2 | ¹H NMR (400 MHz, DMSO-d₆) δ: 8.49 (s, 1H), 7.85-7.70 (m, 2H), 7.58-7.50 (br, 1H), 7.51-7.41 (m, 1H), 7.31-7.08 (m, 3H), 6.62-6.54 (br, 1H), 5.54-5.28 (m, 3H), 3.97- 3.86 (m, 2H), 2.11-1.98 (m, 4H). |
| 155 | 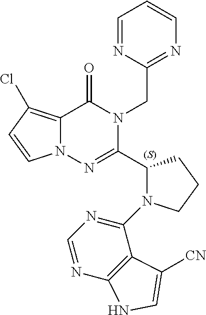 | 473.5 | ¹H NMR (400 MHz, CD₃OD) δ: 8.82 (d, J = 4.9 Hz, 2H), 8.06 (s, 1H), 8.01 (s, 1H), 7.44 (t, J = 4.9 Hz, 1H), 7.33 (d, J = 2.9 Hz, 1H), 6.50 (d, J = 2.9 Hz, 1H), 5.95 (d, J = 17.7 Hz, 1H), 5.77 (d, J = 17.7 Hz, 1H), 5.50 (t, J = 5.6 Hz, 1H), 4.40-4.28 (m, 1H), 4.21-4.07 (m, 1H), 2.55-2.46 (m, 1H), 2.26-2.18 (m, 3H). |
| 156 | 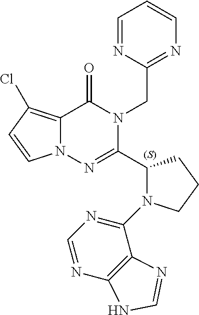 | 449.5 | ¹H NMR (400 MHz, CD₃OD) δ: 8.77 (s, 1H), 8.76 (s, 1H), 8.20-7.77 (m, 2H), 7.39 (t, J = 4.9 Hz, 1H), 7.19 (s, 1H), 6.40 (s, 1H), 5.88-5.76 (m, 2H), 5.70-5.64 (m, 0.5H), 5.42-5.32 (m, 0.5H), 4.40-4.34 (m, 0.5H), 4.23-4.17 (m, 0.5H), 4.07-4.01 (m, 0.5H), 3.85-3.75 (m, 0.5H), 2.35-2.11 (m, 4H). |
| 157 | 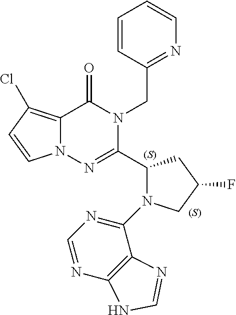 | 466.6 | ¹H NMR (400 MHz, DMSO-d₆) δ: 8.58 (s, 1H), 8.40-8.10 (m, 2H), 7.92 (t, J = 7.3 Hz, 1H), 7.62 (d, J = 7.9 Hz, 1H), 7.55-7.45 (m, 1H), 7.42-7.33 (m, 1H), 6.58 (s, 1H), 5.76-5.23 (m, 4H), 4.75-4.50 (br, 1H), 4.35-4.15 (br, 1H), 2.74-2.64 (m, 1H), 2.47-2.37 (m, 1H). |
| 158 | 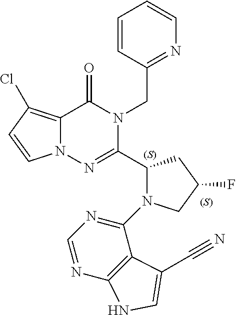 | 490.6 | ¹H NMR (400 MHz, DMSO-d₆) δ: 8.60-8.56 (m, 1H), 8.36 (s, 1H), 8.06 (s, 1H), 7.91 (t, J = 7.4 Hz, 1H), 7.63-7.47 (m, 2H), 7.39-7.33 (m, 1H), 6.62-6.56 (m, 1H), 5.72-5.62 (m, 2H), 5.60-5.50 (m, 1H), 5.31-5.23 (m, 1H), 4.55-4.34 (m, 2H), 2.74-2.66 (m, 1H), 2.59-2.55 (m, 1H). |
| 159 | 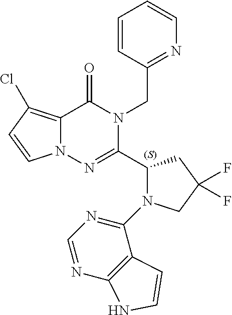 | 484.0 | ¹H NMR (400 MHz, CD₃OD) δ: 8.54 (d, J = 4.8 Hz, 1H), 8.40-8.02 (m, 2H), 7.89 (td, J = 7.8, 1.6 Hz, 1H), 7.58 (d, J = 7.9 Hz, 1H), 7.40-7.33 (m, 1H), 7.23 (s, 1H), 6.45 (s, 1H), 5.78-5.58 (m, 3H), 4.65-4.29 (m, 2H), 2.82-2.62 (m, 2H). |
| 160 | 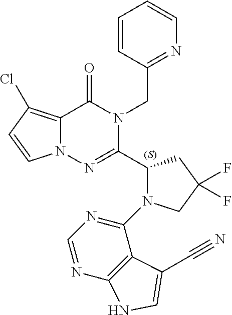 | 508.1 | ¹H NMR (400 MHz, CD₃OD) δ: 8.51 (d, J = 4.7 Hz, 1H), 8.13 (s, 1H), 8.06 (s, 1H), 7.87 (td, J = 7.7, 1.6 Hz, 1H), 7.53 (d, J = 7.9 Hz, 1H), 7.36-7.33 (m, 2H), 6.48 (d, J = 3.0 Hz, 1H), 5.82-5.61 (m, 3H), 4.70-4.54 (m, 2H), 2.86-2.74 (m, 1H), 2.69-2.62 (m, 1H). |
| 161 | 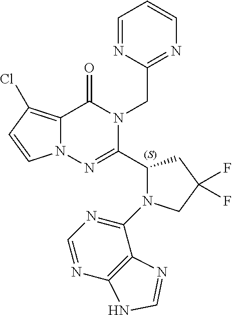 | 485.0 | ¹H NMR (400 MHz, CD₃OD) δ: 8.75 (d, J = 4.9 Hz, 2H), 8.01 (s, 1H), 7.82 (s, 1H), 7.38 (t, J = 5.0 Hz, 1H), 7.28 (d, J = 3.0 Hz, 1H), 6.46 (d, J = 3.0 Hz, 1H), 5.93-5.72 (m, 3H), 4.56-4.49 (m, 1H), 2.84-2.77 (m, 2H). |
| 162 | 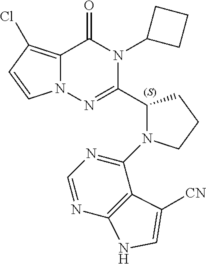 | 435.2 | ¹H NMR (400 MHz, CD₃OD) δ: 8.11 (s, 1H), 7.99 (s, 1H), 7.12 (d, J = 2.9 Hz, 1H), 6.36 (d, J = 2.9 Hz, 1H), 5.79-5.71 (m, 1H), 5.06-5.00 (m, 1H), 4.31-4.25 (m, 1H), 4.21-4.15 (m, 1H), 3.28-3.14 (m, 2H), 2.58-2.48 (m, 2H), 2.45-2.35 (m, 2H), 2.28-2.22 (m, 1H), 2.18-2.12 (m, 1H), 2.06-2.00 (m, 1H), 1.94-1.86 (m, 1H). |
| 251 | 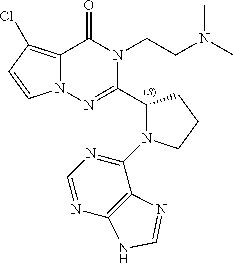 | 428.1 | ¹H NMR (400 MHz, CD₃OD) δ 7.97 (s, 1H), 7.78 (s, 1H), 7.16 (d, J = 3.2 Hz, 1H), 6.37 (d, J = 2.8 Hz, 1H), 4.75-4.65 (m, 2H), 4.38-4.30 (m, 0.5H), 4.19-4.1 (m, 0.5H), 3.99-3.92 (m, 1H), 3.33-3.32 (m, 1H), 2.82-2.75 (m, 1H), 2.52-2.44 (m, 1H), 2.40 (s, 6H), 2.33-2.24 (m, 2H), 2.19-2.12 (m, 2H). |
| 252 | 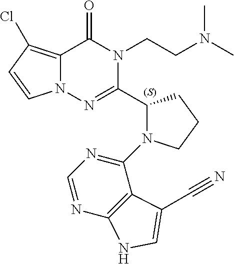 | 452.1 | 1H NMR (400 MHz, CD3OD) δ 8.11 (s, 1H), 7.99 (s, 1H), 7.19 (d, J = 3.2 Hz, 1H), 6.39 (d, J = 2.8 Hz, 1H), 4.75-4.65 (m, 2H), 4.37- 4.31 (m, .0.5H), 4.22-4.16 (m, 0.5H), 3.97-3.90 (m, 1H), 3.34-3.33 (m, 1H), 3.18-3.14 (m, 1H), 2.82-2.78 (m, 1H), 2.50-2.40 (m, 2H), 2.40 (s, 6H), 2.30-2.25 (m, 1H), 2.21-2.15 (m, 1H). |
| 253 | 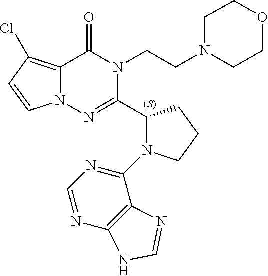 | 470.1 | 1H NMR (400 MHz, DMSO-d6) δ 12.97 (s, 1H), 8.26-8.00 (m, 2H), 7.42 (s, 0.5H), 7.36 (s, 0.5H), 6.51 (d, J = 9.9 Hz, 1H), 6.01 (d, J = 8.6 Hz, 0.5H), 5.47 (d, J = 6.6 Hz, 0.5H), 4.57-4.24 (m, 2H), 3.95-3.75 (m, 2H), 3.58 (br, 4H), 2.94-2.78 (m, 2H), 2.55 (br, 4H), 2.38-1.89 (m, 4H). |
| 255 | 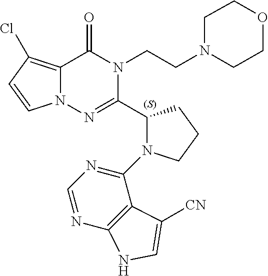 | 494.1 | 1H NMR (400 MHz, DMSO-d6) δ 8.17 (s, 1H), 8.04 (s, 1H), 7.44 (d, J = 3.0 Hz, 1H), 6.52 (d, J = 2.9 Hz, 1H), 5.50 (dd, J = 7.8, 2.8 Hz, 1H), 4.64-4.53 (m, 1H), 4.21-4.16 (m, 1H), 4.09-4.03 (m, 1H), 3.95-3.88 (m, 1H), 3.57 (t, J = 4.6 Hz, 4H), 2.98-2.96 (m, 1H), 2.82-2.72 (m, 1H), 2.57-2.43 (m, 5H), 2.33-2.29 (m, 1H), 2.23-2.08 (m, 2H). |
| 256 | 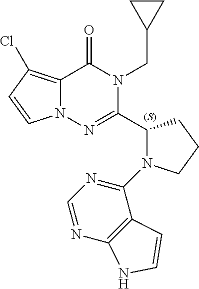 | 411.1 | 1H NMR (400 MHz, DMSO-d6) δ 8.24-7.94 (m, 2H), 7.39 (s, 0.5H), 7.34 (s, 0.5H), 6.50 (s, 0.5H), 6.47 (s, 0.5H), 6.05 (d, J = 8.3 Hz, 0.5H), 5.53 (d, J = 9.1 Hz, 0.5H), 4.41-4.12 (m, 2H), 3.99-3.72 (m, 2H), 2.31-2.00 (m, 4H), 1.70-1.55 (m, 1H), 0.64-0.44 (m, 4H). |
| 257 | 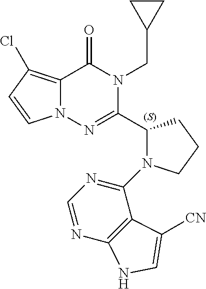 | 435.1 | 1H NMR (400 MHz, DMSO-d6) δ 8.22 (s, 1H), 8.06 (s, 1H), 7.44 (d, J = 3.0 Hz, 1H), 6.52 (d, J = 2.9 Hz, 1H), 5.60 (dd, J = 7.9, 3.0 Hz, 1H), 4.26-4.14 (m, 2H), 4.11-4.05 (m, 1H), 3.96-3.91 (m, 1H), 2.47-2.08 (m, 4H), 1.61- 1.55 (m, 1H), 0.64-0.53 (m, 4H). |
| 258 | 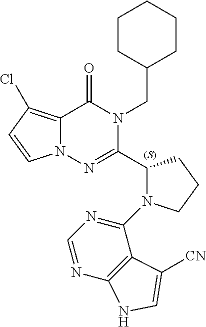 | 477.1 | 1H NMR (400 MHz, DMSO-d6) δ 8.21 (s, 1H), 8.00 (s, 1H), 7.39 (d, J = 3.0 Hz, 1H), 6.48 (d, J = 3.0 Hz, 1H), 5.46 (dd, J = 7.9, 2.7 Hz, 1H), 4.28-4.20 (m, 1H), 4.18-4.13 (m, 1H), 4.04 (dd, J = 16.7, 7.6 Hz, 1H), 3.63-3.58 (m, 1H), 2.45-2.03 (m, 5H), 1.89-1.57 (m, 5H), 1.24-1.00 (m, 5H). |
| 259 | 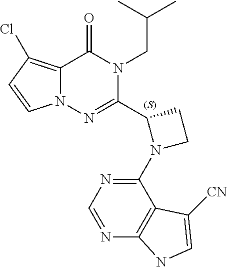 | 422.9 | 1H NMR (400 MHz, CD3OD) δ 8.19 (s, 1H), 7.97 (s, 1H), 7.30 (d, J = 3.2 Hz, 1H), 6.46 (d, J = 2.8 Hz, 1H), 5.86-5.82 (m, 1H), 4.82- 4.78 (m, 1H), 4.54-4.50 (m, 1H), 4.18-4.12 (m, 1H), 3.68-3.63 (m, 1H), 3.05-2.96 (m, 1H), 2.66-2.59 (m, 1H), 2.28-2.18 (m, 1H), 1.06-1.01 (m, 6H). |
| 260 | 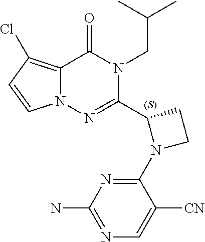 | 398.9 | 1H NMR (400 MHz, CD₃OD) δ 8.11 (s, 1H), 7.35 (s, 1H), 6.50-6.49 (m, 1H), 5.78 (br, 1H), 4.35 (br, 2H), 4.05-3.94 (m, 1H), 3.51 (br, 1H), 2.95 (br, 1H), 2.44 (br, 1H), 2.18- 2.07 (m, 1H), 1.01-0.96 (m, 6H). |

### Example 3

### Compound 70

### 4-((2S,4R)-2-(5-chloro-4-oxo-3-phenyl-3,4-dihydropyrrolo[2,1-f][1,2,4]triazin-2-yl)-4-hydroxypyrrolidin-1-yl)-7H-pyrrolo[2,3-d]pyrimidine-5-carbonitrile

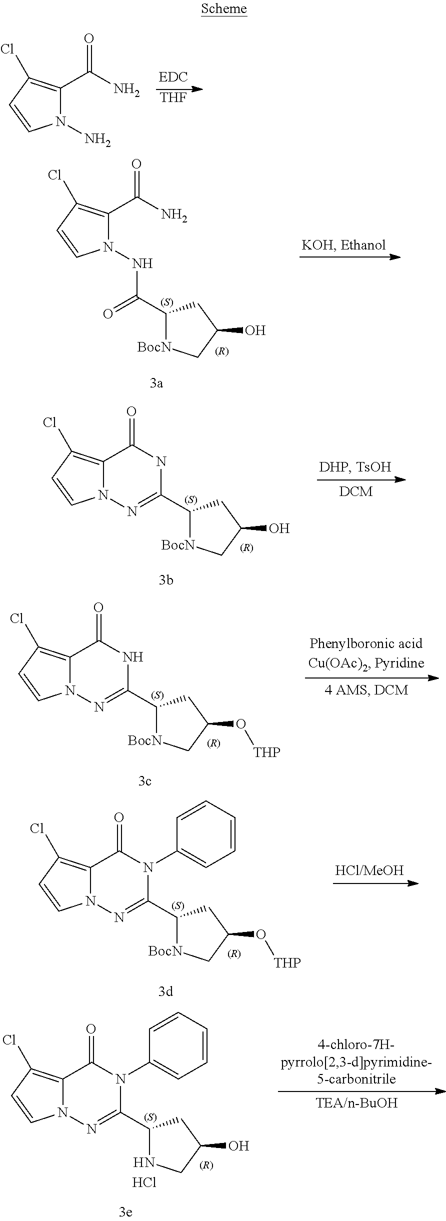

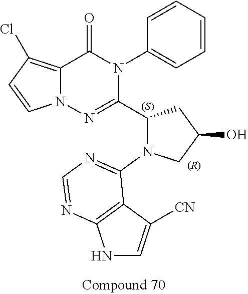

Synthesis of Compound 70 was carried out according to the procedure of Example 1 and the following Step 3-3 using 1-amino-3-chloro-1H-pyrrole-2-carboxamide as the starting material. Compound 70 was got as a pale yellow solid. MS (m/z): 472.6 (M+H)⁺; ¹H NMR (400 MHz, CD₃OD) δ: 8.29 (s, 1H), 7.99 (s, 1H), 7.80 (d, J=7.1 Hz, 1H), 7.67-7.61 (m, 1H), 7.58 (d, J=3.1 Hz, 2H), 7.41 (d, J=6.7 Hz, 1H), 7.35-7.25 (m, 1H), 5.01-4.97 (m, 1H), 4.69-4.65 (m, 1H), 4.34 (dd, J=10.7, 4.1 Hz, 1H), 4.01 (d, J=10.8 Hz, 1H), 2.38-2.28 (m, 1H), 2.20-2.11 (m, 1H).

### Step 3-3 (2S,4R)-tert-butyl 2-(5-chloro-4-oxo-3,4-dihydropyrrolo[2,1-f][1,2,4]triazin-2-yl)-4-(tetrahydro-2H-pyran-2-yloxy)pyrrolidine-1-carboxylate (3C)

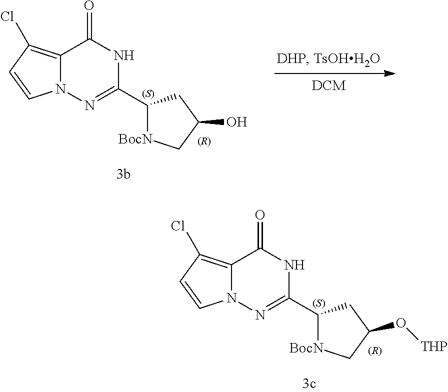

To a solution of 3b (610 mg, 1.72 mmol) in DCM (30 mL) was added DHP (173 mg, 2 mmol) and TsOH.H₂O (65 mg, 0.34 mmol). The reaction mixture was stirred at room temperature for 5 hours. The resulting mixture was concentrated and purified by column chromatography eluting with EtOAc/PE to afford Compound 3c as a pale yellow oil (730 mg, yield: 97%). MS (m/z): 438.7 (M+H)⁺

Compound 71 was prepared according to the procedure of Compound 70 using the corresponding reagents and intermediates under appropriate conditions that will be recognized by one skilled in the art:

| Compd. | | LC/MS | |
|---|---|---|---|
| No. | Structure | (M + H)⁺ | NMR |
| 71 | 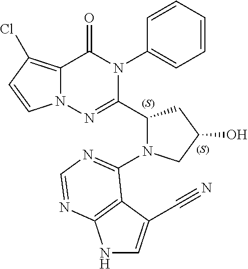 | 472.7 | ¹H NMR (400 MHz, DMSO-d₆) δ 8.39-8.19 (m, 3H), 7.76-7.70 (m, 1H), 7.62-7.46 (m, 5H), 6.60-6.52 (m, 1H), 4.55-4.51 (m, 1H), 4.22-4.18 (m, 1H), 4.17-4.13 (m, 1H), 3.79-3.75 (m, 1H), 2.24-2.20 (m, 1H), 2.07-1.95 (m, 1H). |

### Example 4

### Compound 72

### 5-chloro-2-((2S,4R)-4-methoxy-1-(9H-purin-6-yl)pyrrolidin-2-yl)-3-phenylpyrrolo[2,1-f][1,2,4]triazin-4(3H)-one

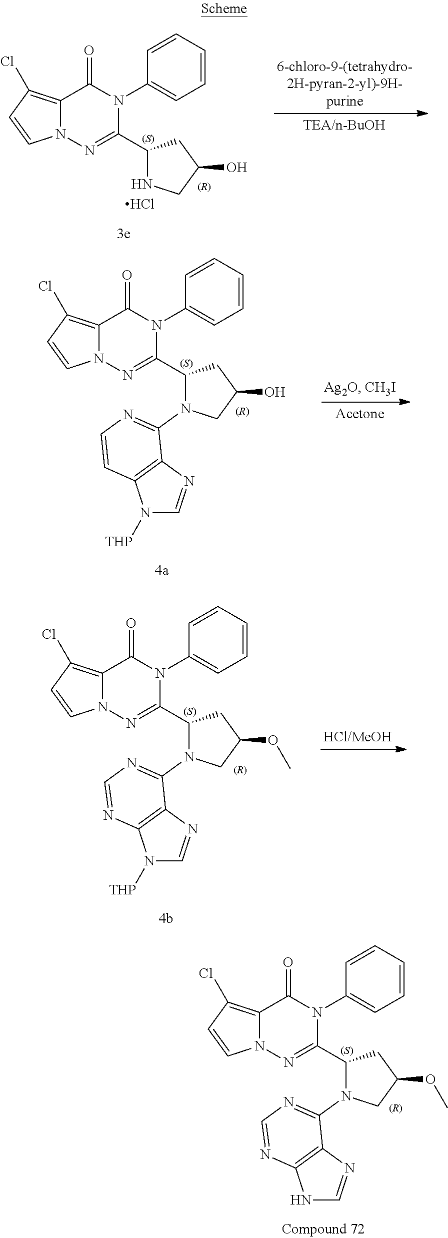

Step 4-1 was carried out according to the procedure in Example 1.

### Step 4-2 5-chloro-2-((2S,4R)-4-methoxy-1-(9-(tetrahydro-2H-pyran-2-yl)-9H-purin-6-yl)pyrrolidin-2-yl)-3-phenylpyrrolo[2,1-f][1,2,4]triazin-4(3H)-one (4b)

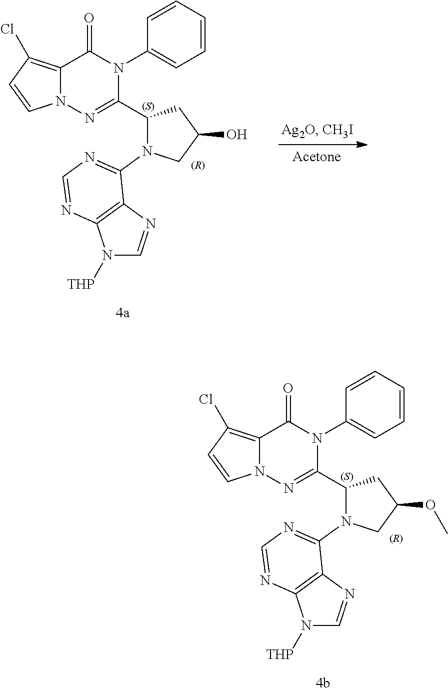

Silver oxide (72 mg, 0.33 mmol) and methyl iodide (62 mg, 0.44 mmol) were added to a solution of 4a (56 mg, 0.11 mmol) in acetone (10 mL) at room temperature. The reaction mixture was stirred in the dark at 60° C. overnight. Then the reaction mixture was filtered and the filtrate was concentrated in vacuo to provide the crude 4b without further purification which is used in the next step reaction. MS (m/z): 547 (M+H)⁺

### Step 4-3 5-chloro-2-((2S,4R)-4-methoxy-1-(9H-purin-6-yl)pyrrolidin-2-yl)-3-phenyl pyrrolo[2,1-f][1,2,4]triazin-4(3H)-one (72)

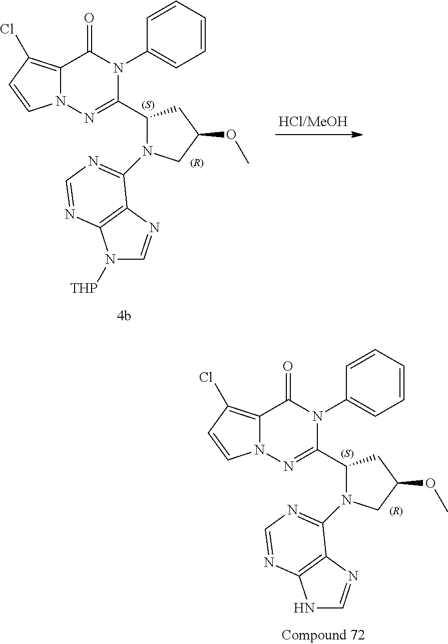

To a solution of 4b (60 mg, 0.11 mmol) in MeOH (2 mL) was added conc.HCl aq (2 mL). The resulting mixture was stirred at 50° C. for one hour. Then the reaction was concentrated and 7N NH₃ in MeOH (5 mL) was added. After concentration in vacuo, the crude product was purified by preparative TLC eluting with MeOH/DCM to afford Compound 72 as a pale yellow solid (16 mg, yield: 31%). MS (m/z): 462.9 (M+H)⁺; ¹H NMR (400 MHz, DMSO-d₆) δ: 8.23-8.08 (m, 2H), 7.73-7.40 (m, 6H), 6.57-6.49 (m, 1H), 5.34-5.24 (m, 1H), 4.64-4.51 (m, 1H), 4.19-4.05 (m, 2H), 3.09 (s, 3H), 2.37-2.29 (m, 1H), 2.04-1.96 (m, 1H).

Compounds 263 and Compounds 265-266 were prepared according to the procedure of Compound 72 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 263 | 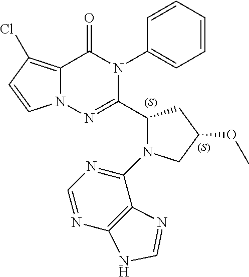 | 463.2 | ¹H NMR (400 MHz, DMSO-d₆) δ: 13.09-12.75 (br, 1H), 8.27-8.09 (m, 2H), 7.76-7.39 (m, 6H), 6.59-6.48 (m, 1H), 5.48-5.38 (m, 0.5H), 4.93-4.81 (br, 0.5H), 4.67-4.55 (m, 0.5H), 4.33-4.22 (m, 0.5H), 4.08-3.99 (m, 0.5H), 3.96-3.89 (br, 0.5H), 3.86-3.77 (m, 0.5H), 3.68-3.59 (m, 0.5H), 3.18 (s, 3H), 2.31-2.06 (m, 2H). |
| 265 | 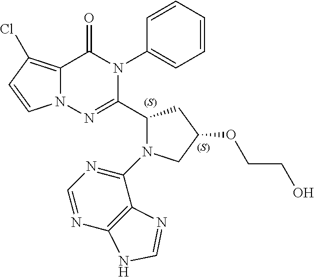 | 493.2 | 1H NMR (400 MHz, CD₃OD) δ 8.09 (s, 1H), 7.93 (br, 1H), 7.85 (s, 1H), 7.62-7.55 (m, 4H), 7.46-7.45 (m, 1H), 7.23 (d, J = 2.8 Hz, 1H), 6.42 (d, J = 3.2 Hz, 1H), 5.47 (s, 1H), 4.52 (s, 1H), 4.20-4.14 (m, 2H), 3.72-3.69 (m, 1H), 3.61-3.51 (m, 4H), 2.20-2.18 (m, 2H) |
| 266 | 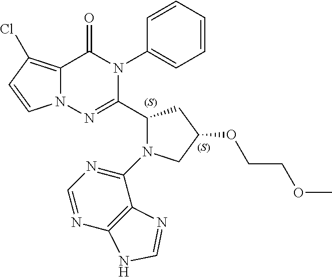 | 507.2 | ¹H NMR (400 MHz, DMSO-d₆) δ 8.05 (s, 1H), 7.82 (s, 1H), 7.62-7.39 (m, 6H), 6.51 (s, 1H), 4.07-3.98 (m, 1H), 3.49-3.13 (m, 10H), 2.08 (br, 2H). |

### Example 5

### Compound 73

### 5-chloro-2-((2S,4S)-4-fluoro-1-(9H-purin-6-yl)pyrrolidin-2-yl)-3-phenylpyrrolo[2,1-f][1,2,4]triazin-4(3H)-one

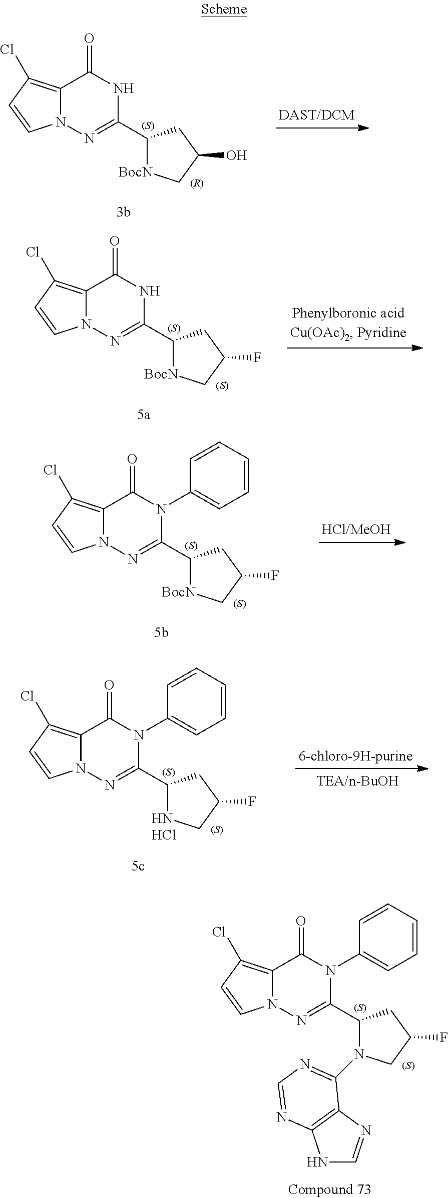

### Step 5-1 (2S,4S)-tert-butyl 2-(5-chloro-4-oxo-3,4-dihydropyrrolo[2,1-f][1,2,4]triazin-2-yl)-4-fluoropyrrolidin-1-carboxylate (5a)

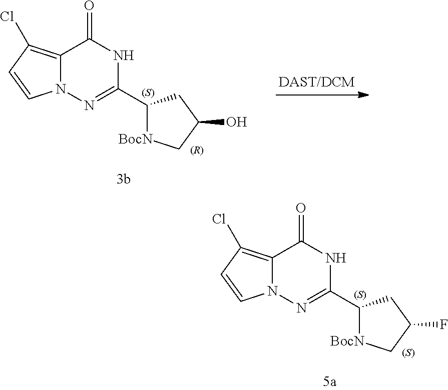

To a solution of 3b (400 mg, 1.13 mmol) in DCM (50 mL) was added DAST (726 mg, 4.52 mmol) at 0° C. The resulting mixture was stirred at 0° C. for one hour, then at room temperature for another one hour. LC-MS showed the starting material disappeared, then NaHCO₃ aq. (10 mL) was added and extracted with DCM three times. The organic layers were combined, dried over Na₂SO₄ and concentrated to give Compound 5a which was used in the next step without further purification. MS (m/z): 257 (M-Boc+H)⁺

Steps 5-2 to 4 were carried out according to the procedure of Example 1. Compound 73 was got as a white solid. MS (m/z): 451.1 (M+H)⁺; ¹H NMR (400 MHz, DMSO-d₆) δ: 8.38-8.10 (m, 3H), 7.71-7.52 (m, 4H), 7.46 (s, 1H), 6.59-6.49 (m, 1H), 5.39-5.29 (m, 1H), 4.88-4.34 (m, 1H), 4.24-3.93 (m, 2H), 2.31-2.17 (m, 2H).

Compound 74 and Compounds 267-268 was prepared according to the procedure of Compound 73 using the corresponding reagents and intermediates under appropriate conditions that will be recognized by one skilled in the art:

| Compd. | | LC/MS | |
|---|---|---|---|
| No. | Structure | (M + H)⁺ | NMR |
| 74 | 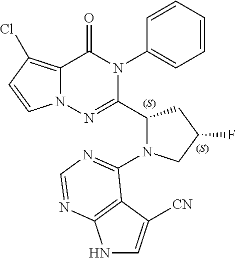 | 475.1 | ¹H NMR (400 MHz, CD₃OD) δ: 8.23 (s, 1H), 7.98 (s, 1H), 7.75 (d, J = 8.0 Hz, 1H), 7.65-7.55 (m, 3H), 7.48 (d, J = 7.6 Hz, 1H), 7.30 (d, J = 3.0 Hz, 1H), 6.46 (d, J = 3.0 Hz, 1H), 5.39-5.31 (m, 1H), 5.22-5.16 (m, 1H), 4.56-4.41 (m, 2H), 2.51-2.41 (m, 1H), 2.22-2.16 (m, 1H). |
| 267 | 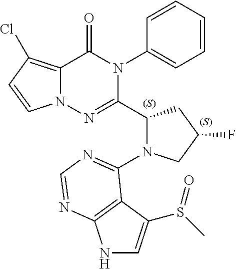 | 512.1 | ¹H NMR (400 MHz, DMSO-d₆) δ: 8.32 (d, J = 5.8 Hz, 1H), 7.96-7.65 (m, 6H), 7.56-7.30 (m, 1H), 6.57 (dd, J = 5.9, 3.0 Hz, 1H), 5.50-5.21 (m, 1H), 4.91-4.82 (m, 1H), 4.15-3.72 (m, 2H), 2.97 (d, J = 2.5 Hz, 3H), 2.31-1.91 (m, 2H). |
| 268 | 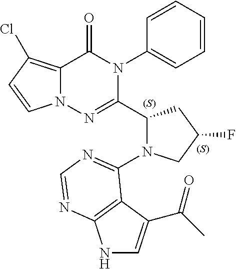 | 492.1 | ¹H NMR (400 MHz, DMSO-d₆) δ: 12.42 (br, 1H), 8.23 (s, 1H), 8.13 (s, 1H), 7.64-7.55 (m, 1H), 7.54-7.45 (m, 5H), 6.59 (d, J = 3.0 Hz, 1H), 5.24-5.02 (m, 1H), 4.74-4.63 (m, 1H), 4.19-3.97 (m, 1H), 3.92-3.83 (m, 1H), 2.51 (s, 3H), 2.44-2.21 (m, 2H). |

### Example 6

### Compound 75

### 3-(1-(9H-purin-6-ylamino)ethyl)-8-chloro-2-(3-fluorophenyl)pyrrolo[1,2-a]pyrazin-1(2H)-one

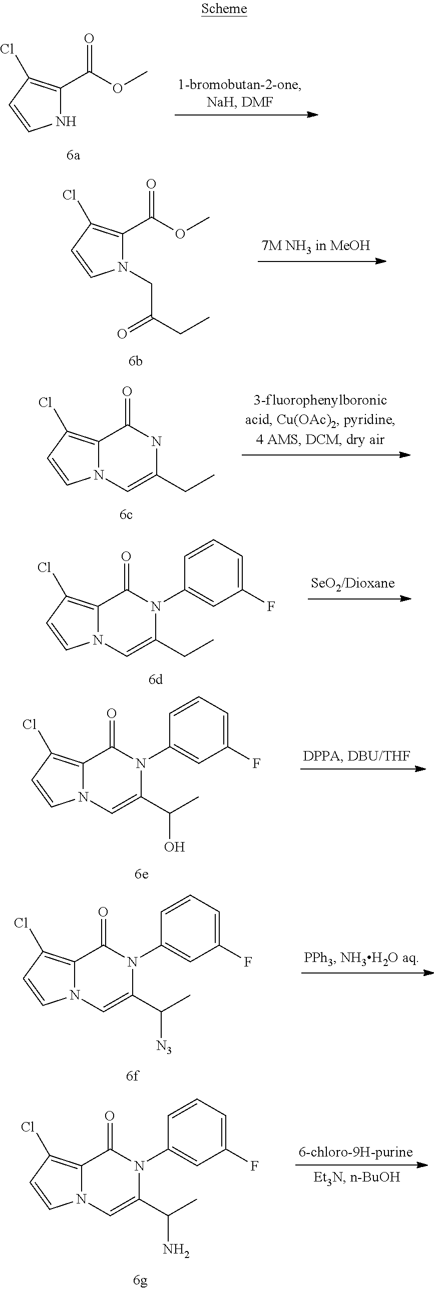

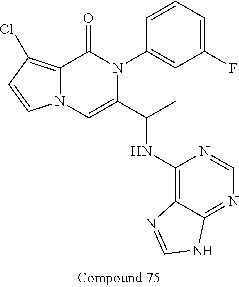 [SMILES: CC(Nc1ncnc2[nH]cnc12)c1cn2ccc(Cl)c2c(=O)n1-c1cccc(F)c1]

### Step 6-1 methyl 3-chloro-1-(2-oxobutyl)-1H-pyrrole-2-carboxylate (6b)

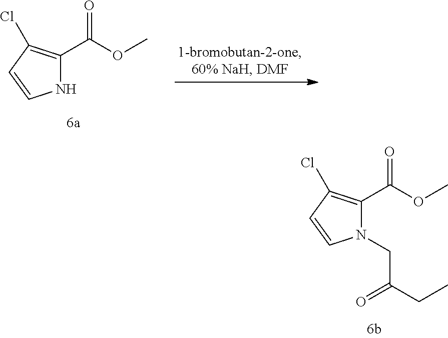

To a solution of 6a (4.8 g, 30.0 mmol) in DMF (40 mL) was added 60% NaH (1.2 g, 30.0 mmol) at 0-5° C. and stirred at 0-5° C. for 30 minutes. Then 1-bromobutan-2-one (5.0 g, 33 mmol) was added and stirred at room temperature for 2 hours. After concentration in vacuo, the residue was used in the next step without further purification. MS (m/z): 230.1 (M+H)⁺

### Step 6-2 8-chloro-3-ethylpyrrolo[1,2-a]pyrazin-1(2H)-one (6c)

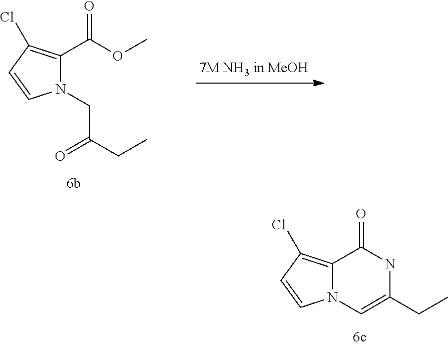

A mixture of the obtained 6b (30.0 mmol) in 7M NH₃/MeOH (80 mL) was stirred in a sealed tube at 130° C. for 16 hours. After concentration, the residue was purified by flash column chromatography eluting with MeOH/H₂O to afford 6c as a white solid (2.67 g, yield: 45%). MS (m/z): 197.1 (M+H)⁺

### Step 6-3 8-chloro-3-ethyl-2-(3-fluorophenyl)pyrrolo[1,2-a]pyrazin-1(2H)-one (6d)

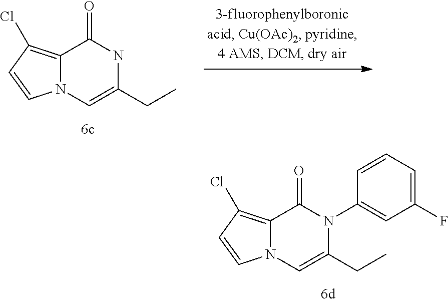 [SMILES: CCc1cn2ccc(Cl)c2c(=O)[nH]1 ; CCc1cn2ccc(Cl)c2c(=O)n1-c1cccc(F)c1]

A mixture of 6c (1.97 g, 10.0 mmol), 3-fluorophenylboronic acid (2.80 g, 20.0 mmol), 4AMS (24 g), Cu(OAc)₂, (3.63 g, 20.0 mmol) and pyridine (3.96 g, 50.0 mmol) in dry DCM (80 mL) was stirred under dry air at room temperature for 16 hours. The mixture was filtered through celite and washed with MeOH/DCM. The filtrate was concentrated and purified by flash column chromatography eluting with MeOH/DCM to afford 6d as a yellow solid (1.53 g, yield: 53%). MS (m/z): 291.0 (M+H)⁺

### Step 6-4 8-chloro-2-(3-fluorophenyl)-3-(1-hydroxyethyl)pyrrolo[1,2-a]pyrazin-1(2H)-one (6e)

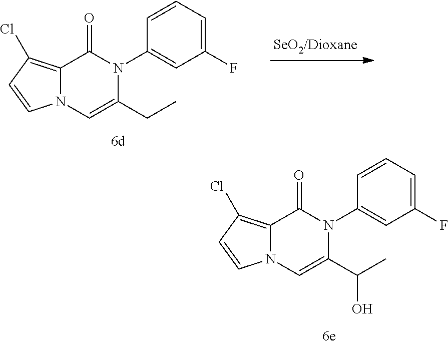 [SMILES: CCc1cn2ccc(Cl)c2c(=O)n1-c1cccc(F)c1 ; CC(O)c1cn2ccc(Cl)c2c(=O)n1-c1cccc(F)c1]

To a solution of 6d (1.53 g, 5.26 mmol) in dioxane (25 mL) was added SeO₂ (584 mg, 5.26 mmol) and stirred under reflux for one hour. After concentration, the residue was purified by flash column chromatography eluting with EtOAc/PE to afford 6e as a yellow solid (1.60 g, yield: 99%). MS (m/z): 307.0 (M+H)⁺

### Step 6-5 3-(1-azidoethyl)-8-chloro-2-(3-fluorophenyl)pyrrolo[1,2-a]pyrazin-1(2H)-one (6f)

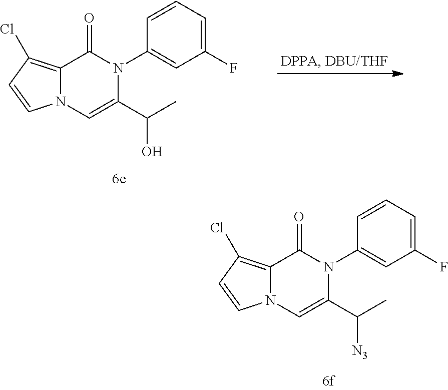 [SMILES: CC(O)c1cn2ccc(Cl)c2c(=O)n1-c1cccc(F)c1 ; CC(N=[N+]=[N-])c1cn2ccc(Cl)c2c(=O)n1-c1cccc(F)c1]

To a solution of 6e (1.60 g, 5.2 mmol) in THF (30 mL) was added DPPA (2.86 g, 10.4 mmol) and DBU (1.58 g, 10.4 mmol), then the mixture was stirred at 50-60° C. overnight. After concentration, the residue was purified by flash column chromatography eluting with EtOAc/PE to afford 6f as a yellow oil (680 mg, yield: 39%). MS (m/z): 332.0 (M+H)⁺

### Step 6-6 3-(1-aminoethyl)-8-chloro-2-(3-fluorophenyl)pyrrolo[1,2-a]pyrazin-1(2H)-one (6g)

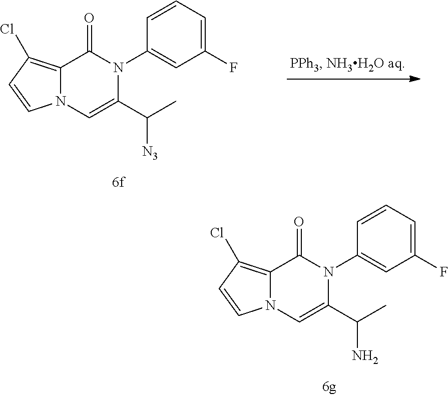 [SMILES: CC(N=[N+]=[N-])c1cn2ccc(Cl)c2c(=O)n1-c1cccc(F)c1 ; CC(N)c1cn2ccc(Cl)c2c(=O)n1-c1cccc(F)c1]

To a mixture of 6f (680 mg, 2.05 mmol) in THF (20 mL) was added PPh₃ (1.08 g, 4.10 mmol) and the reaction was stirred at room temperature for 10 minutes. Then conc. NH₃.H₂O aq. (5 mL) was added and the reaction was stirred at 50-60° C. for another 4 hours. The reaction mixture was concentrated in vacuo and the residue was purified by flash column chromatography eluting with MeOH/H₂O to afford 6g as a white solid (320 mg, yield: 51%). MS (m/z): 306.1 (M+H)⁺

### Step 6-7 3-(1-(9H-purin-6-ylamino)ethyl)-8-chloro-2-(3-fluorophenyl)pyrrolo[1,2-a]pyrazin-1(2H)-one (75)

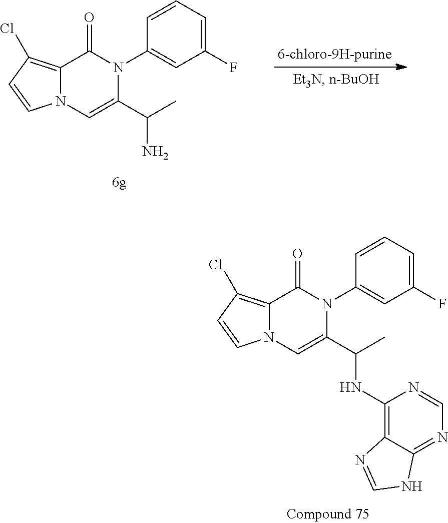 [SMILES: CC(N)c1cn2ccc(Cl)c2c(=O)n1-c1cccc(F)c1 ; CC(Nc1ncnc2[nH]cnc12)c1cn2ccc(Cl)c2c(=O)n1-c1cccc(F)c1]

A mixture of 6g (61 mg, 0.20 mmol), 6-chloro-9H-purine (37 mg, 0.24 mmol) and TEA (40 mg, 0.40 mmol) in n-BuOH (1 mL) was stirred under nitrogen at reflux for 16 hours. The reaction mixture was concentrated in vacuo, and the residue was purified by flash column chromatography eluting with MeOH/H₂O to afford Compound 75 as a yellow solid (44.4 mg, yield: 50%). MS (m/z): 424.1 (M+H)⁺. ¹H NMR (400 MHz, DMSO-d₆) δ: 8.03-7.94 (m, 2H), 7.79 (s, 1H), 7.47 (s, 2H), 7.35-7.12 (m, 3H), 7.00 (s, 2H), 6.60 (s, 1H), 4.81 (m, 1H), 1.35 (br, 3H).

The following Compounds were prepared according to the procedure of Compound 75 using the corresponding reagents and intermediates under appropriate conditions that will be recognized by one skilled in the art:

| Compound | | LC/MS | |
|---|---|---|---|
| No. | Structure | (M + H)⁺ | NMR |
| 76 | 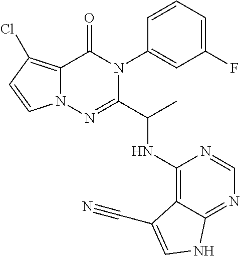 [SMILES: CC(Nc1ncnc2[nH]cc(C#N)c12)c1nn2ccc(Cl)c2c(=O)n1-c1cccc(F)c1] | 448.1 | ¹H NMR (400 MHz, DMSO-d₆) δ: 8.09 (d, J = 4.4 Hz, 1H), 8.09-7.99 (m, 1H), 7.64 (d, J = 9.5 Hz, 1H), 7.50 (dd, J = 6.4, 2.9 Hz, 1H), 7.42-7.36 (m, 1H), 7.23-7.13 (m, 1H), 7.09-6.93 (m, 2H), 6.67-6.61 (m, 1H), 6.57-6.47 (m, 1H), 4.95-4.85 (m, 1H), 1.40 (d, J = 6.0 Hz, 3H). |
| 77 | 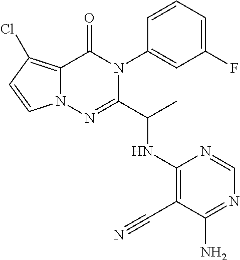 [SMILES: CC(Nc1ncnc(N)c1C#N)c1nn2ccc(Cl)c2c(=O)n1-c1cccc(F)c1] | 424.1 | ¹H NMR (400 MHz, DMSO-d₆) δ: 7.74 (d, J = 19.3 Hz, 1H), 7.57 (d, J = 2.7 Hz, 1H), 7.53 (d, J = 11.0 Hz, 1H), 7.45-7.35 (m, 2H), 7.29-7.03 (m, 5H), 6.67-6.66 (m, 1H), 4.87-4.79 (m, 1H), 1.32 (d, J = 6.6 Hz, 3H). |
| 78 | 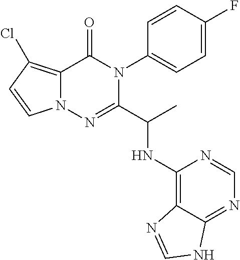 [SMILES: CC(Nc1ncnc2[nH]cnc12)c1nn2ccc(Cl)c2c(=O)n1-c1ccc(F)cc1] | 424.1 | ¹H NMR (400 MHz, DMSO-d₆) δ: 7.91 (s, 1H), 7.67 (s, 2H), 7.54-7.46 (m, 1H), 7.44 (d, J = 2.8 Hz, 1H), 7.40 (s, 1H), 7.30 (td, J = 8.7, 2.9 Hz, 1H), 7.15 (td, J = 8.7, 2.9 Hz, 1H), 6.93 (d, J = 6.8 Hz, 1H), 6.58 (d, J = 2.8 Hz, 1H), 4.90-4.78 (m, 1H), 1.28 (d, J = 6.8 Hz, 3H). |
| 79 | 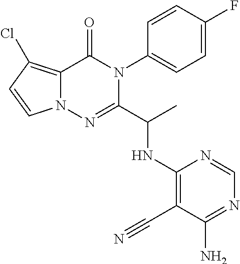 [SMILES: CC(Nc1ncnc(N)c1C#N)c1nn2ccc(Cl)c2c(=O)n1-c1ccc(F)cc1] | 424.1 | ¹H NMR (400 MHz, DMSO-d₆) δ: 7.78 (s, 1H), 7.56 (d, J = 2.9 Hz, 1H), 7.51 (s, 1H), 7.47 (d, J = 7.3 Hz, 1H), 7.41-7.39 (m, 1H), 7.26-7.19 (m, 4H), 7.05 (td, J = 8.7, 3.0 Hz, 1H), 6.66 (d, J = 2.8 Hz, 1H), 4.82-4.75 (m, 1H), 1.31 (d, J = 6.8 Hz, 3H). |
| 80 | 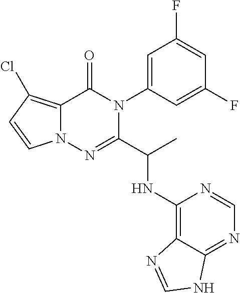 [SMILES: CC(Nc1ncnc2[nH]cnc12)c1nn2ccc(Cl)c2c(=O)n1-c1cc(F)cc(F)c1] | 442.1 | ¹H NMR (400 MHz, CD₃OD) δ: 8.03 (s, 2H), 7.57 (s, 1H), 7.38 (d, J = 2.9 Hz, 1H), 7.07 (d, J = 8.8 Hz, 1H), 6.84 (d, J = 9.3 Hz, 1H), 6.72 (t, J = 8.8 Hz, 1H), 6.59 (d, J = 2.8 Hz, 1H), 5.25-5.13 (m, 1H), 1.54 (d, J = 6.7 Hz, 3H). |
| 81 | 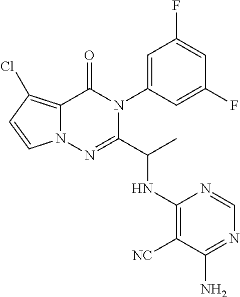 [SMILES: CC(Nc1ncnc(N)c1C#N)c1nn2ccc(Cl)c2c(=O)n1-c1cc(F)cc(F)c1] | 442.1 | ¹H NMR (400 MHz, DMSO-d₆) δ: 7.74 (s, 1H), 7.55 (d, J = 2.7 Hz, 1H), 7.53 (s, 1H), 7.39 (d, J = 7.6 Hz, 1H), 7.26 (d, J = 9.2 Hz, 1H), 7.14 (s, 2H), 7.10 (d, J = 9.5 Hz, 1H), 6.91 (d, J = 9.4 Hz, 1H), 6.65 (d, J = 2.7 Hz, 1H),4.89 (m, 1H), 1.32 (d, J = 6.5 Hz, 3H). |
| 82 | 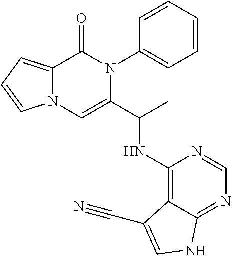 [SMILES: CC(Nc1ncnc2[nH]cc(C#N)c12)c1cn2cccc2c(=O)n1-c1ccccc1] | 396.1 | ¹H NMR (400 MHz, DMSO-d₆) δ: 8.07 (s, 1H), 8.05 (s, 1H), 7.68 (s, 1H), 7.47 (dd, J = 2.5, 1.5 Hz, 1H), 7.42-7.38 (m, 1H), 7.36-7.34 (m, 2H), 7.19 (t, J = 7.4 Hz, 1H), 7.08 (t, J = 8.1 Hz, 1H), 6.89 (d, J = 4.0 Hz, 1H), 6.56 (dd, J = 3.9, 2.6 Hz, 1H), 6.41 (d, J = 7.1 Hz, 1H), 4.85-4.79 (m, 1H), 1.39 (d, J = 6.8 Hz, 3H). |
| 83 | 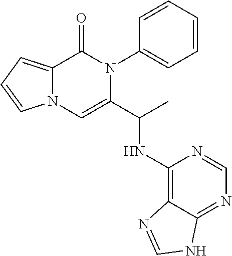 [SMILES: CC(Nc1ncnc2[nH]cnc12)c1cn2cccc2c(=O)n1-c1ccccc1] | 372.1 | ¹H NMR (400 MHz, DMSO-d₆) δ: 12.90 (s, 1H), 8.08-7.93 (m, 3H), 7.50-7.47 (m, 2H), 7.41-7.34 (m, 3H), 7.23 (s, 1H), 7.10 (s, 1H), 6.87 (s, 1H), 6.54 (s, 1H), 4.85-4.75 (m, 1H), 1.34 (d, J = 6.2 Hz, 3H). |
| 84 | 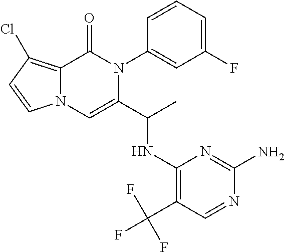 [SMILES: CC(Nc1nc(N)ncc1C(F)(F)F)c1cn2ccc(Cl)c2c(=O)n1-c1cccc(F)c1] | 467.1 | ¹H NMR (400 MHz, DMSO-d₆) δ 7.95-7.89 (m, 1H), 7.42-7.17 (m, 4H), 7.09-6.99 (m, 2H), 6.67-6.46 (m, 4H), 5.03-4.93 (m, 1H), 1.33-1.30 (m, 3H). |
| 269 | 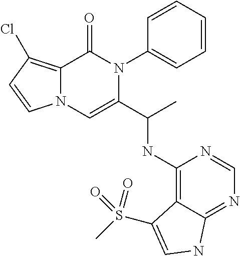 [SMILES: CC(Nc1ncnc2[nH]cc(S(C)(=O)=O)c12)c1cn2ccc(Cl)c2c(=O)n1-c1ccccc1] | 483.1 | 1H NMR (400 MHz, DMSO-d6) δ 8.13 (s, 1H), 7.99 (s, 1H), 7.56 (s, 1H), 7.52-7.43 (m, 3H), 7.44-7.42 (m, 1H), 7.36-7.34 (m, 1H), 7.32-7.28 (m, 1H), 7.167-7.19 (m, 1H), 6.68 (d, J = 2.8 Hz, 1H), 4.80-4.77 (m, 1H), 3.38 (s, 3H), 1.41 (d, J = 6.8 Hz, 3H). |
| 270 | 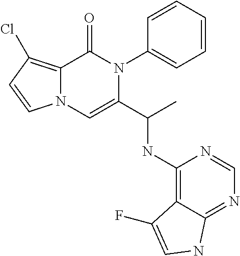 [SMILES: CC(Nc1ncnc2[nH]cc(F)c12)c1cn2ccc(Cl)c2c(=O)n1-c1ccccc1] | 423.1 | 1H NMR (400 MHz, DMSO-d6) δ 7.90 (s, 1H), 7.50 (s, 1H), 7.49 (d, J = 2.8 Hz, 1H), 7.40-7.33 (m, 4H), 7.23-7.20 (m, 1H), 7.12-7.10 (m, 1H), 7.01-6.98 (m, 2H), 6.60 (d, J = 2.8 Hz, 1H), 4.82-4.79 (m, 1H), 1.33 (d, J = 6.4 Hz, 3H). |
| 271 | 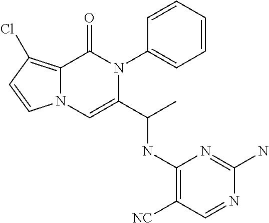 [SMILES: CC(Nc1nc(N)ncc1C#N)c1cn2ccc(Cl)c2c(=O)n1-c1ccccc1] | 406.0 | 1H NMR (400 MHz, DMSO-d6) δ 8.08 (s, 1H), 7.56 (d, J = 2.8 Hz, 1H), 7.49-7.38 (m, 3H), 7.35-7.29 (m, 2H), 7.29-7.22 (m, 2H), 7.11 (br, 2H), 6.65 (d, J = 2.8 Hz, 1H), 4.80-4.63 (m, 1H), 1.27 (d, J = 6.7 Hz, 3H). |
| 272 | 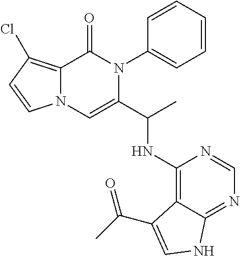 [SMILES: CC(=O)c1c[nH]c2ncnc(NC(C)c3cn4ccc(Cl)c4c(=O)n3-c3ccccc3)c12] | 447.1 | ¹H NMR (400 MHz, CD3OD) δ 9.16 (d, J = 7.6 Hz, 1H), 8.02 (s, 1H), 7.85 (s, 1H), 7.44 (s, 1H), 7.37-7.33 (m, 1H), 7.28 (d, J = 2.8 Hz, 1H), 7.26-7.24 (m, 1H), 7.15-7.10 (m, 2H), 6.28-6.87 (m, 1H), 6.49 (d, J = 2.9 Hz, 1H), 2.44 (s, 3H), 1.39 (d, J = 6.8 Hz, 3H). |
| 273 | 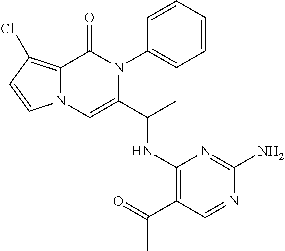 [SMILES: CC(=O)c1cnc(N)nc1NC(C)c1cn2ccc(Cl)c2c(=O)n1-c1ccccc1] | 423.1 | ¹H NMR (400 MHz, CD3OD) δ 8.47 (s, 1H), 7.50-7.47 (m, 2H), 7.36 (d, J = 2.8 Hz, 1H), 7.33-7.15 (m, 4H), 6.58 (d, J = 2.8 Hz, 1H), 5.00-4.95 (m, 1H), 2.42 (s, 3H), 1.38 (d, J = 6.8 Hz, 3H). |
| 274 | 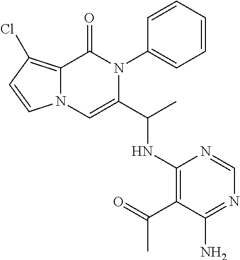 [SMILES: CC(=O)c1c(N)ncnc1NC(C)c1cn2ccc(Cl)c2c(=O)n1-c1ccccc1] | 422.8 | ¹H NMR (400 MHz, CD3OD) δ 7.70 (s, 1H), 7.49-7.54 (m, 1H), 7.44 (s, 1H), 7.35-7.31 (m, 3H), 7.28-7.24 (m, 1H), 7.20-7.18 (m, 1H), 6.58 (dd, J = 2.8, 0.6, 1H), 4.95 (q, J = 8.0 Hz, 1H), 2.56 (s., 3H), 1.40 (d, J = 6.8 Hz, 3H). |
| 275 | 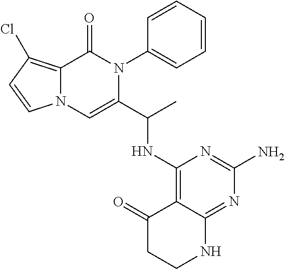 [SMILES: CC(Nc1nc(N)nc2c1C(=O)CCN2)c1cn2ccc(Cl)c2c(=O)n1-c1ccccc1] | 450.1 | ¹H NMR (400 MHz, CD3OD) δ 9.22 (d, J = 7.2 Hz, 0H), 7.50-7.45 (m, 1H), 7.42 (d, J = 0.8 Hz, 1H), 7.35 (d, J = 3.2. Hz 1H), 7.33-7.28 (m, 3H), 7.23-7.20 (m, 1H), 6.57 (d, J = 2.8 Hz, 1H), 4.91-4.78 (m, 1H), 3.51-3.41 (m, 2H), 2.64-2.45 (m, 2H), 1.37 (d, J = 6.8 Hz, 3H). |
| 276 | 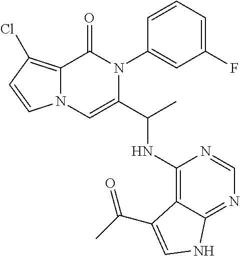 [SMILES: CC(=O)c1c[nH]c2ncnc(NC(C)c3cn4ccc(Cl)c4c(=O)n3-c3cccc(F)c3)c12] | 465.1 | ¹H NMR (400 MHz, DMSO-d6) δ 12.44 (br, 1H), 9.02-8.98 (m, 1H), 8.26 (s, 0.5H), 8.25 (s, 0.5H), 8.01 (s, 0.5H), 7.96 (s, 0.5H), 7.59 (s, 0.5H), 7.55 (s, 0.5H), 7.53-7.50 (m, 1H), 7.46-7.37 (m, 1H), 7.24 (d, J = 8.0 Hz, 0.5H), 7.09-7.00 (m, 2H), 6.94 (d, J = 9.7 Hz, 0.5H), 6.65-6.60 (m, 1H), 4.79-4.74 (m, 1H), 1.40-1.37 (m, 3H). |
| 277 | 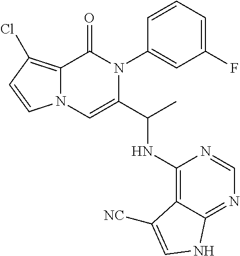 [SMILES: CC(Nc1ncnc2[nH]cc(C#N)c12)c1cn2ccc(Cl)c2c(=O)n1-c1cccc(F)c1] | 448.1 | ¹H NMR (400 MHz, DMSO-d6) δ 8.09-7.97 (m, 2H), 7.63 (d, J = 10.0 Hz, 1H), 7.50 (br, 1H), 7.46-7.35 (m, 1H), 7.24-7.02 (m, 3H), 6.62 (br, 1H), 6.37-6.31 (m, 1H), 4.87 (br, 1H), 1.39 (d, J = 6.1 Hz, 3H). |
| 278 | 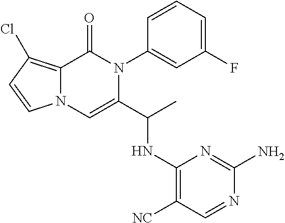 [SMILES: CC(Nc1nc(N)ncc1C#N)c1cn2ccc(Cl)c2c(=O)n1-c1cccc(F)c1] | 424.1 | 1H NMR (400 MHz, DMSO-d6) δ 8.05 (d, J = 0.6 Hz, 1H), 7.54 (d, J = 2.9 Hz, 1H), 7.44- 7.00 (m, 6H), 6.79 (br, 2H), 6.64 (t, J = 2.9 Hz, 1H), 4.78-4.74 (m, 1H), 1.30-1.27 (m, 3H). |
| 327 | 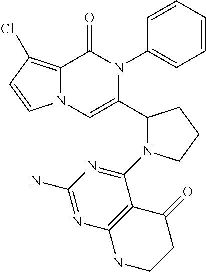 [SMILES: Nc1nc2c(c(N3CCCC3c3cn4ccc(Cl)c4c(=O)n3-c3ccccc3)n1)C(=O)CCN2] | 475.8 | ¹H NMR (400 MHz, DMSO-d6) δ 7.68 (br, 1H), 7.58-7.36 (m, 5H), 7.32 (d, J = 2.6, 1H), 7.02 (s, 1H), 6.55 (d, J = 2.8, 1H), 6.32 (s, 2H), 4.92 (t, J = 7.5, 1H), 3.88 (br, 1H), 3.34 (br, 2H), 2.95 (br, IH), 2.44-2.37 (m, 1H), 2.28-2.23 (m, 1H), 1.97-1.45 (m, 4H). |

### Example 7

### Compound 85

### 3-(1-(9H-purin-6-ylamino)propyl)-8-chloro-2-(3-fluorophenyl)pyrrolo[1,2-a]pyrazin-1(2H)-one

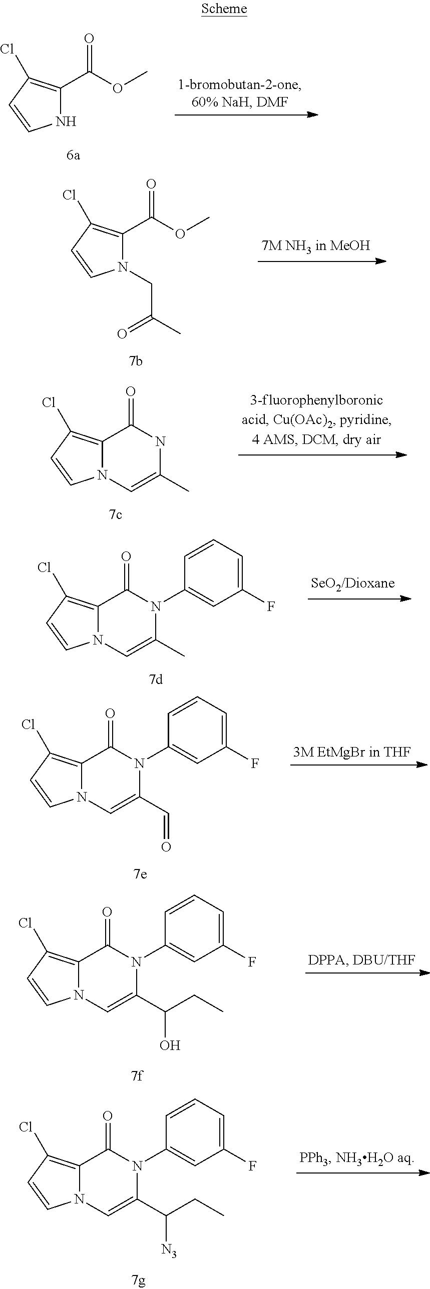

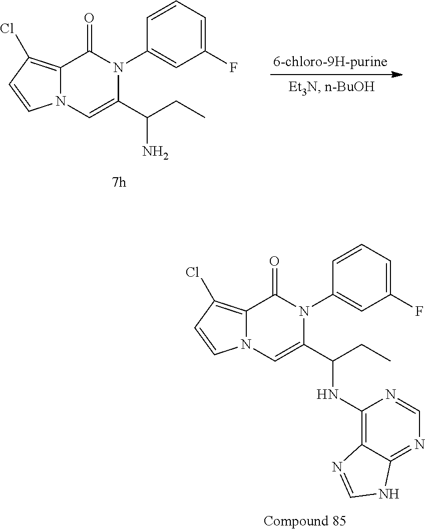 [SMILES: CCC(N)c1cn2ccc(Cl)c2c(=O)n1-c1cccc(F)c1 ; CCC(Nc1ncnc2[nH]cnc12)c1cn2ccc(Cl)c2c(=O)n1-c1cccc(F)c1]

### Step 7-1 methyl 3-chloro-1-(2-oxopropyl)-1H-pyrrole-2-carboxylate (7b)

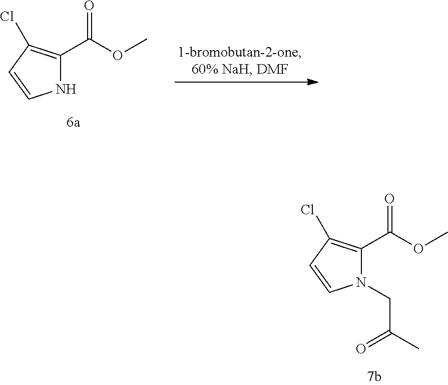

To a solution of 6a (5.85 g, 36.7 mmol) in DMF (70 mL) was added 60% NaH (1.61 g, 40.3 mmol) at 0-5° C. and stirred at 0-5° C. for 30 minutes. Then a solution of 1-bromopropan-2-one (7.54 g, 55 mmol) in DMF (10 mL) was added dropwise at 0-5° C., and the reaction was stirred at room temperature for 30 minutes. After concentration in vacuo, the residue 7b was used in the next step without further purification.

### Step 7-2 8-chloro-3-methylpyrrolo[1,2-a]pyrazin-1(2H)-one (7c)

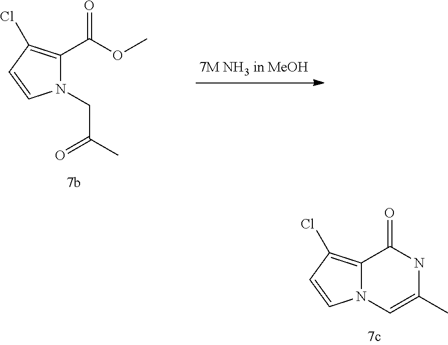

A mixture of obtained 7b (36.7 mmol) in 7M NH₃ in MeOH (80 mL) was stirred in a sealed tube at 130° C. for 16 hours. After concentration in vacuo, the residue was purified by flash column chromatography eluting with MeOH/DCM to afford 7c as a yellow solid (3.59 g, yield: 54%). MS (m/z): 183.1 (M+H)⁺

### Step 7-3 8-chloro-2-(3-fluorophenyl)-3-methylpyrrolo[1,2-a]pyrazin-1(2H)-one (7d)

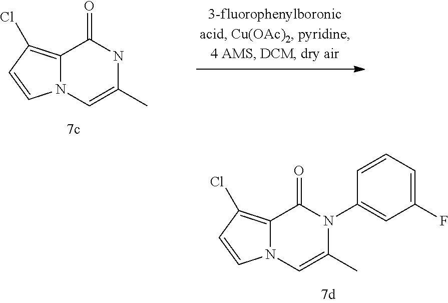 [SMILES: Cc1cn2ccc(Cl)c2c(=O)[nH]1 ; Cc1cn2ccc(Cl)c2c(=O)n1-c1cccc(F)c1]

A mixture of 7c (910 mg, 5.0 mmol), 3-fluorophenylboronic acid (1.40 g, 10.0 mmol), 4AMS (25g), Cu(OAc)₂, (1.82 g, 10.0 mmol) and pyridine (1.98 g, 25.0 mmol) in dry DCM (80 mL) was stirred under dry air at room temperature for 16 hours. The mixture was filtered through celite and washed with MeOH/DCM. The filtrate was concentrated and the residue was purified by flash column chromatography eluting with MeOH/H₂O to afford 7d as a yellow solid (1.38 g, yield: 83%). MS (m/z): 277.1 (M+H)⁺

### Step 7-4 8-chloro-2-(3-fluorophenyl)-1-oxo-1,2-dihydropyrrolo[1,2-a]pyrazine-3-carbaldehyde (7e)

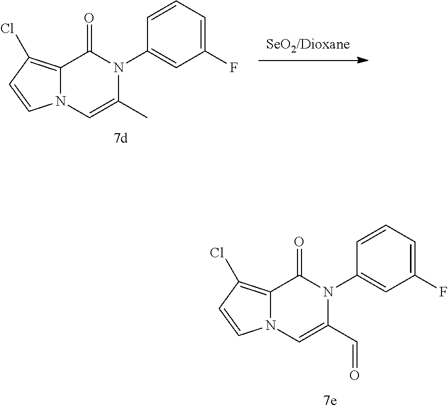 [SMILES: Cc1cn2ccc(Cl)c2c(=O)n1-c1cccc(F)c1 ; O=Cc1cn2ccc(Cl)c2c(=O)n1-c1cccc(F)c1]

To a solution of 7d (1.38 g, 5.0 mmol) in dioxane (30 mL) was added SeO₂ (1.11 g, 10 mmol) and the reaction was stirred at reflux for 2 hours. The mixture was diluted with EtOAc, and filtered through celite. The filtrate was collected, concentrated and purified by flash column chromatography eluting with EtOAc/PE to afford 7e as a yellow solid (1.45 g, yield: 100%). MS (m/z): 291.0 (M+H)⁺

### Step 7-5 8-chloro-2-(3-fluorophenyl)-3-(1-hydroxypropyl)pyrrolo[1,2-a]pyrazin-1(2H)-one (7f)

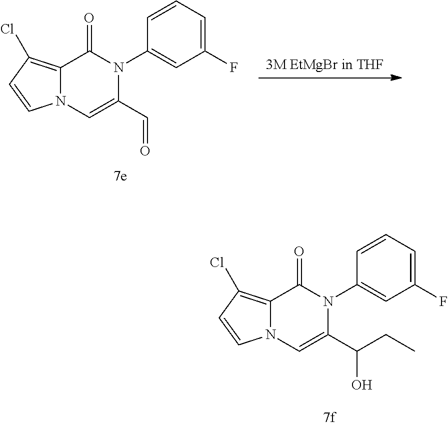 [SMILES: O=Cc1cn2ccc(Cl)c2c(=O)n1-c1cccc(F)c1 ; CCC(O)c1cn2ccc(Cl)c2c(=O)n1-c1cccc(F)c1]

To a solution of 7e (1.01 g, 3.5 mmol) in dry THF (50 mL) was added 3M EtMgBr in THF (7 mL, 21 mmol) at 0-5° C. and the reaction was stirred at room temperature for 30 minutes. The mixture was poured into sat. NH₄Cl aq, and extracted with EtOAc. The organic layer was collected, concentrated and purified by flash column chromatography eluting with EtOAc/PE to afford 7f as a yellow solid (1.06 g, yield: 94%). MS (m/z): 321.0 (M+H)⁺

### Step 7-6 3-(1-azidopropyl)-8-chloro-2-(3-fluorophenyl)pyrrolo[1,2-a]pyrazin-1(2H)-one (7g)

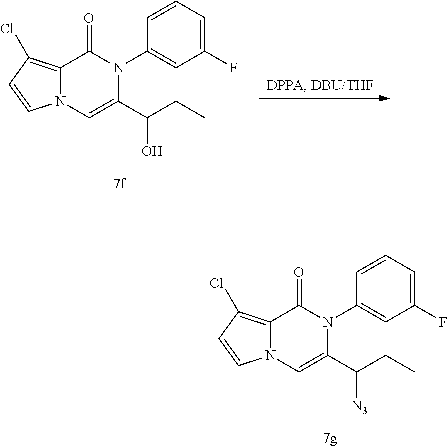 [SMILES: CCC(O)c1cn2ccc(Cl)c2c(=O)n1-c1cccc(F)c1 ; CCC(N=[N+]=[N-])c1cn2ccc(Cl)c2c(=O)n1-c1cccc(F)c1]

To a solution of 7f (1.06 g, 3.3 mmol) in THF (50 mL) was added DPPA (1.82 g, 6.6 mmol) and DBU (1.0 g, 6.6 mmol), then the reaction was stirred at 50-60° C. overnight. After concentration in vacuo, the residue was purified by flash column chromatography eluting with EtOAc/PE to afford 7g as a yellow oil (853 mg, yield: 75%). MS (m/z): 346.1 (M+H)⁺

### Step 7-7 3-(1-aminopropyl)-8-chloro-2-(3-fluorophenyl)pyrrolo[1,2-a]pyrazin-1(2H)-one (7h)

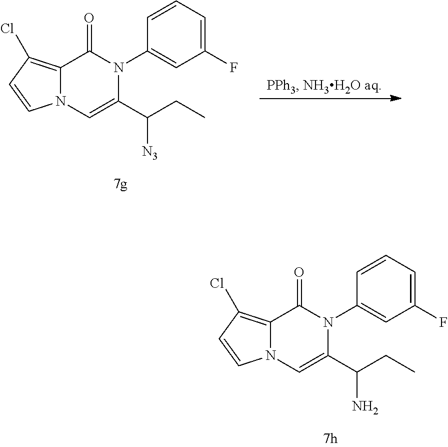 [SMILES: CCC(N=[N+]=[N-])c1cn2ccc(Cl)c2c(=O)n1-c1cccc(F)c1 ; CCC(N)c1cn2ccc(Cl)c2c(=O)n1-c1cccc(F)c1]

To a mixture of 7g (853 mg, 2.46 mmol) in THF (10 mL) was added PPh₃ (1.293 g, 4.92 mmol) and conc. NH₃.H₂O aq. (4.2 mL), then the reaction was stirred at 50-60° C. for 16 hours. After concentration in vacuo, the residue was purified by flash column chromatography eluting with MeOH/H₂O to afford 7h as a yellow solid (600 mg, yield: 76%). MS (m/z): 320.1 (M+H)⁺

### Step 7-8 3-(1-(9H-purin-6-ylamino)propyl)-8-chloro-2-(3-fluorophenyl)pyrrolo[1,2-a]pyrazin-1(2H)-one (85)

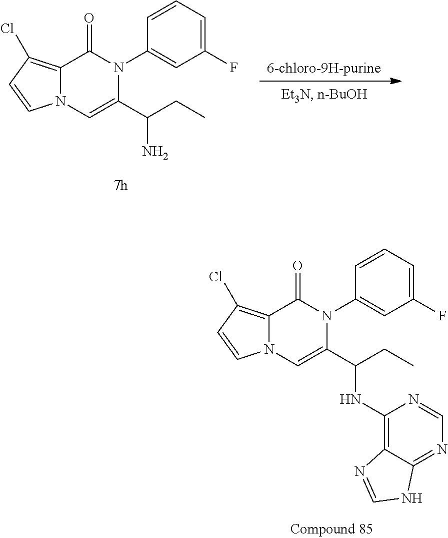 [SMILES: CCC(N)c1cn2ccc(Cl)c2c(=O)n1-c1cccc(F)c1 ; CCC(Nc1ncnc2[nH]cnc12)c1cn2ccc(Cl)c2c(=O)n1-c1cccc(F)c1]

A mixture of 7h (143 mg, 0.45 mmol), 6-chloro-9H-purine (77 mg, 0.50 mmol) and TEA (136 mg, 1.35 mmol) in n-BuOH (2 mL) was stirred under nitrogen at reflux for 16 hours. The reaction mixture was concentrated in vacuo. The residue was purified by flash column chromatography eluting with MeOH/H₂O and further purified by preparative TLC eluting with MeOH/DCM to afford Compound 85 as a yellow solid (16.1 mg, yield: 8.2%). MS (m/z): 438.1 (M+H)⁺. ¹H NMR (400 MHz, DMSO-d₆) δ: 8.00-7.97 (m, 2H), 7.41-7.40 (m, 2H), 7.25-7.23 (m, 1H), 7.13-7.07 (m, 2H), 7.03-6.94 (m, 2H), 6.48-6.47 (m, 1H), 1.93-1.84 (m, 1H), 1.75-1.68 (m, 1H), 0.85-0.82 (m, 3H).

The following Compounds were prepared according to the procedure of Compound 85 using the corresponding reagents and intermediates under appropriate conditions that will be recognized by one skilled in the art:

| Compd. | | LC/MS | |
|---|---|---|---|
| No. | Structure | (M + H)⁺ | NMR |
| 86 | 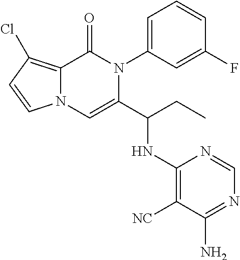 [SMILES: CCC(Nc1ncnc(N)c1C#N)c1cn2ccc(Cl)c2c(=O)n1-c1cccc(F)c1] | 438.1 | ¹H NMR (400 MHz, DMSO-d₆) δ: 7.79-7.73 (m, 1H), 7.53-7.52 (m, 1H), 7.48-7.44 (m, 2H), 7.36-7.32 (m, 2H), 7.20-7.15 (m, 3H), 7.12-7.11 (m, 1H), 6.64-6.62 (m, 1H), 4.60-4.52 (m, 1H), 1.76-1.70 (m, 2H), 0.75-0.70 (m, 3H). |
| 87 | 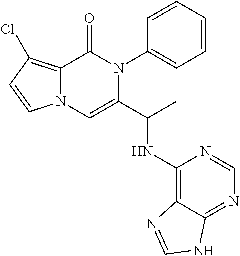 [SMILES: CC(Nc1ncnc2[nH]cnc12)c1cn2ccc(Cl)c2c(=O)n1-c1ccccc1] | 406.1 | ¹H NMR (400 MHz, DMSO-d₆) δ: 8.05-8.03 (m, 2H), 7.83 (s, 1H), 7.48-7.10 (m, 8H), 6.60 (s, 1H), 4.82-4.72 (m, 1H), 1.33 (d, J = 5.9 Hz, 3H). |
| 88 | 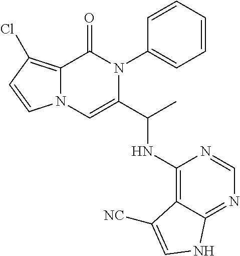 [SMILES: CC(Nc1ncnc2[nH]cc(C#N)c12)c1cn2ccc(Cl)c2c(=O)n1-c1ccccc1] | 430.1 | ¹H NMR (400 MHz, DMSO-d₆) δ: 8.05 (s, 2H), 7.62 (s, 1H), 7.48 (d, J = 2.8 Hz, 1H), 7.43-7.37 (m, 3H), 7.22 (t, J = 7.3 Hz, 1H), 7.10 (t, J = 7.3 Hz, 1H), 6.61 (d, J = 2.8 Hz, 1H), 6.37 (d, J = 6.8 Hz, 1H), 4.78-4.75 (m, 1H), 1.37 (d, 7 = 6.6 Hz, 3H). |
| 89 | 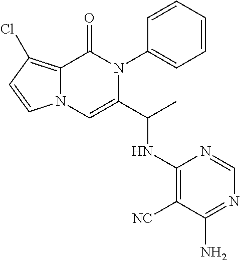 [SMILES: CC(Nc1ncnc(N)c1C#N)c1cn2ccc(Cl)c2c(=O)n1-c1ccccc1] | 406.0 | ¹H NMR (400 MHz, DMSO-d₆) δ: 7.72 (s, 1H), 7.52 (d, J = 2.7 Hz, 1H), 7.47-7.34 (m, 3H), 7.32-7.28 (m, 1H), 7.26-7.22 (m, 3H), 7.17-7.07 (s, 2H), 6.63 (d, J = 2.7 Hz, 1H), 4.77-4.69 (m, 1H), 1.27 (d, J = 6.8 Hz, 3H). |

### Example 8

### Compound 90

### 4-amino-6-(1-(8-methyl-1-oxo-2-phenyl-1,2-dihydropyrrolo[1,2-a]pyrazin-3-yl)ethylamino)pyrimidine-5-carbonitrile

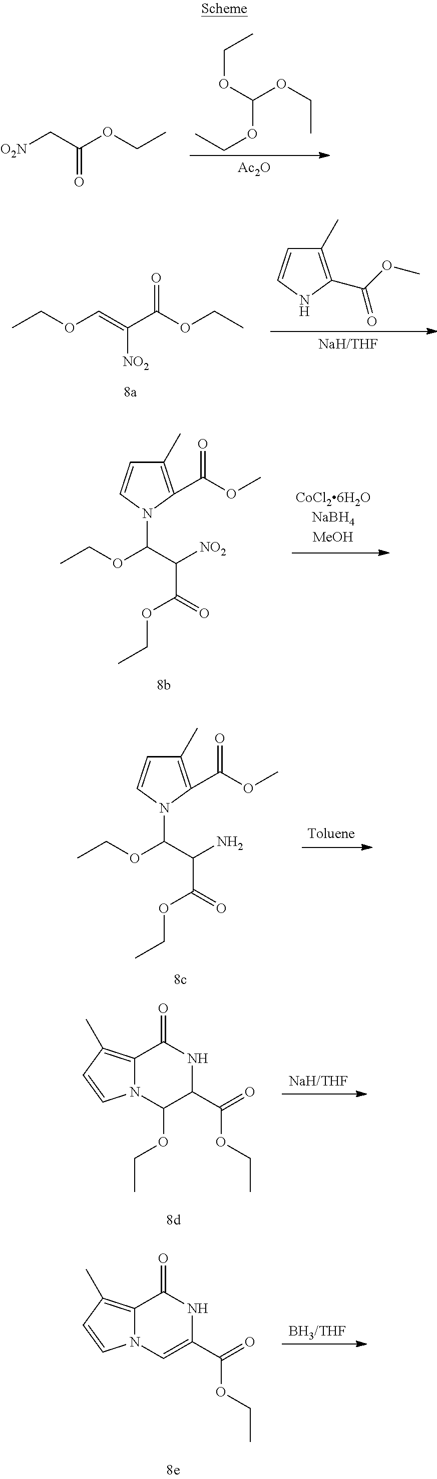

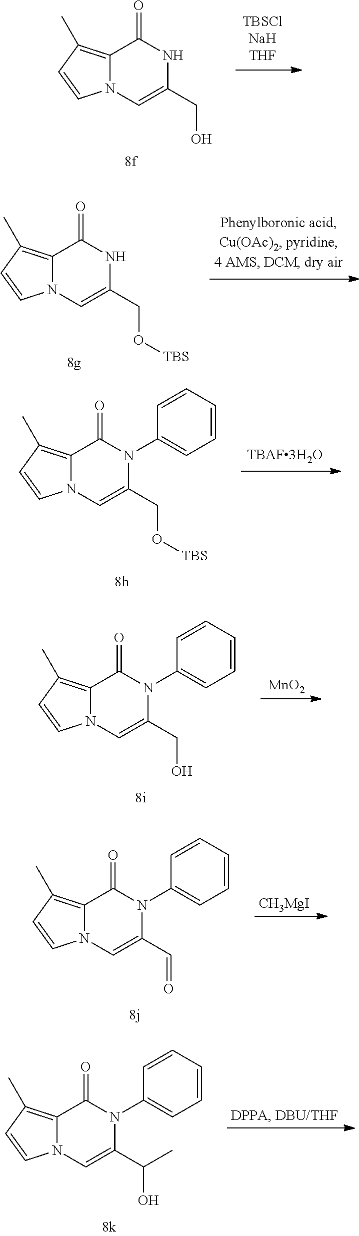

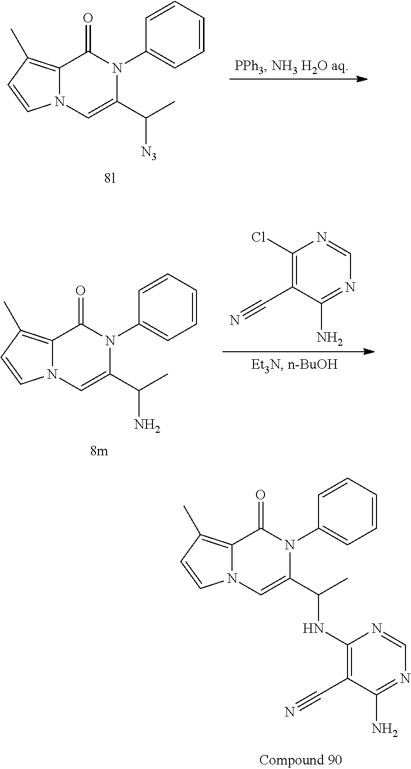

### Step 8-1 (Z)-ethyl 3-ethoxy-2-nitroacrylate (8a)

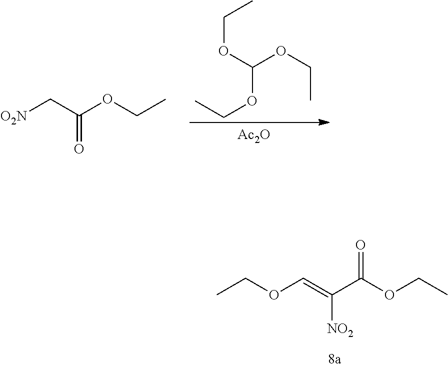

A mixture of ethyl 2-nitroacetate (26.6 g, 200 mmol) and triethoxymethane (44.5 g, 300 mmol) in acetic anhydride (51.5 g, 500 mmol) was stirred at 100° C. for 16 hours. After concentration, the residue was further distilled under reduced pressure to afford 8a as a yellow oil (30.3 g, yield: 82%). MS (m/z): 190 (M+H)⁺.

### Step 8-2 methyl 1-(1,3-diethoxy-2-nitro-3-oxopropyl)-3-methyl-1H-pyrrole-2-carboxylate (8b)

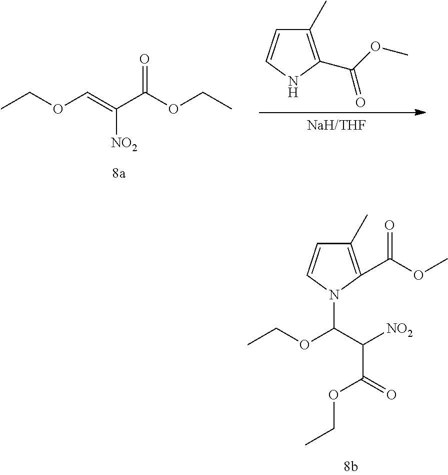

To a solution of methyl 3-methyl-1H-pyrrole-2-carboxylate (13.33 g, 96 mmol) in THF (160 mL) was added 60% NaH (5.76 g, 192 mmol) at 0-5° C. under nitrogen. The mixture was stirred at 0-5° C. for half an hour. Then 8a (27.27 g, 144 mmol) was added and the reaction was stirred at room temperature for one hour. Then the mixture was diluted with EtOAc and brine. The organic layer was collected, concentrated and purified by flash column chromatography eluting with EtOAc/PE to afford 8b as a yellow oil (24.6 g, purity: 60%).

### Step 8-3 methyl 1-(2-amino-1,3-diethoxy-3-oxopropyl)-3-methyl-1H-pyrrole-2-carboxylate (8c)

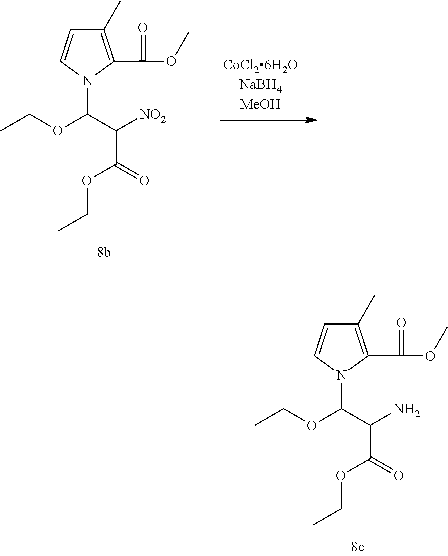 [SMILES: CCOC(=O)C(C(OCC)n1ccc(C)c1C(=O)OC)[N+](=O)[O-] ; CCOC(=O)C(N)C(OCC)n1ccc(C)c1C(=O)OC]

To a solution of 8b (21.3 g, 65 mmol) in MeOH (400 mL) was added CoCl₂.6H₂O (30.9 g, 130 mmol) followed by NaBH₄ (12.3 g, 32.4 mmol) in small portions. H₂ was evolved and the reaction was stirred at room temperature for one hour. 10% HCl aq. was added to dissolve the black precipitate and MeOH was removed by evaporation. Concentrated NH₃.H₂O aq. was added and the mixture was extracted with EtOAc. The organic layer was dried and concentrated in vacuo to afford an orange oil which was purified by flash column chromatography eluting with EtOAc/PE to give 8c as a yellow oil (9.56 g). MS (m/z): 299 (M+H)⁺.

### Step 8-4 ethyl 4-ethoxy-8-methyl-1-oxo-1,2,3,4-tetrahydropyrrolo[1,2-a]pyrazine-3-carboxylate (8d)

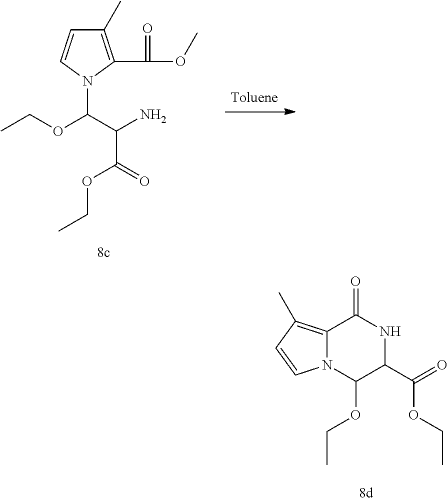 [SMILES: CCOC(=O)C(N)C(OCC)n1ccc(C)c1C(=O)OC ; CCOC(=O)C1NC(=O)c2c(C)ccn2C1OCC]

A solution of the obtained 8c (9.56 g) in toluene (180 mL) was heated at reflux under nitrogen for 40 hours. The mixture was concentrated and the residue was purified by flash column chromatography eluting with EtOAc/PE to give 8d as a brown oil (1.85 g, yield: 10%). MS (m/z): 267 (M+H)⁺.

### Step 8-5 ethyl 8-methyl-1-oxo-1,2-dihydropyrrolo[1,2-a]pyrazine-3-carboxylate (8e)

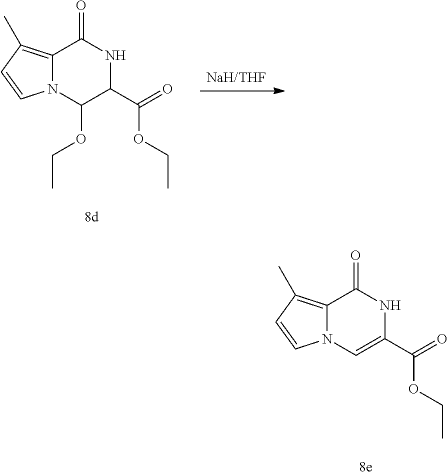 [SMILES: CCOC(=O)C1NC(=O)c2c(C)ccn2C1OCC ; CCOC(=O)c1cn2ccc(C)c2c(=O)[nH]1]

To a solution of 8d (1.85 g, 6.9 mmol) in dry THF (40 mL) cooled in an ice-bath was added 60% NaH (210 mg, 7.0 mmol) and stirred at 0-5° C. for 30 minutes. MeOH was added and followed by water. The mixture was extracted with EtOAc three times. The organic layers were combined and concentrated, the residue was purified by flash column chromatography eluting with PE/EA to give 8e as a white solid (1.60 g, yield: 100%). MS (m/z): 221 (M+H)⁺.

### Step 8-6 3-(hydroxymethyl)-8-methylpyrrolo[1,2-a]pyrazin-1(2H)-one (8f)

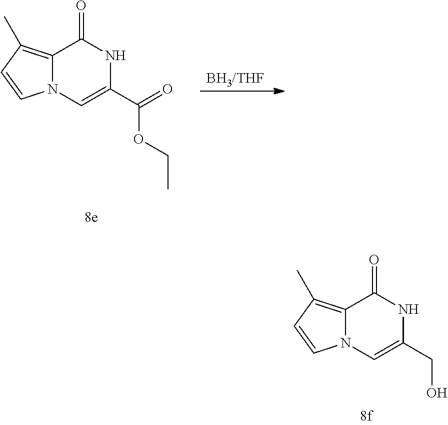 [SMILES: CCOC(=O)c1cn2ccc(C)c2c(=O)[nH]1 ; Cc1ccn2cc(CO)[nH]c(=O)c12]

To a solution of 8e (110 mg, 0.50 mmol) in THF (5 mL) was added 1M BH₃/THF (5 mL, 5 mmol) at 0-5° C. and stirred at room temperature for one hour. Water was added to quench the reaction. The mixture was diluted with EtOAc and brine. The organic layer was collected and concentrated. The residue as a white solid (65 mg, yield: 74%) was used in the next step without further purification. MS (m/z): 179 (M+H)⁺.

### Step 8-7 3-((tert-butyldimethylsilyloxy)methyl)-8-methylpyrrolo[1,2-a]pyrazin-1(2H)-one (8g)

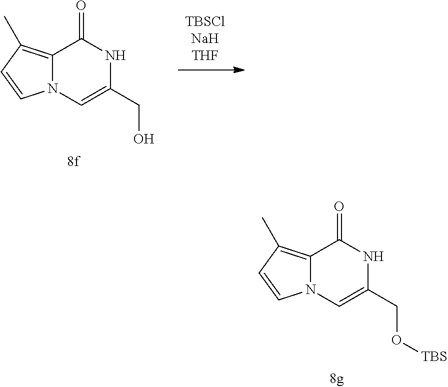

To a solution of 8f (1.78 g, 10 mmol) in dry THF (60 mL) was added 60% NaH (600 mg, 20 mmol) and the reaction was stirred at room temperature for 20 minutes. Then to the mixture was added tert-butylchlorodimethylsilane (3 g, 20 mmol) and the mixture was stirred at room temperature for another 40 minutes. The reaction was quenched by MeOH, and diluted with EtOAc and brine. The organic layer was collected, concentrated and purified by flash column chromatography eluting with EtOAc/PA to give 8g as a white solid (1.12 g, yield: 38%). MS (m/z): 293 (M+H)⁺.

### Step 8-8 3-((tert-butyldimethylsilyloxy)methyl)-8-methyl-2-phenylpyrrolo[1,2-a]pyrazin-1(2H)-one (8h)

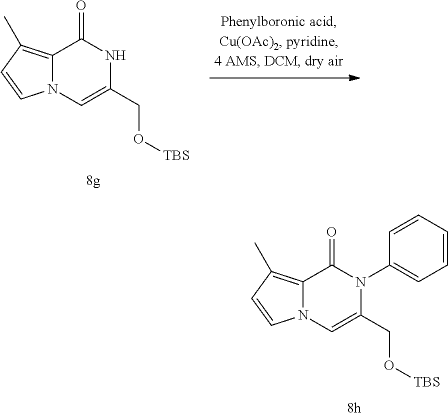

A mixture of 8g (1.03 g, 3.52 mmol), phenylboronic acid (860 mg, 7.04 mmol), diacetoxycopper (1.28 g, 7.04 mmol), pyridine (1.39 g, 17.61 mmol) and 4AMS (15 g) in DCM (60 mL) was stirred at room temperature under dry air for 16 hours. Then the reaction mixture was diluted with DCM and MeOH and filtered through celite. The filtrate was collected, concentrated and purified by flash column chromatography eluting with MeOH/H₂O to give 8h as a white solid (950 mg, yield: 73%). MS (m/z): 369 (M+H)⁺.

### Step 8-9 3-(hydroxymethyl)-8-methyl-2-phenylpyrrolo[1,2-a]pyrazin-1(2H)-one (8i)

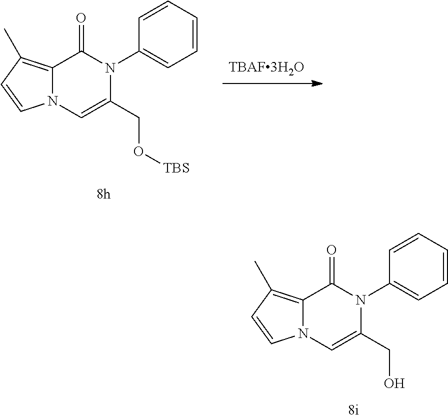

To a solution of 8h (950 mg, 2.58 mmol) in THF (10 mL) was added TBAF.3H₂O (814 mg, 2.58 mmol) and stirred at room temperature for 15 minutes. The mixture was diluted with EtOAc and washed with brine. The organic layer was collected, dried and concentrated to give 8i as a yellow oil (585 mg, yield: 89%). MS (m/z): 255 (M+H)⁺.

### Step 8-10 8-methyl-1-oxo-2-phenyl-1,2-dihydropyrrolo[1,2-a]pyrazine-3-carbaldehyde (8j)

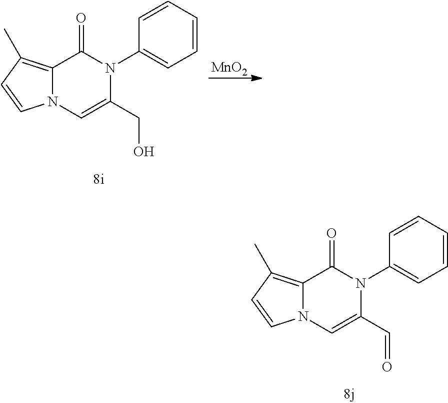

To a solution of 8i (585 mg, 2.30 mmol) in DCM (30 mL) was added MnO₂ (3.0 g, 34.4 mmol) and the reaction was stirred at room temperature overnight. The mixture was filtered through celite. The filtrate was concentrated and purified by flash column chromatography eluting with EtOAc/PE to give 8j as a white solid (366 mg, yield: 63%). MS (m/z): 252.7 (M+H)⁺.

### Step 8-11 3-(1-hydroxyethyl)-8-methyl-2-phenylpyrrolo[1,2-a]pyrazin-1(2H)-one (8k)

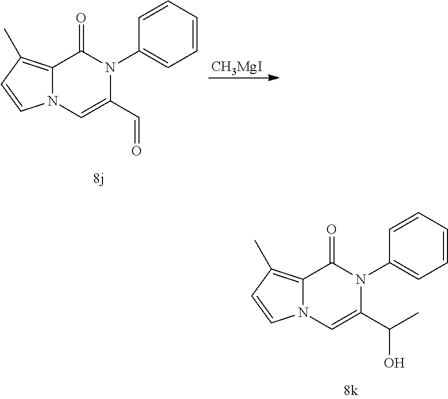

To a solution of 8j (366 mg, 1.45 mmol) in THF (30 mL) was added 2M CH₃MgI in Et₂O (1.45 mL, 2.9 mmol) at −78° C. and stirred for 30 minutes. The mixture was quenched by adding 10 mL of saturated NH₄Cl aq. and extracted with EtOAc. The organic layer was collected and concentrated to afford 8k as a yellow solid (349 mg, yield: 89.7%), which was used in the next step without further purification. MS (m/z): 269 (M+H)⁺.

### Step 8-12 3-(1-azidoethyl)-8-methyl-2-phenylpyrrolo[1,2-a]pyrazin-1(2H)-one (8l)

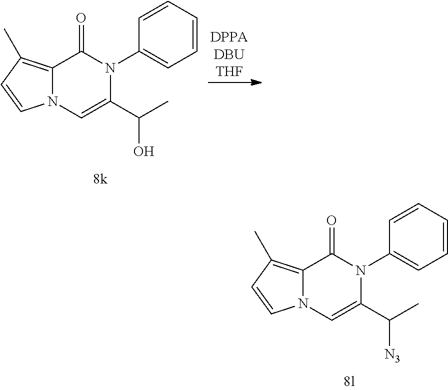 [SMILES: Cc1ccn2cc(C(C)O)n(-c3ccccc3)c(=O)c12 ; Cc1ccn2cc(C(C)N=[N+]=[N-])n(-c3ccccc3)c(=O)c12]

To a solution of 8k (349 mg, 1.3 mmol) in THF (20 mL) was added DPPA (716 mg, 2.6 mmol) at 0-5° C., followed by DBU (396 mg, 2.6 mmol) at 0-5° C. The mixture was stirred at room temperature under nitrogen for 16 hours. The mixture was concentrated and purified by flash column chromatography eluting with EtOAc/PE to give 81 as a white solid (160 mg, yield: 42%). MS (m/z): 294 (M+H)⁺.

### Step 8-13 3-(1-aminoethyl)-8-methyl-2-phenylpyrrolo[1,2-a]pyrazin-1(2H)-one (8m)

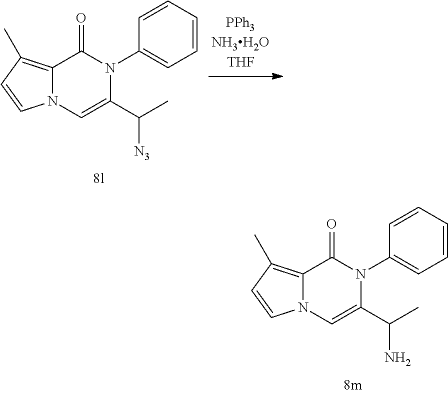 [SMILES: Cc1ccn2cc(C(C)N=[N+]=[N-])n(-c3ccccc3)c(=O)c12 ; Cc1ccn2cc(C(C)N)n(-c3ccccc3)c(=O)c12]

To a solution of 8l (160 mg, 0.54 mmol) in THF (5 mL) was added triphenylphosphine (286 mg, 1.09 mmol) and conc. NH₃.H₂O aq. (1 mL), then the reaction was stirred at 50° C. for 2 hours. The mixture was concentrated and purified by flash column chromatography eluting with MeOH/water to give 8m as a yellow solid (120 mg, yield: 82.6%). MS (m/z): 268 (M+H)⁺.

### Step 8-14 4-amino-6-(1-(8-methyl-1-oxo-2-phenyl-1,2-dihydropyrrolo[1,2-a]pyrazin-3-yl)ethylamino)pyrimidine-5-carbonitrile (90)

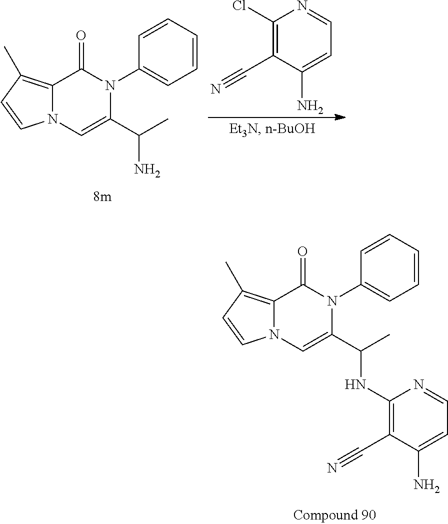

A mixture of 8m (40 mg, 0.15 mmol), 4-amino-6-chloropyrimidine-5-carbonitrile (28 mg, 0.18 mmol) and triethylamine (30 mg, 0.3 mmol) in n-BuOH (1 mL) was reacted under N₂ at reflux for 16 hours. The precipitate was collected by filtration, washed with cold n-BuOH and dried to afford Compound 90 as a white solid (38.2 mg, yield: 55%). MS (m/z): 386 (M+H)⁺. ¹H NMR (400 MHz, DMSO-d₆) δ: 7.72 (s, 1H), 7.43 (d, J=7.2 Hz, 1H), 7.41-7.31 (m, 3H), 7.29-7.19 (m, 4H), 7.10 (s, 2H), 6.37 (s, 1H), 4.77-4.69 (m, 1H), 2.38 (s, 3H), 1.26 (d, J=6.7 Hz, 3H).

The following Compounds 91 and 92 were prepared according to the procedure of Compound 90 using the corresponding reagents under appropriate conditions that will be recognized by one skilled in the art:

| Compd. | | LC/MS | |
|---|---|---|---|
| No. | Structure | (M + H)⁺ | NMR |
| 91 | 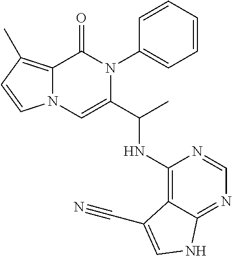 [SMILES: Cc1ccn2cc(C(C)Nc3ncnc4[nH]cc(C#N)c34)n(-c3ccccc3)c(=O)c12] | 410.0 | ¹H NMR (400 MHz, CDCl₃) δ: 8.23 (s, 1H), 7.60 (s, 1H), 7.48-7.38 (m, 2H), 7.36-7.30 (m, 1H), 7.27-7.21 (m, 2H), 7.17-7.11 (m, 1H), 7.05 (s, 1H), 6.99 (d, J = 2.5 Hz, 1H), 6.37 (d, J = 2.5 Hz, 1H), 5.47 (d, J = 7.0 Hz, 1H), 5.17-5.07 (m, 1H), 2.54 (s, 3H), 1.47 (d, J = 6.8 Hz, 3H). |
| 92 | 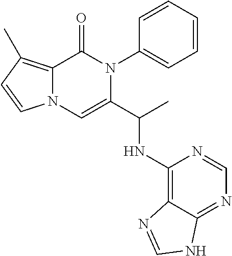 [SMILES: Cc1ccn2cc(C(C)Nc3ncnc4[nH]cnc34)n(-c3ccccc3)c(=O)c12] | 386.0 | ¹H NMR (400 MHz, DMSO-d₆) δ: 8.06-8.02 (m, 2H), 7.81 (s, 1H), 7.36-7.08 (m, 8H), 6.34 (s, 1H), 4.78 (s, 1H), 2.37 (s, 3H), 1.31 (d, J = 6.7 Hz, 3H). |

### Example 9

### Compound 93

### 3-(1-(9H-purin-6-ylamino)ethyl)-8-(1-methyl-1H-pyrazol-4-yl)-2-phenylpyrrolo[1,2-a]pyrazin-1(2H)-one

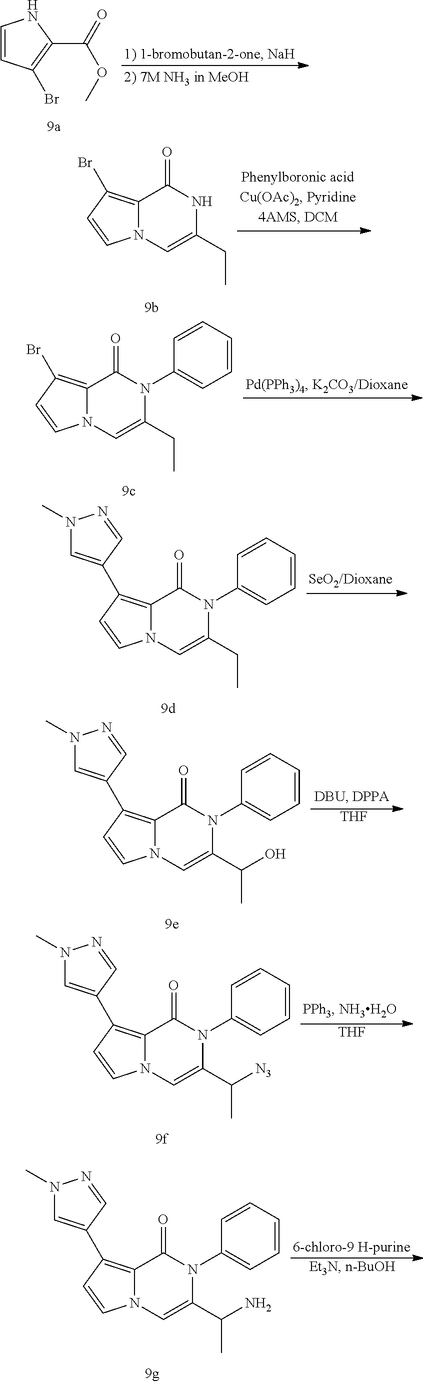

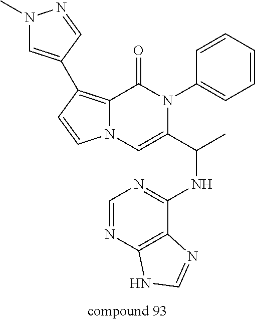 [SMILES: CC(Nc1ncnc2[nH]cnc12)c1cn2ccc(-c3cnn(C)c3)c2c(=O)n1-c1ccccc1]

### Step 9-1 8-bromo-3-ethylpyrrolo[1,2-a]pyrazin-1(2H)-one (9b)

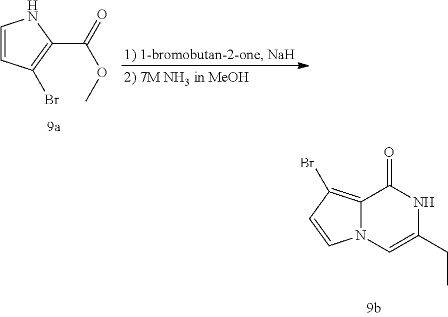

To a solution of 9a (900 mg, 4.4 mmol) in anhydrous DMF (30 mL) was added 60% NaH (246 mg, 6.2 mmol.) at 0° C. The resulting mixture was stirred at 0° C. for 30 min, then 1-bromobutan-2-one (3.3g, 22 mmol.) was added and the reaction was stirred at room temperature overnight. Then the solvent was removed in vacuo and the residue was dissolved in 7M NH₃ in MeOH (50 mL). The resulting mixture was stirred at 130° C. in a sealed tube for 24 hours. The reaction was cooled to room temperature and the solvent was removed in vacuo. The obtained residue was purified by flash column chromatography eluting with EtOAc/PE to give compound 9b as a yellow solid (700 mg, yield: 66%). MS (m/z): 241 (M+H)⁺

### Step 9-2 8-bromo-3-ethyl-2-phenylpyrrolo[1,2-a]pyrazin-1(2H)-one (9c)

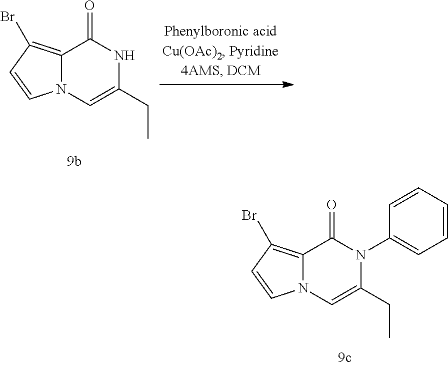 [SMILES: CCc1cn2ccc(Br)c2c(=O)[nH]1 ; CCc1cn2ccc(Br)c2c(=O)n1-c1ccccc1]

A mixture of 9b (700 mg, 2.92 mmol), phenylboronic acid (711 mg, 5.84 mmol), 4AMS (3 g), Cu(OAc)₂ (1.06 g, 5.84 mmol) and Pyridine (1.15 g, 14.6 mmol) in dry DCM (30 mL) was stirred overnight at room temperature under dry air. The mixture was filtered through celite and the filtrate was concentrated and purified by flash column chromatography eluting with MeOH/water to afford 9c as a yellow solid (520 mg, yield: 56%). MS (m/z): 317 (M+H)⁺

### Step 9-3 3-ethyl-8-(1-methyl-1H-pyrazol-4-yl)-2-phenylpyrrolo[1,2-a]pyrazin-1(2H)-one (9d)

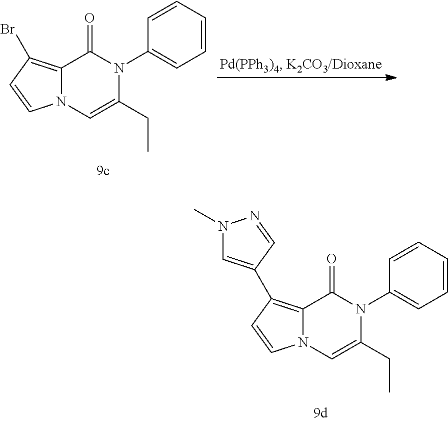 [SMILES: CCc1cn2ccc(Br)c2c(=O)n1-c1ccccc1 ; CCc1cn2ccc(-c3cnn(C)c3)c2c(=O)n1-c1ccccc1]

To a mixture of 9c (500 mg, 1.58 mmol) in 1,4-dioxane (30 mL) and water (3 mL) was added 1-methyl-4-(4,4,5,5-tetramethyl-1,3,2-dioxaborolan-2-yl)-1H-pyrazole (362 mg, 1.74 mmol), Pd(PPh₃)₄ (91 mg, 0.079 mmol) and K₂CO₃ (545 mg, 3.95 mmol). The resulting mixture was heated at reflux under N₂ for 1.5 hours. Then the solvent was removed in vacuo and water was added. The mixture was extracted with DCM three times. The organic layers were combined and concentrated to give the crude product which was purified by flash column chromatography eluting with EtOAc/PE to give 9d as a yellow solid (300 mg, yield: 60%). (m/z): 319 (M+H)⁺

### Steps 9-4 to 7 3-(1-(9H-purin-6-ylamino)ethyl)-8-(1-methyl-1H-pyrazol-4-yl)-2-phenylpyrrolo[1,2-a]pyrazin-1(2H)-one (93)

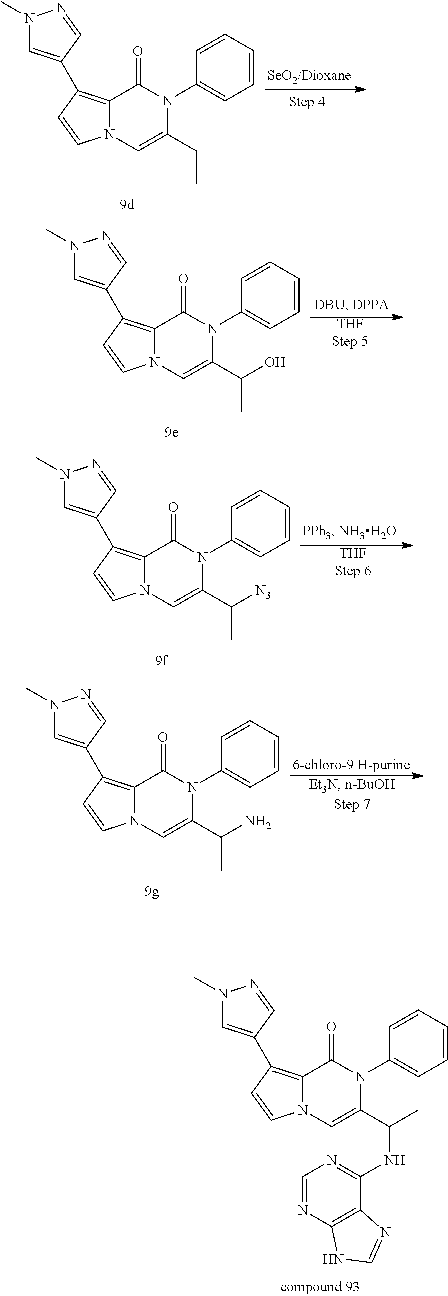

Steps 9-4 to 7 were carried out according to the procedure of Example 6 using 9d instead of 6d. Compound 93 was obtained as a white solid. MS (m/z): 451.9 (M+H)⁺; ¹H NMR (400 MHz, CD₃OD) δ: 8.18 (s, 1H), 8.04 (s, 1H), 7.99 (s, 1H), 7.90 (s, 1H), 7.51 (s, 1H), 7.47-7.39 (m, 1H), 7.36 (d, J=2.2 Hz, 1H), 7.35-7.31 (m, 1H), 7.27-7.21 (m, 1H), 7.20-7.16 (m, 1H), 6.97-6.87 (m, 1H), 6.85-6.79 (m, 1H), 5.07-4.97 (m, 1H), 3.82 (s, 3H), 1.50 (d, J=6.8 Hz, 3H).

### Example 10

### Compound 94 (S)-4-(2-(4-oxo-3-phenyl-3,4-dihydropyrrolo[2,1-f][1,2,4]triazin-2-yl)pyrrolidin-1-yl)-7H-pyrrolo[2,3-d]pyrimidine-5-carboxamide

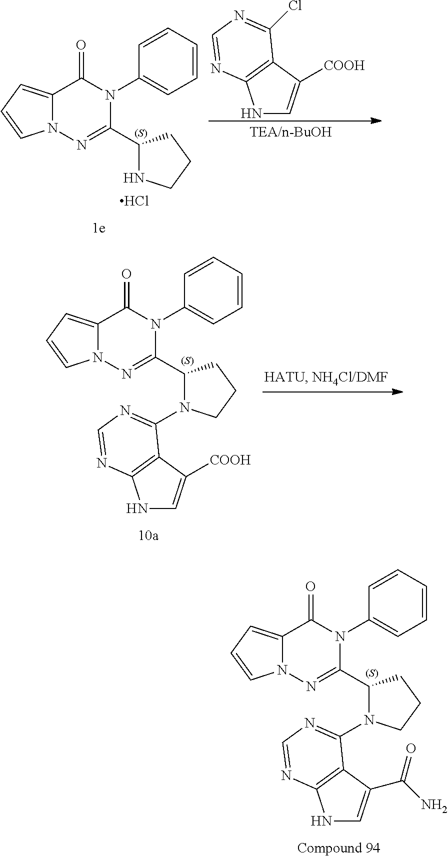

### Step 10-1 (S)-4-(2-(4-oxo-3-phenyl-3,4-dihydropyrrolo[2,1-f][1,2,4]triazin-2-yl) pyrrolidin-1-yl)-7H-pyrrolo[2,3-d]pyrimidine-5-carboxylic acid (10a)

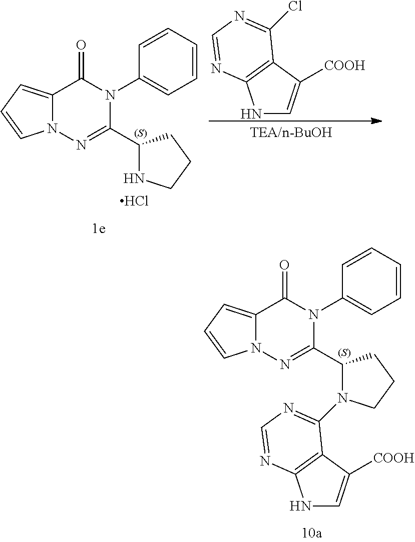

Step 10-1 was carried out according to the procedure of Example 1 using 4-chloro-7H-pyrrolo[2,3-d]pyrimidine-5-carboxylic acid instead of 4-chloro-7H-pyrrolo[2,3-d]pyrimidine-5-carbonitrile.

### Step 10-2 (S)-4-(2-(4-oxo-3-phenyl-3,4-dihydropyrrolo[2,1-f][1,2,4]triazin-2-yl) pyrrolidin-1-yl)-7H-pyrrolo[2,3-d]pyrimidine-5-carboxamide (94)

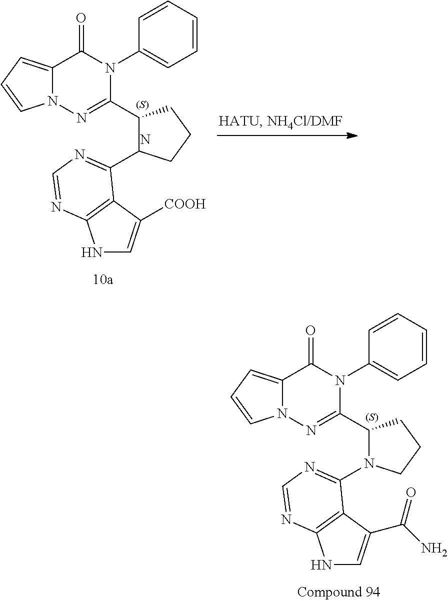

10a (123 mg, 0.28 mmol) was dissolved in DMF (10 mL) and to the solution was added HATU (117 mg, 0.31 mmol) and NH₄Cl (300 mg, 5.6 mmol). The resulting mixture was stirred at room temperature overnight. The reaction was quenched by water and extracted with DCM three times. The organic layers were combined and concentrated to give the crude product which was purified by preparative TLC eluting with DCM/MeOH to give compound 94 as a white solid (49 mg, yield: 40%). MS (m/z): 440.7 (M+H)⁺; ¹H NMR (400 MHz, DMSO-d₆) δ: 12.08 (s, 1H), 8.22 (s, 1H), 7.90-7.70 (m, 2H), 7.65-7.43 (m, 6H), 7.28 (s, 1H), 6.90 (s, 1H), 6.50 (s, 1H), 4.69-4.57 (m, 1H), 4.09-3.99 (m, 1H), 3.90-3.80 (m, 1H), 2.19-2.05 (m, 2H), 1.98-1.88 (m, 1H), 1.81-1.71 (m, 1H).

The following Compounds were prepared according to the procedure of Compound 94 using the corresponding reagents and intermediates under appropriate conditions that will be recognized by one skilled in the art:

| Compd. | | LC/MS | |
|---|---|---|---|
| No. | Structure | (M + H)⁺ | NMR |
| 95 | 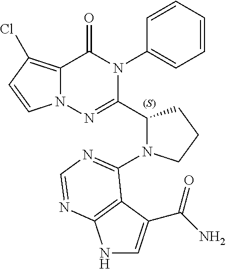 | 474.7 | ¹H NMR (400 MHz, DMSO-d₆) δ: 12.01 (s, 1H), 8.22 (s, 1H), 7.76 (d, J = 7.3 Hz, 2H), 7.67-7.48 (m, 5H), 7.45 (d, J = 2.3 Hz, 1H), 7.26 (s, 1H), 6.60-6.59 (m, 1H), 4.65-4.55 (m, 1H), 4.14-3.97 (m, 1H), 3.90-3.80 (m, 1H), 2.24-2.06 (m, 2H), 2.01-1.85 (m, 1H), 1.85-1.71 (m, 1H). |
| 96 | 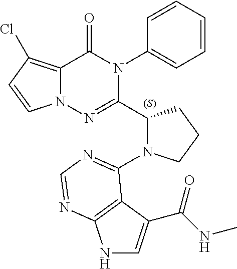 | 488.8 | ¹H NMR (400 MHz, CD₃OD) δ: 8.17 (s, 1H), 7.78-7.72 (m, 1H), 7.69-7.49 (m, 3H), 7.42 (d, J = 5.3 Hz, 2H), 7.28 (d, J = 2.7 Hz, 1H), 6.45 (d, J = 2.8 Hz, 1H), 4.74-4.68 (m, 1H), 4.01-3.91 (m, 1H), 3.83-3.70 (m, 1H), 2.90 (s, 3H), 2.19-1.96 (m, 3H), 1.82-1.72 (m, 1H). |
| 97 | 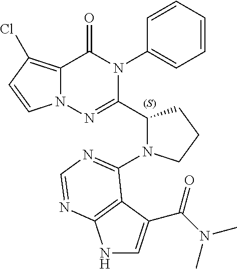 | 502.7 | ¹H NMR (400 MHz, CD₃OD) δ: 8.18 (s, 1H), 7.81-7.76 (m, 1H), 7.65-7.60 (m, 1H), 7.60-7.52 (m, 2H), 7.42 (dt, J = 4.3, 1.9 Hz, 1H), 7.26 (s, 1H), 7.22 (d, J = 3.0 Hz, 1H), 6.44 (d, J = 3.0 Hz, 1H), 4.81-4.77 (m, 1H), 3.80-3.70 (m, 2H), 3.09 (s, 6H), 2.23-2.15 (m, 1H), 2.11-2.01 (m, 2H), 1.84-1.74 (m, 1H). |
| 163 | 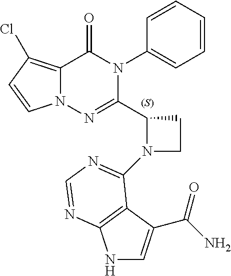 | 461.5 | ¹H NMR (400 MHz, DMSO-d₆) δ: 8.10-7.80 (m, 3H), 7.79-7.38 (m, 6H), 7.08 (s, 1H), 6.68 (d, J = 3.1 Hz, 1H), 5.50-5.30 (m, 1H), 4.25-3.98 (br, 2H), 2.19-1.99 (m, 2H). |
| 164 | 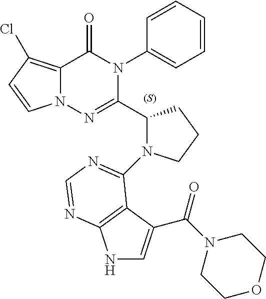 | 545.1 | ¹H NMR (400 MHz, DMSO-d₆) δ 8.19 (s, 1H), 7.77 (d, J = 7.9 Hz, 1H), 7.63-7.53 (m, 5H), 7.41 (s, 1H), 7.33 (s, 1H), 6.61 (d, J = 3.0 Hz, 1H), 4.62-4.54 (m, 1H), 3.81-3.62 (m, 10H), 2.15-2.11 (m, 2H), 1.97-1.89 (m, 1H), 1.84-1.76 (m, 1H). |

### Example 11

### Compound 98

### (S)-3-phenyl-2-(1-(5-vinyl-7H-pyrrolo[2,3-d]pyrimidin-4-yl)pyrrolidin-2-yl) pyrrolo[2,1-f][1,2,4]triazin-4(3H)-one

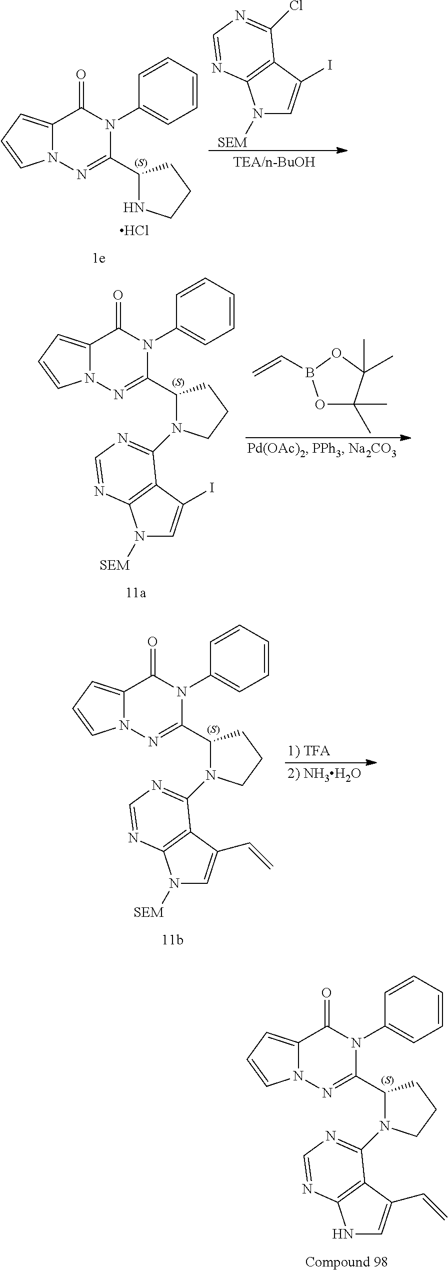

### Step 11-1 (S)-2-(1-(5-iodo-7-((2-(trimethylsilyl)ethoxy)methyl)-7H-pyrrolo[2,3-d]pyrimidin-4-yl)pyrrolidin-2-yl)-3-phenylpyrrolo[2,1-f][1,2,4]triazin-4(3H)-one (11a)

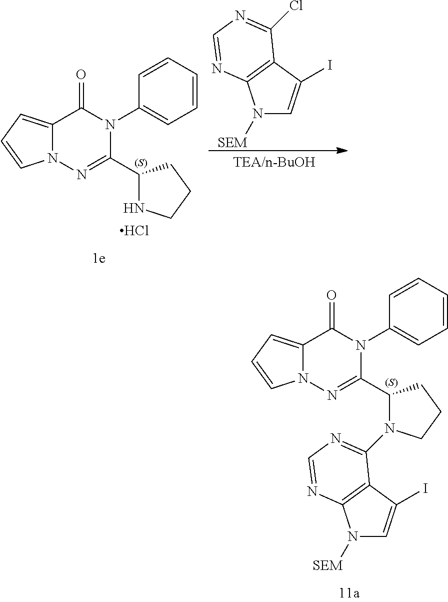

Step 11-1 was carried out according to the procedure of Example 1 using 4-chloro-5-iodo-7-((2-(trimethylsilyl)ethoxy)methyl)-7H-pyrrolo[2,3-d]pyrimidine instead of 4-chloro-7H-pyrrolo[2,3-d]pyrimidine-5-carbonitrile.

### Step 11-2 (S)-3-phenyl-2-(1-(7-((2-(trimethylsilyl)ethoxy)methyl)-5-vinyl-7H-pyrrolo[2,3-d]pyrimidin-4-yl)pyrrolidin-2-yl)pyrrolo[2,1-f][1,2,4]triazin-4(3H)-one (11b)

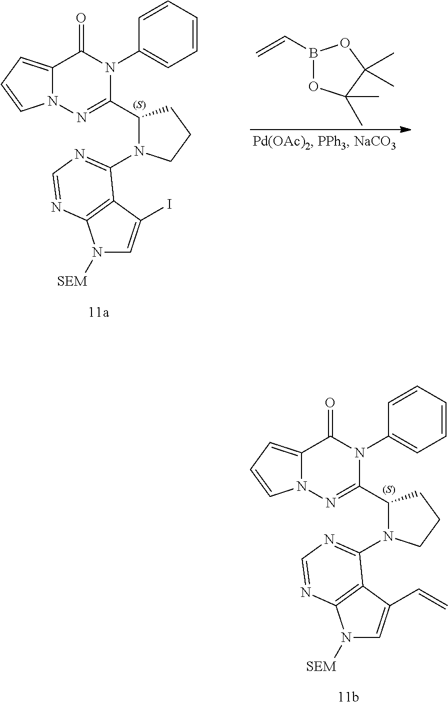

To a solution of 11a (70 mg, 0.11 mmol) in DMF/EtOH/H₂O (4 mL/1 mL/1 mL) were added 4,4,5,5-tetramethyl-2-vinyl-1,3,2-dioxaborolane (51 mg, 0.33 mmol), Pd(OAc)₂ (1.2 mg, 0.006 mmol), PPh₃ (2.8 mg, 0.011 mmol) and Na₂CO₃ (70 mg, 0.66 mmol). Under N₂, the reaction mixture was heated at 100° C. overnight. Then the solvent was removed in reduced pressure and the residue was purified by flash column chromatography eluting with MeOH/water to give 11b as a yellow solid (20 mg, yield: 33%).

### Step 11-3 (S)-3-phenyl-2-(1-(5-vinyl-7H-pyrrolo[2,3-d]pyrimidin-4-yl)pyrrolidin-2-yl)pyrrolo[2,1-f][1,2,4]triazin-4(3H)-one (98)

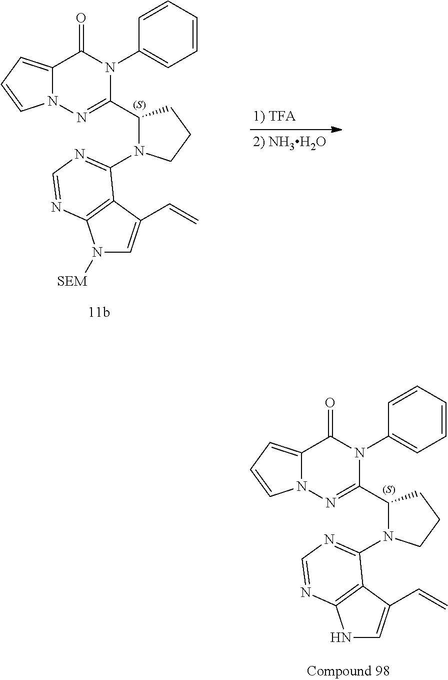

11b (20 mg, 0.036 mmol) was dissolved in TFA (3 mL) cooled in the ice bath. The resulting mixture was stirred at room temperature for 2 hours. Then the solvent was removed in vacuo. The residue was dissolved in MeOH (1 mL) and 7N NH₃ in MeOH (1 mL) was added. The mixture was stirred at room temperature for 2 hours. The solvent was removed in vacuo and the residue was purified by flash column chromatography eluting with MeOH/water to give compound 98 as a white solid (7 mg, yield: 46%). MS (m/z): 423.7 (M+H)⁺; ¹H NMR (400 MHz, CDCl₃) δ: 7.82-7.76 (m, 1H), 7.60-7.52 (m, 3H), 7.28 (s, 1H), 7.26-7.20 (m, 2H), 7.08-7.02 (m, 2H), 6.95-6.88 (m, 1H), 6.51-6.40 (m, 1H), 5.53-5.43 (m, 1H), 5.22-5.12 (m, 1H), 4.99-4.93 (m, 1H), 4.05-3.94 (m, 1H), 3.81-3.71 (m, 1H), 2.31-2.21 (m, 1H), 2.12-1.95 (m, 2H), 1.91-1.82 (m, 1H).

The following Compounds were prepared according to the procedure of Example 98 using the corresponding reagents and intermediates under appropriate conditions that will be recognized by one skilled in the art:

| Compd. | | LC/MS | |
|---|---|---|---|
| No. | Structure | (M + H)⁺ | NMR |
| 100 | 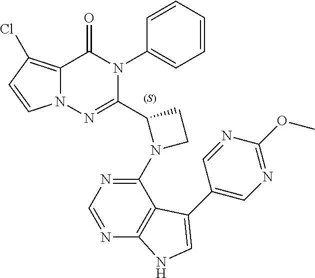 | 525.9 | ¹H NMR (400 MHz, DMSO-d₆) δ 12.16 (s, 1H), 8.73 (s, 2H), 8.25 (s, 1H), 7.67-7.41 (m, 7H), 6.71-6.61 (br, 1H), 5.08-4.98 (m, 1H), 3.95 (s, 3H), 3.30-3.25 (m, 1H), 3.10-3.00 (m, 1H), 2.44-2.36 (m, 1H), 1.75-1.67 (m, 1H). |
| 101 | 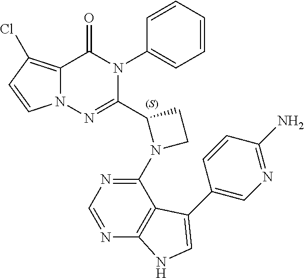 | 509.8 | ¹H NMR (400 MHz, DMSO-d₆) δ 11.84 (s, 1H), 8.18 (s, 1H), 8.05-8.01 (m, 1H), 7.66-7.62 (m, 1H), 7.60-7.56 (m, 1H), 7.54-7.46 (m, 4H), 7.43-7.39 (m, 1H), 7.19-7.15 (m, 1H), 6.67-6.63 (m, 1H), 6.52-6.46 (m, 1H), 5.89 (s, 2H), 5.00-4.92 (m, 1H), 3.29-3.25 (m, 1H), 3.18-3.10 (m, 1H), 2.39-2.23 (m, 1H), 1.76-1.66 (m, 1H). |
| 102 | 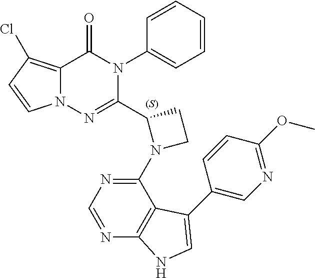 | 524.8 | ¹H NMR (400 MHz, DMSO-d₆) δ 12.01 (s, 1H), 8.37-8.17 (m, 2H), 7.86-7.78 (m, 1H), 7.69-7.65 (m, 1H), 7.63-7.45 (m, 4H), 7.43-7.39 (m, 1H), 7.33-7.29 (m, 1H), 6.89-6.85 (m, 1H), 6.67-6.63 (m, 1H), 5.02-4.94 (m, 1H), 3.88 (s, 3H), 3.28-3.24 (m, 1H), 3.07-2.98 (m, 1H), 2.40-2.29 (m, 1H), 1.74-1.64 (m, 1H). |
| 103 | 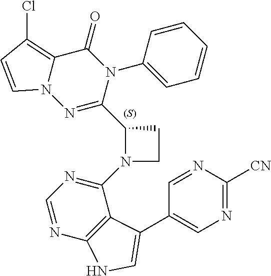 | 520.9 | ¹H NMR (400 MHz, DMSO-d₆) δ 12.54 (s, 1H), 9.27 (s, 2H), 8.45-8.18 (m, 1H), 7.95-7.85 (m, 1H), 7.73-7.18 (m, 6H), 6.80-6.72 (m, 1H), 5.15-4.96 (m, 1H), 3.20-3.14 (m, 2H), 2.42-2.24 (m, 1H), 1.72-1.62 (m, 1H). |
| 104 | 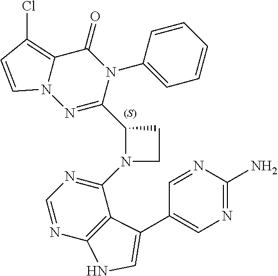 | 510.8 | ¹H NMR (400 MHz, DMSO-d₆) δ 12.04 (s, 1H), 8.37 (s, 2H), 8.22 (s, 1H), 7.68-7.64 (m, 1H), 7.62-7.58 (m, 1H), 7.58-7.48 (m, 3H), 7.46-7.42 (m, 1H), 7.35-7.31 (m, 1H), 6.76-6.59 (m, 3H), 5.06-4.98 (m, 1H), 3.24-3.14 (m, 2H), 2.44-2.38 (m, 1H), 1.78-1.68 (m, 1H). |
| 165 | 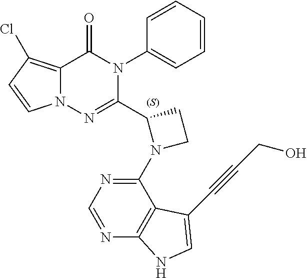 | 472.2 | ¹H NMR (400 MHz, DMSO-d₆) δ: 11.84 (s, 1H), 8.17 (s, 1H), 7.75-7.71 (m, 1H), 7.63-7.53 (m, 5H), 7.49-7.45 (m, 1H), 6.68-6.63 (m, 1H), 5.33-5.23 (m, 1H), 5.00-4.94 (m, 1H), 4.65-4.55 (m, 1H), 4.33-4.27 (m, 2H), 4.15-4.07 (m, 1H), 2.66-2.59 (m, 1H), 2.11-2.03 (m, 1H). |
| 279 | 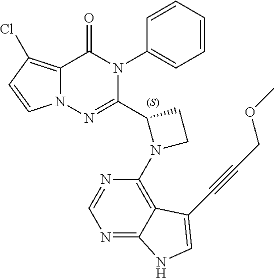 | 486.1 | ¹H NMR (400 MHz, DMSO-d₆) δ: 8.14 (s, 1H), 7.70 (d, J = 3.0 Hz, 1H), 7.67-7.49 (m, 5H), 7.47-7.40 (m, 1H), 6.62 (d, J = 2.9 Hz, 1H), 4.98-4.91 (m, 1H), 4.55-4.45 (m, 1H), 4.28 (s, 2H), 4.12-4.05 (m, 1H), 3.26 (s, 3H), 2.62-2.56 (m, 1H), 2.07-2.00 (m, 1H). |

### Example 12

### Compound 105

### (S)-4-(2-(5-ethynyl-4-oxo-3-phenyl-3,4-dihydropyrrolo[2,1-f][1,2,4]triazin-2-yl) azetidin-1-yl)-7H-pyrrolo[2,3-d]pyrimidine-5-carbonitrile

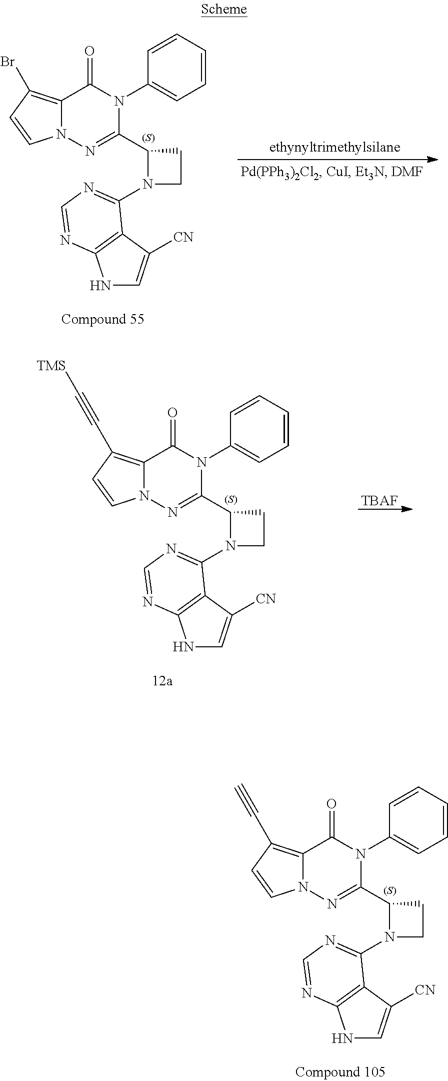

### Step 12-1 (S)-4-(2-(4-oxo-3-phenyl-5-((trimethylsilyl)ethynyl)-3,4-dihydropyrrolo[2,1-f][1,2,4]triazin-2-yl)azetidin-1-yl)-7H-pyrrolo[2,3-d]pyrimidine-5-carbonitrile (12a)

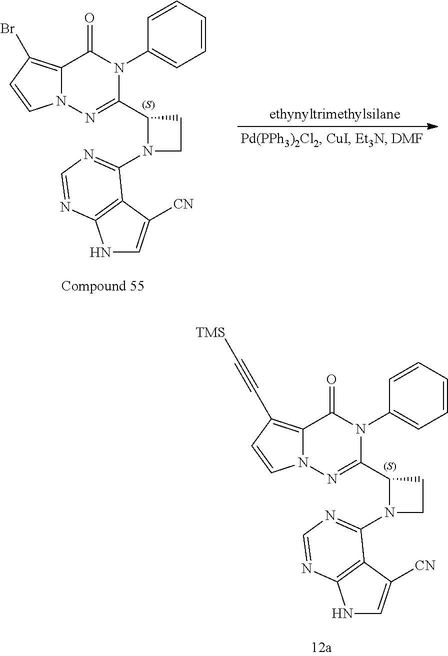

To a mixture of Compound 55 (84 mg, 0.173 mmol), Pd(PPh₃)₂Cl₂ (8 mg, 0.0116 mmol) and CuI (2.2 mg, 0.0116 mmol) in DMF (4 mL) was added Et₃N (0.36 mL, 2.6 mmol) and ethynyltrimethylsilane (44 mg, 0.448 mmol). The reaction was heated under N₂ at 90° C. for 4 hours, then the mixture was cooled to room temperature, filtered and concentrated. The residue was further purified by flash column chromatography eluting with MeOH/water to get 12a (60 mg, yield: 69%). MS (m/z): 505 (M+H)⁺.

### Step 12-2 (S)-4-(2-(5-ethynyl-4-oxo-3-phenyl-3,4-dihydropyrrolo[2,1-f][1,2,4]triazin-2-yl)azetidin-1-yl)-7H-pyrrolo[2,3-d]pyrimidine-5-carbonitrile (105)

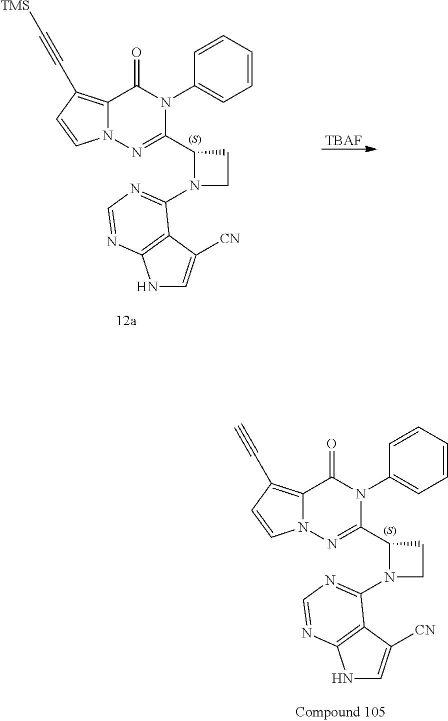

To a solution of 12a (60 mg, 0.12 mmol) in DMF (2 mL) was added 1.0 M TBAF in THF (0.15 mL, 0.15 mmol). After 20 minutes, the reaction mixture was diluted in water and extracted with EtOAc three times. The combined organic layers were dried, filtered and concentrated to give the crude product which was purified by flash column chromatography eluting with MeOH/water to afford Compound 105 as a white solid (2.0 mg, yield: 4%). MS (m/z): 433.2 (M+H)⁺. ¹H NMR (400 MHz, CD₃OD) δ: 8.22 (s, 1H), 7.94 (s, 1H), 7.74 (d, J=7.6 Hz, 1H), 7.66-7.59 (m, 1H), 7.58-7.51 (m, 2H), 7.40-7.30 (m, 2H), 6.64 (d, J=2.8 Hz, 1H), 5.33 (dd, J=9.5, 5.2 Hz, 1H), 4.64-4.60 (m, 1H), 4.32-4.20 (m, 1H), 3.52 (s, 1H), 2.67-2.51 (m, 1H), 2.07-1.97 (m, 1H).

### Example 14

### Compound 107

### (S)-4-(2-(7-fluoro-3-isobutyl-4-oxo-3,4-dihydropyrrolo[2,1-f][1,2,4]triazin-2-yl) pyrrolidin-1-yl)-7H-pyrrolo[2,3-d]pyrimidine-5-carbonitrile

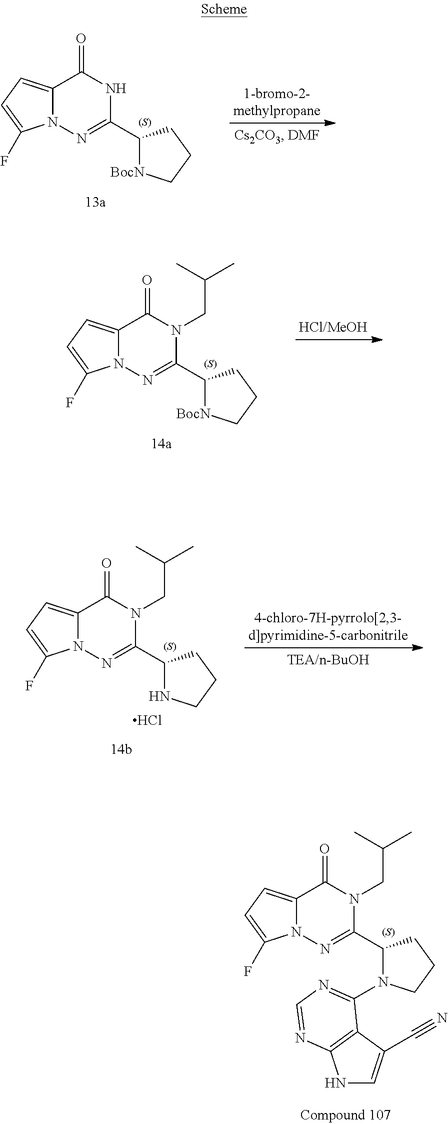

### Step 14-1 (S)-tert-butyl 2-(7-fluoro-3-isobutyl-4-oxo-3,4-dihydropyrrolo[2,1-f][1,2,4]triazin-2-yl)pyrrolidine-1-carboxylate (14a)

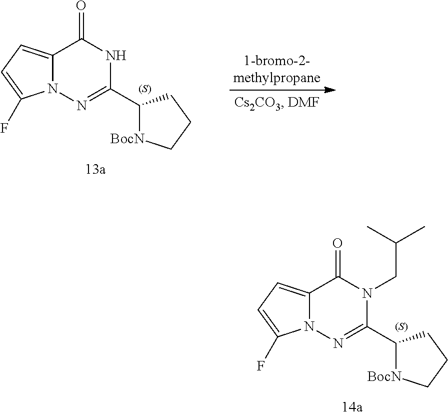

To a mixture of 13a (200 mg, 0.62 mmol) and Cs₂CO₃ (403 mg, 1.24 mmol) in DMF (5 mL) was added 1-bromo-2-methylpropane (170 mg, 1.24 mmol), then the reaction was heated to 80° C. for 2 hours. The mixture was diluted with water and extracted with EtOAc three times. The combined organic layers were washed with brine, dried over MgSO₄, filtered, concentrated and purified by flash column chromatography eluting with MeOH/water to give 14a (50 mg, yield: 21%). MS (m/z): 278.8 (M-Boc+H)⁺.

### Step 14-2 (S)-7-fluoro-3-isobutyl-2-(pyrrolidin-2-yl)pyrrolo[2,1-f][1,2,4]triazin-4(3H)-one hydrochloride (14b)

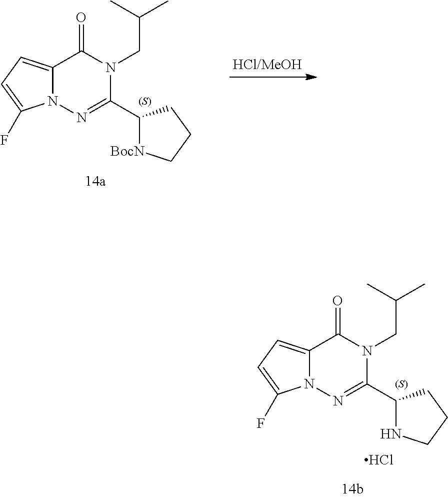

To a mixture of 14a (50 mg, 0.132 mmol) in MeOH (5 mL) was added conc. HCl aq (5 mL), then the reaction was stirred at room temperature for 2 hours. After concentration under reduced pressure, 14b was obtained as a yellow oil which was used directly in the next step without further purification. MS (m/z): 278.8 (M+H)⁺

### Step 14-3 (S)-4-(2-(7-fluoro-3-isobutyl-4-oxo-3,4-dihydropyrrolo[2,1-f][1,2,4]triazin-2-yl)pyrrolidin-1-yl)-7H-pyrrolo[2,3-d]pyrimidine-5-carbonitrile (107)

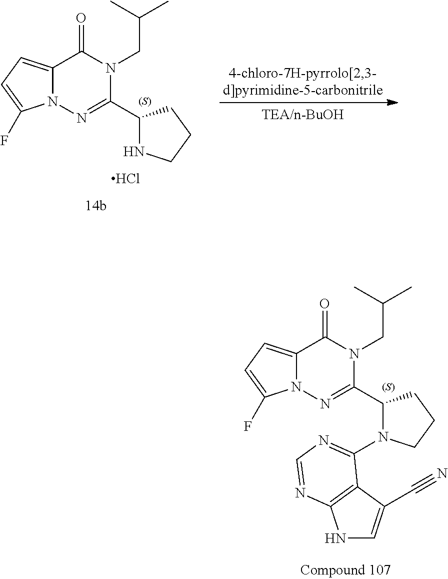

A mixture of 14b (0.132 mmol), 4-chloro-7H-pyrrolo[2,3-d]pyrimidine-5-carbonitrile (24 mg, 0.132 mmol) and TEA (0.09 mL, 0.66 mmol) in n-BuOH (10 mL) was heated at reflux for 2 hours. The reaction mixture was concentrated purified by flash column chromatography eluting with MeOH/water to afford compound 107 as a slight yellow solid (17 mg, yield: 31%). MS (m/z): 420.7 (M+H)⁺. ¹H-NMR (400 MHz, DMSO-d₆) δ: 8.29 (s, 1H), 8.03 (s, 1H), 6.77 (t, J=5.1 Hz, 1H), 6.16 (t, J=4.0 Hz, 1H), 5.55-5.45 (m, 1H), 4.30-4.22 (m, 1H), 4.18-4.05 (m, 2H), 3.71-3.67 (m, 1H), 2.37-2.01 (m, 5H), 1.00 (d, T=6.6 Hz, 3H), 0.93 (d, T=6.5 Hz, 3H).

### Example 15

### Compound 108

### (S)-2-(1-(6-amino-5-(6-methoxypyridin-3-yl)pyrimidin-4-yl)azetidin-2-yl)-5-chloro-3-phenylpyrrolo[2,1-f][1,2,4]triazin-4(3H)-one

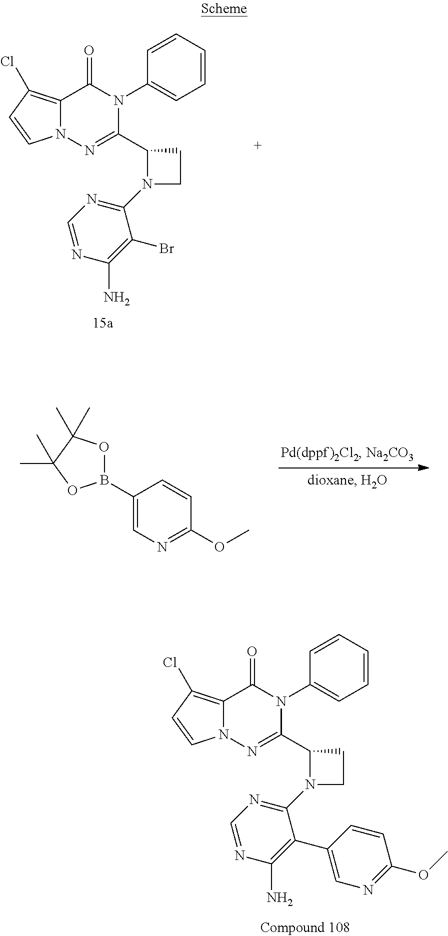

A mixture of 15a (50 mg, 0.106 mmol) (15a was prepared according to the procedure of Example 1), 2-methoxy-5 tetramethyl-1,3,2-dioxaborolan-2-yl)pyridine (28 mg, 0.116 mmol), Pd(dppf)₂Cl₂ (9 mg, 0.0106 mmol) and Na₂CO₃ (23 mg, 0.212 mmol) in dioxane (20 mL) and water (2 mL) was heated at 130° C. under N₂ atmosphere for 3 hours. Then the mixture was filtered, concentrated and purified by flash column chromatography eluting with MeOH/water to give Compound 108 as a white solid (30 mg, yield: 56%). MS (m/z): 500.6 (M+H)⁺. ¹H NMR (400 MHz, DMSO-d₆) δ: 8.18-7.39 (m, 8H), 7.29 (d, J=6.4 Hz, 2H), 6.73-6.57 (m, 1H), 5.82 (s, 2H), 4.55-4.45 (m, 1H), 3.81 (s, 3H), 3.22-3.08 (m, 2H), 2.29-2.19 (m, 1H), 1.80-1.70 (m, 1H).

The following Compounds were prepared according to the procedure of Compound 108 using the corresponding reagents and intermediates under appropriate conditions that will be recognized by one skilled in the art:

| Compd. | | LC/MS | |
|---|---|---|---|
| No. | Structure | (M + H)⁺ | NMR |
| 109 | 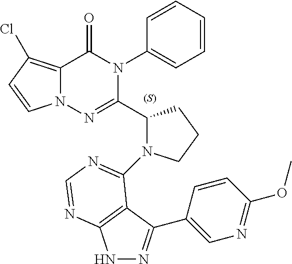 | 539.8 | ¹H NMR (400 MHz, CD₃OD) δ 8.72 (s, 1H), 8.61 (s, 1H), 8.30-8.26 (m, 1H), 8.12-8.08 (m, 1H), 8.03-7.98 (m, 1H), 7.94-7.84 (m, 2H), 7.78-7.68 (m, 2H), 7.28-7.24 (m, 1H), 6.83-6.81 (m, 1H), 5.65-5.63 (m, 1H), 4.29 (s, 3H), 3.83-3.73 (m, 1H), 3.49-3.46 (m, 1H), 2.37-2.22 (m, 4H). |
| 166 | 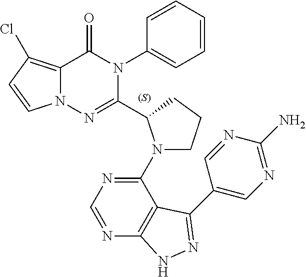 | 526.3 | ¹H NMR (400 MHz, CDCl₃) δ: 12.06 (s, 1H), 8.70 (s, 2H), 8.39 (s, 1H), 7.79-7.73 (m, 1H), 7.68-7.60 (m, 1H), 7.57-7.49 (m, 2H), 7.30-7.22 (m, 2H), 6.45-6.41 (m, 1H), 5.32 (s, 2H), 5.10-5.02 (m, 1H), 3.43-3.35 (m, 1H), 3.28-3.20 (m, 1H), 2.06-1.94 (m, 4H). |
| 167 | 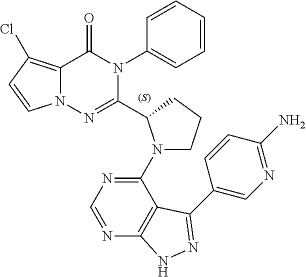 | 525.4 | ¹H NMR (400 MHz, CDCl₃) δ: 12.31 (s, 1H), 8.45-8.35 (m, 2H), 7.84-7.74 (m, 2H), 7.65-7.50 (m, 3H), 7.29-7.25 (m, 1H), 7.22-7.20 (m, 1H), 6.67-6.59 (m, 1H), 6.45-6.39 (m, 1H), 5.03-4.97 (m, 1H), 4.71 (s, 2H), 3.41-3.33 (m, 1H), 3.23-3.15 (m, 1H), 2.00-1.90 (m, 4H). |
| 168 | 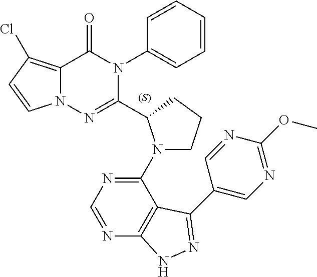 | 541.8 | ¹H NMR (400 MHz, DMSO-d₆) δ: 8.86 (s, 2H), 8.33 (s, 1H), 7.78-7.50 (m, 7H), 6.67-6.59 (m, 1H), 4.78-4.72 (m, 1H), 4.00 (s, 3H), 3.10-3.04 (m, 2H), 2.09-2.01 (m, 1H), 1.95-1.87 (m, 1H), 1.85-1.77 (m, 1H), 1.60-1.52 (m, 1H). |
| 169 | 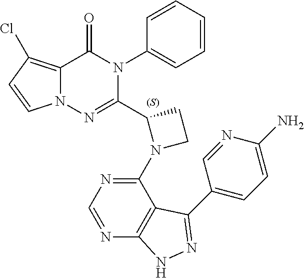 | 511.2 | ¹H NMR (400 MHz, DMSO-d₆) δ: 13.61 (s, 1H), 8.26 (s, 1H), 8.09 (d, J = 2.0 Hz, 1H), 7.75-7.31 (m, 7H), 6.64 (d, J = 2.9 Hz, 1H), 6.51 (d, J = 8.5 Hz, 1H), 6.19 (s, 2H), 5.02-4.88 (m, 1H), 3.45-3.39 (m, 2H), 2.43-2.37 (m, 1H), 1.87-1.81 (m, 1H). |
| 170 | 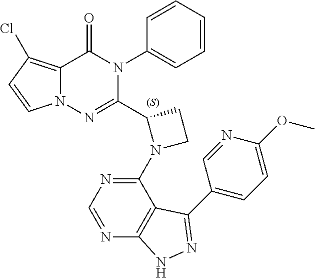 | 526.1 | ¹H NMR (400 MHz, DMSO-d₆) δ: 13.81 (s, 1H), 8.44-8.21 (m, 2H), 7.87 (dd, J = 8.5, 2.3 Hz, 1H), 7.71-7.33 (m, 6H), 6.93 (d, J = 8.5 Hz, 1H), 6.65 (d, J = 3.0 Hz, 1H), 4.98-4.90 (m, 1H), 3.89 (s, 3H), 3.40-3.36 (m, 2H), 2.45-2.35 (m, 1H), 1.88-1.69 (m, 1H). |
| 171 | 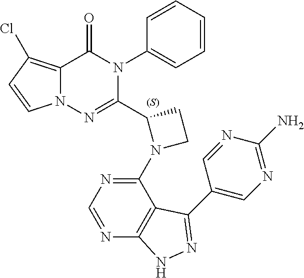 | 512.2 | ¹H NMR (400 MHz, DMSO-d₆) δ: 13.76 (s, 1H), 8.40 (s, 2H), 8.28 (s, 1H), 7.65-7.54 (m, 2H), 7.51-7.36 (m, 4H), 6.93 (s, 2H), 6.64 (d, J = 3.0 Hz, 1H), 5.04-4.90 (m, 1H), 3.59-3.40 (m, 2H), 2.04-1.78 (m, 2H). |
| 172 | 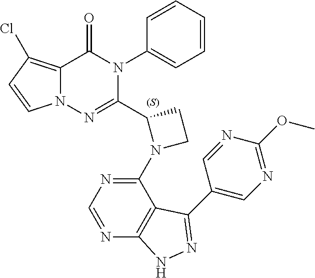 | 527.2 | ¹H NMR (400 MHz, DMSO-d₆) δ: 8.78 (s, 2H), 8.32 (s, 1H), 7.63 (d, J = 2.9 Hz, 1H), 7.60-7.40 (m, 4H), 7.39 (dd, J = 4.8, 2.2 Hz, 1H), 6.65 (d, J = 3.0 Hz, 1H), 5.02-4.90 (m, 1H), 4.12-4.02 (m, 1H), 3.96 (s, 3H), 3.52-3.41 (m, 1H), 2.03-1.76 (m, 2H). |
| 173 | 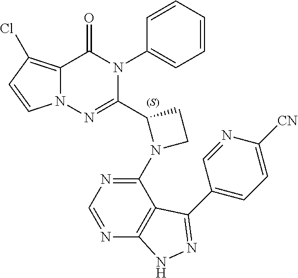 | 521.2 | ¹H NMR (400 MHz, DMSO-d₆) δ: 8.98 (d, J = 1.3 Hz, 1H), 8.35 (s, 1H), 8.26 (dd, J = 8.0, 2.1 Hz, 1H), 8.18 (d, J = 8.1 Hz, 1H), 7.68-7.35 (m, 6H), 6.66 (d, J = 3.0 Hz, 1H), 5.02-4.90 (m, 1H), 3.48-3.36 (m, 2H), 2.04-1.73 (m, 2H). |
| 174 | 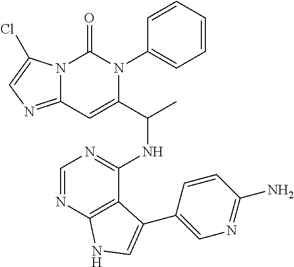 [SMILES: CC(Nc1ncnc2[nH]cc(-c3ccc(N)nc3)c12)c1cc2ncc(Cl)n2c(=O)n1-c1ccccc1] | 498.1 | ¹H NMR (400 MHz, CD₃OD) δ 8.13 (s, 1H), 8.05 (d, J = 2.0 Hz, 1H), 7.70-7.66 (m, 2H), 7.64 (dd, J = 8.6, 2.4 Hz, 1H), 7.59-7.43 (m, 4H), 7.12 (s, 1H), 6.70 (d, J = 8.5 Hz, 1H), 6.65 (s, 1H), 4.84-4.79 (m, 1H), 1.32 (d, J = 6.8 Hz, 4H). |
| 175 | 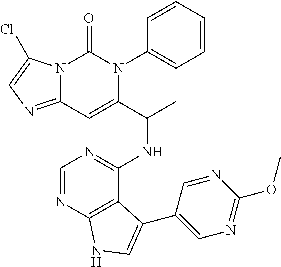 [SMILES: COc1ncc(-c2c[nH]c3ncnc(NC(C)c4cc5ncc(Cl)n5c(=O)n4-c4ccccc4)c23)cn1] | 514.2 | ¹H NMR (400 MHz, CDCl₃) δ 9.69 (s, 1H), 8.69 (s, 2H), 8.34 (s, 1H), 7.79-7.74 (m, 1H), 7.58-7.49 (m, 3H), 7.39-7.34 (m, 1H), 7.17 (s, 1H), 7.11 (d, J = 2.4 Hz, 1H), 6.50 (s, 1H), 4.85-4.78 (m, 1H), 4.74 (d, J = 5.7 Hz, 1H), 4.10 (s, 3H), 1.27 (d, J = 6.7 Hz, 3H). |
| 176 | 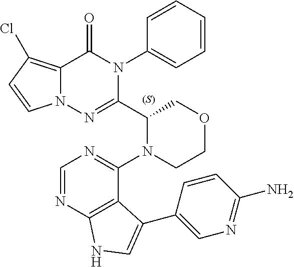 | 540.0 | ¹H NMR (400 MHz, CDCl₃) δ 9.80 (s, 1H), 8.49 (s, 1H), 8.45 ((d, J = 2.0 Hz, 1H), 7.92 (d, J = 8.0 Hz, 1H), 7.83-7.81 (dd, J = 8.4, 2.4 Hz, 1H), 7.62-7.53 (m, 3H), 7.30-7.27 (m, 1H), 7.21 (d, J = 2.0 Hz, 1H), 7.12 (d, J = 3.2 Hz, 1H), 6.64 (d, J = 8.4 Hz, 1H), 6.39 (d, J = 2.8 Hz, 1H), 4.55 (s, 2H), 4.48-4.45 (m, 1H), 3.92-3.80 (m, 2H), 3.53-3.48 (m, 1H), 3.39-3.33 (m, 2H), 2.89-2.83 (m, 1H). |
| 280 | 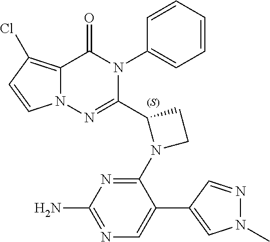 | 474.1 | ¹H NMR (400 MHz, DMSO-d₆) δ 7.63 (d, J = 3.0 Hz, 1H), 7.58-7.38 (m, 6H), 7.34-7.29 (m, 1H), 7.23 (s, 1H), 6.65 (d, J = 3.0 Hz, 1H), 6.07 (s, 2H), 4.57 (t, J = 7.5 Hz, 1H), 3.76 (s, 3H), 3.68-3.60 (m, 1H), 2.36-2.28 (m, 1H), 1.86-1.80 (m, 1H). |

### Example 16

### Compound 111

### (S)-4-(2-(5-chloro-4-oxo-3-phenyl-3,4-dihydropyrrolo[2,1-f][1,2,4]triazin-2-yl)pyrrolidin-1-yl)-1H-pyrazolo[3,4-d]pyrimidine-3-carbonitrile

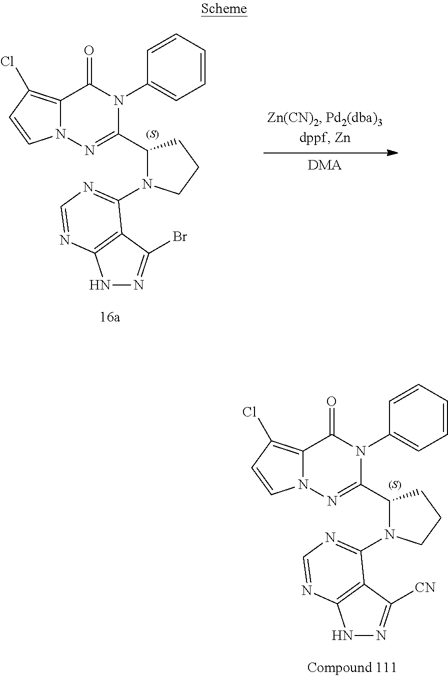

A mixture of 16a (120 mg, 0.23 mmol), Zn(CN)₂ (560 mg, 4.77 mmol), dppf (120 mg, 0.22 mmol), Pd₂(dba)₃ (120 mg, 0.13 mmol) and Zinc powder (120 mg, 1.83 mmol) in DMA (4 mL) was stirred at 150° C. for 30 min under microwave condition. The reaction mixture was diluted with 200 mL of DCM and washed with water. The organic layer was separated, concentrated and purified by preparative TLC and chromatography to give Compound 111 as a white solid (8 mg, yield: 7%). MS (m/z): 457.7 (M+H)⁺. ¹H NMR (400 MHz, DMSO-d₆) δ 8.24 (s, 1H), 7.74 (d, J=7.5 Hz, 1H), 7.64-7.47 (m, 6H), 6.56 (d, J=2.9 Hz, 1H), 4.70-4.62 (m, 1H), 4.15-4.07 (m, 1H), 3.99-3.93 (m, 1H), 2.33-2.27 (m, 1H), 2.25-2.17 (m, 1H), 2.08-2.04 (m, 1H), 1.96-1.93 (m, 1H).

### Example 17

### Compound 497

### (S)-2-(1-(2-amino-5-cyano-6-methylpyrimidin-4-yl)azetidin-2-yl)-4-oxo-3-phenyl-3,4-dihydropyrrolo[2,1-f][1,2,4]triazine-5-carbonitrile

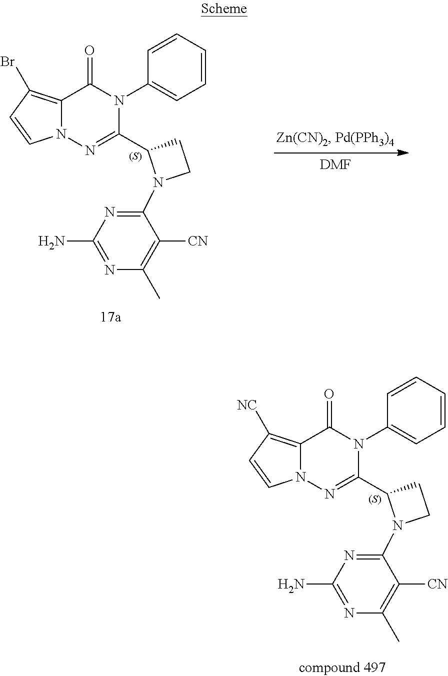

Under N₂ atmosphere, to a solution of 17a (300 mg, 0.63 mmol) (17a was prepared according to the procedure of Example 1) in DMF (20 mL) was added Zn(CN)₂ (945 mg, 3.15 mmol), followed by Pd(PPh₃)₄ (655 mg, 0.567 mmol), the reaction was stirred at 140° C. overnight under N₂. After concentration, the residue was purified by column chromatography to give Compound 497 as a white solid (150 mg, yield: 56%). MS (m/z): 424.4 (M+H)⁺. ¹H NMR (400 MHz, CD₃OD) δ 7.54-7.45 (m, 5H), 7.27-7.23 (m, 1H), 6.90 (d, J=3.2 Hz, 1H), 5.15-5.02 (m, 1H), 4.27-4.16 (m, 1H), 4.08-4.01 (m, 1H), 2.46-2.38 (m, 1H), 2.21 (s, 3H), 2.19-2.12 (m, 1H).

The following compounds were prepared according to the procedure of Compound 497 using the corresponding reagents and intermediates under appropriate conditions that will be recognized by one skilled in the art:

| Compd. | | LC/MS | |
|---|---|---|---|
| No. | Structure | (M + H)⁺ | NMR |
| 498 | 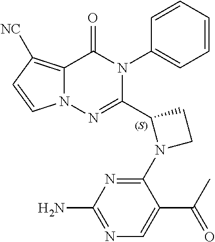 | 427.2 | ¹H NMR (400 MHz, DMSO-d₆) δ 8.43 (s, 1H), 7.83-7.77 (m, 1H), 7.72- 7.66 (m, 1H), 7.61-7.59 (m, 1H), 7.55-7.52 (m, 2H), 7.43-7.40 (m, 1H), 7.11 (d, J = 3.2 Hz, 1H), 6.78 (s, 2H), 5.00-4.75 (m, 1H), 4.19-4.08 (m, 1H), 2.45-2.35 (m, 2H), 1.24 (s, 3H), 1.91-1.86 (m, 1H). |
| 499 | 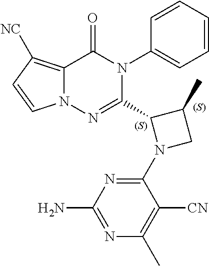 | 438.1 | ¹H NMR (400 MHz, CD₃OD) δ 7.58- 7.46 (m, 5H), 7.33-7.30 (m, 1H), 6.89 (dd, J = 3.0, 0.7 Hz, 1H), 4.76 (brs, 1H), 4.35 (brs, 1H), 3.61 (brs, 1H), 2.64 (brs, 1H), 2.21 (s, 3H), 0.65 (d, J = 6.8 Hz, 3H). |
| 500 | 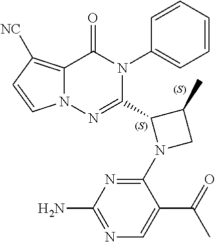 | 441.1 | ¹H NMR (400 MHz, CD₃OD) δ 8.37 (brs, 1H), 7.66-7.52 (m, 4H), 7.37- 7.31 (m, 2H), 6.87 (d, J = 3.0 Hz, 1H), 4.92 (brs, 1H), 4.34 (brs, 1H), 3.29 (brs, 1H), 2.52 (brs, 1H), 2.21 (brs, 3H), 0.62 (d, J = 6.8 Hz, 3H). |
| 501 | 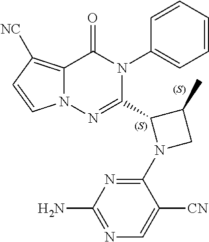 | 424.1 | ¹H NMR (400 MHz, CD₃OD) δ 8.09 (s, 1H), 7.64-7.60 (m, 4H), 7.55 (d, J = 2.9 Hz, 1H), 7.41-7.39 (m, 1H), 6.98 (dd, J = 3.0, 0.5 Hz, 1H), 4.85 (brs, 1H), 4.41 (brs, 1H), 3.69 (brs, 1H), 2.74 (brs, 1H), 0.74 (d, J = 6.7 Hz, 3H). |

### Example 18

### Compound 114

### (S)-5-chloro-2-(1-(2-morpholino-9H-purin-6-yl)azetidin-2-yl)-3-phenylpyrrolo[2,1-f][1,2,4]triazin-4(3H)-one

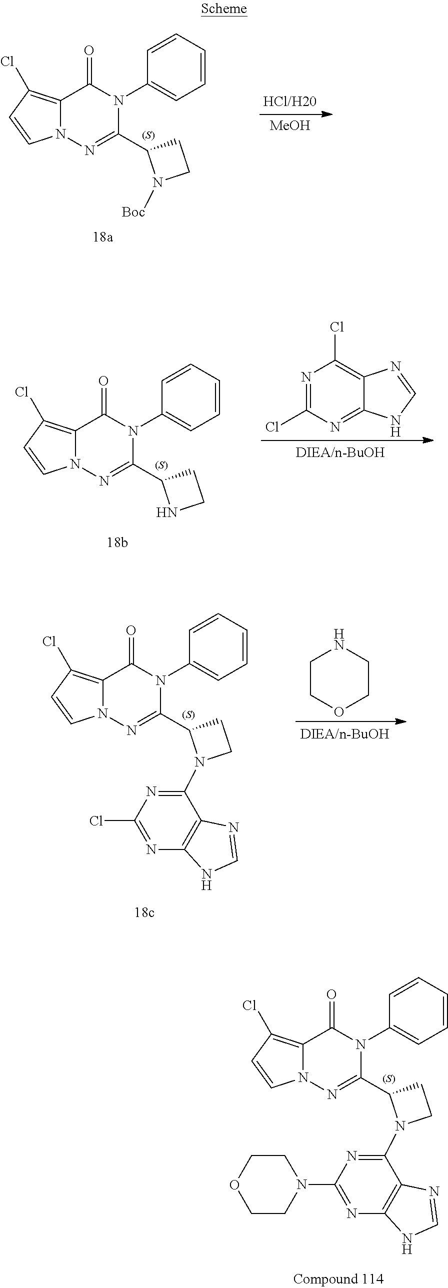

### Step 18-1 (S)-2-(azetidin-2-yl)-5-chloro-3-phenylpyrrolo[2,1-f][1,2,4]triazin-4(3H)-one hydrochloride (18b)

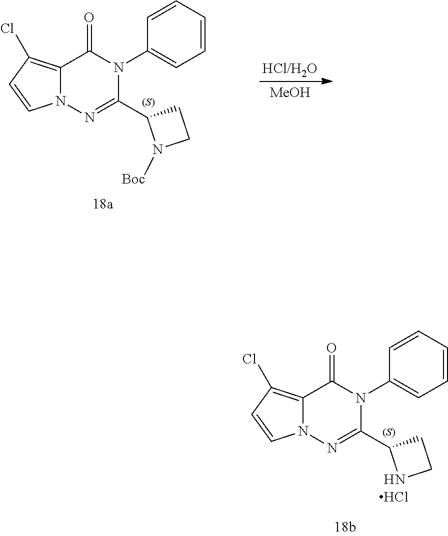

To a mixture of 18a (185 mg, 0.462 mmol) (18a was prepared according to the procedure of Example 1) in MeOH (1 mL) was added conc. HCl (1 mL) at r.t. The mixture was stirred at r.t for 30 min. The mixture was concentrated to give 18b as a brown solid which was used in the next step without purification.

### Steps 18-2 and 18-3 (S)-5-chloro-2-(1-(2-morpholino-9H-purin-6-yl)azetidin-2-yl)-3-phenylpyrrolo[2,1-f][1,2,4]triazin-4(3H)-one (114)

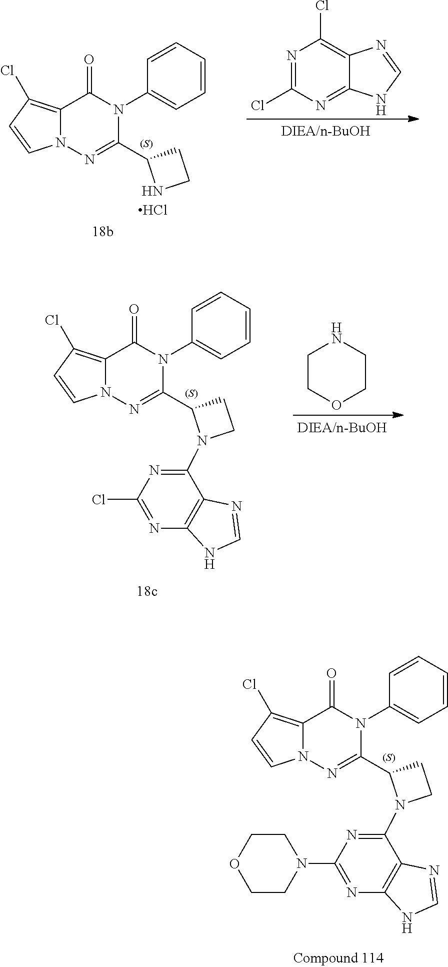

To a mixture of 18-b (0.462 mmol) in n-BuOH (5 mL) were added 2,6-dichloro-9H-purine (87 mg, 0.462 mmol) and DIEA (298 mg, 2.31 mmol) at r.t. The mixture was stirred at 80° C. for 3 h, then morpholine (1 mL) was added, the mixture was stirred at 130° C. overnight. The reaction was concentrated and purified by flash column chromatography to afford Compound 114 as a yellow solid (180 mg, 77%). Yield: MS (m/z): 503.8 (M+H)⁺. ¹H NMR (400 MHz, DMSO-d₆) δ 12.26 (s, 1H), 7.71 (s, 1H), 7.64 (s, 1H), 7.59-7.46 (m, 4H), 7.39 (d, J=6.6 Hz, 1H), 6.61 (d, J=2.6 Hz, 1H), 5.05 (s, 1H), 4.05 (s, 2H), 3.63-3.45 (m, 8H), 2.65-2.54 (m, 1H), 2.27-2.13 (m, 1H).

Compounds 281-284 was prepared according to the procedure of Compound 114 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 281 | 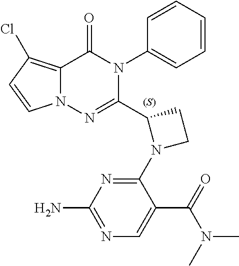 | 465.0 | ¹HNMR (400 MHz, DMSO-d₆) δ 7.67 (d, J = 2.6 Hz, 1H), 7.65 (s, 1H), 7.61-7.55 (m, 2H), 7.52-7.50 (m, 2H), 7.38-7.35 (m, 1H), 6.63 (dd, J = 3.0, 0.5 Hz, 1H), 6.31 (s, 2H), 4.90-4.80 (m, 1H), 3.86-3.74 (m, 2H), 2.67 (s, 6H), 2.44-2.38 (m, 1H), 1.97-1.91 (m, 1H). |
| 282 | 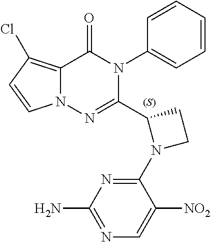 | 439.0 | ¹H NMR (400 MHz, DMSO-d₆) δ 8.67 (s, 1H), 7.73 (s, 1H), 7.64-7.30 (m, 7H), 6.63 (s, 1H), 5.26-5.22 (m, 0.3H), 4.83-4.77 (m, 0.7H), 4.24-4.09 (m, 1H), 3.89-3.67 (m, 0.3H), 3.68 (s, 0.7H), 2.46-2.41 (m, 1H), 1.95-1.80 (m, 1H). |
| 283 | 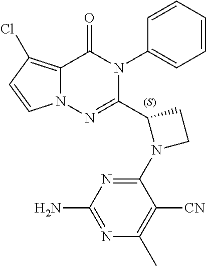 | 433.0 | ¹H NMR (400 MHz, DMSO-d₆) δ 7.73 (d, J = 2.6 Hz, 1H), 7.61-7.49 (m, 4H), 7.42-7.38 (m, 1H), 7.04 (br, 2H), 6.65 (d, J = 3.0 Hz, 1H), 5.06-4.71 (m, 1H), 4.16-3.89 (m, 2H), 2.55-2.48 (m, 1H), 2.19 (s, 3H), 2.05-1.98 (m, 1H). |
| 284 | 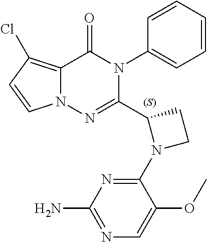 | 424.0 | ¹H NMR (400 MHz, CD₃OD) δ 7.64-7.53 (m, 3H), 7.48 (d, J = 7.3 Hz, 1H), 7.41 (d, J = 3.0 Hz, 1H), 7.38 (s, 1H), 7.30-7.26 (m, 1H), 6.53 (d, J = 3.0 Hz, 1H), 4.96-4.94 (m, 1H), 4.23-4.17 (m, 1H), 3.99-3.95 (m, 1H), 3.60 (s, 3H), 2.47-2.39 (m, 1H), 2.31-2.18 (m, 1H). |

### Example 19

### Compound 115

### 7-(1-(9H-purin-6-ylamino)ethyl)-3-chloro-6-phenylimidazo[1,2-c]pyrimidin-5(6H)-one

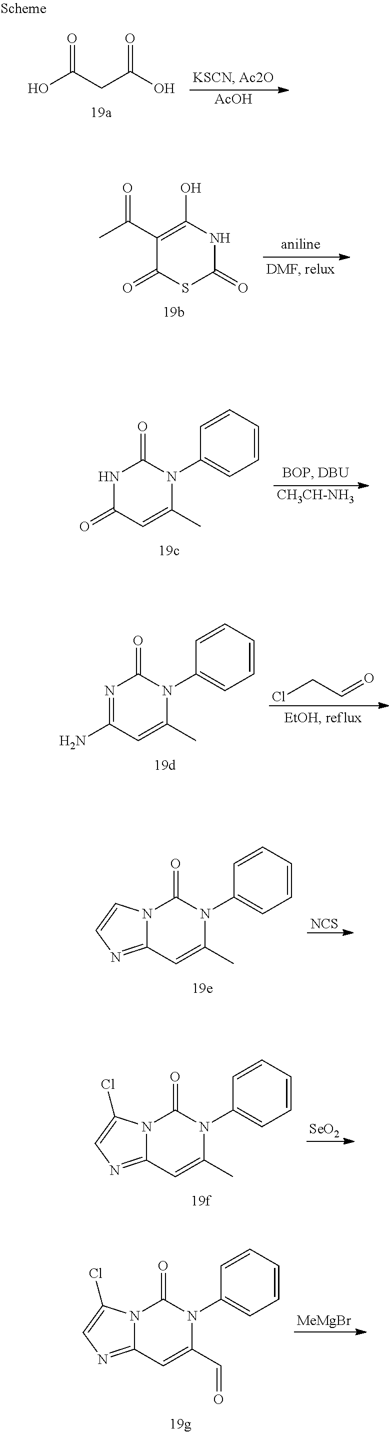

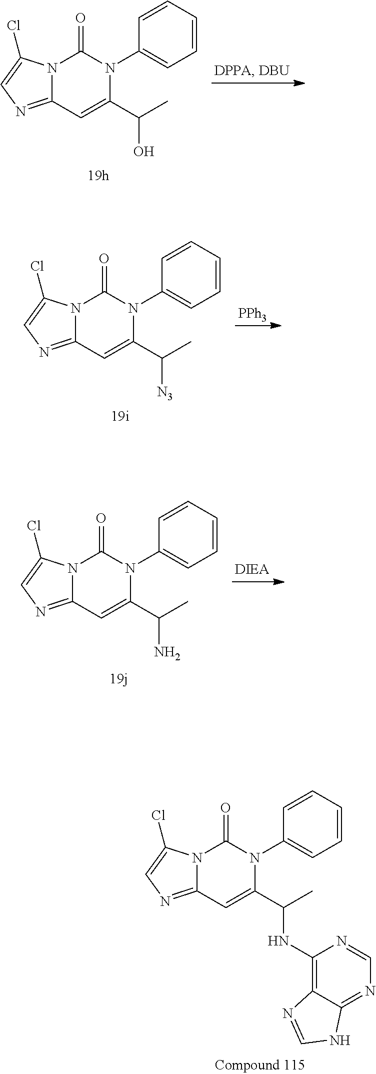 [SMILES: CC(O)c1cc2ncc(Cl)n2c(=O)n1-c1ccccc1 ; CC(N=[N+]=[N-])c1cc2ncc(Cl)n2c(=O)n1-c1ccccc1 ; CC(N)c1cc2ncc(Cl)n2c(=O)n1-c1ccccc1 ; CC(Nc1ncnc2[nH]cnc12)c1cc2ncc(Cl)n2c(=O)n1-c1ccccc1 ; "PPh3"]

### Step 19-1. 5-acetyl-4-hydroxy-2H-1,3-thiazine-2,6(3H)-dione (19b)

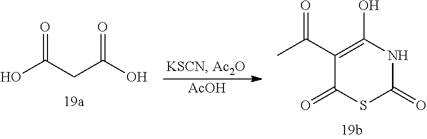

The mixture of 19a (20.8 g, 200 mmol), KSCN (20.0 g, 206 mmol), Ac₂O (20.0 mL) and AcOH (80 mL) was stirred at r.t. overnight. Then H₂O (100 mL) was added and extracted with DCM: MeOH=9:1, the organic layer was dried and concentrated to give 19b as a yellow solid which was used in the next step without further purification (2.0 g, yield: 53%)

### Step 19-2. 6-methyl-1-phenylpyrimidine-2,4(1H,3H)-dione (19c)

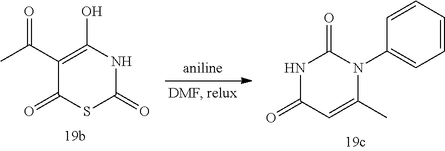

To a solution of 19b (20 g, 106 mmol) in DMF (15 mL) was added aniline (9.2 mL) at r.t., the reaction was stirred at reflux until 19b disappeared by TLC. The mixture was concentrated, the residue was washed with EtOH, and filtered to give 19c as a yellow solid (880 mg, yield: 40.7%). MS (m/z): 203.1 (M+1)⁺.

### Step 19-3. 4-amino-6-methyl-1-phenylpyrimidin-2(1H)-one (19d)

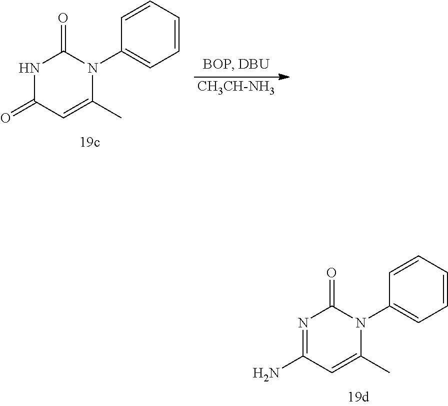

The solution of 19c (7.29 g, 36 mmol) in CH₃CN (120 mL) was purged by NH₃ for 5 min, then BOP (20.7 g, 46.8 mmol) and DBU (8.21 g, 54 mmol) were added, the reaction was stirred overnight. The mixture was filtered to give 19d was as a white solid (7.24 g). MS (m/z): 201.7 (M+1)⁺.

### Step 19-4. 7-methyl-6-phenylimidazo[1,2-c]pyrimidin-5(6H)-one (19e)

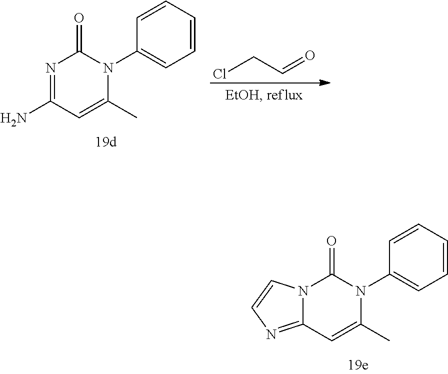

To a solution of 19d (7.24 g, 36 mmol) in EtOH (100 mL) was added 40% 2-chloroacetaldehyde in water (17.8 mL, 108 mmol), the reaction was stirred at 100° C. overnight. The mixture was concentrated and purified by flash column chromatography to give 19e as a white solid (6.2 g, yield: 77%). MS (m/z): 225.9 (M+1)⁺.

### Step 19-5. 3-chloro-7-methyl-6-phenylimidazo[1,2-c]pyrimidin-5(6H)-one (19f)

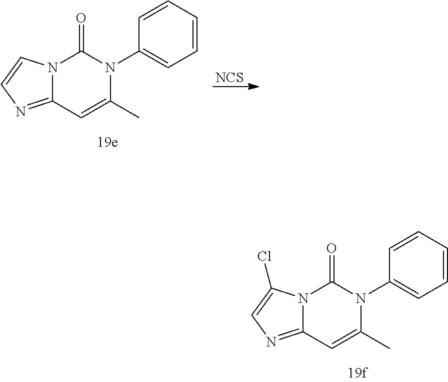

19e (2.25 g, 10 mmol) and NCS (700 mg, 5.26 mmol) were dissolved in DMF (10 mL), the reaction was stirred at r.t. for 3 h. The mixture was poured into H₂O (100 mL), and extracted with EtOAc, the organic layers were washed with brine, dried over anhydrous Na₂SO₄ and concentrated. The resulting residue was washed with MeOH to give 19f as a white solid (600 mg, yield: 23%). MS (m/z): 260.1 (M+1)⁺.

### Step 19-6. 3-chloro-5-oxo-6-phenyl-5,6-dihydroimidazo[1,2-c]pyrimidine-7-carbaldehyde (19g)

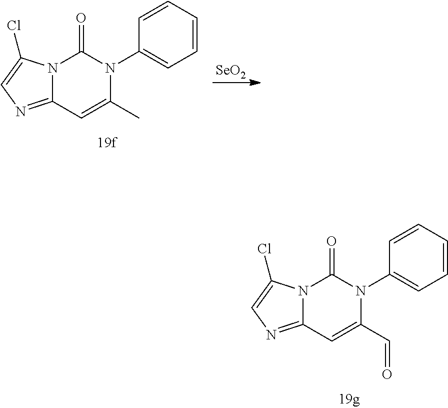

19f (600 mg, 2.3 mmol) and SeO₂ (257 mg, 2.3 mmol) were dissolved in dioxane (20 mL), the reaction was stirred at reflux overnight, then concentrated and purified by flash column chromatography to give 19g as a white solid (250 mg, yield: 39%). MS (m/z): 274.1 (M+1)⁺.

### Step 19-7. 3-chloro-7-(1-hydroxyethyl)-6-phenylimidazo[1,2-c]pyrimidin-5(6H)-one (19h)

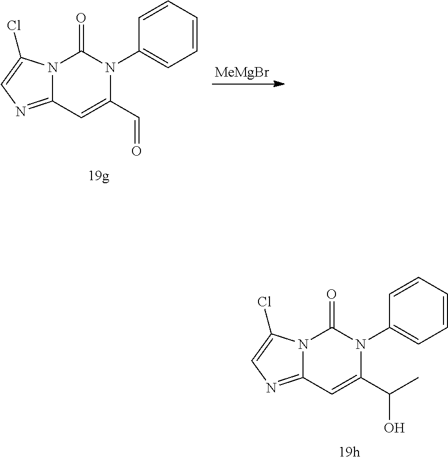 [SMILES: "MeMgBr" ; O=Cc1cc2ncc(Cl)n2c(=O)n1-c1ccccc1 ; CC(O)c1cc2ncc(Cl)n2c(=O)n1-c1ccccc1]

To a solution of 19g (250 mg, 0.9 mmol) in THF (10 mL) cooled to −78° C. was added MeMgBr (3M in ether, 1.2 mL) dropwise under N₂, the reaction was stirred at −78° C. for 30 min. Then MeOH (3 mL) was added dropwise, the resulting mixture was concentrated and purified by flash column chromatography to give 19h as a white solid (220 mg, yield: 83%). MS (m/z): 290.1 (M+1)⁺.

### Step 19-8. 7-(1-azidoethyl)-3-chloro-6-phenylimidazo[1,2-c]pyrimidin-5(6H)-one (19i)

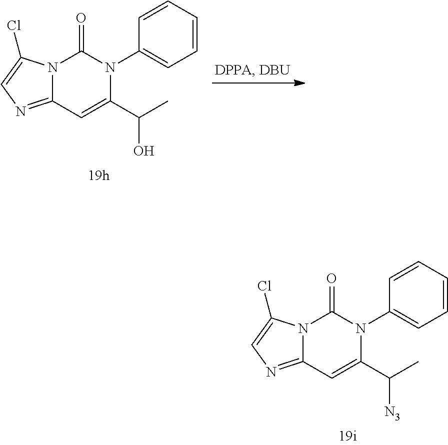 [SMILES: CC(O)c1cc2ncc(Cl)n2c(=O)n1-c1ccccc1 ; CC(N=[N+]=[N-])c1cc2ncc(Cl)n2c(=O)n1-c1ccccc1]

To a solution of 19h (200 mg, 0.69 mmol) in THF (20 mL) was added DPPA (630 mg, 2.29 mmol), followed by DBU (300 mg, 1.97 mmol) at r.t., the reaction was stirred at reflux for 3 h, then concentrated and purified by flash column chromatography to give 19i as a yellow oil (130 mg, yield: 59.9%). MS (m/z): 315.1 (M+1)⁺.

### Step 19-9. 7-(1-aminoethyl)-3-chloro-6-phenylimidazo[1,2-c]pyrimidin-5(6H)-one (19j)

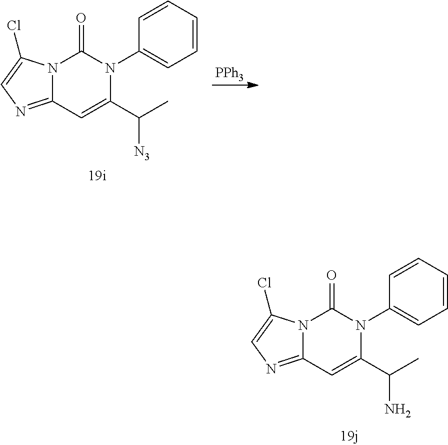 [SMILES: CC(N=[N+]=[N-])c1cc2ncc(Cl)n2c(=O)n1-c1ccccc1 ; CC(N)c1cc2ncc(Cl)n2c(=O)n1-c1ccccc1 ; "PPh3"]

To a solution of 19i (130 mg, 0.4 mmol) in THF (10 mL) was added NH₃.H₂O (25% aq., 1 mL), followed by PPh₃ (200 mg, 0.76 mmol), the reaction was stirred at r.t. for 30 min, then warmed to 60° C. for another 2 hours. The mixture was concentrated and purified by flash column chromatography to give 19j as a white solid (60 mg, yield: 50%). MS (m/z): 288.9 (M+1)⁺.

### Step 19-10. 7-(1-(9H-purin-6-ylamino)ethyl)-3-chloro-6-phenylimidazo[1,2-c]pyrimidin-5(6H)-one (115)

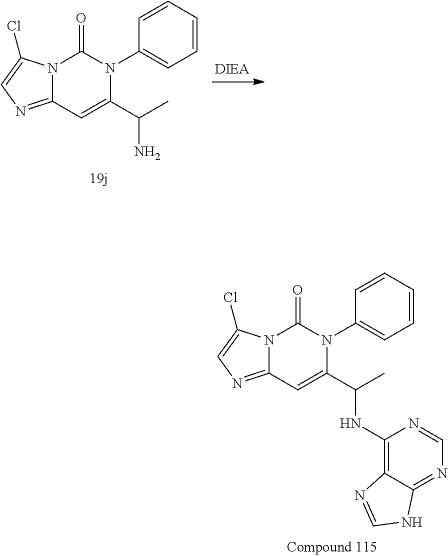 [SMILES: CC(N)c1cc2ncc(Cl)n2c(=O)n1-c1ccccc1 ; CC(Nc1ncnc2[nH]cnc12)c1cc2ncc(Cl)n2c(=O)n1-c1ccccc1]

To a solution of 19j (30 mg, 0.104 mmol) in n-BuOH (3 mL) were added DIEA (0.052 mL, 0.312 mmol) and 6-chloro-9H-purine (19.3 mg, 0.125 mmol), the reaction was stirred at 130° C. overnight. The mixture was concentrated and purified by preparative thin layer chromatography to give Compound 115 as a white solid (3.6 mg, yield: 9%). MS (m/z): 406.9 (M+1)⁺. ¹H NMR (400 MHz, CD₃OD) δ: 8.06 (s, 1H), 7.96 (s, 1H), 7.59-7.47 (m, 3H), 7.38 (t, J=7.3 Hz, 1H), 7.27-7.24 (m, 2H), 6.76 (s, 1H), 4.93-4.89 (m, 1H), 1.47 (d, T=6.7 Hz, 3H).

The following Compounds were prepared according to the procedure of Compound 115 using the corresponding reagents and intermediates under appropriate conditions that will be recognized by one skilled in the art:

| Compd. | | LC/MS | |
|---|---|---|---|
| No. | Structure | (M + H)⁺ | NMR |
| 116 | 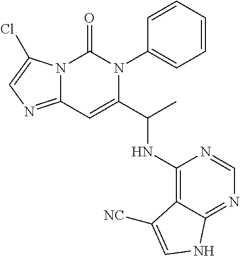 [SMILES: CC(Nc1ncnc2[nH]cc(C#N)c12)c1cc2ncc(Cl)n2c(=O)n1-c1ccccc1] | 431.0 | ¹H NMR (400 MHz, DMSO-d₆) δ 8.11 (s, 1H), 7.95 (s, 1H), 7.70-7.68 (m, 1H), 7.59- 7.51 (m, 2H), 7.46-7.39 (m, 2H), 7.34 (s, 1H), 6.88 (s, 1H), 6.42 (d, J = 6.3 Hz, 1H), 4.60-4.57 (m, 1H), 1.36 (d, J = 6.7 Hz, 3H). |
| 117 | 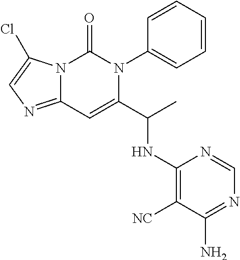 [SMILES: CC(Nc1ncnc(N)c1C#N)c1cc2ncc(Cl)n2c(=O)n1-c1ccccc1] | 406.99 | ¹H NMR (400 MHz, DMSO-d₆) δ 7.88 (s, 1H), 7.67-7.32 (m, 7H), 7.20 (s, 2H), 6.75 (s, 1H), 4.64-4.54 (m, 1H), 1.29 (d, J = 6.6 Hz, 3H). |
| 118 | 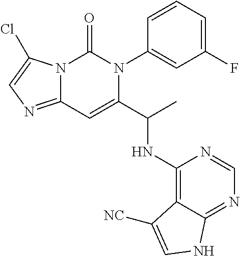 [SMILES: CC(Nc1ncnc2[nH]cc(C#N)c12)c1cc2ncc(Cl)n2c(=O)n1-c1cccc(F)c1] | 448.9 | ¹H NMR (400 MHz, CD₃OD) δ 8.09 (s, 1 H), 7.74 (s, 1H), 7.18-6.96 (m, 3H), 6.74 (s, 1H), 6.66-6.58 (m, 2H), 5.70 (s, 1H), 5.43-5.38 (m, 1H), 1.48 (d, J = 6.8 Hz, 3H). |
| 154 | 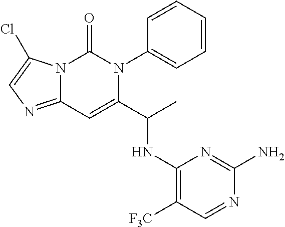 [SMILES: CC(Nc1nc(N)ncc1C(F)(F)F)c1cc2ncc(Cl)n2c(=O)n1-c1ccccc1] | 450.1 | ¹H NMR (400 MHz, CDCl₃) δ 8.07 (s, 1H), 7.55 (t, J = 7.4 Hz, 1H), 7.49-7.34 (m, 4H), 7.25 (s, 1H), 6.65 (s, 1H), 5.00 (d, J = 6.4 Hz, 1H), 4.93 (q, J = 6.9 Hz, 1H), 4.88 (s, 2H), 1.38 (d, J = 6.6 Hz, 3H). |
| 285 | 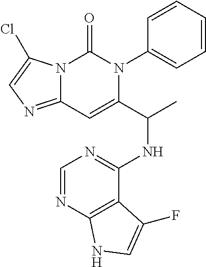 [SMILES: CC(Nc1ncnc2[nH]cc(F)c12)c1cc2ncc(Cl)n2c(=O)n1-c1ccccc1] | 424.1 | ¹H NMR (400 MHz, DMSO-d₆) δ 8.03 (s, 1H), 7.67-7.63 (m, 1H), 7.57-7.48 (m, 3H), 7.43-7.39 (m, 2H), 7.34 (s, 1H), 7.24 (d, J = 7.2 Hz, 1H), 7.06 (d, J = 2.8 Hz, 1H), 6.82 (s, 1H), 4.71-4.62 (m, 1H), 1.35 (d, J = 6.8 Hz, 3H). |
| 286 | 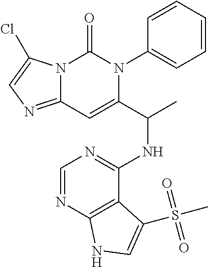 [SMILES: CC(Nc1ncnc2[nH]cc(S(C)(=O)=O)c12)c1cc2ncc(Cl)n2c(=O)n1-c1ccccc1] | 484.2 | ¹H NMR (400 MHz, DMSO-d₆) δ 8.12 (s, 1H), 7.79 (s, 1H), 7.72-7.66 (m, 1H), 7.63-7.59 (m, 1H), 7.58-7.51 (m, 2H), 7.49-7.46 (m, 2H), 7.34 (s, 1H), 6.55 (s, 1H), 4.61-4.53 (m, 1H), 3.20 (s, 3H), 1.28 (d, J = 6.8 Hz, 3H). |
| 287 | 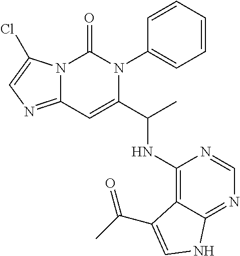 [SMILES: CC(=O)c1c[nH]c2ncnc(NC(C)c3cc4ncc(Cl)n4c(=O)n3-c3ccccc3)c12] | 447.8 | ¹H NMR (400 MHz, DMSO-d₆) δ 9.30 (d, J = 8.0 Hz, 1H), 8.31 (s, 1H), 8.13 (s, 1H), 7.64-7.55 (m, 3H), 7.49-7.41 (m, 2H), 7.39 (s, 1H), 6.59 (s, 1H), 4.68-4.62 (m, 1H), 2.52 (s, 3H), 1.37 (d, J = 6.8 Hz, 3H). |
| 290 | 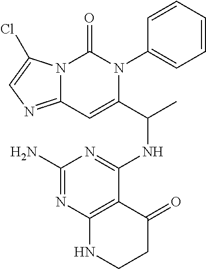 [SMILES: CC(Nc1nc(N)nc2c1C(=O)CCN2)c1cc2ncc(Cl)n2c(=O)n1-c1ccccc1] | 451.1 | ¹H NMR (400 MHz, CD₃OD) δ 9.40 (d, J = 7.2 Hz, 1H), 7.57-7.52 (m, 2H), 7.49-7.45 (m, 3H), 7.25 (s, 1H), 6.60 (s, 1H), 4.82-4.79 (m, 1H), 3.54-3.41 (m, 2H), 2.64-2.48 (m, 2H), 1.34 (d, J = 6.8 Hz, 3H). |

### Example 20

### Compound 119

### 3-(1-(9H-purin-6-ylamino)ethyl)-2-phenylpyrrolo[1,2-c]pyrimidin-1(2H)-one

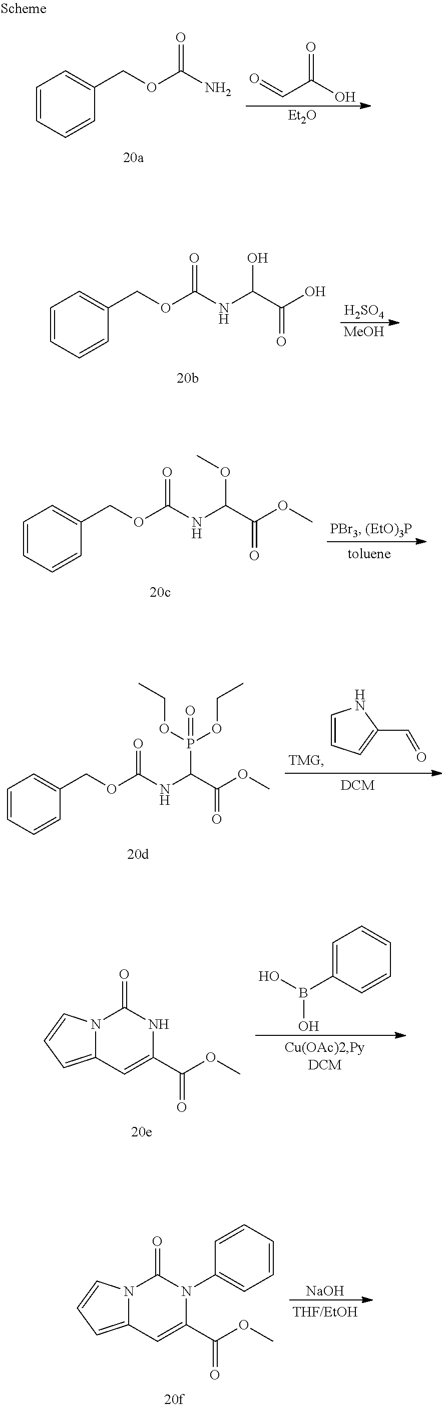

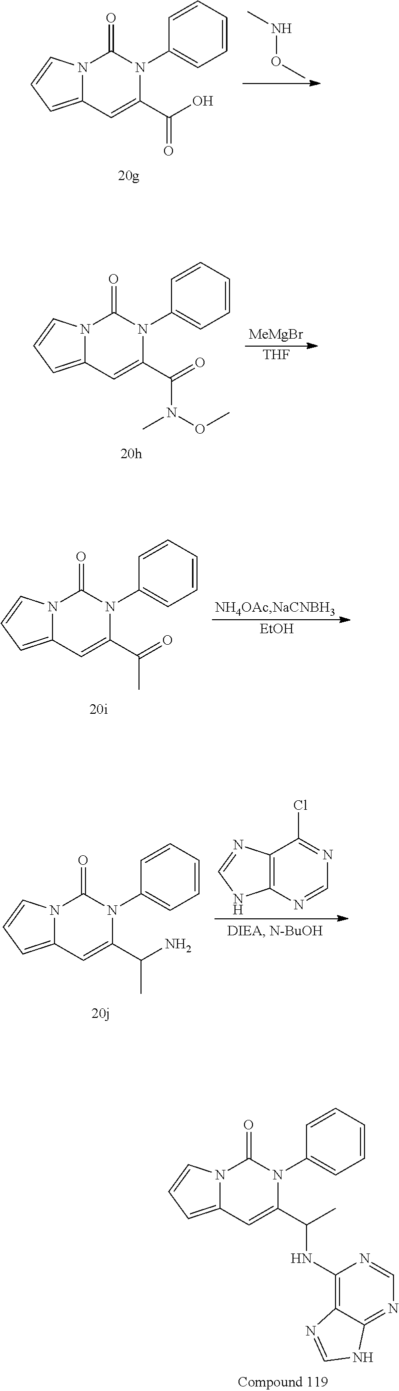

### Step 20-1. 2-(benzyloxycarbonylamino)-2-hydroxyacetic acid (20b)

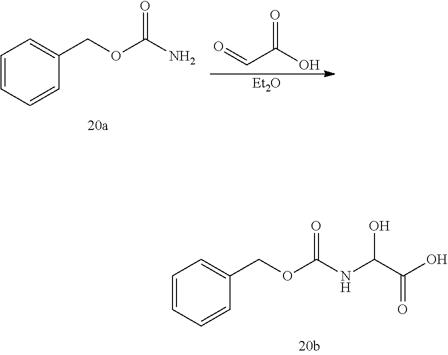

To a mixture of 20a (7.55 g, 50 mmol) in Et₂O (80 mL) was added 2-oxoacetic acid.1H₂O (5.05 g, 55 mmol), the reaction was stirred at r.t. overnight. The mixture was concentrated in vacuo to give 20b as a white solid which was used in the next step without further purification.

### Step 20-2. Methyl 2-(benzyloxycarbonylamino)-2-methoxyacetate (20c)

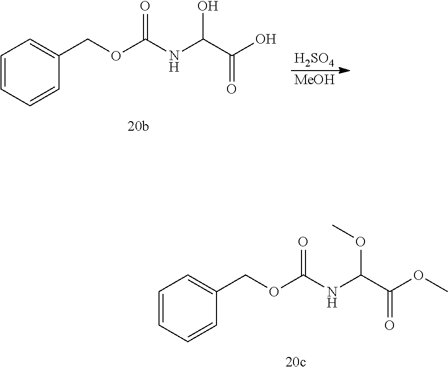

To a solution of 20b (about 11.25 g, 50 mmol) in MeOH (150 mL) was added concentrated sulfuric acid (2 mL) dropwise at 0° C. After the addition, the reaction mixture was stirred at r.t. for 90 h, then poured into the iced sat. NaHCO₃ aq. (300 mL), the resulting mixture was extracted with EtOAc, the organic layers were dried over anhydrous Na₂SO₄, concentrated and purified by column chromatography to give 20c as a white solid (12 g, yield: 95%). MS (m/z): 275.7 (M+23)⁺.

### Step 20-3. Methyl 2-(benzyloxycarbonylamino)-2-(diethoxyphosphoryl)acetate (20d)

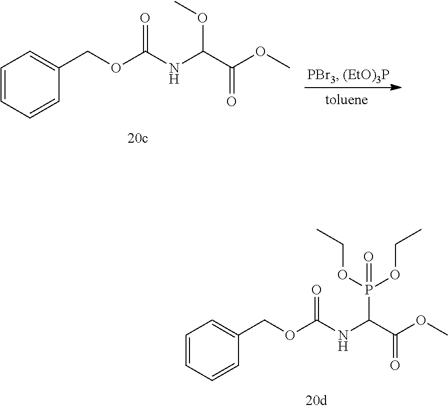 [SMILES: COC(=O)C(NC(=O)OCc1ccccc1)OC ; CCOP(=O)(OCC)C(NC(=O)OCc1ccccc1)C(=O)OC]

To a solution of 20c (12 g, 47.4 mmol) in toluene (60 mL) was added PBr₃ (12.8 g, 47.4 mmol) at 70° C., the reaction was stirred at 70° C. for 20 h, then triethyl phosphate (7.87 g, 47.4 mmol) was added dropwise and stirred at 70° C. for another 2 h. The mixture was concentrated, diluted with EtOAc, and washed with sat. NaHCO₃ aq. The organic layers were dried over anhydrous Na₂SO₄, filtered and concentrated. The resulting residue was dissolved in EtOAc, petroleum ether was added with vigorous stirring, then filtrated to give 20d as a white solid (8 g, yield: 47%).

### Step 20-4. Methyl 1-oxo-1,2-dihydropyrrolo[1,2-c]pyrimidine-3-carboxylate (20e)

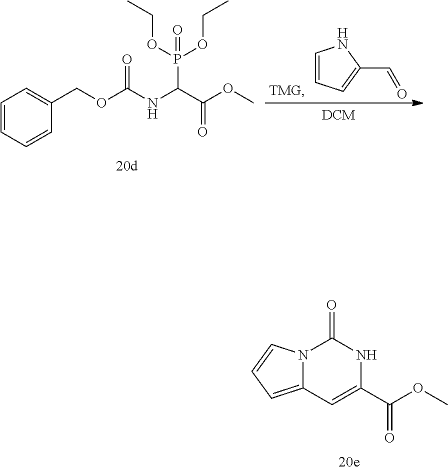 [SMILES: CCOP(=O)(OCC)C(NC(=O)OCc1ccccc1)C(=O)OC ; O=Cc1ccc[nH]1 ; COC(=O)c1cc2cccn2c(=O)[nH]1]

To a solution of 20d (8 g, 22.3 mmol) in DCM (80 mL) was added 1,1,3,3-tetramethylguanidine (2.44 g, 21.2 mmol) at r.t., the reaction was stirred at r.t for 15 min, then a solution of 1H-pyrrole-2-carbaldehyde (1.92 g, 20.2 mmol) in DCM (5 mL) was added dropwise at −30° C., the reaction mixture was stirred at −30° C. for 45 min, then warmed to r.t. and stirred for 48 h. The mixture was concentrated and purified by column chromatography to give 20e as a white solid (2 g, yield: 51%). MS (m/z): 192.9 (M+1)⁺.

### Step 20-5. Methyl 1-oxo-2-phenyl-1,2-dihydropyrrolo[1,2-c]pyrimidine-3-carboxylate (20f)

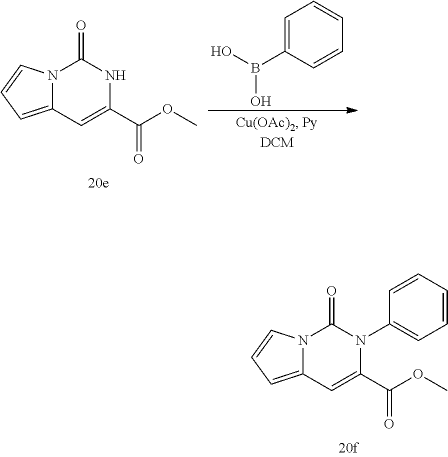 [SMILES: COC(=O)c1cc2cccn2c(=O)[nH]1 ; OB(O)c1ccccc1 ; COC(=O)c1cc2cccn2c(=O)n1-c1ccccc1]

To a solution of 20e (576 mg, 3 mmol) in DCM (20 mL) was added phenylboronic acid (732 mg, 6 mmol), copper(II) acetate (1.08 g, 6 mmol), pyridine (1.18 g, 15 mmol) and 4 Å molecular sieve at r.t., the reaction was stirred at r.t. for 20 h. The mixture was filtered, concentrated and purified by column chromatography to give 20f as a white solid (650 mg, yield: 81%). MS (m/z): 268.8 (M+1)⁺.

### Step 20-6. 1-oxo-2-phenyl-1,2-dihydropyrrolo[1,2-c]pyrimidine-3-carboxylic acid (20g)

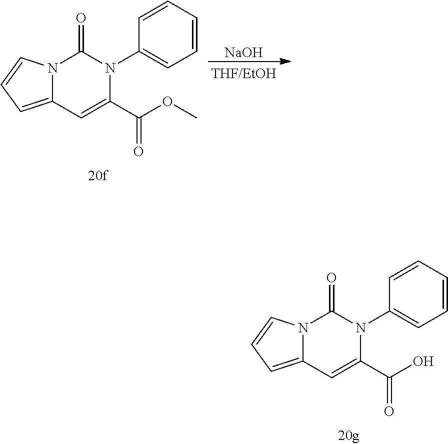

To a solution of 20f (1 g, 3.73 mmol) in EtOH (30 mL) and THF (30 mL) was added NaOH aq. (11.19 mL, 1N) at 0° C., the reaction was stirred at 0° C. for 30 min. The mixture was concentrated, diluted with H₂O (10 mL), adjusted to pH=6 with HCl aq. (1N) and concentrated in vacuo to give 20g as a brown solid which was used in the next step without further purification. MS (m/z): 254.7 (M+1)⁺.

### Step 20-7. N-methoxy-N-methyl-1-oxo-2-phenyl-1,2-dihydropyrrolo[1,2-c]pyrimidine-3-carboxamide (20h)

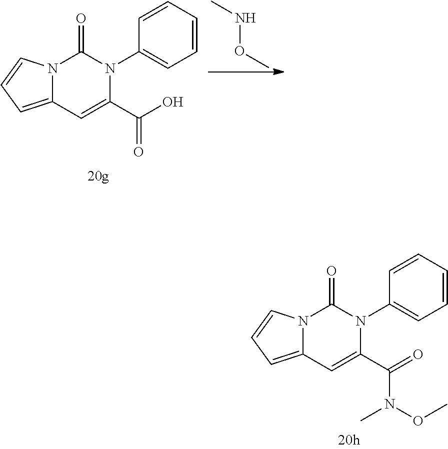

To a solution of 20g (about 950 mg, 3.73 mmol) in DMF (10 mL) were added DIEA (1.44 g, 11.19 mmol) and HBTU (1.70 g, 4.48 mmol), the mixture was stirred at r.t for 5 min, then N,O-dimethylhydroxylamine hydrochloride (438 mg, 4.48 mmol) was added, the reaction was stirred at r.t overnight. The mixture was concentrated and purified by column chromatography to give 20h as a white solid (550 mg, yield: 50%). MS (m/z): 297.7 (M+1)⁺.

### Step 20-8. 3-acetyl-2-phenylpyrrolo[1,2-c]pyrimidin-1(2H)-one (20i)

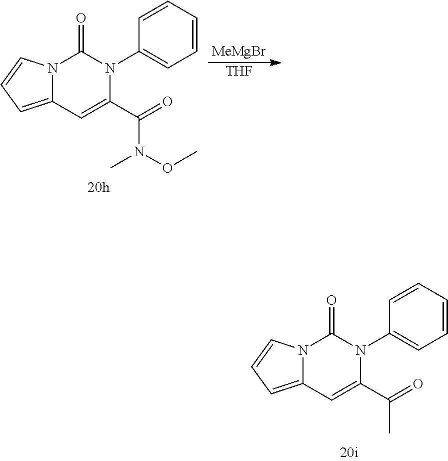 [SMILES: "MeMgBr" ; CON(C)C(=O)c1cc2cccn2c(=O)n1-c1ccccc1 ; CC(=O)c1cc2cccn2c(=O)n1-c1ccccc1 ; "THF"]

To a solution of 20h (550 mg, 1.85 mmol) in THF (5 mL) was added a solution of Methylmagnesium bromide in Et₂O (1.23 mL, 3N) at 0° C. under N₂, the reaction was stirred at 0° C. for 1 h. The mixture was quenched with sat. NH₄Cl aq., concentrated and purified by column chromatography to give 20i as a yellow solid (220 mg, yield: 47%). MS (m/z): 252.7 (M+1)⁺.

### Step 20-9. 3-(1-aminoethyl)-2-phenylpyrrolo[1,2-c]pyrimidin-1(2H)-one (20j)

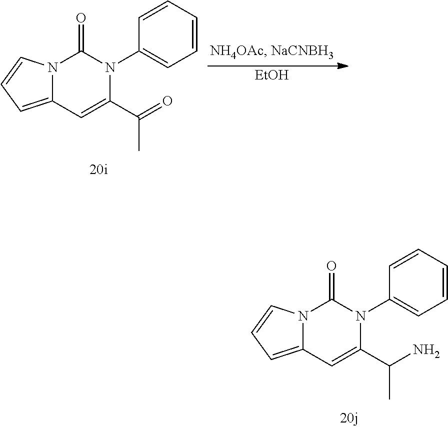 [SMILES: "EtOH" ; CC(=O)c1cc2cccn2c(=O)n1-c1ccccc1 ; CC(N)c1cc2cccn2c(=O)n1-c1ccccc1]

To a solution of 20i (50.4 mg, 0.2 mmol) in EtOH (6 mL) were added ammonium acetate (550 mg, 7.1 mmol) and sodium cyanoborohydride (126 mg, 2 mmol), the reaction was stirred at 130° C. for 2 h under Microwave condition, then another part of ammonium acetate (550 mg, 7.1 mmol) and sodium cyanoborohydride (126 mg, 2 mmol) was added, the reaction was stirred at 90° C. for 20 h. After cooling to r.t, aq. HCl (0.5 mL, 1 N) was added, the mixture was stirred for 30 min, followed by conc. NH₃.H₂O (3 mL), the mixture was stirred for 10 min, then NaBH₄ (30 mg, 0.79 mmol) was added, the mixture was stirred for another 30 min. The mixture was concentrated and purified by flash column chromatography to give 20j as a yellow solid (32 mg, yield: 63%). MS (m/z): 236.7 (M-16)⁺.

### Step 20-10. 3-(1-(9H-purin-6-ylamino)ethyl)-2-phenylpyrrolo[1,2-c]pyrimidin-1(2H)-one (Compound 119)

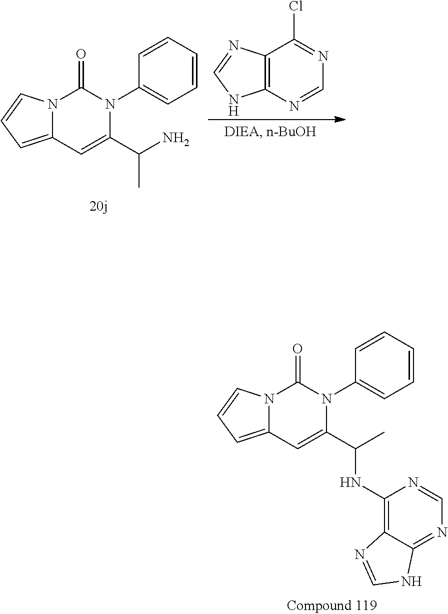

To a solution of 20j (40 mg, 0.158 mmol) in n-BuOH (8 mL) was added 6-chloro-9H-purine (29 mg, 0.190 mmol) and DIEA (61 mg, 0.474 mmol) at r.t., the reaction was stirred at 130° C. overnight. The mixture was concentrated and purified by flash column chromatography to give Compound 119 as a yellow solid (10 mg, yield: 17%). MS (m/z): 371.6 (M+1)⁺. ¹H NMR (400 MHz, DMSO-d₆) δ 8.05 (s, 1H), 7.97 (s, 1H), 7.72 (s, 1H), 7.66 (s, 1H), 7.57-7.30 (m, 6H), 6.71 (s, 1H), 6.63 (s, 1H), 6.29 (s, 1H), 4.78 (s, 1H), 1.32 (d, J=6.5 Hz, 3H).

The following Compounds 120 and 121 were prepared according to the procedures of Compound 119 using the corresponding reagents and intermediates under appropriate conditions that will be recognized by one skilled in the art:

| Compd. | | LC/MS | |
|---|---|---|---|
| No. | Structure | (M + H)⁺ | NMR |
| 120 | 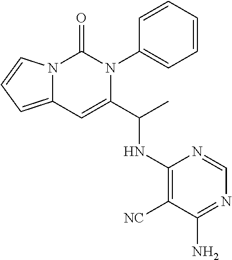 [SMILES: CC(Nc1ncnc(N)c1C#N)c1cc2cccn2c(=O)n1-c1ccccc1] | 371.7 | ¹H NMR (400 MHz, DMSO-d₆) δ 7.80 (s, 1H), 7.54 (d, J = 7.3 Hz, 1H), 7.48-7.39 (m, 4H), 7.34 (s, 2H), 7.16 (s, 2H), 6.70 (s, 1H), 6.65 (s, 1H), 6.38 (s, 1H), 4.71-4.62 (m, 1H), 1.29 (d, J = 6.6 Hz, 3H). |
| 121 | 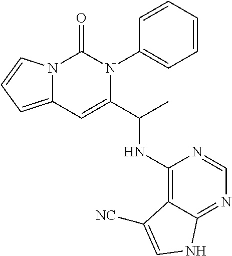 [SMILES: CC(Nc1ncnc2[nH]cc(C#N)c12)c1cc2cccn2c(=O)n1-c1ccccc1] | 395.6 | ¹H NMR (400 MHz, DMSO-d₆) δ 8.03 (s, 1H), 7.91 (s, 1H), 7.57 (d, J = 8.2 Hz, 1H), 7.54-7.25 (m, 5H), 6.80 (s, 1H), 6.63 (s, 1H), 6.31 (s, 1H), 6.08 (s, 1H), 4.67 (m, 1H), 1.35 (d, J = 6.4 Hz, 3H). |

### Example 21

### Compounds 122 and 123

### 3-(1-(9H-purin-6-ylamino)ethyl)-7-chloro-2-phenylpyrrolo[1,2-c]pyrimidin-1(2H)-one and 3-(1-(9H-purin-6-ylamino)ethyl)-5-chloro-2-phenylpyrrolo[1,2-c]pyrimidin-1(2H)-one

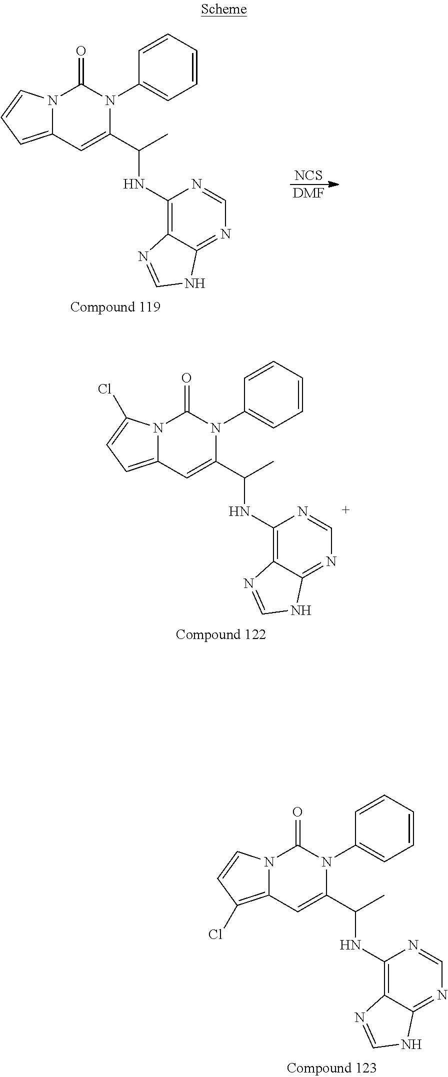

To a solution of Compound 119 (60 mg, 0.16 mmol) in DMF (3 mL) was added NCS (21 mg, 0.16 mmol) at r.t., the reaction was stirred at 70° C. for 30 min, then another part of NCS (6 mg, 0.045 mmol) was added, the reaction was stirred at 70° C. for another 30 min. The mixture was concentrated and purified by flash column chromatography to give Compound 122 as a white solid (15 mg, yield: 23%) and Compound 123 as a white solid (5 mg, yield: 7.7%)). Compound 122: MS (m/z): 406.1 (M+1)⁺. ¹H NMR (400 MHz, DMSO-d₆) δ 8.03 (s, 1H), 7.89 (s, 1H), 7.69 (s, 1H), 7.56 (s, 1H), 7.56-7.34 (m, 5H), 6.64-6.55 (m, 2H), 6.25 (d, J=3.7 Hz, 1H), 4.87-4.57 (m, 1H), 1.28 (d, J=6.6 Hz, 3H). Compound 123: MS (m/z): 405.7 (M+1)⁺. ¹H NMR (400 MHz, CD₃OD) δ 7.90 (s, 1H), 7.83 (s, 1H), 7.49 (d, J=3.2 Hz, 1H), 7.46 (d, J=7.6 Hz, 1H), 7.42-7.35 (m, 2H), 7.28 (t, J=7.1 Hz, 1H), 7.03 (t, J=7.4 Hz, 1H), 6.77 (s, 1H), 6.65 (d, J=3.0 Hz, 1H), 1.49 (d, J=6.7 Hz, 3H).

The following Compounds were prepared according to the procedures of Compound 122 and 123 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 124 | 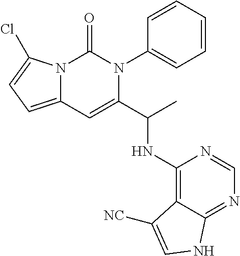 [SMILES: CC(Nc1ncnc2[nH]cc(C#N)c12)c1cc2ccc(Cl)n2c(=O)n1-c1ccccc1] | 429.7 | ¹H NMR (400 MHz, DMSO-d₆) δ 7.96 (s, 1H), 7.67 (s, 1H), 7.65 (d, J = 5.3 Hz, 1H), 7.58-7.35 (m, 5H), 6.64 (s, 1H), 6.60 (d, J = 3.8 Hz, 1H), 6.27 (d, J = 3.8 Hz, 1H), 5.47 (d, J = 6.7 Hz, 1H), 4.58-4.51 (m, 1H), 1.30 (d, J = 6.7 Hz, 3H). |
| 125 | 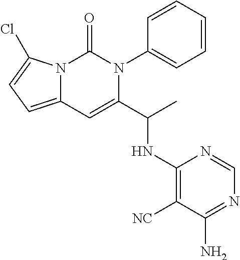 [SMILES: CC(Nc1ncnc(N)c1C#N)c1cc2ccc(Cl)n2c(=O)n1-c1ccccc1] | 405.7 | ¹H NMR (400 MHz, DMSO-d₆) δ 7.80 (s, 1H), 7.47 (d, J = 7.2 Hz, 1H), 7.44-7.36 (m, 3H), 7.35-7.28 (m, 2H), 7.13 (s, 2H), 6.62 (d, J = 3.8 Hz, 1H), 6.61 (s, 1H), 6.35 (d, J = 3.8 Hz, 1H), 4.74-4.43 (m, 1H), 1.26 (d, J = 6.7 Hz, 3H). |
| 126 | 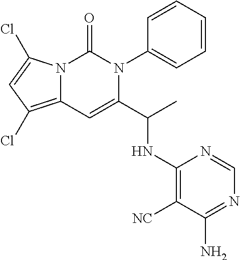 [SMILES: CC(Nc1ncnc(N)c1C#N)c1cc2c(Cl)cc(Cl)n2c(=O)n1-c1ccccc1] | 439.6 | ¹H NMR (400 MHz, DMSO-d₆) δ 7.88 (s, 1H), 7.60 (d, J = 7.3 Hz, 1H), 7.52-7.43 (m, 3H), 7.42-7.36 (m, 2H), 7.20 (s, 2H), 6.87 (s, 1H), 6.60 (s, 1H), 4.62-4.53 (m, 1H), 1.29 (d, J = 6.8 Hz, 3H). |
| 127 | 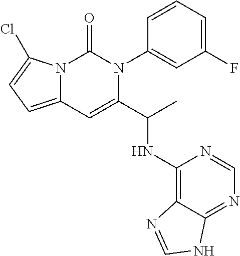 [SMILES: CC(Nc1ncnc2[nH]cnc12)c1cc2ccc(Cl)n2c(=O)n1-c1cccc(F)c1] | 424.1 | ¹H NMR (400 MHz, CD₃OD) δ 7.95 (s, 0.5H), 7.93 (s, 0.5H), 7.834 (s, 0.5H), 7.83 (s, 0.5H), 7.52 (dd, J = 14.4, 8.0 Hz, 1H), 7.32-7.27 (m, 1H), 7.24-7.18 (m, 1H), 7.10-6.91 (m, 2H), 6.81 (s, 0.5H), 6.80 (s, 0.5H), 6.59 (d, J = 1.7 Hz, 0.5H), 6.58 (d, J = 1.7 Hz, 0.5H), 6.40 (d, J = 4.1 Hz, 0.5H), 6.38 (d, J = 4.1 Hz, 0.5H), 5.46-5.33 (m, 1H), 1.56 (d, J = 5.3 Hz, 1.5H)-1.54 (d, J = 5.3 Hz, 1.5H) |
| 128 | 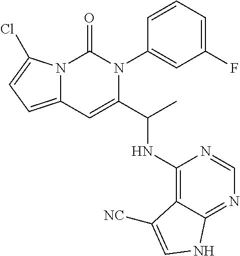 [SMILES: CC(Nc1ncnc2[nH]cc(C#N)c12)c1cc2ccc(Cl)n2c(=O)n1-c1cccc(F)c1] | 448.1 | ¹H NMR (400 MHz, DMSO-d₆) δ 8.29 (s, 1H), 8.13-8.07 (m, 1.5H), 8.04 (s, 0.5H), 7.57-7.40 (m, 1H), 7.39-7.02 (m, 3H), 6.80 (s, 0.5H), 6.78 (s, 0.5H), 6.64 (d, J = 3.6 Hz, 0.5H ), 6.63 (d, J = 3.6 Hz, 0.5H), 6.56 (s, 0.5H), 6.54 (s, 0.5H), 6.35 (d, J = 3.8 Hz, 0.5H), 6.33 (d, J = 3.8 Hz, 0.5H), 4.85-4.62 (m, 1H), 1.39 (d, J = 6.7 Hz, 3H) |
| 129 | 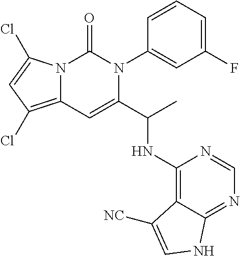 [SMILES: CC(Nc1ncnc2[nH]cc(C#N)c12)c1cc2c(Cl)cc(Cl)n2c(=O)n1-c1cccc(F)c1] | 482.1 | ¹H NMR (400 MHz, DMSO-d₆) δ 8.32 (s, 1H), 8.15 (s, 0.5H), 8.12 (br, 1H), 8.09 (s, 0.5H), 7.57-7.45 (m, 1H), 7.43-7.12 (m, 3H), 6.85 (d, J = 1.0 Hz, 0.5H), 6.84 (d, J = 1.1 Hz, 0.5H), 6.82-6.76 (m, 1H), 6.75 (br, 1H), 4.73-4.60 (m, 1H), 1.40 (d, J = 4.3 Hz, 3H) |

### Example 24

### Compound 132

### 5-fluoro-2-((2S,4S)-4-fluoro-1-(9H-purin-6-yl)pyrrolidin-2-yl)-3-phenylpyrrolo[2,1-f][1,2,4]triazin-4(3H)-one

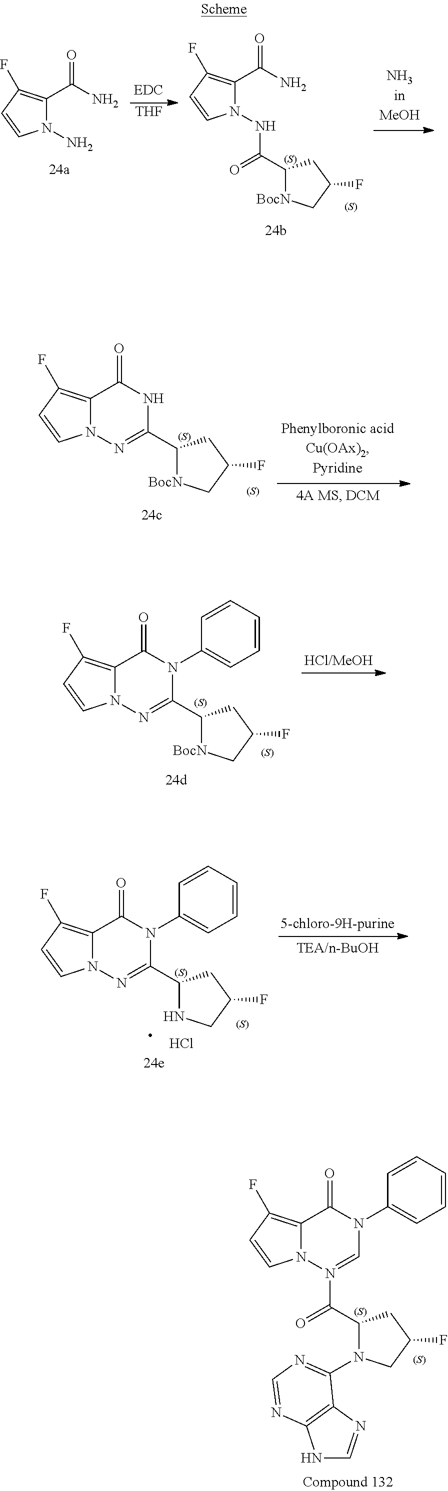

Compound 132 was prepared according to the procedures of Example 1 and the following Steps 24-1 and 2. Compound 132 was got as a white solid. MS (m/z): 434.8 (M+H)⁺; ¹H NMR (400 MHz, CD₃OD) δ: 8.27 (s, 1H), 8.16-7.93 (m, 2H), 7.65-7.49 (m, 4H), 7.15-7.05 (br, 1H), 6.24-6.20 (m, 1H), 5.41 (s, 0.5H), 5.30-5.26 (m, 0.5H), 4.61-4.20 (br, 2H), 4.02-3.94 (m, 1H), 2.58-2.44 (m, 1H), 2.32-2.14 (m, 1H).

### Steps 24-1 and 2 (2S,4S)-tert-butyl 4-fluoro-2-(5-fluoro-4-oxo-3,4-dihydropyrrolo[2,1-f][1,2,4]triazin-2-yl)pyrrolidine-1-carboxylate (24c)

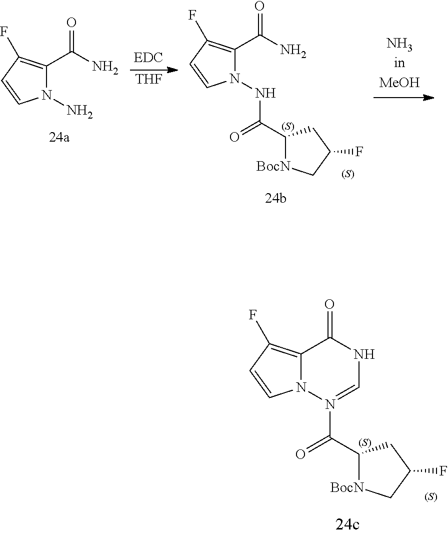

To a solution of 24a (400 mg, 2.94 mmol) and (2S,4S)-1-(tert-butoxycarbonyl)-4-fluoropyrrolidine-2-carboxylic acid (889 mg, 3.82 mmol) in THF (35 mL) was added EDC (729 mg, 3.82 mmol). The reaction mixture was stirred at r.t. for 2 hours, then the solvent was removed in vacuo and water was added. The mixture was extracted with EtOAc three times. The organic layers were combined, died over anhydrous Na₂SO₄ and concentrated to give 24b.

24b was dissolved in 7N NH₃ in MeOH (100 mL) and the mixture was stirred in a sealed tube at 130° C. overnight. The solvent was removed in vacuo and the residue was purified by flash column chromatography eluting with EtOAc/PE to give 24c as a white solid (110 mg, yield: 11%). MS (m/z): 341 (M+H)⁺

### Example 25

### Compound 133

### (S)-4-(2-(5-ethyl-4-oxo-3-phenyl-3,4-dihydropyrrolo[2,1-f][1,2,4]triazin-2-yl)azetidin-1-yl)-7H-pyrrolo[2,3-d]pyrimidine-5-carbonitrile

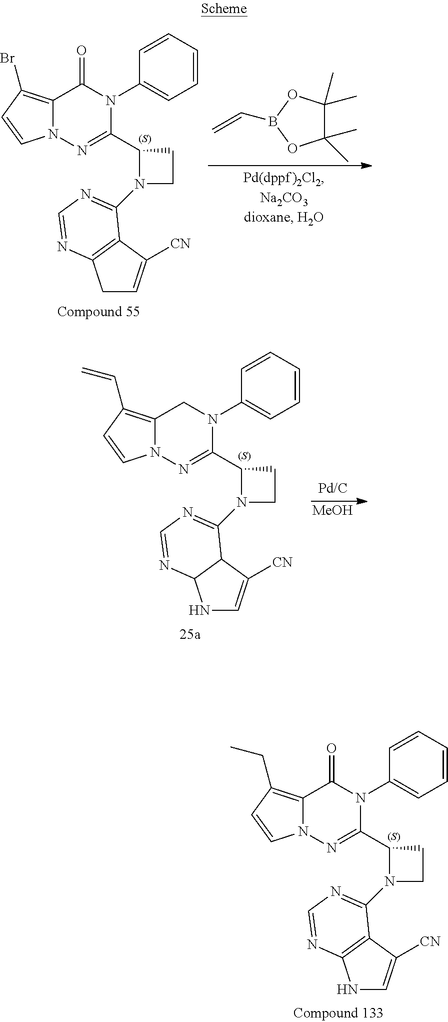

### Step 1 (S)-4-(2-(4-oxo-3-phenyl-5-vinyl-3,4-dihydropyrrolo[2,1-f][1,2,4]triazin-2-yl)azetidin-1-yl)-7H-pyrrolo[2,3-d]pyrimidine-5-carbonitrile (25a)

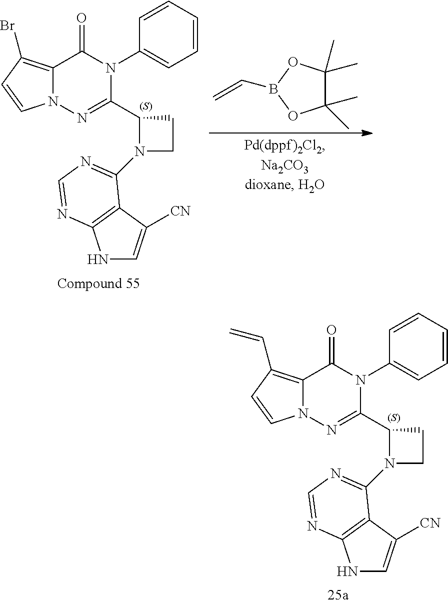

A mixture of Compound 55 (308 mg, 0.632 mmol), 4,4,5,5-tetramethyl-2-vinyl-1,3,2-dioxaborolane (200 mg, 1.265 mmol), Pd(dppf)₂Cl₂ (52 mg, 0.0632 mmol) and Na₂CO₃ (201 mg, 1.896 mmol) in dioxane (20 mL) and water (2 mL) was reacted at 130° C. under N₂ atmosphere in a microwave oven for 30 min. Then the mixture was filtered, concentrated and purified by flash column chromatography eluting with MeOH/DCM to give 25a as a slight yellow solid (120 mg, yield: 44%). MS (m/z): 435.1 (M+H)⁺.

### Step 2 (S)-4-(2-(5-ethyl-4-oxo-3-phenyl-3,4-dihydropyrrolo[2,1-f][1,2,4]triazin-2-yl)azetidin-1-yl)-7H-pyrrolo[2,3-d]pyrimidine-5-carbonitrile (Compound 133)

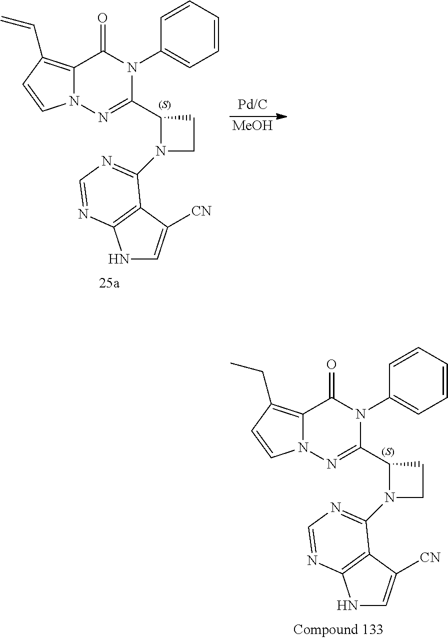

To a solution of 25a (60 mg, 0.138 mmol) in methanol (10 mL) was added Pd/C (6 mg), the mixture was stirred at r.t. under H₂ atmosphere for 2.5 hours, then the mixture was filtered, concentrated and purified by flash column chromatography eluting with MeOH/water to give Compound 133 as a white solid (41 mg, yield: 68%). MS (m/z): 436.8 (M+H)⁺. ¹H NMR (400 MHz, DMSO-d₆) δ 8.31 (s, 2H), 7.78-7.42 (m, 6H), 6.47 (s, 1H), 5.18-5.08 (br, 1H), 4.49-4.15 (m, 2H), 2.88 (q, J=7.4 Hz, 2H), 2.73-2.63 (m, 1H), 2.19-2.09 (m, 1H), 1.21 (t, J=7.5 Hz, 3H).

The following Compounds 291-292 was prepared according to the procedure of Compound 133 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 291 | 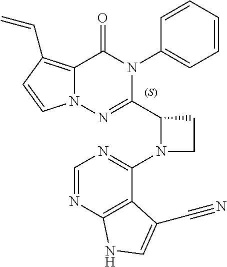 | 434.9 | ¹H NMR (400 MHz, DMSO-d₆) δ: 8.36 (d, J = 9.3 Hz, 2H), 7.80-7.49 (m, 6H), 7.38-7.31 (m, 1H), 6.96 (d, J = 2.2 Hz, 1H), 5.87 (d, J = 17.8 Hz, 1H), 5.31 (d, J = 11.3 Hz, 1H), 5.32-5.21 (m, 1H), 4.51-4.46 (m, 1H), 4.34-4.23 (m, 1H), 2.86-2.74 (m, 1H), 2.29-2.21 (m, 1H). |
| 292 | 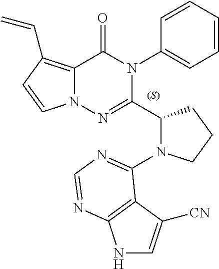 | 448.9 | ¹H NMR (400 MHz, DMSO-d₆) δ: 8.50-8.40 (m, 2H), 7.72-7.47 (m, 5H), 7.39-7.33 (m, 1H), 7.20-7.15 (m, 1H), 6.73-6.70 (m, 1H), 5.68-5.62 (m, 1H), 5.26-5.23 (m, 1H), 5.13-5.10 (m, 1H), 4.78-4.71 (m, 1H), 4.07-4.01 (m, 1H), 2.23-2.11 (m, 2H), 2.00-1.85 (m, 2H). |

### Example 26

### Compound 134

### (S)-2-(1-(2-aminopyrazolo[1,5-a][1,3,5]triazin-4-yl)pyrrolidin-2-yl)-5-chloro-3-phenylpyrrolo[2,1-f][1,2,4]triazin-4(3H)-one

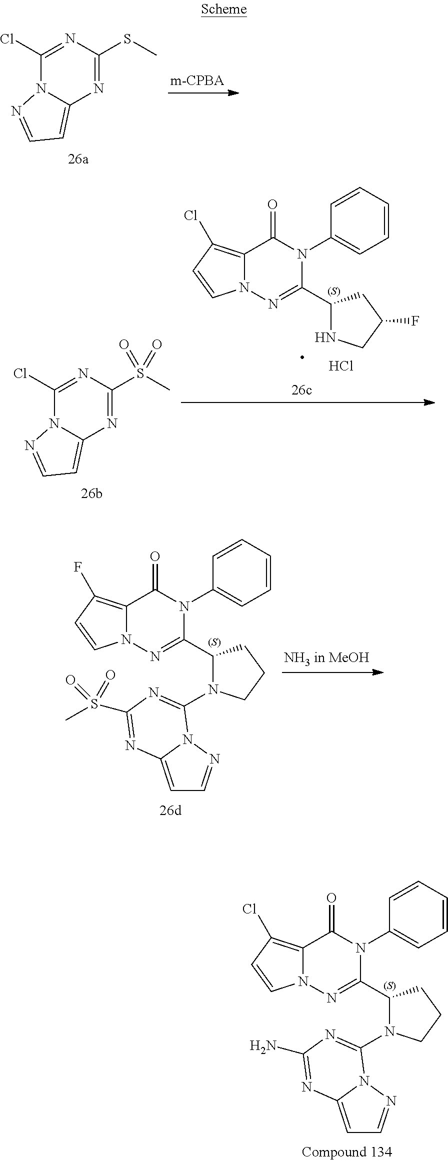

### Step 26-1 4-chloro-2-(methylsulfonyl)pyrazolo[1,5-a][1,3,5]triazine (26b)

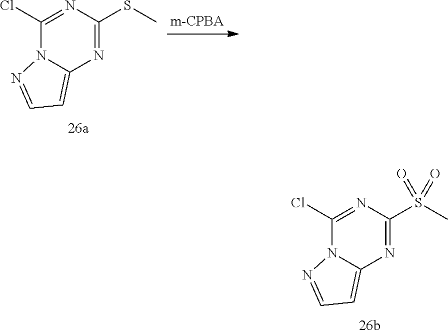

To a solution of 26a (250 mg, 1.25 mmol) in 20 mL of dry DCM was added m-CPBA (473 mg, 2.75 mmol) and stirred at r.t. for 16 hours. The solution was used forward next step without further purification.

### Step 26-2 (S)-5-chloro-2-(1-(2-(methylsulfonyl)pyrazolo[1,5-a][1,3,5]triazin-4-yl)pyrrolidin-2-yl)-3-phenylpyrrolo[2,1-f][1,2,4]triazin-4(3H)-one (26d)

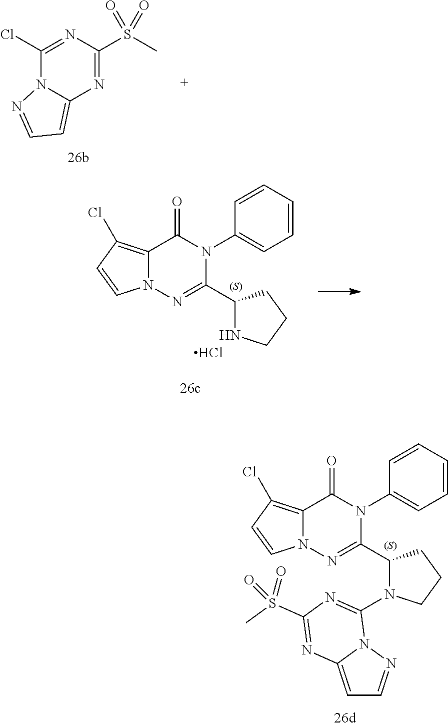

To the solution 26b was added 26c (63 mg, 0.18 mmol) (26c was prepared according to the procedure of Example 1) and DIEA (78 mg, 0.60 mmol), then the mixture was stirred at r.t. overnight. The mixture was concentrated and purified by flash column chromatography eluting with MeOH/H₂O to afford 26d as a yellow solid (85 mg, yield: 49%). MS (m/z): 511.0 (M+H)⁺.

### Step 26-3 (S)-2-(1-(2-aminopyrazolo[1,5-a][1,3,5]triazin-4-yl)pyrrolidin-2-yl)-5-chloro-3-phenylpyrrolo[2,1-f][1,2,4]triazin-4(3H)-one (Compound 134)

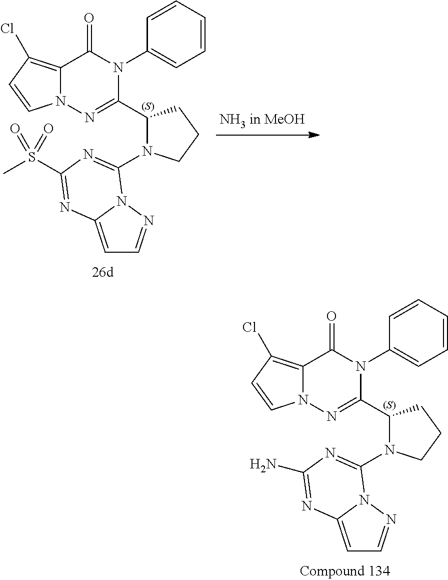

To a solution of 26d (82 mg, 0.16 mmol) in 5 mL of THF was added 4 mL of 7N NH₃ in MeOH, then the mixture was stirred at r.t. overnight. After concentration, the residue was purified by flash column chromatography, eluting with MeOH/H₂O, and further purified by preparative TLC, eluting with MeOH/DCM=1/80, to give Compound 134 as a white solid (28.8 mg, yield: 40%). MS (m/z): 448.1 (M+H)⁺. ¹H NMR (400 MHz, DMSO-d₆) δ 7.93-7.78 (m, 2H), 7.63-7.54 (m, 5H), 6.62-6.36 (m, 3H), 5.70-5.59 (m, 1H), 4.71-4.31 (m, 1H), 3.95-3.83 (m, 1H), 3.72-3.64 (m, 1H), 2.12-1.74 (m, 4H).

The following Compounds was prepared according to the procedure of Compound 134 using the corresponding reagents and intermediates under appropriate conditions that will be recognized by one skilled in the art:

| Compd. | | LC/MS | |
|---|---|---|---|
| No. | Structure | (M + H)⁺ | NMR |
| 135 | 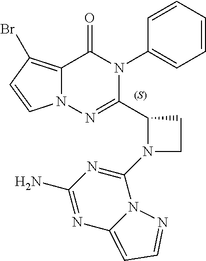 | 478.2 | ¹H NMR (400 MHz, DMSO-d₆) δ: 7.90 (s, 1H), 7.80-7.62 (m, 5H), 7.61-7.46 (m, 2H), 6.82 (s, 1H), 6.60 (s, 2H), 5.78-5.66 (br, 1H), 4.40-4.11 (m, 2H), 2.75-2.69 (m, 1H), 2.50-2.12 (m, 1H). |
| 136 | 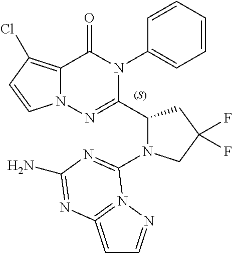 | 484.1 | ¹H NMR (400 MHz, DMSO-d₆) δ: 7.98-7.77 (m, 2H), 7.63-7.54 (m, 5H), 6.61-6.55 (m, 3H), 5.92-5.71 (m, 1H), 4.98-4.81 (m, 1H), 4.28-4.19 (m, 1H), 3.13-2.90 (m, 2H). |
| 137 | 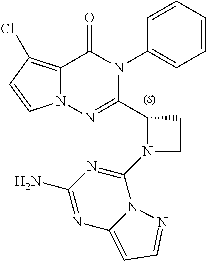 | 434.1 | ¹H NMR (400 MHz, DMSO-d₆) δ: 7.98-7.48 (m, 7H), 6.82-6.53 (m, 3H), 5.88-5.61 (m, 1H), 5.61-4.95 (m, 1H), 4.68-4.06 (m, 2H), 2.72-2.64 (m, 1H), 2.52-2.05 (m, 1H). |
| 434 | 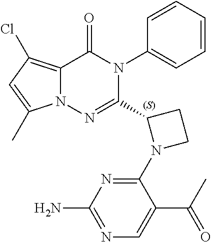 | 450.1 | ¹H NMR (400 MHz, CD₃OD) δ 8.40 (s, 1H), 7.66-7.61 (m, 1H), 7.58-7.52 (m, 3H), 7.28-7.26 (m, 1H), 6.90 (s, 1H), 5.39-5.29 (m, 1H), 4.34-4.27 (m, 1H), 3.89-3.78 (m, 1H), 2.32-2.24 (m, 1H), 2.22-2.2.19 (m, 1H), 2.17 (s, 3H), 2.14 (s, 3H). |
| 483** | 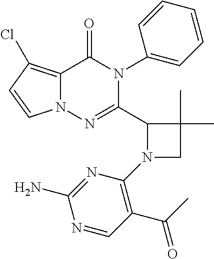 [SMILES: CC(=O)c1cnc(N)nc1N1CC(C)(C)C1c1nn2ccc(Cl)c2c(=O)n1-c1ccccc1] | 464.2 | ¹H NMR (400 MHz, CD₃OD) δ 8.32 (br, 1H), 8.10 (br, 1H), 7.76 (d, J = 6.0 Hz, 1H), 7.59-7.47 (m, 3H), 7.28-7.26 (m, 2H), 6.46 (d, J = 3.0 Hz, 1H), 5.07 (br, 1H), 4.67 (br, 1H), 3.88 (br, 1H), 2.15 (br, 3H), 1.10 (s, 3H), 0.64 (s, 3H).) |

**prepared from (S)-methyl 3,3-dimethylazetidine-2-carboxylate

### Example 27

### Compound 138

### (S)-2-(1-(4-amino-1,3,5-triazin-2-yl)pyrrolidin-2-yl)-5-chloro-3-phenylpyrrolo[2,1-f][1,2,4]triazin-4(3H)-one

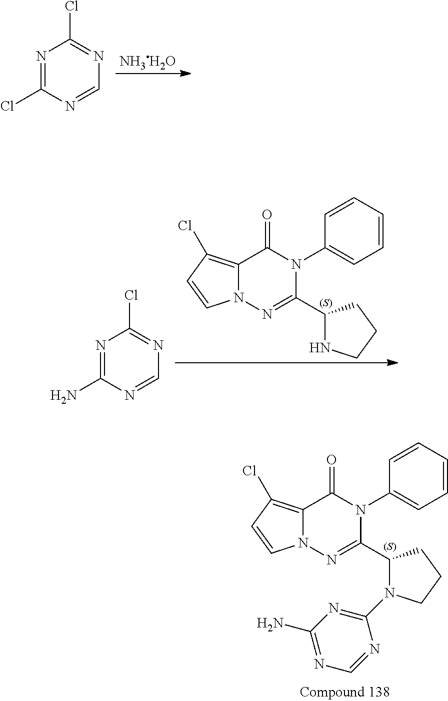

2,4-dichloro-1,3,5-triazine (45 mg, 0.3 mmol) was added to 2 mL of NH₃.H₂O aq., the reaction was stirred at −20° C. for 10 min, then filtered, washed with water and dried to give 4-chloro-1,3,5-triazin-2-amine (18 mg, yield: 46%) as a yellow solid which was used in the next step without further purification. MS (m/z): 131.0 (M+H)⁺.

(S)-2-(1-(4-amino-1,3,5-triazin-2-yl)pyrrolidin-2-yl)-5-chloro-3-phenylpyrrolo[2,1-f][1,2,4]triazin-4(3H)-one was prepared with 4-chloro-1,3,5-triazin-2-amine as the material according to the procedure of Example 1 from 1e to Compound 1. MS (m/z): 409.1 (M+H)⁺. ¹H NMR (400 MHz, CD₃OD) δ: 8.02 (d, J=1.6 Hz, 1H), 7.81 (d, J=7.6 Hz, 1H), 7.64-7.54 (m, 3H), 7.42-7.39 (m, 1H), 7.37-7.35 (m, 1H), 6.50-6.49 (m, 1H), 4.67-4.64 (m, 1H), 3.81-3.73 (m, 1H), 3.59-3.53 (m, 1H), 2.20-2.08 (m, 2H), 1.97-1.85 (m, 2H).

### Example 28

### Compound 139

### (S)-2-(1-(9H-purin-6-yl)pyrrolidin-2-yl)-4-oxo-3-phenyl-3,4-dihydropyrrolo[2,1-f][1,2,4]triazine-5-carboxamide

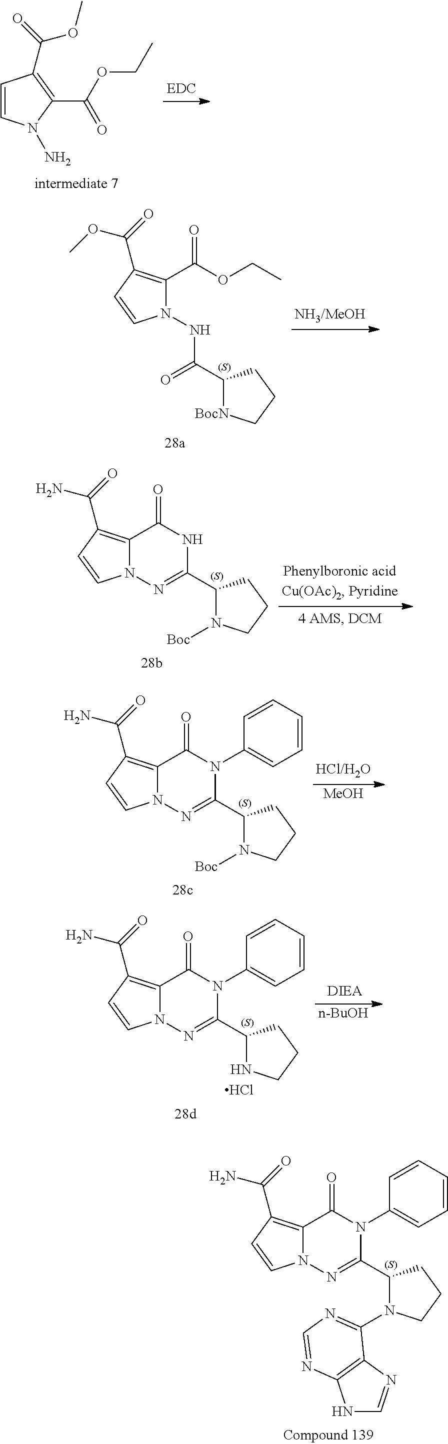

### Step 28-1 (S)-2-ethyl 3-methyl 1-(1-(tert-butoxycarbonyl)pyrrolidine-2-carboxamido)-1H-pyrrole-2,3-dicarboxylate (28a)

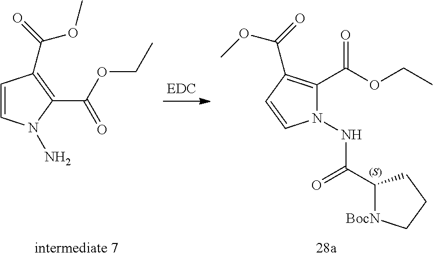

To a mixture of Intermediate 7 (500 mg, 2.36 mmol) in THF (40 mL) were added BOC-L-Proline (557 mg 2.59 mmol) and EDC (497 mg 2.59 mmol) at r.t. The reaction was stirred at r.t overnight. The mixture was concentrated and purified by flash chromatography to afford 28a as a yellow oil (800 mg, yield: 83%). MS (m/z): 410.5 (M+1)⁺.

### Step 28-2 (S)-tert-butyl 2-(5-carbamoyl-4-oxo-3,4-dihydropyrrolo[2,1-f][1,2,4]triazin-2-yl)pyrrolidine-1-carboxylate (28b)

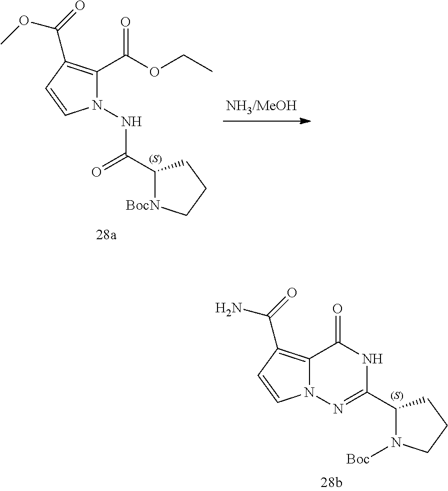

The mixture of 28a (800 mg 1.96 mmol) in a solution of NH₃ in MeOH (7N, 50 mL) was stirred at 130° C. for 36 h in a sealed tube. The reaction was concentrated and purified by chromatography to afford 28b as a yellow solid (580 mg, yield: 75%). MS (m/z):

348.5 (M+1)⁺.

Compound 139 was prepared from 28b according to the procedure of Example 1.

MS (m/z): 442.2 (M+1)⁺. ¹H NMR (400 MHz, DMSO-d₆) δ 9.22 (s, 1H), 8.23-8.18 (m, 1.5H), 8.10 (s, 0.5H), 7.87-7.42 (m, 6H), 7.35 (s, 1H), 6.95 (s, 0.5H), 6.92 (s, 0.5H), 5.37-5.25 (m, 0.5H), 4.74-4.45 (m, 0.5H), 4.38-4.26 (m, 0.5H), 4.15-4.01 (m, 0.5H), 3.94-3.84 (m, 0.5H), 3.74-3.63 (m, 0.5H), 2.35-2.21 (m, 2H), 2.01-1.93 (m, 1H), 1.90-1.82 (m, 1H).

Compound 140 was prepared according to the procedure of Compound 139 using the corresponding reagents and intermediates under appropriate conditions that will be recognized by one skilled in the art.

| Compd. | | LC/MS | |
|---|---|---|---|
| No. | Structure | (M + H)⁺ | NMR |
| 140 | 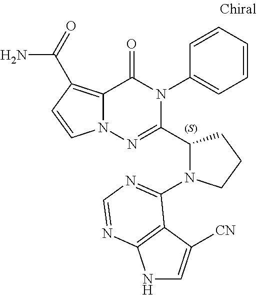 | 466.2 | ¹H NMR (400 MHz, DMSO-d₆) δ 9.2 (s, 1H), 8.29 (s, 1H), 8.27 (s, 1H), 7.79 (d, J = 7.9 Hz, 1H), 7.65-7.52 (m, 5H), 7.35 (s, 1H), 6.96 (d, J = 2.9 Hz, 1H), 4.67 (dd, J = 8.0, 3.5 Hz, 1H), 4.16-4.05 (m, 1H), 3.94 (m, 1H), 2.32-2.19 (m, 2H), 2.04-1.92 (m, 2H). |

### Example 29

### Compound 177

### (S)-2-(1-(9H-purin-6-yl)pyrrolidin-2-yl)-5-(hydroxymethyl)-3-phenylpyrrolo[2,1-f][1,2,4]triazin-4(3H)-one

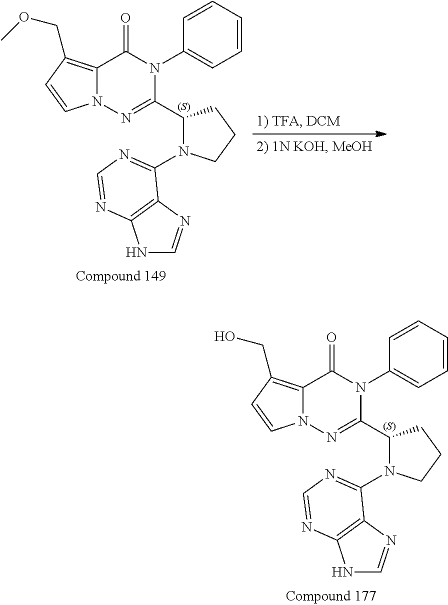

To a solution of Compound 149 (30 mg, 0.068 mmol) in CH₂Cl₂ (1 mL) was added TFA (2 mL) at 0° C., the reaction was stirred at r.t. for 30 min, then concentrated at r.t. The residue was dissolved in MeOH (2 mL), and treated with 1N KOH (2 mL), then stirred at r.t. for another 1 h. The mixture was adjusted to pH=7.0, then concentrated and purified by chromatography to give the title compound as a white solid (12 mg, yield: 41%). MS (m/z): 429.6 (M+1)⁺¹H NMR (400 MHz, CD₃OD) δ 8.21 (s, 1H), 8.14 (s, 1H), 7.95 (s, 0.5H), 7.91 (s, 0.5H), 7.69-7.43 (m, 4H), 7.37 (br, 1H), 7.17 (s, 0.5H), 7.09 (s, 0.5H), 6.43 (s, 0.5H), 6.40 (s, 0.5H), 5.51 (br, 0.5H), 4.48 (s, 2H), 4.31 (br, 0.5H), 4.09 (br, 0.5H), 3.92 (br, 0.5H), 3.71 (br, 0.5H), 2.29-1.88 (m, 4H).

The following Compounds 178-179 were prepared according to the procedure of Compound 177 using the corresponding reagents and intermediates under appropriate conditions that will be recognized by one skilled in the art.

| Compd. | | LC/MS | |
|---|---|---|---|
| No. | Structure | (M + H)⁺ | NMR |
| 178 | 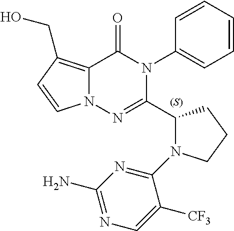 | 472.5 | ¹H NMR (400 MHz, CD₃OD) δ 8.27 (s, 1H), 7.85 (d, J = 7.8 Hz, 1H), 7.74-7.63 (m, 3H), 7.52 (d, J = 7.2 Hz, 1H), 7.39 (d, J = 2.5 Hz, 1H), 6.65 (d, J = 2.4 Hz, 1H), 5.04-5.01 (m, 1H), 4.95 (s, 2H), 3.97-3.87 (m, 1H), 3.83-3.73 (m, 1H), 2.34-2.28 (m, 1H), 2.14-2.13 (m, 1H), 2.02-1.91 (m, 2H). |
| 179 | 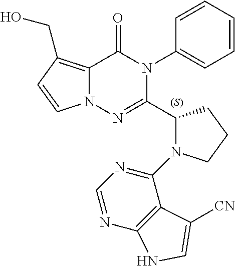 | 453.6 | ¹H NMR (400 MHz, CD₃OD) δ 8.23 (s, 1H), 7.97 (s, 1H), 7.77 (d, J = 7.8 Hz, 1H), 7.65-7.53 (m, 3H), 7.43 (d, J = 7.3 Hz, 1H), 7.28 (d, J = 2.6 Hz, 1H), 6.52 (d, J = 2.6 Hz, 1H), 4.92-4.90 (m, 1H), 4.56 (s, 2H), 4.30-4.24 (m, 1H), 4.10-4.04 (m, 1H), 2.47-2.41 (m, 1H), 2.20- 2.15 (m, 1H), 2.12-1.99 (m, 2H). |

### Example 30

### Compound 180

### (S)-2-(1-(5-(2-aminopyrimidin-5-yl)-7H-pyrrolo[2,3-d]pyrimidin-4-yl)azetidin-2-yl)-5-fluoro-3-phenylpyrrolo[2,1-f][1,2,4]triazin-4(3H)-one

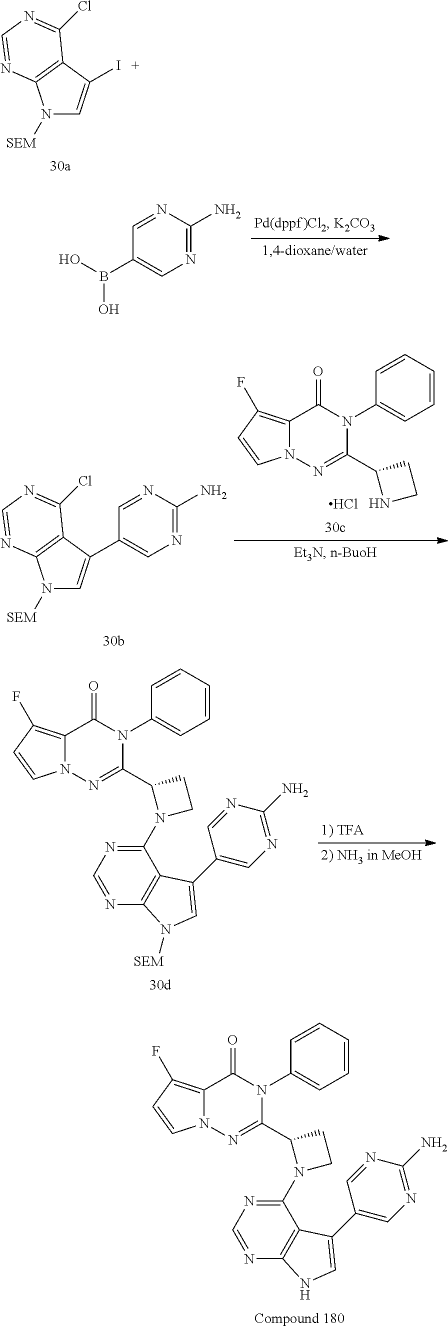

### Step 30-1 5-(4-chloro-7-((2-(trimethylsilyl)ethoxy)methyl)-7H-pyrrolo[2,3-d]pyrimidin-5-yl)pyrimidin-2-amine (30b)

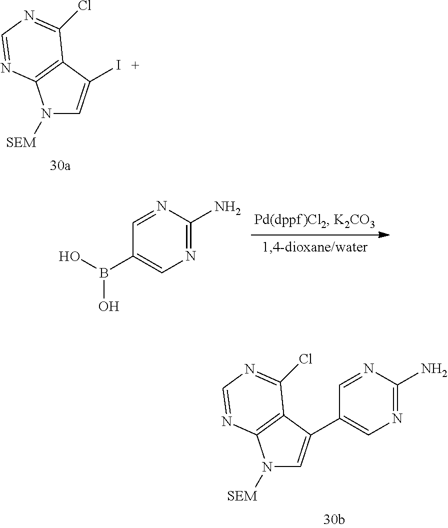 [SMILES: C[Si](C)(C)CCOCn1cc(I)c2c(Cl)ncnc21 ; Nc1ncc(B(O)O)cn1 ; C[Si](C)(C)CCOCn1cc(-c2cnc(N)nc2)c2c(Cl)ncnc21]

To a solution of 30a (409 mg, 1 mmol) in 1,4-dioxane/water (10 mL/1 mL) was added 2-aminopyrimidin-5-ylboronic acid (139 mg, 1 mmol), Pd(dppf)Cl₂ (81.6 mg, 0.1 mmol) and K₂CO₃ (414 mg, 3 mmol). Under N₂, the reaction mixture was heated at 100° C. for 2 h. Then the solvent was removed in reduced pressure and the residue was purified by flash column chromatography eluting with MeOH/DCM to give 30b as a yellow solid (310 mg, yield: 82.4%). MS (m/z): 377.1 (M+H)⁺

### Steps 30-2 to 4 (S)-2-(1-(5-(2-aminopyrimidin-5-yl)-7H-pyrrolo[2,3-d]pyrimidin-4-yl)azetidin-2-yl)-5-fluoro-3-phenylpyrrolo[2,1-f][1,2,4]triazin-4(3H)-one (Compound 180)

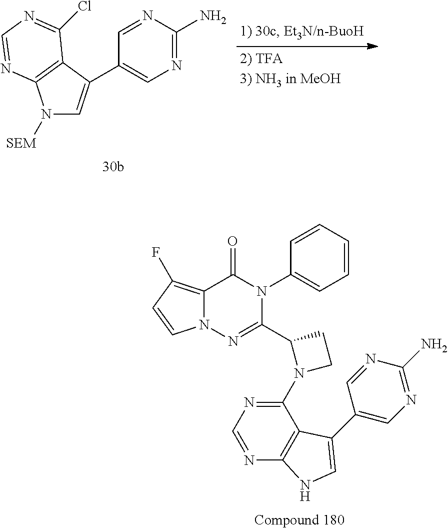 [SMILES: C[Si](C)(C)CCOCn1cc(-c2cnc(N)nc2)c2c(Cl)ncnc21 ; Nc1ncc(-c2c[nH]c3ncnc(N4CC[C@H]4c4nn5ccc(F)c5c(=O)n4-c4ccccc4)c23)cn1]

A mixture of 30c (64 mg 0.2 mmol) (The intermediate was synthesized according to the procedure of Example 1), 30b (68 mg, 0.18 mmol) and Et₃N (80 mg, 0.8 mmol) in n-BuOH (2 mL) was stirred at 100° C. for 1 h. The reaction solution was concentrated and the residue was dissolved in TFA (3 mL). The resulting mixture was stirred at r.t. for 30 min. Then the solvent was removed in vacuo. To the residue was added a solution of NH₃ in MeOH (7N, 3 mL). The mixture was stirred at r.t. for 30 min. The solvent was evaporated and the residue was purified by flash column chromatography eluting with MeOH/water to give Compound 180 as a white solid (37 mg, yield: 37.4%). MS (m/z): 495.1 (M+H)⁺; ¹H NMR (400 MHz, DMSO-d₆) δ: 12.00 (s, 1H), 8.37 (s, 2H), 8.23 (s, 1H), 7.66-7.57 (m, 1H), 7.57-7.48 (m, 4H), 7.43 (d, J=2.7 Hz, 1H), 7.32 (d, J=2.4 Hz, 1H), 6.65 (s, 2H), 6.49 (d, J=3.2 Hz, 1H), 5.06-5.00 (m, 1H), 3.20-3.16 (m, 1H), 3.13-2.99 (m, 1H), 2.42-2.38 (m, 1H), 1.78-1.68 (m, 1H).

Compounds 181-184 were prepared according to the procedure of Compound 180 using the corresponding reagents and intermediates under appropriate conditions that will be recognized by one skilled in the art:

| Compd. | | LC/MS | |
|---|---|---|---|
| No. | Structure | (M + H)⁺ | NMR |
| 181 | 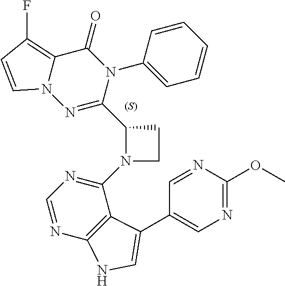 | 510.2 | ¹H NMR (400 MHz, CD₃OD) δ: 8.75 (s, 2H), 8.23 (s, 1H), 7.62-7.45 (m, 4H), 7.38-7.25 (m, 3H), 6.38-6.26 (br, 1H), 5.17-5.09 (m, 1H), 4.03 (s, 3H), 3.43-3.33 (m, 1H), 3.23-3.17 (br, 1H), 2.37-2.17 (br, 1H), 1.86-1.76 (br, 1H). |
| 182 | 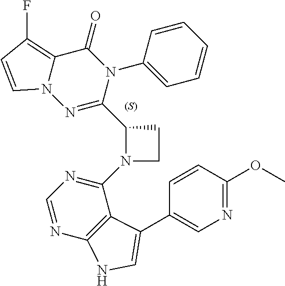 | 509.3 | ¹H NMR (400 MHz, CD₃OD) δ 8.26 (d, J = 2.0 Hz, 1H), 8.19 (s, 1H), 7.83 (dd, J = 8.5, 2.3 Hz, 1H), 7.56-7.52 (m, 2H), 7.47 (d, J = 7.7 Hz, 2H), 7.35-7.30 (m, 1H), 7.26 (d, J = 7.5 Hz, 1H), 7.17 (s, 1H), 6.84 (d, J = 8.5 Hz, 1H), 6.31 (d, J = 3.2 Hz, 1H), 5.12-5.00 (m, 1H), 3.91 (s, 3H), 3.38-3.31 (m, 1H), 3.22-3.12 (m, 1H), 2.30-2.19 (m, 1H), 1.81-1.69 (m, 1H). |
| 183 | 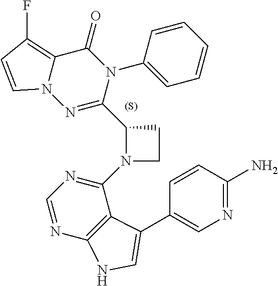 | 494.2 | ¹H NMR (400 MHz, CD₃OD) δ: 8.17 (s, 1H), 8.04 (s, 1H), 7.64-7.47 (m, 5H), 7.37-7.24 (m, 2H), 7.10 (s, 1H), 6.63 (d, J = 8.5 Hz, 1H), 6.31 (d, J = 3.1 Hz, 1H), 5.08-5.03 (m, 1H), 3.41-3.31 (m, 2H), 2.28-2.20 (m, 1H), 1.80-1.72 (m, 1H). |
| 184 | 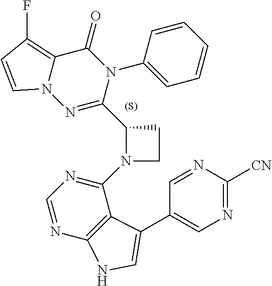 | 505.3 | ¹H NMR (400 MHz, DMSO-d₆) δ: 9.27 (s, 2H), 8.33 (s, 1H), 7.89 (s, 1H), 7.65-7.43 (m, 6H), 6.51 (d, J = 2.6 Hz, 1H), 5.20-5.08 (m, 1H), 3.20-3.14 (m, 2H), 2.49-2.43 (m, 1H), 1.75-1.61 (m, 1H). |

### Example 31

### Compound 185

### (S)-2-(1-(5-acetyl-7H-pyrrolo[2,3-d]pyrimidin-4-yl)azetidin-2-yl)-5-chloro-3-phenylpyrrolo[2,1-f][1,2,4]triazin-4(3H)-one

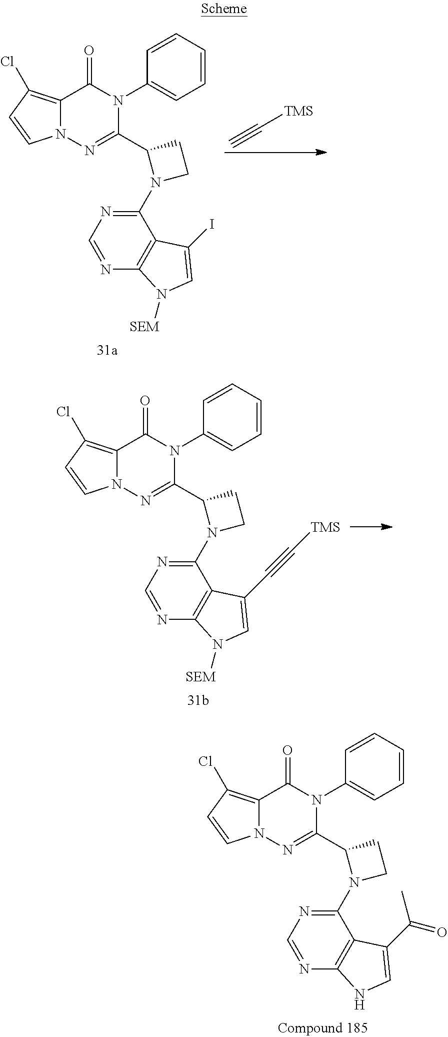

### Step 31-1

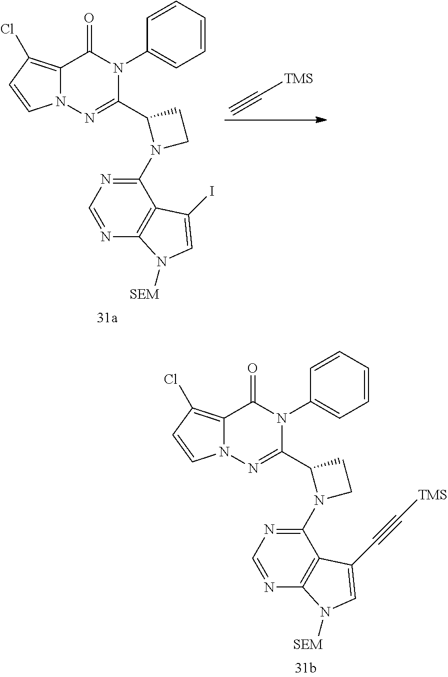

A mixture of 31a (60 mg, 0.09 mmol) (The intermediate was synthesized according to the procedure of Example 1), CuI (10 mg, 0.05 mmol), Pd(PPh₃)₂Cl₂ (50 mg, 0.05 mmol), DIEA (0.2 mL) and (trimethylsilyl)acetylene (0.5 mL) were stirred at r.t. in DMF (5 mL) under N₂ for 3 h. The mixture was diluted with DCM and washed with water three times and brine once, dried over Na₂SO₄, filtered and concentrated. The residue was purified by flash chromatography to give 31b as a brown solid (30 mg, yield: 52%).

### Step 31-2

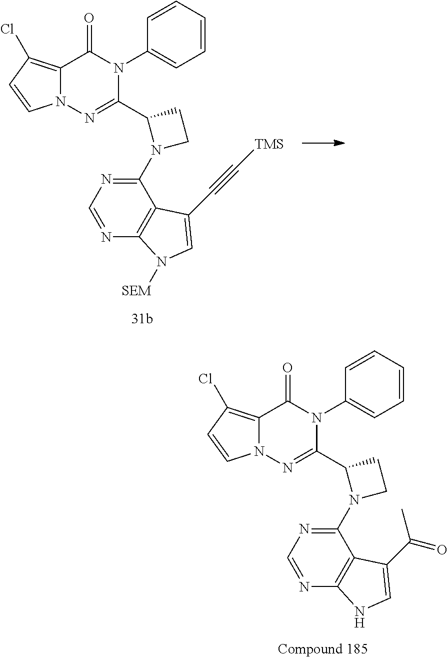 [SMILES: C[Si](C)(C)C#Cc1cn(COCC[Si](C)(C)C)c2ncnc(N3CC[C@H]3c3nn4ccc(Cl)c4c(=O)n3-c3ccccc3)c12 ; CC(=O)c1c[nH]c2ncnc(N3CC[C@H]3c3nn4ccc(Cl)c4c(=O)n3-c3ccccc3)c12]

Cooled in ice-batch, to 31b (30 mg, 0.046 mmol) was added TFA (5 mL) and the mixture was stirred 0.5 h at 0° C., then 1.5 h at r.t. The reaction mixture was concentrated and the resulting residue was diluted with MeOH (10 mL). Then Conc. NH₃.H₂O aq. (5 mL) was added and the mixture was stirred for another 2 h. After concentration, the residue was purified by chromatography eluting with MeOH/water to give Compound 185 as a solid (12 mg, yield: 56%). MS (m/z): 460.2 (M+H)⁺; ¹H NMR (400 MHz, DMSO-d₆) δ: 12.41 (s, 1H), 8.21 (s, 1H), 8.14 (s, 1H), 7.67-7.52 (m, 5H), 7.49-7.43 (m, 1H), 6.66-6.62 (m, 1H), 5.05-4.95 (br, 1H), 4.33-4.23 (m, 1H), 3.78-3.72 (m, 1H), 2.49-2.44 (m, 1H), 2.40 (s, 3H), 1.89-1.79 (m, 1H).

### Example 33

### Compound 293

### 5-chloro-2-((4R)-1-oxido-3-(9H-purin-6-yl)thiazolidin-4-yl)-3-phenylpyrrolo[2,1-f][1,2,4]triazin-4(3H)-one

### Compound 294

### (R)-2-(3-(9H-purin-6-yl)thiazolidin-4-yl)-5-chloro-3-phenylpyrrolo[2,1-f][1,2,4]triazin-4(3H)-one

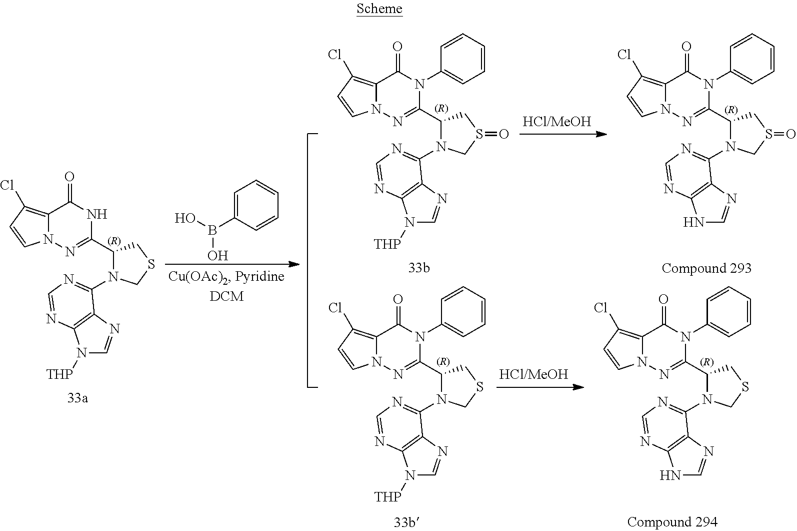

### Step 33-1 5-chloro-2-((4R)-1-oxido-3-(9-(tetrahydro-2H-pyran-2-yl)-9H-purin-6-yl)thiazolidin-4-yl)-3-phenylpyrrolo[2,1-f][1,2,4]triazin-4(3H)-one (33b)

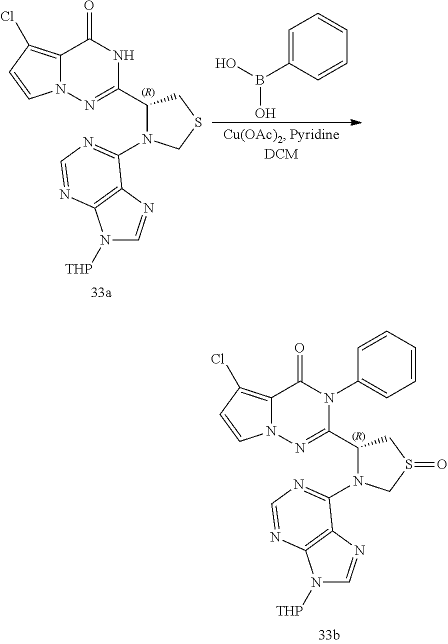

A mixture of 33a (prepared according to the procedures described in Example 41 using the corresponding reagents and intermediates) (180 mg, 0.392 mmol), phenylboronic acid (96 mg, 0.784 mmol), Cu(OAc)₂ (143 mg, 0.784 mmol) and pyridine (0.125 mL, 1.568 mmol) in DCM (20 mL) was stirred at r.t. overnight, then filtered and concentrated. The residue was further purified by flash chromatography eluting with water and methanol to give 33-b as a white solid. Yield: 4.6%. MS (m/z): 551.1 (M+1)⁺

### Step 33-2 5-chloro-2-((4R)-1-oxido-3-(9H-purin-6-yl)thiazolidin-4-yl)-3-phenyl-pyrrolo[2,1-f][1,2,4]triazin-4(3H)-one (Compound 293)

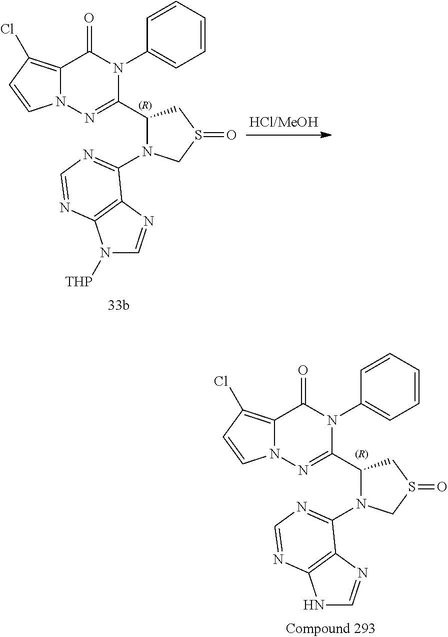

A solution of 33b (10 mg, 0.0181 mmol) in HCl/MeOH (2 N, 2 mL) was stirred at r.t. for 15 min, then neutralized with aq. NaHCO₃ and extracted with EtOAc three times. The combined organic layers were dried, concentrated and purified by flash chromatography to give Compound 293 as a white solid. Yield: 51%. ¹H NMR (400 MHz, CD₃OD) δ 8.34 (s, 1H), 8.19-7.89 (m, 2H), 7.82-7.44 (m, 4H), 7.36-7.23 (m, 1H), 6.48-6.41 (m, 1H), 4.59-4.51 (m, 3H), 3.36-3.32 (m, 2H). MS (m/z): 467.1 (M+H)⁺.

### Step 33-3 5-chloro-3-phenyl-2-((4R)-3-(9-(tetrahydro-2H-pyran-2-yl)-9H-purin-6-yl)thiazolidin-4-yl)pyrrolo[2,1-f][1,2,4]triazin-4(3H)-one (33-b′)

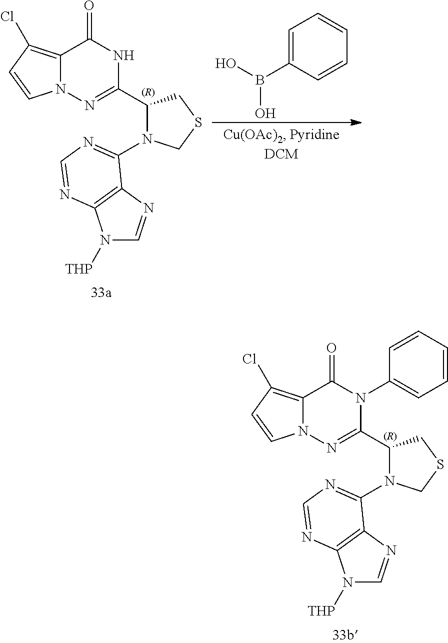

A mixture of 33a (2.5 g, 5.45 mmol), phenylboronic acid (1.33 g, 10.9 mmol), Cu(OAc)₂ (1.98 g, 10.9 mmol), pyridine (2.2 mL, 27.25 mmol) and 4 Å molecular sieves in DCM (60 mL) was stirred at r.t. under O₂ overnight, then filtered and concentrated. The residue was purified by flash chromatography to give 33b′ as a white solid. Yield: 0.7%. MS (m/z): 535.5 (M+1)⁺.

### Step 33-4 (R)-2-(3-(9H-purin-6-yl)thiazolidin-4-yl)-5-chloro-3-phenylpyrrolo[2,1-f][1,2,4]triazin-4(3H)-one (Compound 294)

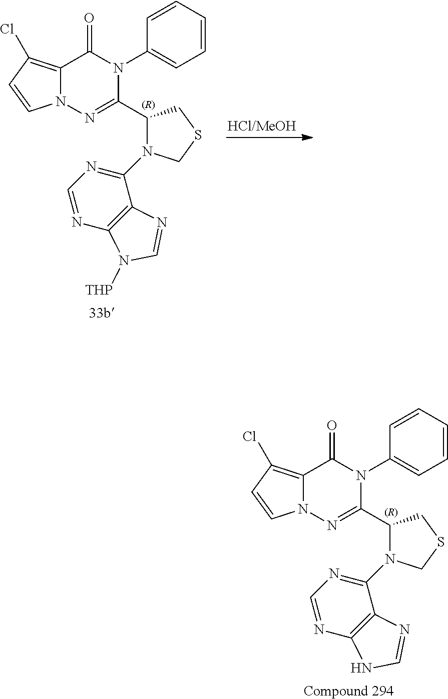

A solution of 33b′ (20 mg, 0.0374 mmol) in HCl/MeOH (2 N, 2 mL) was stirred at r.t. for 10 min, then neutralized with aq. NaHCO₃ and concentrated and purified by flash chromatography to give Compound 294 as a white solid. Yield: 80%. ¹H NMR (400 MHz, DMSO-d₆) δ: 12.94 (br, 1H), 8.12-7.93 (m, 2H), 7.62-7.20 (m, 6H), 6.44-6.35 (m, 1H), 5.80-5.46 (m, 1H), 4.98-4.65 (m, 2H), 2.91-2.77 (m, 2H). MS (m/z): 451.4 (M+1)⁺.

### Example 34

### Compound 296

### (S)-2-(1-(5-acetyl-7H-pyrrolo[2,3-d]pyrimidin-4-yl)azetidin-2-yl)-5-chloro-3-(4-fluorophenyl)pyrrolo[2,1-f][1,2,4]triazin-4(3H)-one

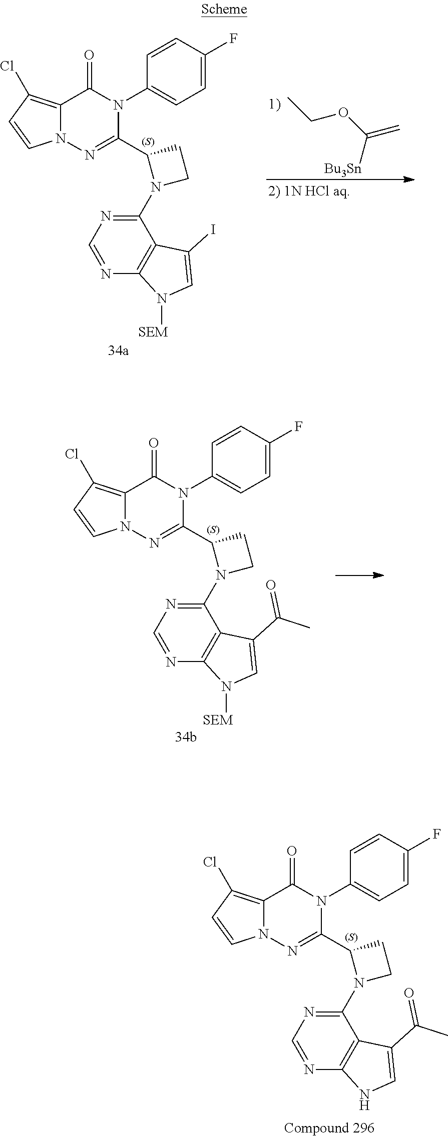

### Step 34-1 (S)-2-(1-(5-acetyl-7-((2-(trimethylsilyl)ethoxy)methyl)-7H-pyrrolo[2,3-d]pyrimidin-4-yl)azetidin-2-yl)-5-chloro-3-(4-fluorophenyl)pyrrolo[2,1-f][1,2,4]triazin-4(3H)-one (34b)

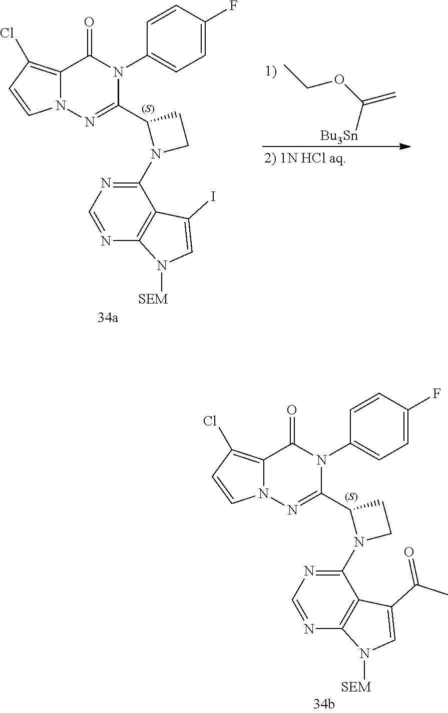

Under N₂, a mixture of 34a (prepared according to the procedures described in Example 1 using the corresponding reagents and intermediates) (50 mg, 0.07 mmol), tributyl(1-ethoxyvinyl)stannane (100 mg, 0.28 mmol) and Pd(PPh₃)₂Cl₂ (100 mg, 0.14 mmol) in 5 mL of dioxane was stirred at reflux for 3 h. After cooling to r.t., to the reaction was added 0.5 mL of aq. 1N HCl. The mixture was stirred at r.t. for 3 h. Then the mixture was diluted with DCM, washed with water, brine, dried over Na₂SO₄, filtered and concentrated. The residue was purified by flash chromatography to give 34b as a brown solid. Yield: 46%. MS (m/z): 608.2 (M+1)⁺

### Step 34-2 (S)-2-(1-(5-acetyl-7H-pyrrolo[2,3-d]pyrimidin-4-yl)azetidin-2-yl)-5-chloro-3-(4-fluorophenyl)pyrrolo[2,1-f][1,2,4]triazin-4(3H)-one (Compound 296)

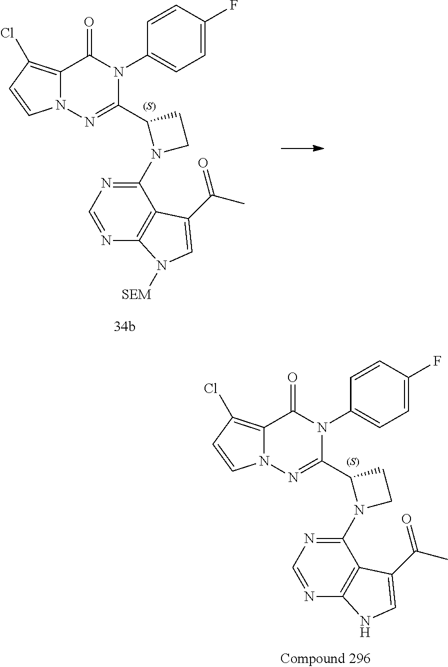

The mixture of 34b (20 mg, 0.03 mmol) in TFA (5 mL) was stirred at 0° C. for 0.5 h, then concentrated, the resulting residue was diluted with MeOH (10 mL), followed by conc. NH₃.H₂O aq. (5 mL), the mixture was stirred for 2 h. After concentration, the residue was purified by p-TLC to give Compound 296 as a white solid (3 mg, yield: 19%). ¹H NMR (400 MHz, DMSO-d₆) δ: 8.09 (s, 1H), 8.03 (s, 1H), 7.74-7.09 (m, 5H), 6.67-6.57 (m, 1H), 4.98-4.84 (br, 1H), 4.31-4.18 (m, 1H), 3.71-3.61 (m, 1H), 2.31 (s, 3H), 1.96-1.90 (m, 1H), 1.80-1.75 (m, 1H). MS (m/z): 478.2 (M+1)⁺;

The following Compounds were prepared according to the procedure of Compound 296 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 297 | 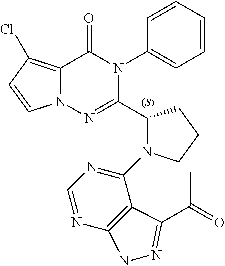 | 475.1 | ¹H NMR (400 MHz, DMSO-d₆) δ 7.97 (s, 1H), 7.77 (d, J = 7.6 Hz, 1H), 7.63-7.50 (m, 5H), 6.58 (d, J = 2.8 Hz, 1H), 4.46 (t, J = 6.4 Hz, 1H), 4.06-4.00 (m, 1H), 3.81- 3.75 (m, 1H), 2.61 (s, 3H), 2.11-1.93 (m, 3H), 1.63-1.58 (m, 1H). |
| 298 | 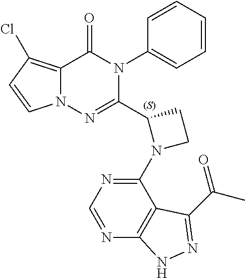 | 461.1 | ¹H NMR (400 MHz, CD₃OD) δ 8.54 (s, 1H), 8.07-8.00 (m, 1H), 7.94-7.89 (m, 2H), 7.85-7.81 (m, 2H), 7.65-7.63 (m, 1H), 6.77 (br, 1H), 4.88-4.81 (m, 0.5H), 4.32-4.22 (m, 0.5H), 2.75 (s, 3H), 2.40-2.31 (m, 1H), 1.95-1.87 (m, 0.5H), 1.67-1.62 (m, 0.5H) |
| 299 | 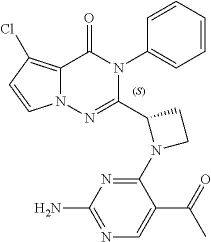 | 436.0 | ¹H NMR (400 MHz, DMSO-d₆) δ 8.42 (s, 1H), 7.71-7.47 (m, 5H), 7.39-7.36 (m, 1H), 6.82 (s, 2H), 6.62 (d, J = 3.0 Hz, 1H), 4.87-4.75 (m, 1H), 4.15-4.08 (m, 1H), 3.29-3.28 (m, 1H), 2.43-2.35 (m, 1H), 2.23 (s, 3H), 2.03-1.75 (m, 1H). |
| 300 | 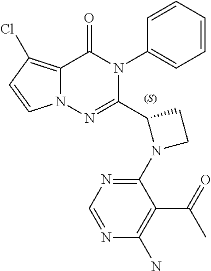 | 436.3 | ¹H NMR (400 MHz, DMSO-d₆) δ 7.93 (s, 1H), 7.63-7.43 (m, 6H), 7.20 (s, 2H), 6.66 (d, J = 2.8 Hz, 1H), 4.96-4.92 (m, 1H), 4.00-3.99 (m, 2H), 2.41 (s, 3H), 2.02-1.89 (m, 2H). |
| 301 | 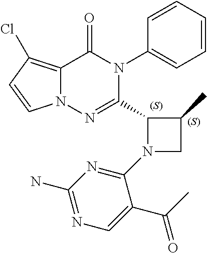 | 450.3 | ¹H NMR (400 MHz, CD₃OD) δ 8.43 (s, 1H), 7.76-7.53 (m, 4H), 7.44-7.25 (m, 2H), 6.52 (d, J = 2.8 Hz, 1H), 4.99-4.93 (m, 1H), 4.50-4.28 (m, 1H), 3.41-3.34 (m, 1H), 2.69-2.40 (m, 1H), 2.26 (s, 3H), 0.67 (d, J = 6.8 Hz, 3H). |
| 302 | 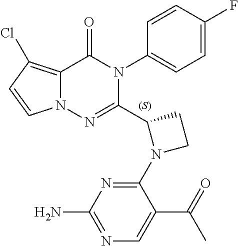 | 454.4 | ¹H NMR (400 MHz, CD₃OD) δ 8.42 (s, 1H), 7.70-7.64 (m, 1H), 7.37-7.26 (m, 4H), 6.53 (d, J = 3.2 Hz, 1H), 5.31-5.14 (m, 1H), 4.33-4.27 (m, 1H), 3.83-3.59 (m, 1H), 2.39-2.31 (m, 1H), 2.27 (s, 3H), 2.19-2.10 (m, 1H). |
| 398 | 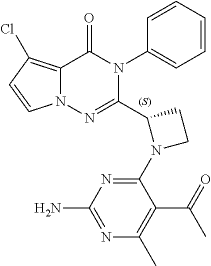 | 450.4 | ¹H NMR (400 MHz, DMSO-d₆) δ 7.66 (d, J = 3.0 Hz, 1H), 7.64-7.49 (m, 4H), 7.42-7.36 (m, 1H), 6.65 (d, J = 3.0 Hz, 1H), 6.43 (s, 2H), 4.73-4.69 (m, 1H), 3.80-3.75 (m, 1H), 2.49-2.39 (m, 1H), 2.32 (s, 3H), 2.10 (s, 3H), 1.93-1.86 (m, 1H). |
| 472 | 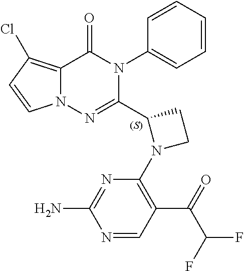 | 447.2 | ¹H NMR (400 MHz, CD3OD) δ 8.10 (s, 1H), 7.75 (s, 1H), 7.63-7.54 (m, 3H), 7.47 (s, 1H), 7.38 (d, J = 6.4, 1H), 6.56 (dd, J = 3.0, 1.7, 1H), 5.34-4.84 (m, 1H), 4.25-3.60 (m, 2H), 1.23 (s, 3H), 0.76 (s, 3H). |

### Example 35

### Compound 303

### (S)-5-chloro-2-(1-(5-(4,5-dihydrooxazol-2-yl)-7H-pyrrolo[2,3-d]pyrimidin-4-yl) pyrrolidin-2-yl)-3-phenylpyrrolo[2,1-f][1,2,4]triazin-4(3H)-one

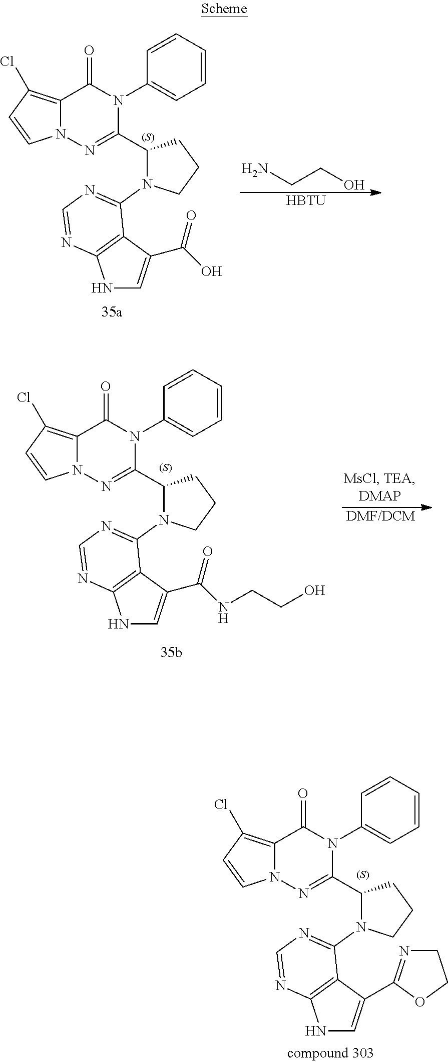

### Step 35-1 (S)-4-(2-(5-chloro-4-oxo-3-phenyl-3,4-dihydropyrrolo[2,1-f][1,2,4]triazin-2-yl)pyrrolidin-1-yl)-N-(2-hydroxyethyl)-7H-pyrrolo[2,3-d]pyrimidine-5-carboxamide (35b)

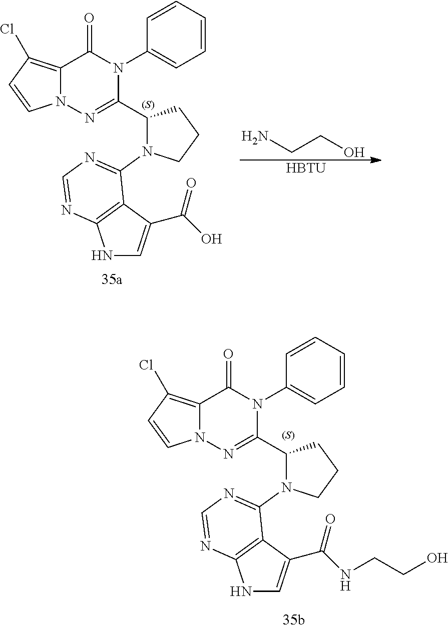

A mixture of 35a (prepared according to the procedures described in Example 1 using the corresponding reagents and intermediates) (100 mg, 0.21 mmol), 2-aminoethanolin (13 mg, 0.21 mmol), HBTU (88 mg, 0.23 mmol) and DIEA (54 mg, 0.42 mmol) in DMF (25 mL) was stirred at r.t. for 6 h. Then the reaction was diluted with water and extracted with EtOAc. The organic layers were dried, concentrated and purified by flash chromatography to give 35b as a white solid. Yield: 50%. MS (m/z): 519.0 (M+1)⁺.

### Step 35-2 (S)-5-chloro-2-(1-(5-(4,5-dihydrooxazol-2-yl)-7H-pyrrolo[2,3-d]pyrimidin-4-yl)pyrrolidin-2-yl)-3-phenylpyrrolo[2,1-f][1,2,4]triazin-4(3H)-one (Compound 303)

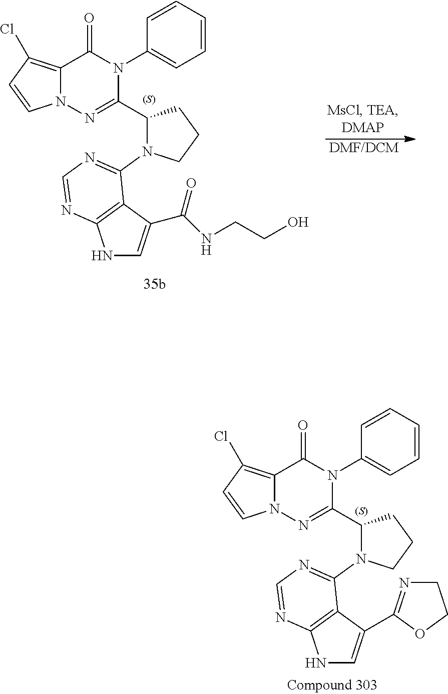

To a mixture of 35b (54 mg, 0.104 mmol), Et₃N (0.115 mL, 0.832 mmol) and DMAP (25 mg, 0.208 mmol) in DCM/DMF (4 mL/1 mL) at 0° C. was added MsCl (0.021 mL, 0.260 mmol). The mixture was stirred at r.t. for 3 h, then quenched by water and extracted with EtOAc. The combined organic layer was concentrated and purified by flash chromatography to give Compound 303 as a white solid. Yield: 38%. ¹H NMR (400 MHz, DMSO-d₆) δ 12.12 (br, 1H), 8.17 (s, 1H), 7.57-7.46 (m, 7H), 6.55 (d, T=2.9 Hz, 1H), 4.55 (br, 1H), 4.31-4.26 (m, 1H), 3.91-3.82 (m, 2H), 3.80-3.71 (m, 1H), 2.11-1.78 (m, 6H). MS (m/z): 501.2 (M+1)⁺.

The following Compounds were prepared according to the procedure of Compound 303 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 304 | 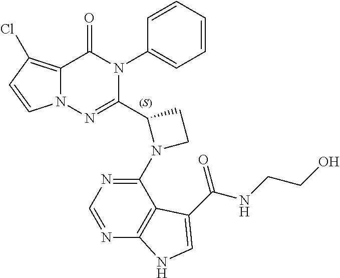 | 505.1 | ¹H NMR (400 MHz, DMSO-d₆) δ 11.94 (brs, 1H), 8.19 (s, 1H), 8.08 (t, J = 5.6 Hz, 1H), 7.65-7.61 (m, 3H), 7.55-7.52 (m, 2H), 7.49-7.46 (m, 2H), 6.65 (d, J = 3.0 Hz, 1H), 5.08-5.04 (m, 1H), 4.67 (brs, 1H), 4.30-4.18 (m, 1H), 3.78 (m, 1H), 3.47-3.44 (m, 2H), 3.28-3.18 (m, 2H), 2.56-2.52 (m, 1H), 1.88-1.85 (m, 1H). |
| 305 | 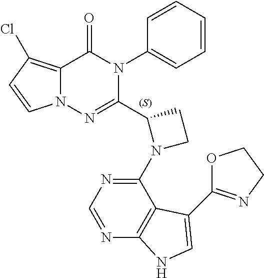 | 487.0 | ¹H NMR (400 MHz, DMSO-d₆) δ 12.22 (brs, 1H), 8.21 (s, 1H), 7.70 (d, J = 3.0 Hz, 1H), 7.64-7.59 (m, 2H), 7.58-7.52 (m, 3H), 7.47-7.44 (m, 1H), 6.66 (d, J = 3.0 Hz, 1H), 4.94-4.92 (m, 1H), 4.41-4.33 (m, 2H), 4.30-4.23 (m, 1H), 3.97-3.85 (m, 2H), 3.82-3.73 (m, 1H), 2.58-2.53 (m, 1H), 1.94-1.87 (m, 1H). |

### Example 36

### Compound 306

### (S)-5-chloro-2-(1-(5-(1-(hydroxyimino)ethyl)-7H-pyrrolo[2,3-d]pyrimidin-4-yl)pyrrolidin-2-yl)-3-phenylpyrrolo[2,1-f][1,2,4]triazin-4(3H)-one

### Compound 307

### (S)—N-(4-(2-(5-chloro-4-oxo-3-phenyl-3,4-dihydropyrrolo[2,1-f][1,2,4]triazin-2-yl)pyrrolidin-1-yl)-7H-pyrrolo[2,3-d]pyrimidin-5-yl)acetamide

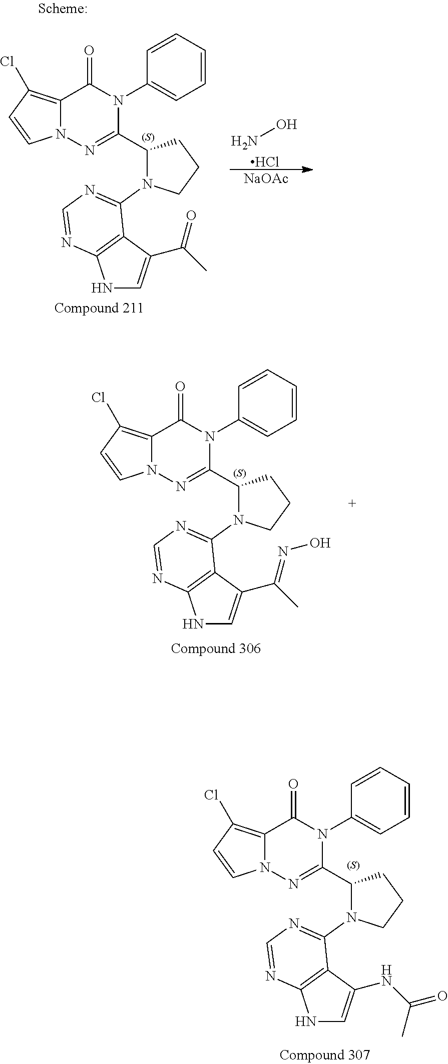

A mixture of Compound 211 (100 mg, 0.211 mmol), hydroxylamine hydrochloride (44 mg, 0.633 mmol), sodium acetate (42 mg, 0.506 mmol) in ethanol (7.5 mL) and water (5 mL) was stirred at reflux overnight, then concentrated. The residue was purified by flash chromatography to give Compound 306 (Yield: 55%) and Compound 307

Compound 306: ¹H NMR (400 MHz, DMSO-d₆) δ 11.81 (s, 1H), 10.80 (s, 1H), 8.15 (s, 1H), 7.73 (d, J=8.0 Hz, 1H), 7.58-7.43 (m, 4H), 7.40 (d, J=2.8 Hz, 1H), 7.16 (s, 1H), 6.56 (d, J=2.7 Hz, 1H), 4.66-4.62 (m, 1H), 3.67-3.64 (m, 2H), 2.15 (s, 3H), 2.10-2.04 (m, 2H), 1.96-1.61 (m, 2H); MS (m/z): 489.2 (M+1)⁺.

Compound 307: ¹H NMR (400 MHz, DMSO-d₆) δ 11.80 (s, 1H), 10.35 (s, 1H), 8.09 (s, 1H), 7.74-7.56 (m, 1H), 7.69-7.38 (m, 5H), 7.18 (s, 1H), 6.57 (d, J=2.9 Hz, 1H), 4.57-4.51 (m, 1H), 3.81-3.72 (m, 1H), 3.70-3.58 (m, 1H), 2.19 (s, 3H), 2.12-2.02 (m, 2H), 1.87-1.72 (m, 2H). MS (m/z): 489.2 (M+1)⁺.

The following Compound 308 were prepared according to the procedure of Compound 306 using the corresponding reagents and intermediates under appropriate conditions that will be recognized by one skilled in the art:

| Compd. | | LC/MS | |
|---|---|---|---|
| No. | Structure | (M + 1)+ | NMR |
| 308 | 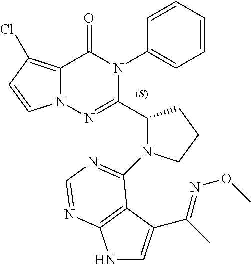 | 503.2 | ¹H NMR (400 MHz, DMSO-d₆) δ: 11.90 (s, 1H), 8.15 (s, 1H), 7.59-7.54 (m, 1H), 7.51-7.27 (m, 4H), 7.37 (d, J = 2.7 Hz, 1H), 7.27 (s, 1H), 6.56 (d, J = 2.7 Hz, 1H), 4.69-4.62 (m, 1H), 3.85 (s, 3H), 3.72-3.61 (m, 1H), 3.60-3.48 (m, 1H), 2.18 (s, 3H), 2.09-2.01 (m, 2H), 1.97-1.85 (m, 1H), 1.71-1.62 (m, 1H). |

### Example 37

### Compound 309

### (S)-4-(2-(5-chloro-4-oxo-3-(pyridin-2-yl)-3,4-dihydropyrrolo[2,1-f][1,2,4]triazin-2-yl)azetidin-1-yl)-7H-pyrrolo[2,3-d]pyrimidine-5-carbonitrile

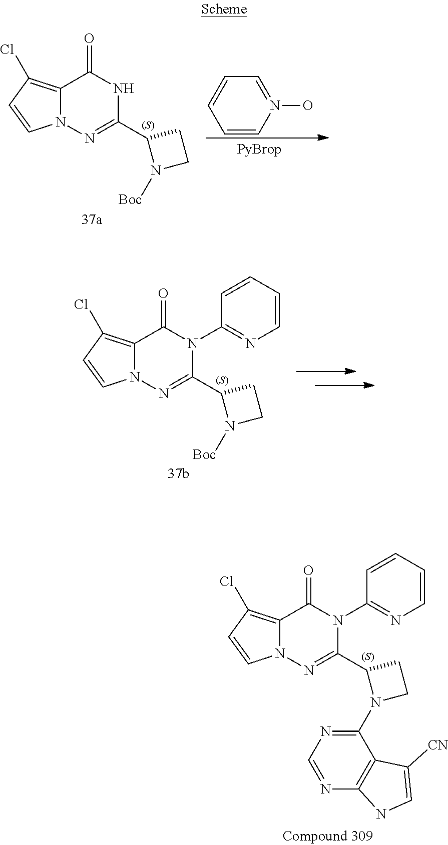

### Step 37-1 (S)-tert-butyl 2-(5-chloro-4-oxo-3-(pyridin-2-yl)-3,4-dihydropyrrolo[2,1-f][1,2,4]triazin-2-yl)azetidine-1-carboxylate (37b)

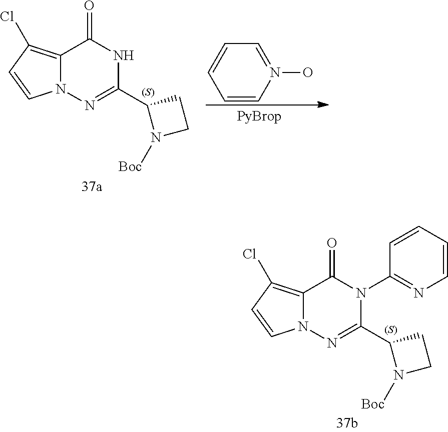

37a (prepared according to the procedures described in Example 1 using the corresponding reagents and intermediates) (407 mg, 1.25 mmol) was dissolved in DCM (3 mL), DIPEA (674 uL) was added, the mixture was stirred at r.t. for 2 min, Pyridine-N-oxide (95 mg, 1 mmol) was added, followed by PyBrOP (620 mg, 1.33 mmol), the reaction was stirred at r.t. overnight, then concentrated and purified by flash column chromatography to give product 37b as a white solid. Yield: 12%, Ms: 402.1 (M+1)⁺.

### Step 37-2 (S)-4-(2-(5-chloro-4-oxo-3-(pyridin-2-yl)-3,4-dihydropyrrolo[2,1-f][1,2,4]triazin-2-yl)azetidin-1-yl)-7H-pyrrolo[2,3-d]pyrimidine-5-carbonitrile (Compound 309)

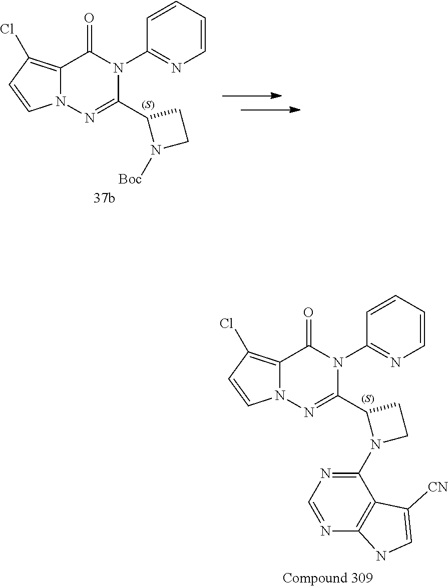

Compound 309 was prepared according to the procedures described in Example 1 from 37b. ¹H NMR (400 MHz, DMSO-d6) δ 8.69-8.68 (m, 1H), 8.28 (s, 1H), 8.27 (s, 1H), 8.09-8.06 (m, 1H), 7.73 (d, J=2.8 Hz, 1H), 7.71 (d, J=8.0 Hz, 1H), 7.63-7.59 (m, 1H), 6.69 (d, J=3.2 Hz, 1H), 5.18-5.14 (m, 1H), 4.41-4.36 (m, 1H), 4.19-4.13 (m, 1H), 2.67-2.61 (m, 1H), 2.12-2.06 (m, 1H). MS (m/z): 444.1 (M+1)⁺.

The following Compounds were prepared according to the procedure of Compound 309 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 312 | 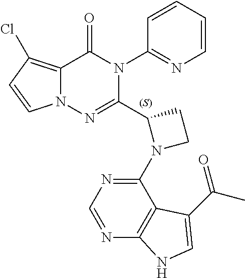 | 461.1 | ¹H NMR (400 MHz, DMSO-d₆) δ 8.68-8.67 (m, 1H), 8.16 (s, 1H), 8.11-8.06 (m, 2H), 7.72-7.70 (m, 2H), 7.61-7.58 (m, 1H), 6.69 (d, J = 3.2 Hz, 1H), 5.01 (br, 1H), 4.33 (br, 1H), 3.68-3.67 (m, 1H), 2.46 (br, 1H), 2.41 (s, 3H), 1.73 (br, 1H). |
| 379 | 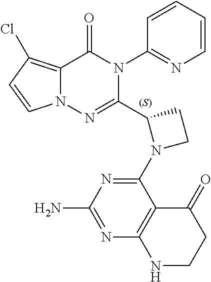 | 463.8 | ¹H NMR (400 MHz, CD₃OD) δ 8.65 (d, J = 4.2 Hz, 1H), 8.10-8.06 (m, 1H), 7.67 (d, J = 8.0 Hz, 1H), 7.61-7.58 (m, 1H), 7.44 (brs, 1H), 6.54 (d, J = 2.8 Hz, 1H), 4.62-4.42 (m, 1H), 3.65 (br, 1H), 3.43-3.35 (m, 3H), 2.55-1.97 (m, 4H). |
| 473 | 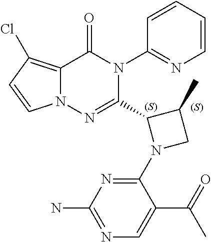 | 450.9 | ¹H NMR (400 MHz, CD3OD) δ 8.70 (d, J = 4.1, 1H), 8.114-8.09 (m, 1H), 7.75 (d, J = 7.8, 1H), 7.64 (dd, J = 7.5, 4.9, 1H), 7.37 (s, 1H), 6.54 (d, J = 2.9, 1H), 4.89 (br, 1H), 4.46-4.41 (m, 1H), 3.36 (br, 1H), 2.68 (br, 1H), 2.29 (s, 3H). |
| 474 | 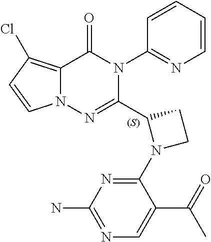 | 436.8 | ¹H NMR (400 MHz, CD3OD) δ 8.57 (dd, J = 4.9, 1.9, 1H), 8.33 (s, 1H), 8.00 (td, J = 7.8, 1.9, 1H), 7.60 (d, J = 7.9, 1H), 7.52 (dd, J = 7.5, 4.9, 1H), 7.32 (d, J = 2.8, 1H), 6.46 (d, J = 3.0, 1H), 4.94 (br, 1H), 4.26-4.19 (m, 1H), 3.63 (br, 1H), 2.38-2.29 (m, 1H), 2.20 (s, 3H), 2.14-2.07 (m, 1H). |
| 475 | 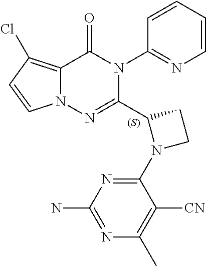 | 433.8 | 1H NMR (400 MHz, CD3OD) δ 8.66-8.63 (m, 1H), 8.08-8.03 (m, 1H), 7.63 (d, J = 8.0, 1H), 7.61- 7.57 (m, 1H), 7.48 (d, J = 3.0, 1H), 6.56 (d, J = 3.0, 1H), 5.00-4.95 (m, 1H), 4.33-4.26 (m, 1H), 4.17-4.10 (m, 1H), 2.56-2.49 (m, 1H), 2.37-2.30 (m, 1H), 2.29 (s, 3H). |
| 476 | 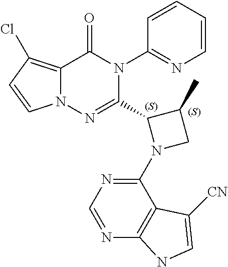 | 457.8 | 1H NMR (400 MHz, CD3OD) δ 8.61-8.58 (m, 1H), 8.07 (s, 1H), 7.94-7.90 (m, 1H), 7.80 (s, 1H), 7.66 (d, J = 7.8, 1H), 7.52-7.48 (m, 1H), 7.28 (d, J = 3.0, 1H), 6.43 (d, J = 3.0, 1H), 4.72- 4.70 (m, 1H), 4.59-4.54 (m, 1H), 3.74-3.70 (m, 1H), 2.92-2.85 (m, 1H), 0.77 (d, J = 7.2, 3H). |
| 477 | 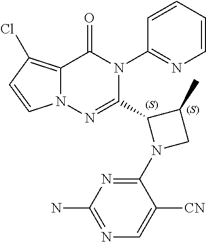 | 434.1 | ¹H NMR (400 MHz, CD3OD) δ 8.62 (dd, J = 4.9, 1.9, 1H), 8.07-7.95 (m, 2H), 7.62 (d, J = 7.9, 1H), 7.55 (dd, J = 7.5, 4.9, 1H), 7.38 (d, J = 2.9, 1H), 6.48 (dd, J = 3.0, 0.5, 1H), 4.60-4.44 (m, 1H), 4.39- 4.30 (m, 1H), 3.75-3.53 (m, 1H), 2.86-2.65 (m, 1H), 0.81 (d, J = 6.9, 3H). |
| 478 | 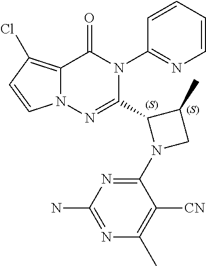 | 448.1 | ¹H NMR (400 MHz, CD3OD) δ 8.61 (dd, J = 4.9, 1.2, 1H), 7.99 (td, J = 7.7, 1.9, 1H), 7.62 (d, J = 7.9, 1H), 7.54 (ddd, J = 7.5, 4.9, 0.9, 1H), 7.37 (d, J = 3.0, 1H), 6.48 (d, J = 3.2, 1H), 4.57-4.47 (m, 1H), 4.42-4.25 (m, 1H), 3.79-3.52 (m, 1H), 2.80-2.66 (m, 1H), 2.21 (s, 3H), 0.80 (d, J = 6.9, 3H). |
| 479 | 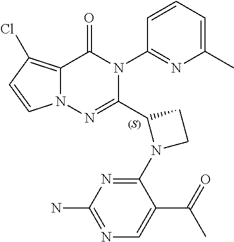 | 452.1 | 1H NMR (400 MHz, CD3OD) δ 8.40-838 (m, 1H), 8.32 (s, 1H), 7.75-7.69 (m, 1H), 7.35 (d, J = 8.0, 1H), 7.24-7.20 (m, 2H), 6.42 (d, J = 2.8, 1H), 5.59 (br, 1H), 5.42-5.29 (m, 2H), 4.30-4.23 (m, 1H), 3.68 (br, 1H), 2.27-2.09 (m, 6H). |

### Example 38

### Compound 314

### (S)-2-(1-(2-amino-8-chloropyrazolo[1,5-a][1,3,5]triazin-4-yl)azetidin-2-yl)-5-chloro-3-phenylpyrrolo[2,1-f][1,2,4]triazin-4(3H)-one

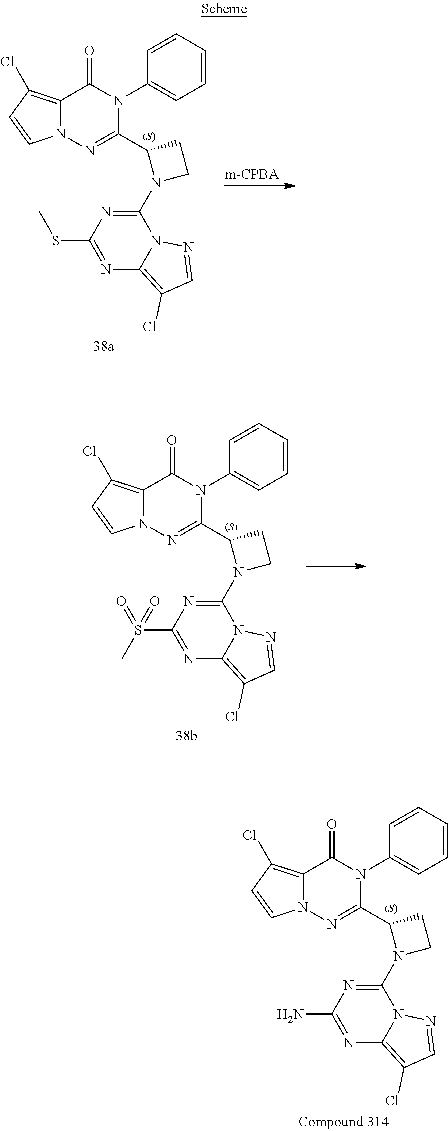

### Step 38-1 (S)-5-chloro-2-(1-(8-chloro-2-(methylsulfonyl)pyrazolo[1,5-a][1,3,5]triazin-4-yl)azetidin-2-yl)-3-phenylpyrrolo[2,1-f][1,2,4]triazin-4(3H)-one (38b)

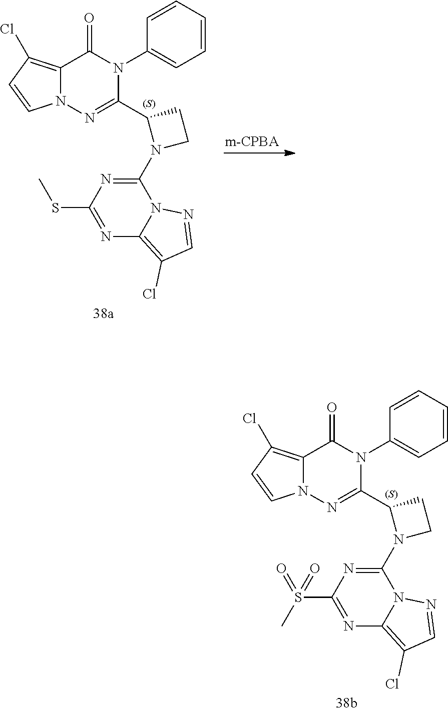

38a (prepared according to the procedure of Example 1 using the corresponding reagents and intermediates) (40 mg, 0.08 mmol) and m-CPBA (37 mg, 75%, 0.16 mmol) were dissolved in DCM (3 mL), the reaction was stirred at r.t. overnight. The mixture was used for the next step without purification. MS (m/z): 531.0 (M+1)⁺.

### Step 38-2 (S)-2-(1-(2-amino-8-chloropyrazolo[1,5-a][1,3,5]triazin-4-yl)azetidin-2-yl)-5-chloro-3-phenylpyrrolo[2,1-f][1,2,4]triazin-4(3H)-one (Compound 314)

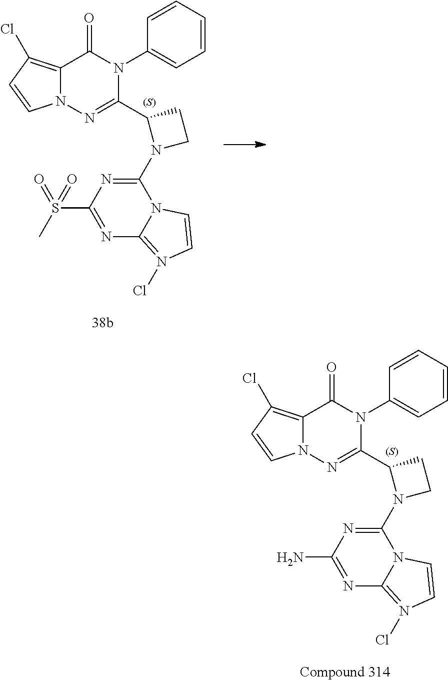

To the mixture above was added NH₃/THF (0.4 N, 3 mL), the reaction was stirred at r.t. for 2 h, then concentrated and purified by TLC to give Compound 314 as a white solid. Yield: 10.8%. ¹H NMR (400 MHz, DMSO-d6) δ 7.88-7.14 (m, 1H), 7.57-7.52 (m, 5H), 7.39 (br, 1H), 6.83-6.59 (m, 3H), 5.34 (br, 0.5H), 4.88 (br, 0.5H), 4.45 (br, 0.5H), 4.17 (br, 0.5H), 4.03 (br, 0.5H), 2.64-2.52 (m, 2H), 2.33 (br, 0.5H). MS (m/z): 468.0 (M+1)⁺.

The following Compounds were prepared according to the procedure of Compound 314 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 316 | 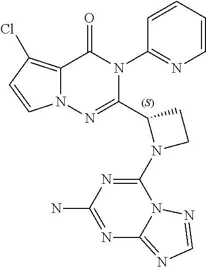 | 435.5 | ¹H NMR (400 MHz, DMSO-d₆) δ 8.00 (s, 1H), 7.76-7.42 (m, 6H), 7.05 (br, 2H), 6.61 (br, 1H), 5.34 (br, 0.5H), 4.90 (br, 0.5H), 4.44 (br, 1H), 4.15 (br, 1.H), 2.65-2.53 (m, 2H). |
| 317 | 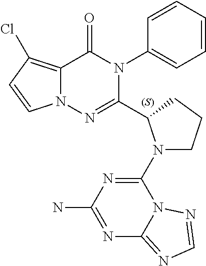 | 449.5 | ¹H NMR (400 MHz, CD₃OD) δ 7.98 (s, 0.7H), 7.87 (s, 0.3H), 7.85-7.70 (m, 3H), 7.58-7.43 (m, 3H), 7.40 (d, J = 7.3 Hz, 1H), 7.32 (d, J = 8.9 Hz, 1H), 7.27-7.20 (m, 1H), 6.48-6.33 (m, 1H), 5.67-5.49 (m, 1H), 4.01-3.88 (m, 1H), 3.80-3.65 (m, 1H), 2.25-2.16 (m, 1H), 2.00-1.91 (m, 2H), 1.88-1.80 (m, 1H). |
| 320 | 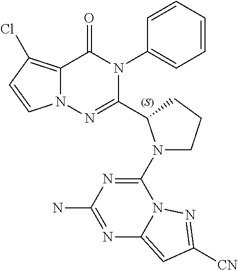 | 473.1 | ¹H NMR (400 MHz, DMSO-d₆) δ 7.94 (d, J = 8.0 Hz, 0.5H), 7.85 (d, J = 8.0 Hz, 0.5H), 7.68-7.54 (m, 4H), 6.93 (s, 1H), 6.78 (s, 0.5H), 6.63-6.61 (m, 1H), 6.39 (d, J = 4.0 Hz, 0.5H), 5.64 (d, J = 4.0 Hz, 0.5H), 4.72 (d, J = 8.0 Hz, 0.5H), 4.54-4.42 (m, 0.5H), 4.35-4.18 (m, 0.5H), 3.96-3.88 (m, 0.5H), 3.75-3.67 (m, 0.5H), 2.37-2.28 (m, 1H), 2.21-2.11 (m, 1H), 2.04-1.88 (m, 2H). |
| 321 | 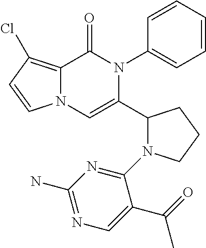 [SMILES: CC(=O)c1cnc(N)nc1N1CCCC1c1cn2ccc(Cl)c2c(=O)n1-c1ccccc1] | 448.9 | ¹H NMR (400 MHz, DMSO-d₆) δ 7.69 (s, 1H), 7.15-7.10 (m, 1H), 6.94-6.91 (m, 1H), 6.84-6.57 (m, 5H), 6.40-6.37 (m, 2H), 5.71 (d, J = 2.9, 1H), 4.18 (t, J = 7.6, 1H), 2.85-2.79 (m, 1H), 2.09-2.00 (m, 1H), 1.70 (s, 3H), 1.13-1.08 (m, 1H), 1.00- 0.94 (m, 2H), 0.81-0.701 (m, 1H). |
| 322 | 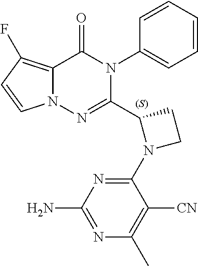 | 417.1 | ¹H NMR (400 MHz, DMSO-d₆) δ 7.68-7.63 (m, 1H), 7.62-7.50 (m, 4H), 7.45-7.39 (m, 1H), 7.15 (br, 2H), 6.51 (d, J = 3.2 Hz, 1H), 5.09-4.72 (m, 1H), 4.25-3.91 (m, 2H), 2.22 (s, 3H), 2.12-1.95 (m, 2H). |
| 323 | 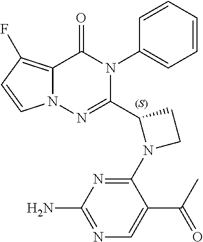 | 420.1 | ¹H NMR (400 MHz, CD₃OD) δ 7.67-7.47 (m, 5H), 7.33-7.28 (m, 1H), 7.25 (s, 1H), 6.32 (d, J = 3.1 Hz, 1H), 5.20 (br, 1H), 4.27 (sbr, 1H), 3.73 (br, 1H), 2.38-2.31 (m, 1H), 2.27 (s, 3H), 2.15-2.04 (m, 1H). |
| 324 | 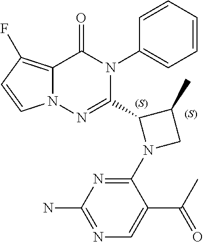 | 434.2 | ¹H NMR (400 MHz, CDCl₃) δ 8.32 (s, 1H), 7.68-7.59 (m, 2H), 7.57-7.46 (m, 2H), 7.20-7.15 (m, 1H), 6.99 (br, 1H), 6.20 (d, J = 3.1 Hz, 1H), 5.22-5.13 (m, 1H), 4.47-4.30 (m, 1H), 3.52-3.28 (m, 1H), 2.48-2.32 (m, 1H), 2.24 (s, 3H), 0.70 (d, J = 6.9 Hz, 3H). |
| 325 | 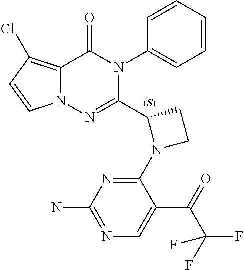 | 489.8 | ¹H NMR (400 MHz, CD₃OD) δ 8.41 (br, 1H), 7.30-7.64 (m, 7H), 6.51 (s, 1H), 5.33 (br, 1H), 4.35-3.81 (m, 2H), 2.39 (br, 1H), 2.20-2.16 (m, 1H). |
| 326 | 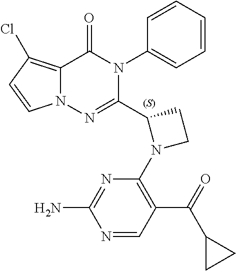 | 462..2 | ¹H NMR (400 MHz, DMSO-d₆) δ 8.51 (s, 1H), 7.60-7.38 (m, 5H), 7.39-7.38 (m, 1H), 6.74 (s, 2H), 6.61 (d, J = 2.9 Hz, 1H), 4.89 (brs, 1H), 4.06-4.00 (m, 1H), 2.44-2.35 (m, 2H), 1.90 br (s, 1H), 0.85-0.78 (m, 4H). |
| 288 | 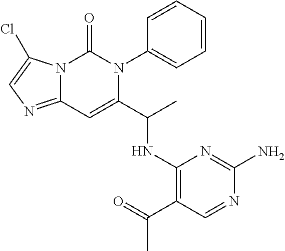 [SMILES: CC(=O)c1cnc(N)nc1NC(C)c1cc2ncc(Cl)n2c(=O)n1-c1ccccc1] | 424.1 | ¹H NMR (400 MHz, CD₃OD) δ 8.52 (s, 1H), 7.58-7.54 (m, 1H), 7.521-7.46 (m, 2H), 7.45-7.39 (m, 2H), 7.27 (s, 1H), 6.62 (s, 1H), 2.44 (s, 3H), 1.38 (d, J = 6.8 Hz, 3H). |
| 362 | 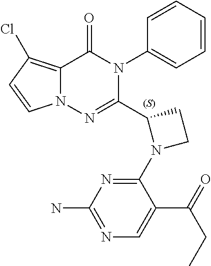 | 449.9 | ¹H NMR (400 MHz, CD₃OD) δ 8.36 (s, 1H), 7.68-7.54 (m, 4H), 7.34-7.29 (m, 2H), 6.53-6.52 (m, 1H), 5.26 (br, 1H), 4.28-4.22 (m, 1H), 3.80 (br, 1H), 2.76- 2.70 (m, 1H), 2.54 (br, 1H), 2.39-2.31 (m, 1H), 2.20-2.10 (m, 1H), 0.89 (br, 3H). |
| 435 | 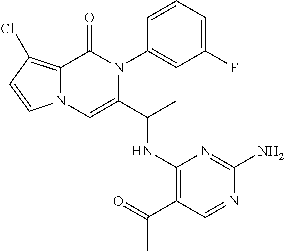 [SMILES: CC(=O)c1cnc(N)nc1NC(C)c1cn2ccc(Cl)c2c(=O)n1-c1cccc(F)c1] | 441.1 | ¹H NMR (400 MHz, CD₃OD) δ 8.46 (s, 1H), 7.49 (s, 1H), 7.46-7.43 (m, 1H), 7.37-7.35 (m, 1H), 7.24-7.14 (m, 1H), 7.06-6.97 (m, 1H), 6.88-6.85 (m, 1H), 6.59-6.57 (m, 1H), 5.06-5.01 (m, 1H), 2.41 (s, 3H), 1.42-1.40 (m, 3H). |
| 436 | 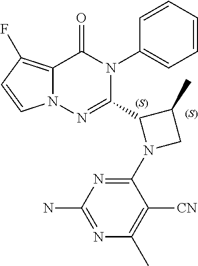 | 431.2 | ¹H NMR (400 MHz, CD₃OD) δ 7.62- 7.56 (m, 4H), 7.37-7.34 (m, 1H), 7.297 (br, 1H), 6.33 (d, J = 3.1, 1H), 4.83-4.81 (m, 1H), 4.40 (br, 1H), 3.64 (br, 1H), 2.65 (br, 1H), 2.29 (s, 3H), 0.70 (d, J = 6.7, 3H). |
| 437 | 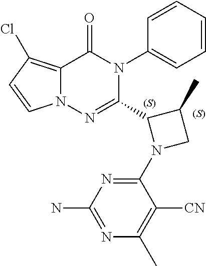 | 447.2 | ¹H NMR (400 MHz, CDCl₃) δ 7.53-7.44 (m, 4H), 7.16 (br, 1H), 7.13-7.10 (m, 1H), 6.41 (d, J = 2.9, 1H), 5.09 (s, 2H), 4.75 (br, 1H), 4.38 (br, 1H), 3.60 (br, 1H), 2.47 (br, 1H), 2.31 (s, 3H), 0.70 (d, J = 6.3, 3H). |
| 438 | 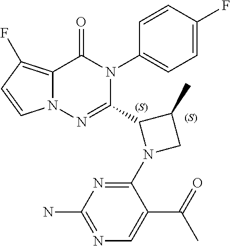 | 451.9 | ¹H NMR (400 MHz, CD₃OD) δ 8.41 (s, 1H), 7.71_(br, 1H), 7.40-7.28 (m, 3H), 7.18 (br, 1H), 6.30 (d, J = 2.1, 1H), 4.91 (br, 1H), 4.41-4.36 (m, 1H), 3.36 (br, 1H), 2.55 (br, 1H), 2.25 (s, 3H), 0.75 (d, J = 6.8, 3H). |
| 439 | 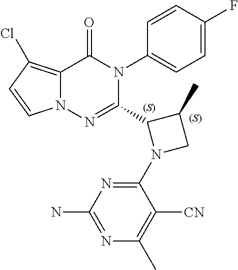 | 464.8 | ¹H NMR (400 MHz, CD₃OD) δ 7.69-7.63 (m, 1H), 7.43-7.29 (m, 4H), 6.54 (d, J = 3.0, 1H), 4.87 (br, 1H), 4.40 (br, 1H), 3.67 (br, 1H), 2.69 (br, 1H), 2.29 (s, 3H), 0.80 (d, J = 6.8, 3H). |
| 440 | 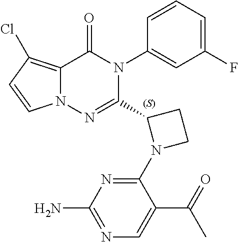 | 454.5 | ¹H NMR (400 MHz, DMSO-d₆) δ 8.42 (s, 1H), 7.66-7.49 (m, 3H), 7.40-7.36 (m, 1H), 7.26 (d, J = 7.6 Hz, 1H), 6.83 (d, J = 7.6 Hz, 2H), 6.62 (d, J = 2.8 Hz, 1H), 4.97-4.67 (m, 1H), 4.16-4.09 (m, 1H), 3.45-3.40 (m, 1H), 2.43-2.35 (m, 1H), 2.24 (s, 3H), 2.00-1.88 (m, 1H). |
| 441 | 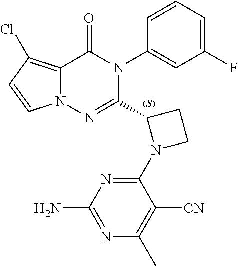 | 451.2 | ¹H NMR (400 MHz, CDCl₃) δ 7.58-7.45 (m, 1H), 7.41-7.31 (m, 1H), 7.29-7.21 (m, 2H), 6.95-6.88 (m, 1H), 6.51 (d, J = 3.2 Hz, 1H), 5.12 (s, 2H), 4.47-4.31 (m, 1H), 4.20-4.07 (m, 1H), 2.38 (s, 3H), 2.35-2.31 (m, 1H), 1.79-1.42 (m, 2H). |
| 442 | 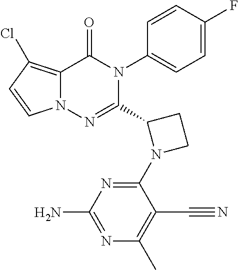 [SMILES: Cc1nc(N)nc(N2CC[C@H]2c2nn3ccc(Cl)c3c(=O)n2-c2ccc(F)cc2)c1C#N] | 451.3 | ¹H NMR (400 MHz, CDCl₃) δ 7.59-7.52 (m, 1H), 7.28-7.09 (m, 4H), 6.50-6.49 (m, 1H), 5.14 (br, 2H), 4.48-4.32 (m, 1H), 421-4.07 (m, 1H), 2.37 (s, 3H), 2.34-2.31 (m, 1H), 1.60-1.49 (m, 2H). |
| 443 | 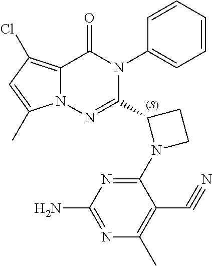 | 447.2 | ¹H NMR (400 MHz, CD₃OD) δ 7.60-7.52 (m, 4H), 7.33-7.29 (m, 1H), 6.94 (s, 1H), 5.24-5.17 (m, 1H), 4.35-4.26 (m, 1H), 4.09-4.01 (m, 1H), 2.45-2.38 (m, 1H), 2.35-2.30 (m, 1H), 2.28 (s, 3H), 2.24 (s, 3H). |
| 444* | 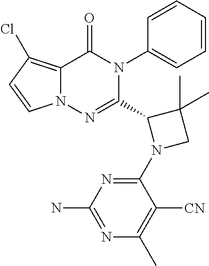 [SMILES: Cc1nc(N)nc(N2CC(C)(C)[C@H]2c2nn3ccc(Cl)c3c(=O)n2-c2ccccc2)c1C#N] | 460.9 | ¹H NMR (400 MHz, CD₃OD) δ 7.67 (br, 1H), 7.53-7.38 (m, 4H), 7.29 (d, J = 6.8, 1H), 6.48 (d, J = 3.0, 1H), 4.72 (br, 1H), 4.66 (br, 1H), 3.74 (br, 1H), 2.23 (s, 3H), 1.15 (s, 3H), 0.67 (s, 3H). |
| 480 | 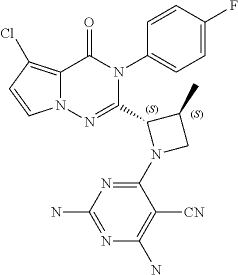 | 466.1 | ¹H NMR (400 MHz, DMSO-d₆) δ 7.71 (d, J = 2.8, 1H), 7.66 (br, 1H), 7.56-7.51 (m, 1H), 7.44-7.39 (m, 2H), 6.64 (d, J = 3.0, 1H), 6.49 (s, 2H), 6.36 (s, 2H), 4.50 (br, 1H), 4.21 (br, 1H), 3.53 (br, 1H), 2.74- 2.69 (m, 1H), 0.70 (d, J = 6.4, 3H). |
| 481 | 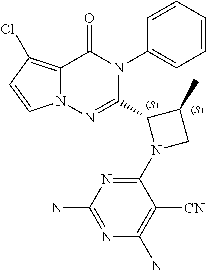 | 448.1 | ¹H NMR (400 MHz, DMSO-d₆) δ 7.69 (d, J = 2.9, 1H), 7.57-7.52 (m, 4H), 7.47- 7.44 (m, 1H), 6.63 (d, J = 3.0, 1H), 6.47 (s, 2H), 6.34 (s, 2H), 4.51 (br, 1H), 4.21 (br, 1H), 3.51 (br, 1H), 2.69 (br, 1H), 0.61 (d, J = 6.4, 3H). |
| 482 | 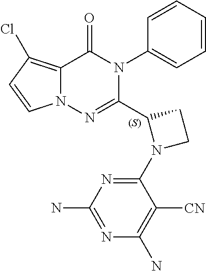 | 434 | ¹H NMR (400 MHz, DMSO-d₆) δ 7.73 (d, J = 3.0, 1H), 7.58-7.50 (m, 4H), 7.39- 7.36 (m, 1H), 6.64 (d, J = 3.0, 1H), 6.48 (s, 2H), 6.35 (s, 2H), 4.83 (br, 1H), 4.01 (br, 1H), 3.96-3.89 (m, 1H), 3.47-3.38 (m, 1H), 2.00 (br, 1H). |
| 502 | 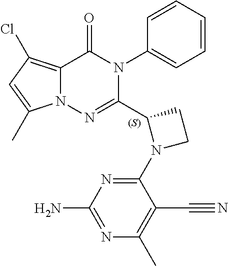 | 447.2 | ¹H NMR (400 MHz, CD₃OD) δ 7.60- 7.52 (m, 4H), 7.33-7.29 (m, 1H), 6.94 (s, 1H), 5.24-5.17 (m, 1H), 4.35-4.26 (m, 1H), 4.09-4.01 (m, 1H), 2.45-2.30 (m, 2H), 2.28 (s, 3H), 2.24 (s, 3H). |
| 503 | 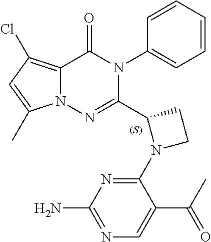 | 450.1 | ¹H NMR (400 MHz, CD₃OD) δ 8.40 (s, 1H), 7.66-7.61 (m, 1H), 7.58-7.52 (m, 3H), 7.28-7.26 (m, 1H), 6.90 (s, 1H), 5.39-5.29 (m, 1H), 4.34-4.27 (m, 1H), 3.89-3.78 (m, 1H), 2.32-.2.19 (m, 2H), 2.17 (s, 3H), 2.14 (s, 3H). |
| 504 | 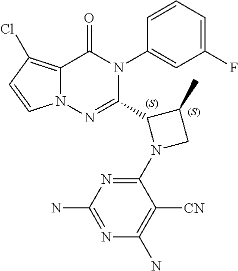 | 466.1 | ¹H NMR (400 MHz, DMSO-d₆) δ 7.87- 7.33 (m, 5H), 6.65 (d, J = 3.2, 1H), 6.49 (s, 2H), 6.38 (s, 1H), 6.36 (s, 1H), 4.48 (br, 1H), 4.22 (br, 1H), 3.54 (br, 1H), 2.75 (br, 1H), 0.75-0.69 (m, 3H). |

*prepared from (S)-methyl 3,3-dimethylazetidine-2-carboxylate.

### Example 39

### Compound 329

### (S)-2-(1-(2-aminopyrrolo[2,1-f][1,2,4]triazin-4-yl)azetidin-2-yl)-5-chloro-3-phenyl-pyrrolo[2,1-f][1,2,4]triazin-4(3H)-one

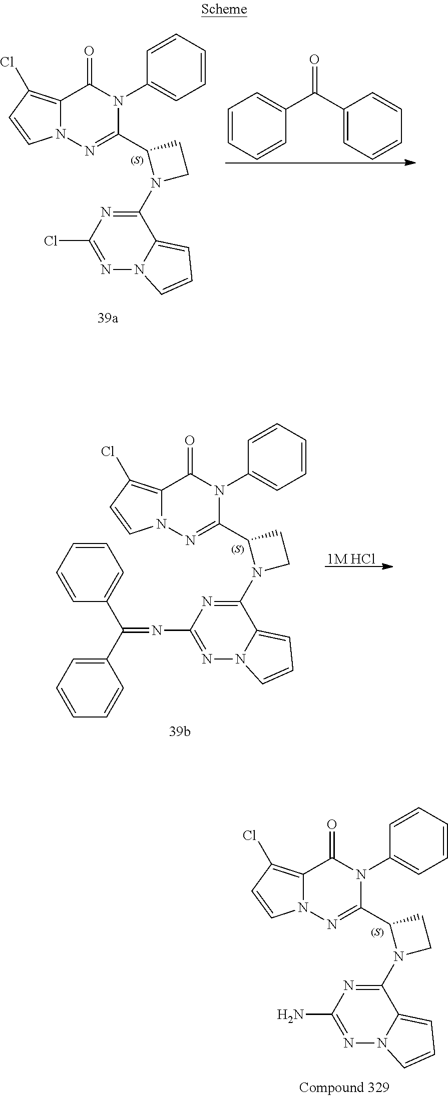

To a mixture of 39a (prepared according to the procedure of Example 1 using the corresponding reagents and intermediates) (23 mg, 0.051 mmol) in dioxane (4 mL) were added diphenylmethanimine (18 mg, 0.102 mmol), Pd(OAc)₂ (2.2 mg, 0.001 mmol), 2,2′-bis(diphenylphosphino)-1,1′-binaphthyl (6.2 mg, 0.001 mmol) and Cs₂CO₃ (41 mg, 0.128 mmol) at r.t., the reaction was stirred at 110° C. overnight under N₂.

After cooling to the r.t., 1M HCl (1 mL) was added to the mixture, the reaction was stirred at r.t. for 20 min, then concentrated, the resulting residue was dissolved in MeOH, and adjusted to PH˜7 with DIEA, the mixture was concentrated and purified by flash column chromatography to give Compound 329 as a yellow solid. Yield: 36%. 1H NMR (400 MHz, CDCl3) δ 7.63-7.56 (m, 1H), 7.55-7.44 (m, 3H), 7.30-7.27 (m, 1H), 7.28 (d, J=3.0 Hz, 1H), 7.18-7.13 (m, 1H), 6.48 (d, J=2.9 Hz, 1H), 6.44 (dd, J=4.4, 2.4 Hz, 1H), 6.37 (s, 1H), 5.11 (dd, J=8.5, 5.9 Hz, 1H), 4.55-4.36 (m, 1H), 4.34-4.24 (m, 1H), 4.19 (s, 2H), 2.59-2.45 (m, 1H), 2.44-2.30 (m, 1H). MS (m/z): 433.1 (M+1)⁺.

The following Compounds were prepared according to the procedure of Compound 329 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 330 | 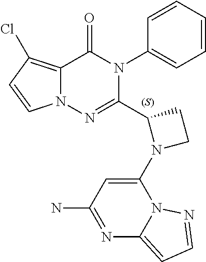 | 433.1 | ¹H NMR (400 MHz, CD₃OD) δ 7.69 (s, 1H), 7.65-7.59 (m, 1H), 7.58-7.50 (m, 4H), 7.29-7.28 (m, 1H), 7.27 (d, J = 3.0 Hz, 1H), 6.46 (d, J = 3.0 Hz, 1H), 5.82 (s, 1H), 5.38 (dd, J = 8.6, 5.1 Hz, 1H), 5.10 (s, 1H), 4.41-4.27 (m, 1H), 4.14-3.98 (m, 1H), 2.58-2.37 (m, 2H). |
| 331 | 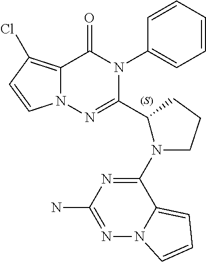 | 447.5 | ¹H NMR (400 MHz, CD₃OD) δ 7.54 (d, J = 8.2 Hz, 1H), 7.40 (t, J = 7.3 Hz, 1H), 7.37-7.27 (m, 2H), 7.20 (d, J = 7.3 Hz, 1H), 7.07 (br, 1H), 7.00 (br, 1H), 6.53 (br, 1H), 6.23 (br, 1H), 6.20 (br, 1H), 4.01-3.91 (m, 1H), 3.78-3.67 (m, 1H), 2.15-2.05 (m, 1H), 1.95-1.77 (m, 2H), 1.71-1.58 (m, 1H). |
| 332 | 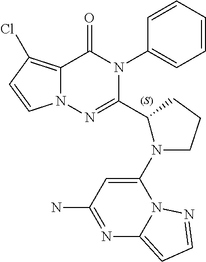 | 447.5 | ¹H NMR (400 MHz, CDCl₃) δ 7.66 (d, J = 7.9 Hz, 1H), 7.55 (br, 1H), 7.44-7.36 (m, 1H), 7.35-7.27 (m, 2H), 6.87-6.80 (m, 1H), 6.18 (d, J = 2.8 Hz, 1H), 5.83 (br, 1H), 5.75 (br, 1H), 5.62 (br, 2H), 4.96 (br, 1H), 4.38-4.18 (m, 1H), 3.64-3.40 (m, 1H), 3.37-3.21 (m, 1H), 2.04-1.67 (m, 4H). |
| 333 | 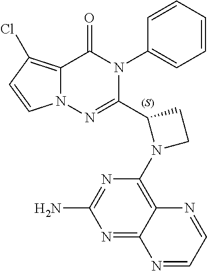 | 446.4 | ¹H NMR (400 MHz, CD₃OD) δ 8.59-8.52 (m, 1H), 8.31 (d, J = 2.0 Hz, 1H), 7.65-7.63 (m, 2H), 7.60-7.58 (m, 2H), 7.36-7.33 (m, 1H), 7.27 (d, J = 2.9 Hz, 1H), 6.46 (d, J = 3.2 Hz, 1H), 5.57-5.54 (m, 1H), 4.40-4.38 (m, 1H), 4.23-4.20 (m, 1H), 2.60-2.54 (m, 2H). |
| 334 | 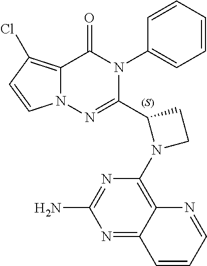 | 445.5 | ¹H NMR (400 MHz, CD₃OD) δ 8.36 (s, 1H), 7.63-7.55 (m, 5H), 7.42-7.26 (m, 3H), 6.45 (brs, 1H), 5.59 (brs, 1H), 4.38 (brs, 1H), 4.19 (brs, 1H), 2.57 (brs, 2H). |
| 335 | 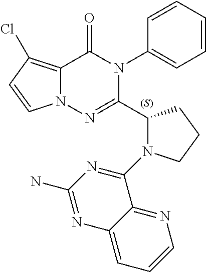 | 459.2 | ¹H NMR (400 MHz, CDCl₃) δ 8.44 (s, 1H), 7.86 (d, J = 7.9 Hz, 0.6H), 7.72-7.47 (m, 4.4H), 7.45-7.30 (m, 3H), 7.24-6.98 (m, 2H), 6.49-6.31 (m, 1H), 5.97 (d, J = 6.8 Hz, 0.6H), 4.98 (s, 0.4H), 4.77-4.63 (m, 0.4H), 4.55-4.40 (m, 0.4H), 4.12-3.97 (m, 0.6H), 3.95-3.80 (m, 0.6H), 2.14-1.84 (m, 4H). |

### Example 41

### Compound 337

### (S)-4-(2-(5-chloro-4-oxo-3-(pyridin-2-yl)-3,4-dihydropyrrolo[2,1-f][1,2,4]triazin-2-yl)pyrrolidin-1-yl)-7H-pyrrolo[2,3-d]pyrimidine-5-carbonitrile

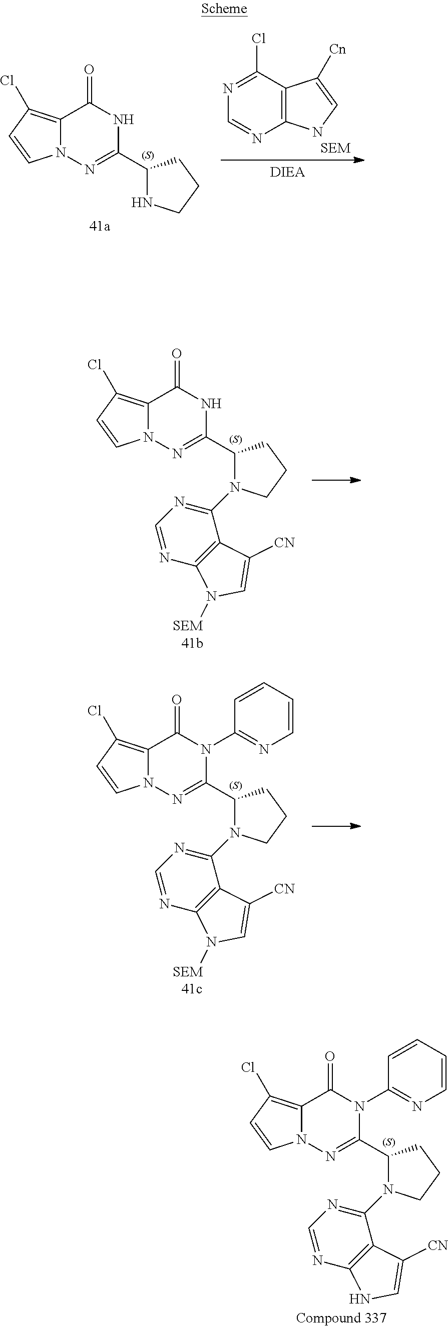

### Step 41-1 (S)-4-(2-(5-chloro-4-oxo-3,4-dihydropyrrolo[2,1-f][1,2,4]triazin-2-yl) pyrrolidin-1-yl)-7-((2-(trimethylsilyl)ethoxy)methyl)-7H-pyrrolo[2,3-d]pyrimidine-5-carbonitrile (41b)

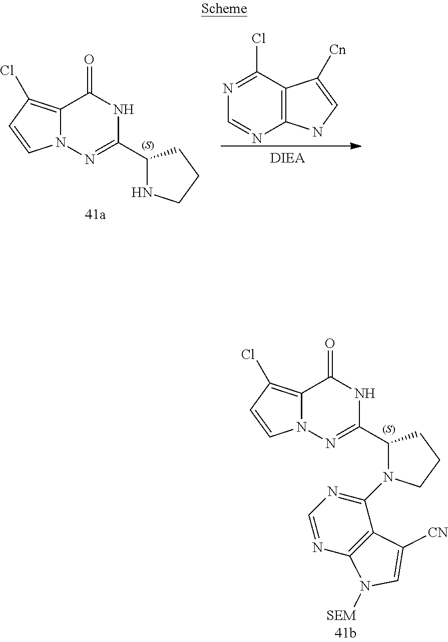

To a solution of 41a (prepared according to the procedures described in Example 1 using the corresponding reagents and intermediates) (155 mg, 0.65 mmol) in CH₃CN (15 mL) were added DIEA (0.32 mL, 1.95 mmol) and 4-chloro-7-((2-(trimethylsilyl)ethoxy)methyl)-7H-pyrrolo[2,3-d]pyrimidine-5-carbonitrile (201 mg, 0.65 mmol), the reaction was stirred at 90° C. overnight. The mixture was concentrated and purified by flash column chromatography to give 41b as a yellow solid. Yield: 45%. MS (m/z): 511.2 (M+1)⁺.

### Step 41-2 (S)-4-(2-(5-chloro-4-oxo-3-(pyridin-2-yl)-3,4-dihydropyrrolo[2,1-f][1,2,4]triazin-2-yl)pyrrolidin-1-yl)-7-((2-(trimethylsilyl)ethoxy)methyl)-7H-pyrrolo[2,3-d]pyrimidine-5-carbonitrile (41c)

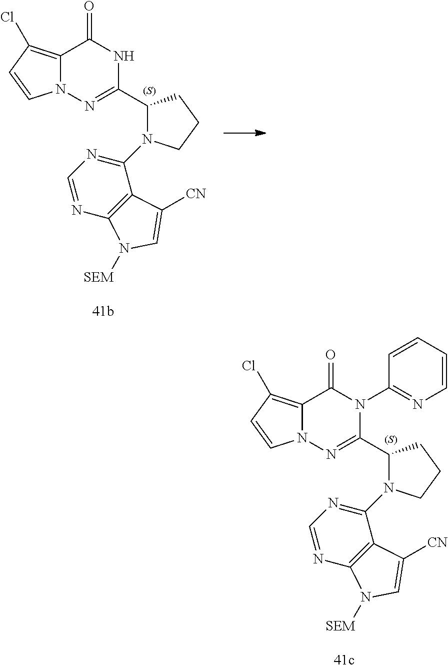

To a solution of 41b (150 mg, 0.29 mmol) in CH₂Cl₂ (3 mL) was added DIEA (0.15 mL, 0.87 mmol), the reaction was stirred at r.t. for 3 min, then treated with the stock solution of 1M Pyridine-N-oxide in CH₂Cl₂ (0.232 mL, 0.232 mmol) followed by PyBrOP (135 mg, 0.29 mmol). The reaction was capped and stirred at r.t. overnight. The mixture was concentrated and purified by flash column chromatography to give 41c as a yellow solid. Yield: 17%. MS (m/z): 588.3 (M+1)⁺.

### Step 41-3 (S)-4-(2-(5-chloro-4-oxo-3-(pyridin-2-yl)-3,4-dihydropyrrolo[2,1-f][1,2,4]triazin-2-yl)pyrrolidin-1-yl)-7H-pyrrolo[2,3-d]pyrimidine-5-carbonitrile (Compound 337)

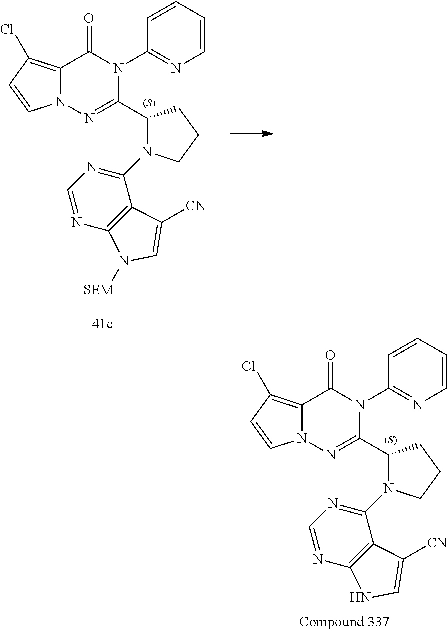

The solution of 41c dissolved in CF₃CO₂H (2 mL) was stirred at r.t. for 1 h, then concentrated, the resulting residue was dissolved in MeOH (3 mL), and treated with NH₃.H₂O (1 mL). The mixture was stirred at r.t. for another 1 h, then concentrated and purified by p-TLC to give Compound 337 as a white solid. Yield: 51%. ¹H NMR (400 MHz, DMSO-d6) δ 8.68 (dd, J=4.8, 1.4 Hz, 1H), 8.24 (s, 2H), 8.21 (s, 0.4H), 8.147 (dd, J=4.6, 1.7 Hz, 0.4H), 8.09-8.06 (m, 1H), 8.04 (d, J=2.9 Hz, 0.3H), 8.00 (s, 0.3H), 7.82 (brs, 1H), 7.73-7.69 (m, 0.4H), 7.60-7.57 (m, 2H), 7.28-7.25 (dd, J=4.8, 1.6 Hz, 0.4H), 7.09 (d, J=8.2 Hz, 0.4H), 6.97 (d, J=2.9 Hz, 0.4H), 6.60 (d, J=3.0 Hz, 1H), 5.30-5.26 (m, 1H), 4.49 (s, 1H), 4.02-3.97 (m, 1.4H), 3.94-3.86 (m, 1.4H), 2.30-2.27 (m, 1H), 2.26-2.18 (m, 2H), 2.13-2.06 (m, 1.5H), 2.03-1.95 (m, 3H). MS (m/z): 458.1 (M+1)⁺.

The following Compounds were prepared according to the procedure of Compound 337 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 311 | 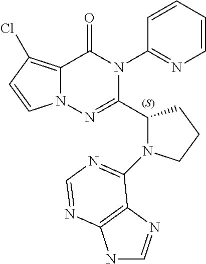 | 434.2 | ¹H NMR (400 MHz, DMSO-d₆) δ 12.94 (s, 1H), 8.67 (s, 1H), 8.20-8.08 (m, 3.5H), 7.82-7.80 (m, 0.5H), 7.60-7.49 (m, 2H), 6.58-6.55 (m, 1H), 5.06-5.05 (m, 0.5H), 4.50 (br, 0.5H), 4.20 (br, 0.5H), 4.11-4.07 (m, 0.5H), 3.90-3.85 (m, 0.5H), 3.64-3.62 (m, 0.5H), 2.18-2.16 (m, 1H), 1.99-1.88 (m, 3H). |
| 313 | 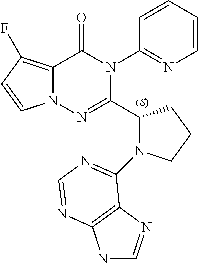 | 418.1 | ¹H NMR (400 MHz, DMSO-d₆) δ 8.08 (d, J = 4.8 Hz, 1H), 8.13-7.99 (m, 4H), 7.62-7.59 (m, 1H), 7.43 (br, 1H), 6.42 (d, J = 3.2 Hz, 1H), 5.07 (br, 1H), 4.07 (br, 2H), 2.33 (br, 1H), 2.12 (br, 1H), 1.99-1.94 (m, 2H). |
| 339 | 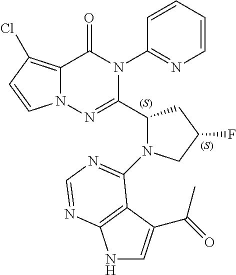 | 493.1 | ¹H NMR (400 MHz, DMSO-d₆) δ 8.64 (d, J = 4.8 Hz, 1H), 8.08-7.99 (m, 3H), 7.73 (d, J = 8.0 Hz, 1H), 7.55-7.50 (m, 2H), 6.59 (dd, J = 3.0, 1.0 Hz, 1H), 5.23-5.09 (m, 1H), 4.72 (brs, 1H), 4.21-4.13 (m, 1H), 3.92-3.82 (m, 1H), 2.41 (s, 3H), 2.38-2.28 (m, 2H). |
| 340 | 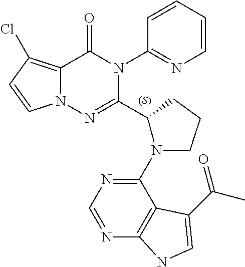 | 475.1 | 1H NMR (400 MHz, DMSO-d6) δ 8.68 (dd, J = 4.9, 1.2 Hz, 1H), 8.12 (s, 1H), 8.09 (td, J = 7.7, 1.9 Hz, 1H), 8.04 (s, 1H), 7.85 (d, J = 7.9 Hz, 1H), 7.62-7.53 (m, 2H), 6.64 (d, J = 3.0 Hz, 1H), 4.45-4.26 (m, 1H), 3.94-3.81 (m, 1H), 3.70-3.61 (m, 1H), 2.48 (s, 3H), 2.17-2.06 (m, 2H), 2.02-1.96 (m, 1H), 1.68-1.55 (m, 1H). |
| 342 | 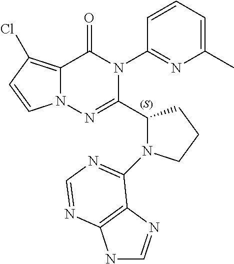 | 448.1 | ¹H NMR (400 MHz, DMSO-d₆) δ 8.50 (d, J = 4.8 Hz, 1H), 7.85-7.81 (m, 1H), 7.56 (d, J = 8.4 Hz, 1H), 7.42 (d, J = 2.4 Hz, 1H), 7.31 (dd, J = 6.4, 2.4 Hz, 1H), 6.52 (d, J = 6.8 Hz, 1H), 5.67- 5.53 (m, 2H), 4.00 (br, 2H), 1.98- 1.95 (m, 4H). |
| 344 | 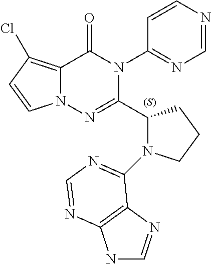 | 435.1 | ¹H NMR (400 MHz, CD₃OD) δ 9.32-9.32 (m, 1H), 9.04 (d, J = 5.2 Hz, 1H), 8.04 (br, 2H), 7.82 (s, 1H), 7.32 (d, J = 3.2 Hz, 1H), 6.47 (d, J = 2.8 Hz, 1H), 4.17 (br, 1H), 4.02 (br, 1H), 2.42 (br, 1H), 2.32-2.14 (m, 3H), 2.08-2.03 (m, 1H). |
| 345 | 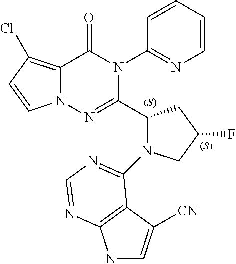 | 476.0 | ¹H NMR (400 MHz, CD₃OD) δ 8.71 (dd, J = 5.1, 1.5 Hz, 1H), 8.28 (s, 1H), 8.12 (td, J = 7.7, 1.9 Hz, 1H), 8.03 (s, 1H), 7.81 (d, J = 8.0 Hz, 1H), 7.61 (dd, J = 7.5, 4.9 Hz, 1H), 7.33 (d, J = 2.9 Hz, 1H), 6.48 (d, J = 3.0 Hz, 1H), 5.52-5.39 (m, J1H), 5.03 (d, J = 7.5 Hz, 1H), 4.55-4.34 (m, 2H), 2.72-2.52 (m, 1H), 2.44-2.25 (m, 1H). |
| 346 | 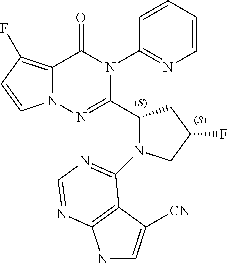 | 460.1 | ¹H NMR (400 MHz, DMSO-d₆) δ 8.76-8.68 (m, 1H), 8.34 (s, 1H), 8.30 (s, 1H), 8.12 (td, J = 7.7, 1.9 Hz, 1H), 7.84 (d, J = 7.9 Hz, 1H), 7.66-7.59 (m, 1H), 7.51 (dd, J = 4.6, 3.3 Hz, 1H), 6.45 (d, J = 3.2 Hz, 1H), 5.53-5.52 (m, 1H), 4.87 (s, 1H), 4.35 (d, J = 3.7 Hz, 1H), 4.29 (d, J = 3.7 Hz, 1H), 2.47-2.27 (m, 2H). |

### Example 42

### Compound 347

### (3S,5S)-5-(5-chloro-3-(3-fluorophenyl)-4-oxo-3,4-dihydropyrrolo[2,1-f][1,2,4]triazin-2-yl)-1-(9H-purin-6-yl)pyrrolidine-3-carbonitrile

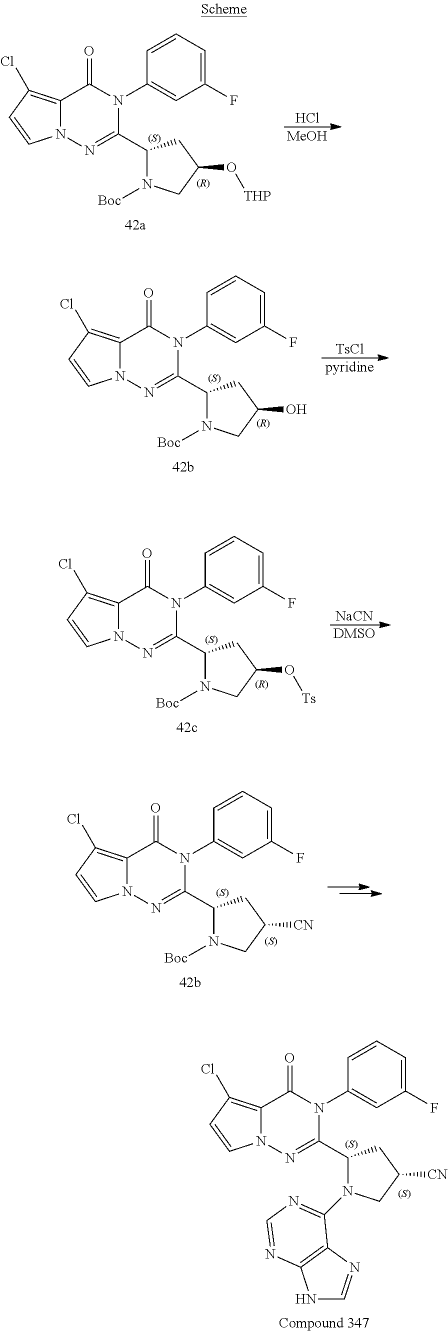

### Step 42-1 (2S,4R)-tert-butyl 2-(5-chloro-3-(3-fluorophenyl)-4-oxo-3,4-dihydropyrrolo[2,1-f][1,2,4]triazin-2-yl)-4-hydroxypyrrolidine-1-carboxylate (42b)

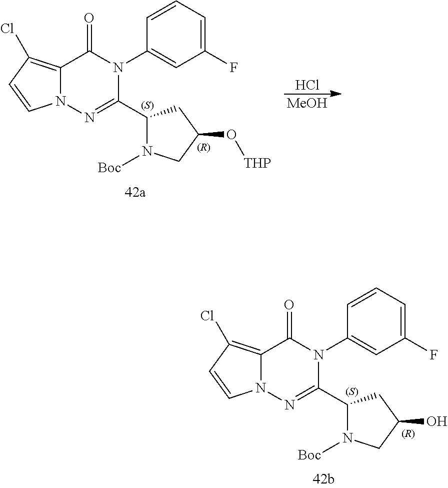

To a solution of 42a (prepared according to the procedures described in Example 3 using the corresponding reagents and intermediates) (1.32 g, 2.48 mmol) in MeOH (10 mL) was added HCl (3 drops). The mixture was concentrated to give the product 42b as a yellow solid.

### Step 42-2 (2S,4R)-tert-butyl 2-(5-chloro-3-(3-fluorophenyl)-4-oxo-3,4-dihydro pyrrolo[2,1-f][1,2,4]triazin-2-yl)-4-(tosyloxy)pyrrolidine-1-carboxylate (42c)

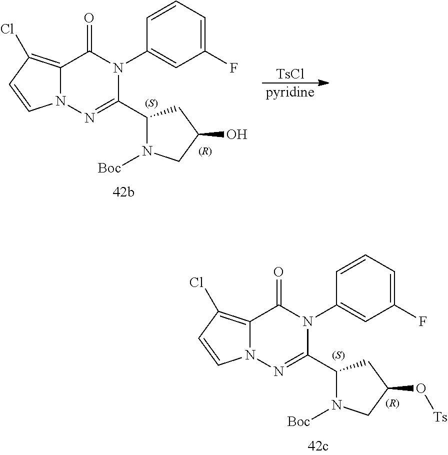

To a solution of 42b (1.1 g, 2.45 mmol) in pyridine (10 mL) was added TsCl (0.94 g, 4.9 mmol), the reaction was stirred at r.t overnight under N₂, then concentrated and purified by flash column chromatography to give 42c as a yellow solid. Yield 72%. MS (m/z): 603.1 (M+1)⁺.

### Step 42-3 (2S,4S)-tert-butyl 2-(5-chloro-3-(3-fluorophenyl)-4-oxo-3,4-dihydro pyrrolo[2,1-f][1,2,4]triazin-2-yl)-4-cyanopyrrolidine-1-carboxylate (42d)

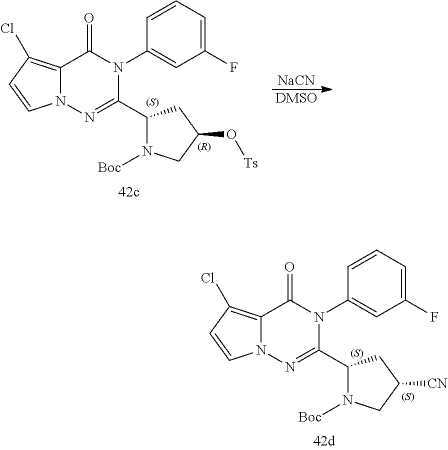

To a solution of 42c (1.07 g, 1.77 mmol) in DMSO (10 mL) was added NaCN (435 mg, 8.87 mmol). The reaction was stirred under N₂ at 80° C. overnight, then poured into water, and extracted with EtOAc, the organic layers were washed with water, brine, dried, concentrated and purified by flash column chromatography to give 42d as a yellow solid. Yield 56%. MS (m/z): 458.1 (M+1)⁺.

### Step 42-4 (3S,5S)-5-(5-chloro-3-(3-fluorophenyl)-4-oxo-3,4-dihydropyrrolo[2,1-f][1,2,4]triazin-2-yl)-1-(9H-purin-6-yl)pyrrolidine-3-carbonitrile (Compound 347)

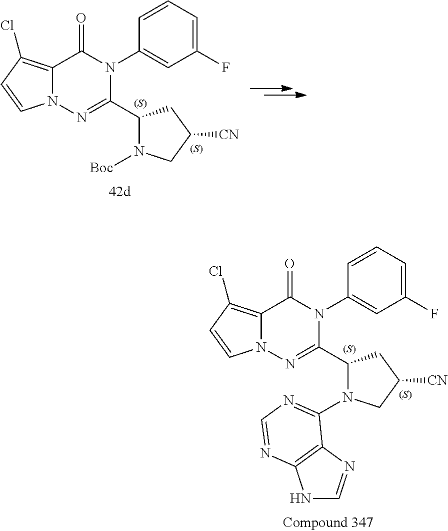

Compound 347 was prepared according to the procedures described in Example 1 from 42d using the corresponding reagents and intermediates. ¹H NMR (400 MHz, CD₃OD) δ 8.23 (s, 0.5H), 8.22 (s, 0.5H), 8.00 (s, 0.5H), 7.99 (s, 0.5H), 7.84 (brs, 1H), 7.67-7.59 (m, 1H), 7.41-7.29 (m, 2H), 7.25 (d, J=3.0 Hz, 1H), 6.44 (d, J=3.0 Hz, 1H), 5.34-5.27 (m, 1H), 4.30-4.25 (m, 1H), 3.55-3.45 (m, 1H), 3.35-3.33 (m, 1H), 2.53-2.48 (m, 2H). MS (m/z): 476.1 (M+1)⁺.

The following Compounds were prepared according to the procedure of Compound 347 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 350 | 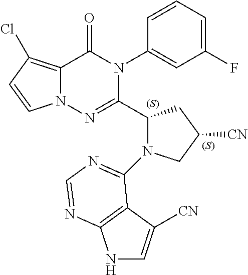 | 499.9 | ¹H NMR (400 MHz, CD₃OD) δ 8.20 (s, 0.5H), 8.20 (s, 0.5H), 7.97 (s, 1H), 7.61-7.53 (m, 2H), 7.37 (d, J = 2.8 Hz, 0.5H), 7.360 (d, J = 2.8 Hz, 0.5H), 7.33-7.26 (m, 2H), 6.47 (d, J = 3.0 Hz, 1H), 5.10-5.01 (m, 1H), 4.58-4.51 (m, 1H), 4.36-4.29 (m, 1H), 3.53-3.44 (m, 1H), 2.60-2.50 (m, 2H). |
| 351 | 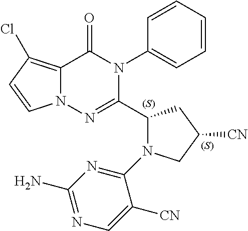 | 458.0 | ¹H NMR (400 MHz, DMSO-d₆) δ 8.25 (s, 1H), 7.84 (dd, J = 7.6, 1.6 Hz, 1H), 7.58-7.51 (m, 5H), 7.19-7.11 (br, 2H), 6.60 (d, J = 3.0 Hz, 1H), 4.70 (brs, 1H), 4.34-4.32 (m, 1H), 3.94 (brs, 1H), 2.41-2.35 (m, 1H), 2.18-2.08 (m, 1H), 2.00-1.94 (m, 1H). |

### Example 43

### Compound 352

### 5-chloro-2-((2S)-1-(3-(methylsulfinyl)-1H-pyrazolo[3,4-d]pyrimidin-4-yl)pyrrolidin-2-yl)-3-phenylpyrrolo[2,1-f][1,2,4]triazin-4(3H)-one

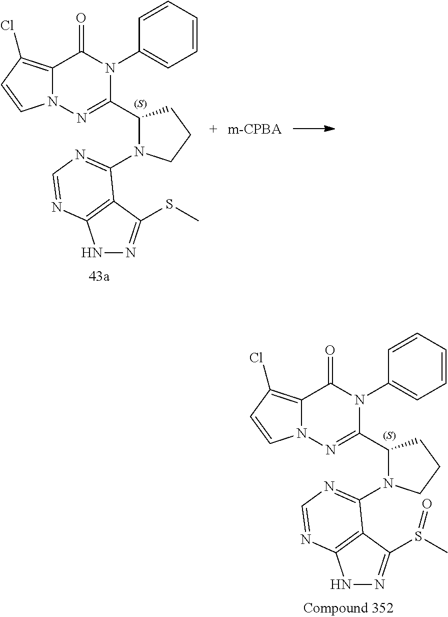

43a (prepared according to the procedures described in Example 1 using the corresponding reagents and intermediates) (40 mg, 0.08 mmol) and m-CPBA (19 mg, 75%, 0.08 mmol) were dissolved in DCM, the mixture was stirred at r.t. for 10 min, then concentrated and purified by TLC to give Compound 352 as a white solid. Yield: 61%. ¹H NMR (400 MHz, DMSO-d₆) δ 8.38 (d, J=2.8 Hz, 1H), 7.80-7.77 (m, 1H), 7.61-7.55 (m, 4.5H), 7.46 (d, J=2.8 Hz, 0.5H), 6.60 (d, J=2.8 Hz, 1H), 4.747-4.66 (m, 1H), 4.42-4.38 (m, 0.5H), 4.24-4.21 (m, 1H), 4.10-4.06 (m, 0.5H), 3.11 (s, 1.5H), 3.86 (s, 1.5H), 2.36-2.24 (m, 2H), 2.07-1.96 (m, 2H). MS (m/z): 495.1 (M+1)⁺.

The following Compound 353 and Compound 399 were prepared according to the procedure of Compound 352 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 353 | 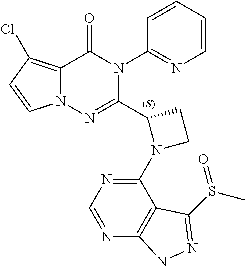 | 481.0 | ¹H NMR (400 MHz, DMSO-d₆) δ 8.19 (s, 0.5H), 8.18 (s, 0.5H), 7.65-7.54 (m, 6H), 6.63 (d, J = 3.2 Hz, 0.5H), 6.62 (d, J = 2.8 Hz, 0.5H), 5.14-5.09 (m, 1H), 4.58-4.47 (m, 1H), 4.26-4.15 (m, 1H), 3.05 (s, 1.5H), 3.018 (s, 1.5H), 2.68-2.60 (m, 1H), 2.20- 2.13 (m, 1H). |
| 399 | 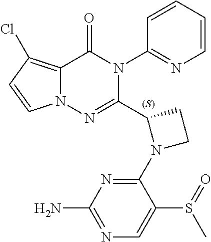 | 455.9 | ¹H NMR (400 MHz, CD₃OD) δ 8.36 (s, 0.5H), 8.30 (s, 0.5H), 7.66-7.52 (m, 4H), 7.44 (d, J = 3.0 Hz, 0.5H), 7.40 (d, J = 3.0 Hz, 0.5H), 7.34-7.29 (m, 1H), 6.55 (d, J = 3.0 Hz, 0.5H), 6.54 (d, J = 3.0 Hz, 0.5H), 5.09-5.05 (m, 0.5H), 5.01-4.95 (m, 0.5H), 4.30-4.15 (m, 1H), 4.06-3.97 (m, 1H), 2.83 (s, 1.5H), 2.76 (s, 1.5H), 2.53-2.44 (m, 1H), 2.28-2.18 (m, 1H). |

### Example 47

### Compound 357

### 2-((2S)-1-(2-amino-5-(1-hydroxyethyl)pyrimidin-4-yl)azetidin-2-yl)-5-chloro-3-phenylpyrrolo[2,1-f][1,2,4]triazin-4(3H)-one

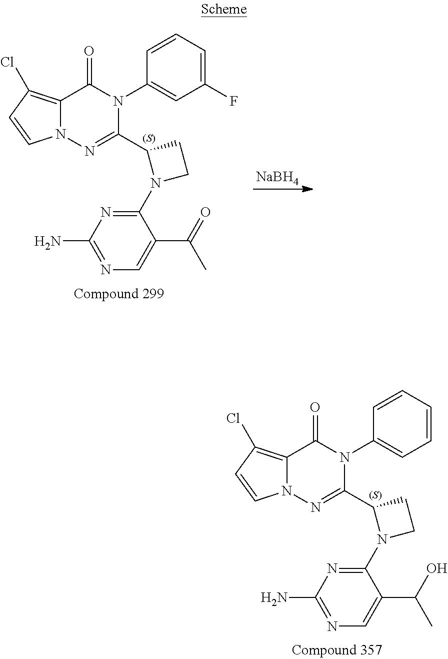

To a solution of Compound 299 (52 mg, 0.12 mmol) in MeOH (20 mL) was added NaBH₄ (9 mg, 0.24 mmol), the reaction was stirred at r.t. overnight, then quenched with water, the mixture was concentrated and purified by flash column chromatography to give Compound 357 as a white solid. Yield: 32%. ¹H NMR (400 MHz, DMSO-d₆) δ 8.19 (brs, 1H), 7.84 (brs, 1H), 7.73 (d, J=7.7 Hz, 1H), 7.69 (d, J=2.9 Hz, 1H), 7.62-7.51 (m, 3H), 7.42-7.39 (m, 1H), 6.66 (d, J=2.9 Hz, 1H), 6.07 (s, 2H), 4.77-4.74 (m, 1H), 4.62-4.60 (m, 1H), 4.15-4.10 (m, 1H), 3.99-3.93 (m, 1H), 2.48-2.41 (m, 1H), 1.99-1.91 (m, 1H), 1.30 (d, J=6.3 Hz, 3H). MS (m/z): 438.3 (M+1)⁺.

### Example 48

### Compound 358

### (3R,5S)-5-(5-chloro-4-oxo-3-phenyl-3,4-dihydropyrrolo[2,1-f][1,2,4]triazin-2-yl)-1-(9H-purin-6-yl)pyrrolidine-3-carbonitrile

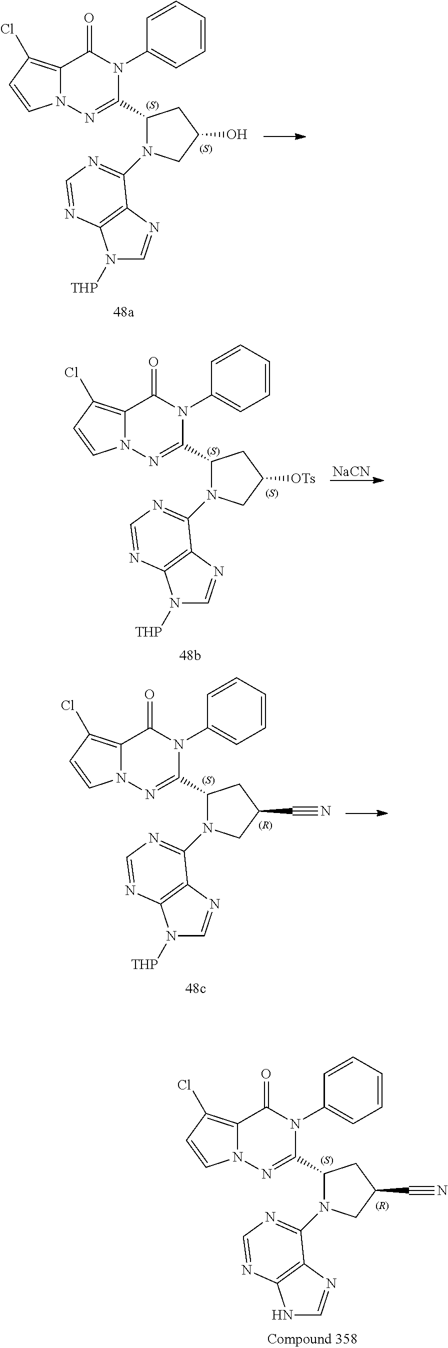

### Step 48-1 (3S,5S)-5-(5-chloro-4-oxo-3-phenyl-3,4-dihydropyrrolo[2,1-f][1,2,4]triazin-2-yl)-1-(9-(tetrahydro-2H-pyran-2-yl)-9H-purin-6-yl)pyrrolidin-3-yl-4-methyl benzenesulfonate (48b)

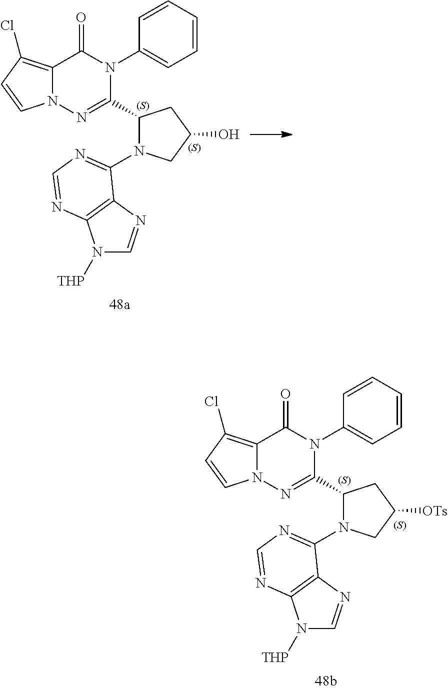

To a solution of 48a (prepared according to the procedures described in Example 3 using the corresponding reagents and intermediates) (107 mg, 0.2 mmol) in dry THF (5 ml) was added NaH (12 mg, 0.3 mmol), the mixture was stirred at 0° C. for 0.5 h under N₂, then TsCl (760 mg, 0.4 mmol) was added, the reaction was stirred for another 0.5 h. The mixture was concentrated and purified by chromatography to give 48b. Yield: 94%. MS (m/z): 687.3 (M+1)⁺.

### Step 48-2 (3R,5S)-5-(5-chloro-4-oxo-3-phenyl-3,4-dihydropyrrolo[2,1-f][1,2,4]triazin-2-yl)-1-(9-(tetrahydro-2H-pyran-2-yl)-9H-purin-6-yl)pyrrolidine-3-carbonitrile (48c)

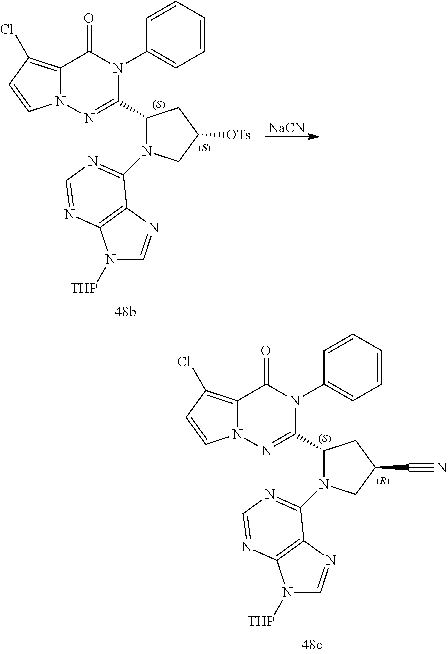

The mixture of 48b (120 mg, 0.188 mmol) and NaCN (460 mg, 0.94 mmol) in dry DMSO (10 mL) was stirred at 55° C. overnight under N₂. After reaction, the mixture was cooled to r.t. and poured into water, extracted with EtOAc, the organic layers were concentrated to give 48c, which was used for the next without further purification. MS (m/z): 542.1 (M+1)⁺.

### Step 48-3 (3R,5S)-5-(5-chloro-4-oxo-3-phenyl-3,4-dihydropyrrolo[2,1-f][1,2,4]triazin-2-yl)-1-(9H-purin-6-yl)pyrrolidine-3-carbonitrile (Compound 358)

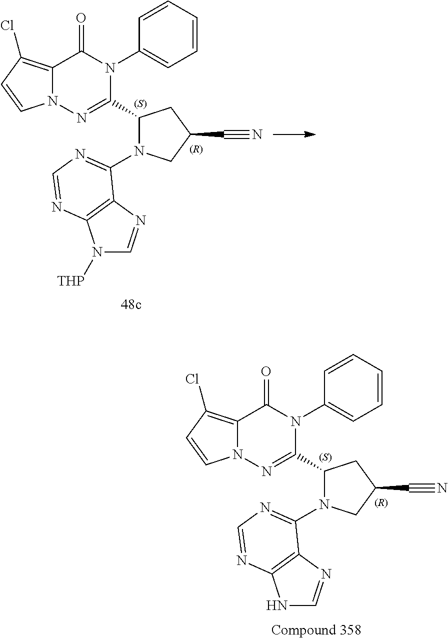

To a mixture of 48c (100 mg, 0.185 mmol) in methanol (5 mL) was added HCl (1 mL) stirred at 60° C. for 1 h. After reaction, the mixture was concentrated and purified by flash column chromatography to give Compound 358 as a white solid. Yield: 66%. ¹H NMR (400 MHz, DMSO-d₆) δ 8.15 (s, 1H), 7.98 (s, 1H), 7.62-7.55 (m, 5H), 7.46 (s, 1H), 6.57 (d, J=2.8 Hz, 1H), 2.73-2.65 (m, 2H), 2.569-2.54 (m, 0.5H), 2.46-2.44 (m, 0.5H), 2.23-2.15 (m, 2H), 2.03-1.95 (m, 1H). MS (m/z): 458 (M+1)⁺

The following Compounds 359-361 were prepared according to the procedure of Compound 358 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 359 | 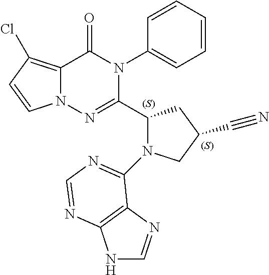 | 458.2 | ¹H NMR (400 MHz, CD₃OD) δ 8.10 (s, 1H), 7.94 (d, J = 7.2 Hz, 1H), 7.85 (s, 1H), 7..66-7.53 (m, 3H), 7.46-7.43 (m, 1H), 7.28 (d, J = 3.2 Hz, 1H), 6.45 (d, J = 3.2 Hz, 1H), 5.19-5.13 (m, 1H), 4.36-4.32 (m, 1H), 3.49-3.43 (m, 1H), 3.36-3.33 (m, 1H), 2.47-2.43 (m, 2H). |
| 360 | 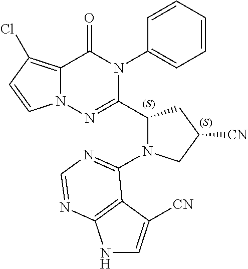 | 482.1 | ¹H NMR (400 MHz, CD₃OD) δ 8.22 (s, 1H), 7.95 (s, 1H), 7.69-7.66 (m, 1H), 7.55-7.44 (m, 3H), 7.37-7.35 (m, 1H), 7.2553 (d, J = 3.2 Hz, 1H), 6.38 (d, J = 3.2 Hz, 1H), 4.95-4.91 (m, 1H), 4.49-4.44 (m, 1H), 4.29-4.24 (m, 1H), 3.45-3.37 (m, 1H), 2.52-2.38 (m, 2H). |
| 361 | 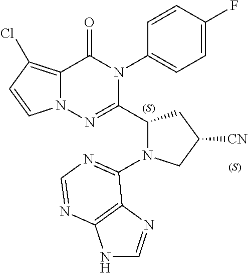 | 476.1 | ¹H NMR (400 MHz, CD₃OD) δ 8.29 (s, 1H), 8.08 (s, 1H), 7.56-7.32 (m, 3H), 7.26-7.22 (m, 1H), 6.44 (br, 1H), 5.17 (br, 1H), 4.56-4.51 (m, 2H), 3.57-3.50 (br, 1H), 2.55-2.49 (br, 2H). |

### Example 49

### Compound 264

### 4-((2S,4S)-2-(5-chloro-4-oxo-3-phenyl-3,4-dihydropyrrolo[2,1-f][1,2,4]triazin-2-yl)-4-(2-methoxyethoxyl)pyrrolidin-1-yl)-7H-pyrrolo[2,3-d]pyrimidine-5-carbonitrile

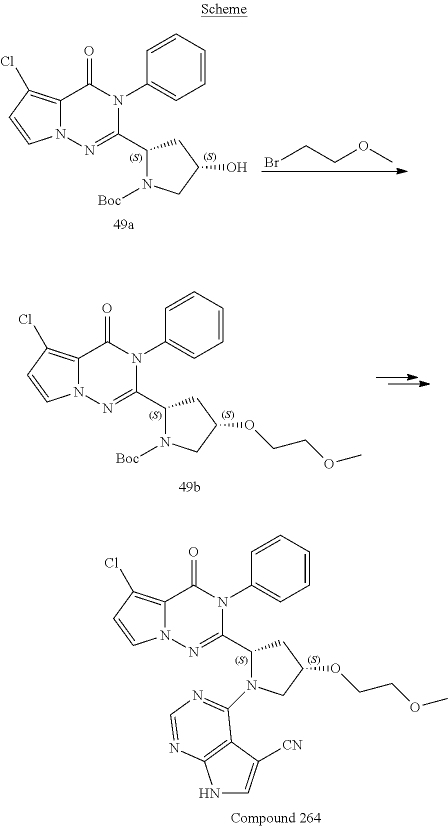

### Step 49-1 (2S,4S)-tert-butyl 2-(5-chloro-4-oxo-3-phenyl-3,4-dihydropyrrolo[2,1-f][1,2,4]triazin-2-yl)-4-(2-methoxyethoxyl)pyrrolidine-1-carboxylate (49b)

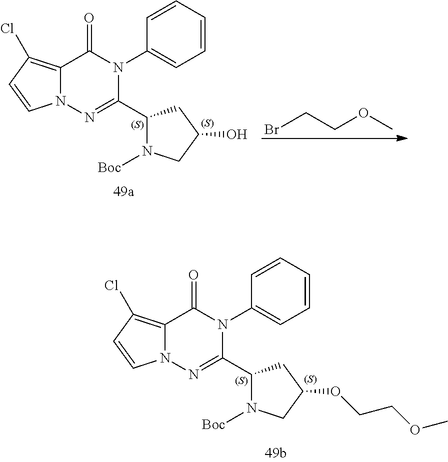

To a solution of 49a ((prepared according to the procedures described in Example 1 using the corresponding reagents and intermediates) (55 mg, 0.128 mmol) in DMF (1 mL) was added NaH (8 mg, 0.19 mmol) at 0° C., the reaction was stirred at 0° C. for 0.5 h, then 1-bromo-2-methoxyethane (36 mg, 0.256 mmol) was added, the mixture was stirred in a sealed tube at 130° C. overnight. After cooling to r.t., the reaction was quenched with water, then concentrated and purified by flash column chromatography to give 49b. Yield: 27%. MS (m/z): 489.1 (M+1)⁺.

### Step 49-2 4-((2S,4S)-2-(5-chloro-4-oxo-3-phenyl-3,4-dihydropyrrolo[2,1-f][1,2,4]triazin-2-yl)-4-(2-methoxyethoxyl)pyrrolidin-1-yl)-7H-pyrrolo[2,3-d]pyrimidine-5-carbonitrile (Compound 264)

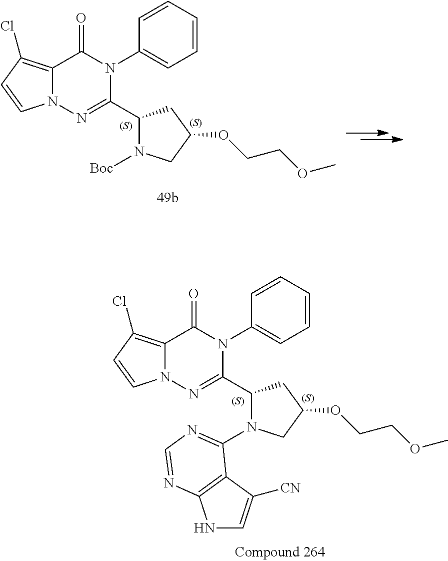

Compound 264 was prepared according to the procedures described in Example 1 from 49b using the corresponding reagents and intermediates. ¹H NMR (400 MHz, DMSO-d₆) δ 8.31 (s, 1H), 8.28 (s, 1H), 7.73 (d, J=7.2 Hz, 1H), 7.61-7.49 (m, 5H), 6.56 (d, J=2.8 Hz, 1H), 4.59 (t, J=8.2 Hz, 1H), 4.31 (t, J=7.8 Hz, 1H), 4.17-4.10 (m, 1H), 3.83-3.79 (m, 1H), 3.54-3.48 (m, 2H), 3.42-3.38 (m, 2H), 3.19 (s, 3H), 2.41-2.28 (m, 2H). MS (m/z): 531.3 (M+1)⁺.

### Example 50

### Compound 363

### 3-(1-(9H-purin-6-yl)pyrrolidin-2-yl)-8-chloro-2-phenylpyrrolo[1,2-a]pyrazin-1(2H)-one

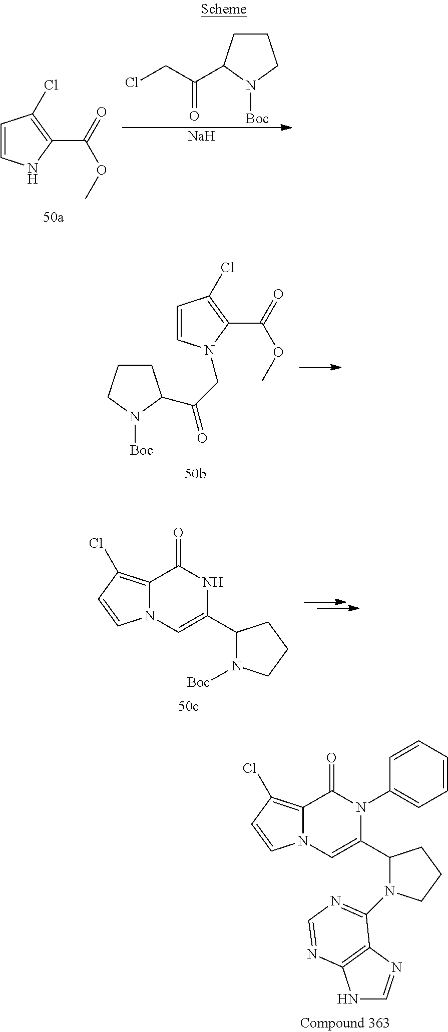

### Step 50-1 methyl 1-(2-(1-(tert-butoxycarbonyl)pyrrolidin-2-yl)-2-oxoethyl)-3-chloro-1H-pyrrole-2-carboxylate (50b)

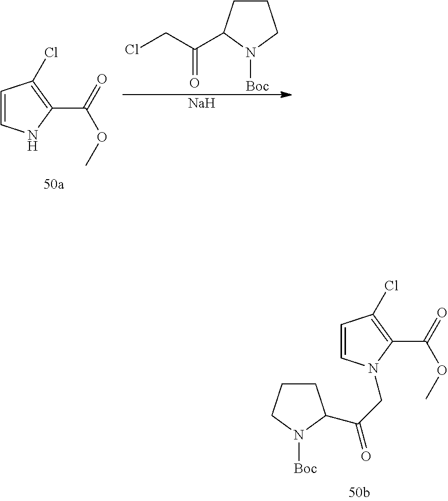

To a solution of NaH (500 mg, 60%, 12.5 mmol) in DMF was added 50a (1.59 g, 10 mmol in 10 mL of DMF) dropwise at 0° C., the reaction was stirred at r.t. for 30 min, then tert-butyl 2-(2-chloroacetyl)pyrrolidine-1-carboxylate (3.0 g, 12 mmol in 10 mL of DMF) was added dropwise at 0° C., the reaction was warmed to r.t. and stirred for 2 h. The mixture was poured into water, extracted with EtOAc, the organic layers were washed with brine, dried over Na₂SO₄, concentrated to give 50b as a dark oil, which was used for the next step without purification. MS (m/z): 271.1 (M-100+1)⁺.

### Step 50-2 tert-butyl 2-(8-chloro-1-oxo-1,2-dihydropyrrolo[1,2-a]pyrazin-3-yl) pyrrolidine-1-carboxylate (50c)

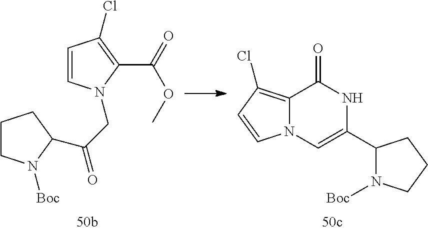 [SMILES: COC(=O)c1c(Cl)ccn1CC(=O)C1CCCN1C(=O)OC(C)(C)C ; CC(C)(C)OC(=O)N1CCCC1c1cn2ccc(Cl)c2c(=O)[nH]1]

50b (3.7 g, 10 mmol) was dissolved in NH₃/MeOH (7 N, 100 mL), the reaction was stirred at 130° C. overnight. The mixture was concentrated to about 30 mL, the resulting precipitate was filtered, and poured into water, then 1N HCl (3 mL) was added, the resulting mixture was stirred at r.t. for 5 min, DCM was added until the precipitate was dissolved. The resulting solution was washed with water, dried over Na₂SO₄, concentrated to give 50c as a brown solid, which was used for next step without purification. Yield: 53%, MS (m/z): 337.9 (M+1)⁺.

### Step 50-3 3-(1-(9H-purin-6-yl)pyrrolidin-2-yl)-8-chloro-2-phenylpyrrolo[1,2-a]pyrazin-1(2H)-one (Compound 363)

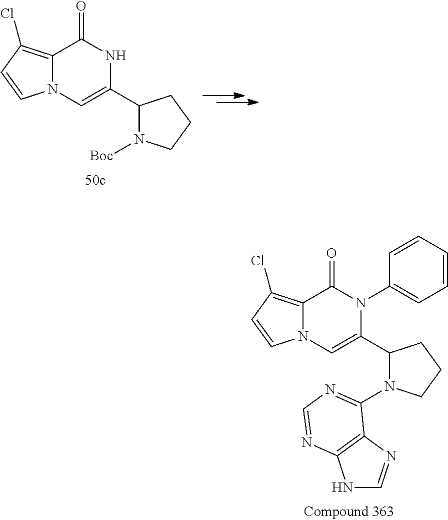 [SMILES: CC(C)(C)OC(=O)N1CCCC1c1cn2ccc(Cl)c2c(=O)[nH]1 ; O=c1c2c(Cl)ccn2cc(C2CCCN2c2ncnc3[nH]cnc23)n1-c1ccccc1]

Compound 363 was prepared according to the procedures described in Example 1 from 50c using the corresponding reagents and intermediates. ¹H NMR (400 MHz, DMSO-d₆) δ 12.94 (br, 1H), 8.27 (s, 1H), 8.21 (br, 1H), 7.57-7.49 (m, 5H), 7.37 (d, J=2.8, 1H), 7.08 (br, 1H), 6.54 (s, 1H), 5.41 (br, 0.5H), 4.79-4.47 (m, 0.5H), 4.10-3.97 (m, 1H), 3.62 (s, 1H), 1.94 (br, 3H), 1.70-1.65 (m, 1H). MS (m/z): 432.4 (M+1)¹.

The following Compound 364 was prepared according to the procedure of Compound 363 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 364 | 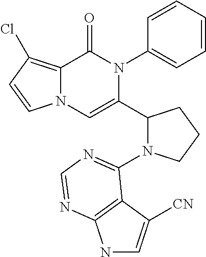 [SMILES: N#Cc1c[nH]c2ncnc(N3CCCC3c3cn4ccc(Cl)c4c(=O)n3-c3ccccc3)c12] | 455.8 | ¹H NMR (400 MHz, DMSO-d₆) δ 8.31 (s, 1H), 8.30 (s, 1H), 7.66-7.46 (m, 5H), 7.33-7.32 (m, 2H), 6.57 (d, J = 2.8, 1H), 4.81 (dd, J = 7.9, 2.9, 1H), 4.39-4.27 (m, 1H), 3.81-3.78 (m, 1H), 2.24-2.10 (m, 1H), 2.01-1.95 (m, 1H), 1.92-1.86 (m, 1H), 1.80-1.70 (m, 1H). |

### Example 51

### Compound 365

### 4-((2S,4S)-2-(5-chloro-4-oxo-3-phenyl-3,4-dihydropyrrolo[2,1-f][1,2,4]triazin-2-yl)-4-(methylsulfonyl)pyrrolidin-1-yl)-7H-pyrrolo[2,3-d]pyrimidine-5-carbonitrile

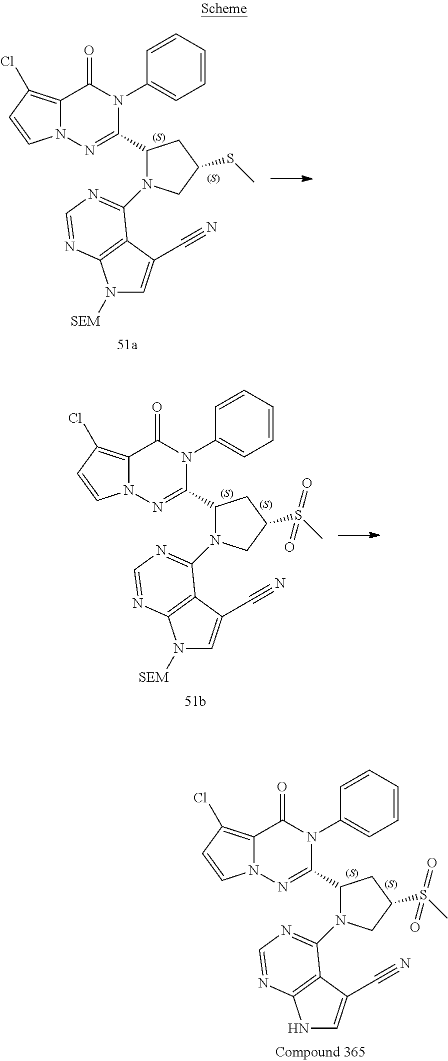

### Step 51-1 4-((2S,4S)-2-(5-chloro-4-oxo-3-phenyl-3,4-dihydropyrrolo[2,1-f][1,2,4]triazin-2-yl)-4-(methylthio)pyrrolidin-1-yl)-7-((2-(trimethylsilyl)ethoxy)methyl)-7H-pyrrolo[2,3-d]pyrimidine-5-carbonitrile (51b)

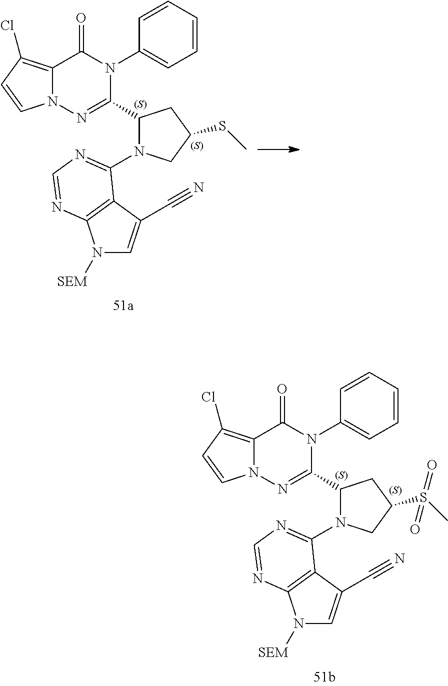

To a mixture of 51a (prepared according to the procedures described in Example 48 using the corresponding reagents and intermediates) (50 mg, 0.08 mmol) in dry DCM (5 mL) was added m-CPBA (26 mg, 0.15 mmol), the reaction was stirred at r.t. for 24 h. The mixture was concentrated to give 51b as a solid, which was used for the next step without further purification. MS (m/z): 677.1 (M+1)⁺.

### Step 51-2 4-((2S,4S)-2-(5-chloro-4-oxo-3-phenyl-3,4-dihydropyrrolo[2,1-f][1,2,4]triazin-2-yl)-4-(methylsulfonyl)pyrrolidin-1-yl)-7H-pyrrolo[2,3-d]pyrimidine-5-carbonitrile (Compound 365)

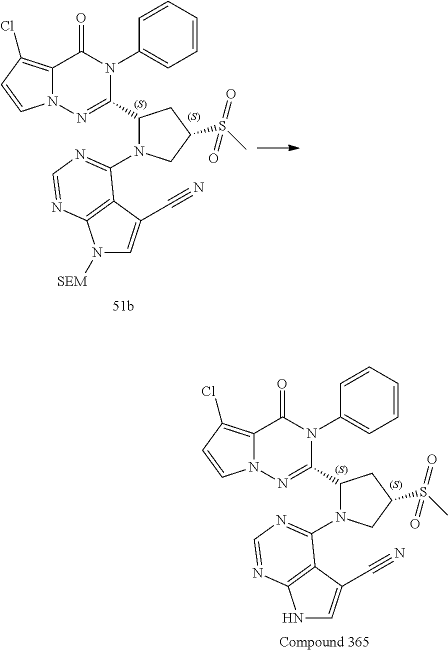

The mixture of 51b (52 mg, 0.079 mmol) in CF₃COOH (1 mL) was stirred for 1 h, then concentrated, the resulting residue was added NH₃.H₂O (1 mL) in MeOH, the mixture was stirred for another 1 h, then concentrated and purified by flash column chromatography to give Compound 365 as a white solid. Yield: 47%. ¹H NMR (400 MHz, CD₃OD) δ 8.13 (s, 1H), 7.93 (s, 1H), 7.85 (d, J=7.6 Hz, 1H), 7.69-7.64 (m, 1H), 7.59-7.57 (m, 2H), 7.42-7.37 (m, 2H), 6.49 ((d, J=2.4 Hz, 1H), 4.53-4.49 (m, 1H), 4.41-4.36 (m, 1H), 4.09-4.00 (m, 1H), 3.66-3.61 (m, 1H), 3.38 (s, 3H), 2.66-2.54 (m, 2H). MS (m/z): 535.1 (M+1)⁺.

The following Compound 366 was prepared according to the procedure of Compound 365 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 366 | 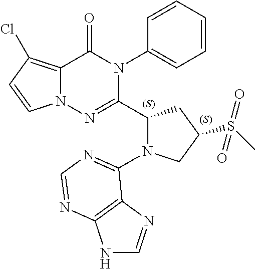 | 511.1 | 1H NMR (400 MHz, CD₃OD) δ 8.08 (s, 1H), 7.90 (d, J = 8.4 Hz, 1H), 7.83 (s, 1H), 7.64-7.55 (m, 3H), 7.43-7.40 (m, 1H), 7.25 (d, J = 3.2 Hz, 1H), 6.43 (d, J = 2.1 Hz, 1H), 5.12-5.07 (m, 2H), 4.31-4.26 (m, 1H), 4.04-3.95 (m, 1H), 3.05 (s, 3H), 2.591-2.414 (m, 2H). |

### Example 52

### Compound 367

### (S)-2-(1-(2-aminoimidazo[1,2-a][1,3,5]triazin-4-yl)pyrrolidin-2-yl)-5-chloro-3-phenylpyrrolo[2,1-f][1,2,4]triazin-4(3H)-one

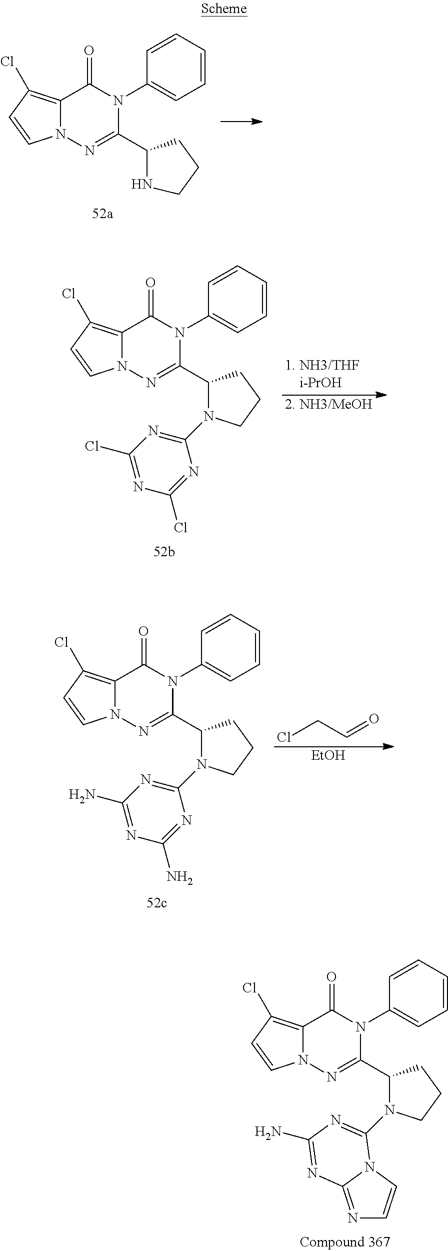

### Step 52-1 (S)-5-chloro-2-(1-(4,6-dichloro-1,3,5-triazin-2-yl)pyrrolidin-2-yl)-3-phenylpyrrolo[2,1-f][1,2,4]triazin-4(3H)-one (52b)

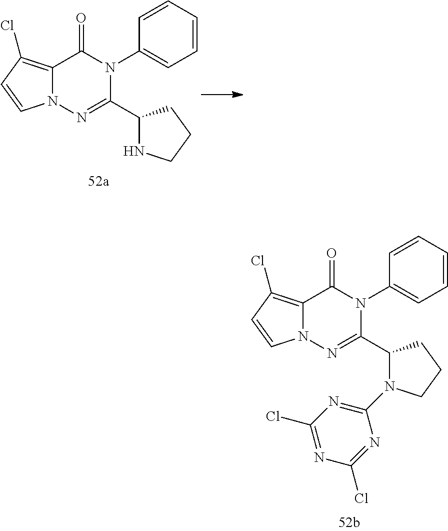 [SMILES: O=c1c2c(Cl)ccn2nc([C@@H]2CCCN2)n1-c1ccccc1 ; O=c1c2c(Cl)ccn2nc([C@@H]2CCCN2c2nc(Cl)nc(Cl)n2)n1-c1ccccc1]

To a solution of 2,4,6-trichloro-1,3,5-triazine (36.8 mg, 0.2 mmol) in THF (3 mL) were added DIEA (51.6 mg, 0.4 mmol) and a solution of 52a (prepared according to the procedures described in Example 1 using the corresponding reagents and intermediates, about 0.1 mmol) in THF (4 mL) at r.t. The reaction was stirred at r.t for 2 h. The mixture was used directly for next step without purification.

### Step 52-2 (S)-5-chloro-2-(1-(4,6-diamino-1,3,5-triazin-2-yl)pyrrolidin-2-yl)-3-phenyl pyrrolo[2,1-f][1,2,4]triazin-4(3H)-one (52c)

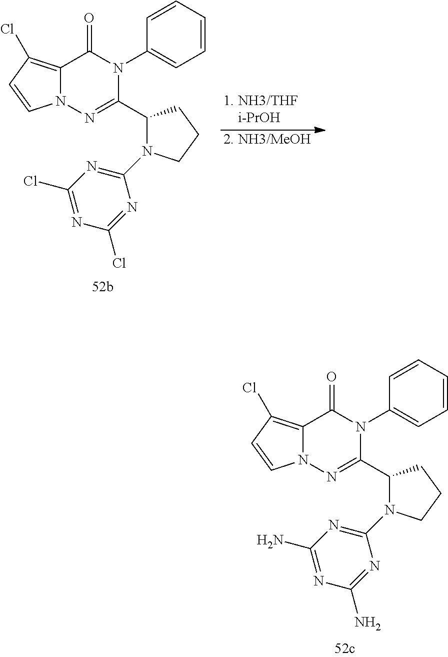

To the above mixture of 52b in THF was added a solution of NH₃ in THF (7 N, 3 mL) at r.t., the reaction was stirred at r.t. overnight, then a solution of NH₃ in MeOH (7 N, 5 mL) was added, the resulting mixture was stirred at 100° C. overnight in a sealed tube. The mixture was concentrated and purified by flash column chromatography to give 52c as a yellow solid. Yield: 94.6%. MS (m/z): 424.5 (M+1)⁺.

### Step 52-3 (S)-2-(1-(2-aminoimidazo[1,2-a][1,3,5]triazin-4-yl)pyrrolidin-2-yl)-5-chloro-3-phenylpyrrolo[2,1-f][1,2,4]triazin-4(3H)-one (Compound 367)

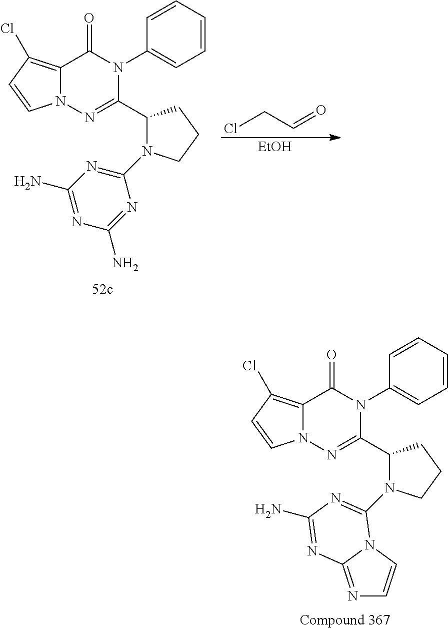

To a solution of 52c (40 mg, 0.09 mmol) in EtOH (2 mL) was added a solution of 2-chloroacetaldehyde in H₂O (40%, 18.4 mg) at r.t., the reaction was stirred at 100° C. overnight. The reaction was concentrated and purified by flash column chromatography and p-TLC to give Compound 367 as a white solid. Yield: 52%. ¹H NMR (400 MHz, CD₃OD) δ 8.03 (s, 0.4H), 7.86 (s, 0.4H), 7.68-7.62 (m, 1H), 7.56 (br, 2H), 7.46-7.37 (m, 1H), 7.34 (br, 2H), 7.24 (m, 0.4H), 7.09 (br, 1H), 6.47 (br, 1H), 3.92-3.80 (m, 1.4H), 3.68-3.57 (m, 1.4H), 2.24-2.09 (m, 2.8H), 2.00-1.80 (m, 2.8H). MS (m/z): 448.2 (M+1)⁺.

The following Compound 368 was prepared according to the procedure of Compound 367 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 368 | 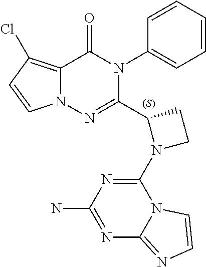 | 434.1 | ¹H NMR (400 MHz, DMSO-d₆) δ 8.05 (s, 2H), 7.74 (d, J = 2.6 Hz, 1H), 7.63- 7.48 (m, 5H), 7.47-7.41 (m, 1H), 7.10 (s, 1H), 6.68 (d, J = 2.5 Hz, 1H), 4.75-4.64 (m, 1H), 3.92-3.81 (m, 2H), 2.20- 1.79 (m, 2H). |

### Example 53

### Compound 369

### (S)-2-(1-(5-acetyl-2-aminopyrimidin-4-yl)pyrrolidin-2-yl)-5-chloro-3-phenylpyrrolo[2,1-f][1,2,4]triazin-4(3H)-one

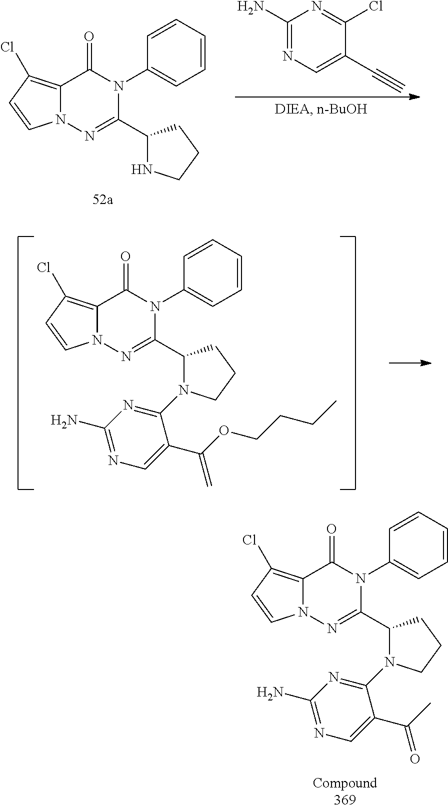

To a solution of 52a (about 0.2 mmol) in n-BuOH (10 mL) was added DIEA (103 mg, 0.8 mmol) and 4-chloro-5-ethynylpyrimidin-2-amine (34 mg, 0.22 mmol) at r.t., the reaction was stirred at 120° C. overnight. The mixture was concentrated and purified by flash column chromatography and p-TLC to afford Compound 369 as a white solid. Yield: 39%. ¹H NMR (400 MHz, CD₃OD) δ 8.40 (s, 1H), 7.79 (d, J=7.7 Hz, 1H), 7.65-7.50 (m, 3H), 7.45-7.39 (m, 1H), 7.32 (d, J=2.9 Hz, 1H), 6.48 (d, J=3.0 Hz, 1H), 4.81-4.76 (m, 1H), 3.45-3.36 (m, 1H), 3.25-3.14 (m, 1H), 2.48 (s, 3H), 2.17-1.99 (m, 2H), 1.96-1.85 (m, 1H), 1.81-1.67 (m, 1H). MS (m/z): 450.1 (M+1)⁺.

### Example 55

### Compound 371 (S)-4-(2-(5-chloro-3-(cyclopropylmethyl)-4-oxo-3,4-dihydropyrrolo[2,1-f][1,2,4]triazin-2-yl)azetidin-1-yl)-7H-pyrrolo[2,3-d]pyrimidine-5-carbonitrile

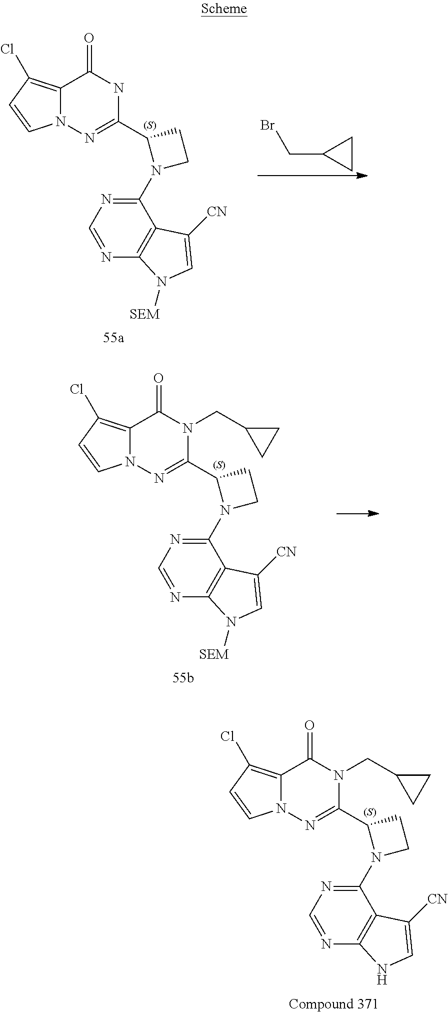

### Step 55-1 (S)-4-(2-(5-chloro-3-(cyclopropylmethyl)-4-oxo-3,4-dihydropyrrolo[2,1-f][1,2,4]triazin-2-yl)azetidin-1-yl)-7-((2-(trimethylsilyl)ethoxy)methyl)-7H-pyrrolo[2,3-d]pyrimidine-5-carbonitrile (55b)

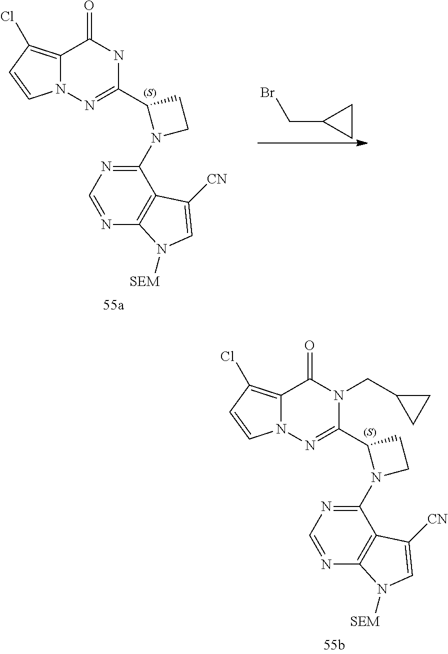

The mixture of 55a (prepared according to the procedures described in Example 41 using the corresponding reagents and intermediates) (99 mg, 0.2 mmol) and bromomethylcyclopropane (135 mg, 1 mmol) and Cs₂CO₃ (325 mg, 1 mmol) in DMF (5 mL) was stirred at 120° C. overnight in a sealed flask. After reaction, the reaction mixture was concentrated and purified by flash column chromatography to give 55b as a yellow solid. Yield: 68%. MS (m/z): 551.2 (M+1)⁺.

### Step 55-2 (S)-4-(2-(5-chloro-3-(cyclopropylmethyl)-4-oxo-3,4-dihydropyrrolo[2,1-f][1,2,4]triazin-2-yl)azetidin-1-yl)-7H-pyrrolo[2,3-d]pyrimidine-5-carbonitrile (Compound 371)

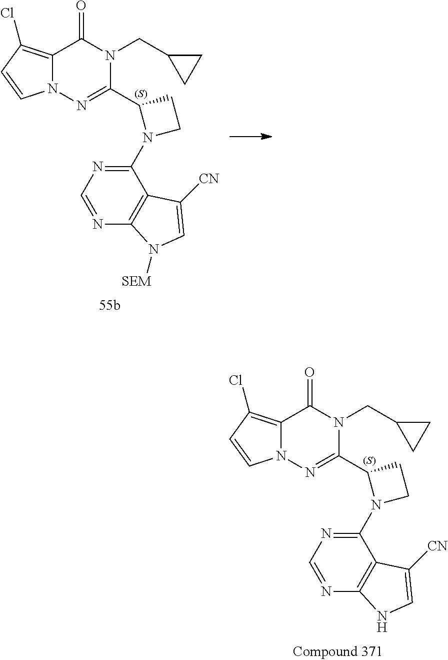

Compound 371 was prepared according to the procedures described in Example 41 using 55b instead of 41c. ¹H NMR (400 MHz, CD₃OD) δ 8.11 (s, 1H), 7.91 (s, 1H), 7.30 (d, J=3.2, 1H), 6.45 (d, J=3.2, 1H), 5.90-5.85 (m, 1H), 4.48-4.42 (m, 1H), 4.18-4.13 (m, 1H), 3.81-3.76 (m, 1H), 3.06-2.97 (m, 1H), 2.66-2.57 (m, 1H), 1.34-1.27 (m, 2H), 0.63-0.506 (m, 4H). MS (m/z): 421.0 (M+1)⁺.

The following Compounds were prepared according to the procedure of Compound 371 using the corresponding reagents and intermediates under appropriate conditions that will be recognized by one skilled in the art:

| Compd. | | LC/MS | |
|---|---|---|---|
| No. | Structure | (M + 1)+ | NMR |
| 372 | 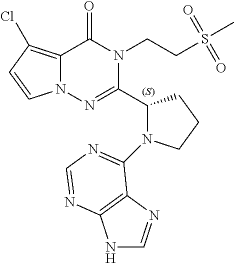 | 463.0 | ¹H NMR (400 MHz, CD₃OD) δ 8.23 (s, 1H), 7.90 (s, 1H), 7.15 (s, 1H), 6.38 (d, J = 2.8 Hz, 1H), 6.14-6.07 (m, 1H), 4.52-4.36 (m, 1H), 4.28-4.20 (m, 2H), 4.02-3.86 (m, 3H), 3.15 (s, 3H), 2.69- 2.53 (m, 1H), 2.39-2.26 (m, 1H), 2.24-2.09 (m, 2H). |
| 373 | 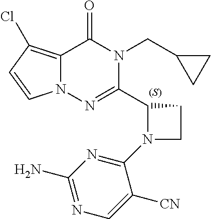 | 397.0 | ¹H NMR (400 MHz, CD₃OD) 8.12 (s, 1H), 7.35 (d, J = 2.0 Hz, 1H), 6.49 (d, J = 3.2 Hz, 1H), 5.88 (s, 1H), 4.45- 4.32 (m, 1H), 4.08-4.00 (m, 1H), 3.70-3.63 (m, 1H), 3.03-2.94 (m, 1H), 2.51-2.42 (m, 1H), 1.32-1.14 (m, 2H), 0.60-0.43 (m, 4H). |
| 374 | 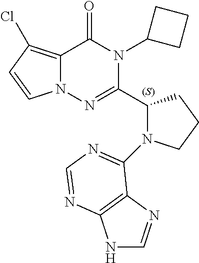 | 411.1 | ¹H NMR (400 MHz, CD₃OD) δ 7.94 (s, 1H), 7.78 (s, 1H), 7.17 (d, J = 2.8, 1H), 6.38 (d, J = 3.2, 1H), 4.36-4.31 (m, 1H), 4.22-4.15 (m, 1H), 4.07-4.02 (m, 1H), 2.54-2.43 (m, 1H), 2.37-2.28 (m, 1H), 2.19-2.13 (m, 2H), 1.69-1.62 (m, 1H), 1.33-1.25 (m, 2H), 0.69-0.55 (m, 4H). |
| 375 | 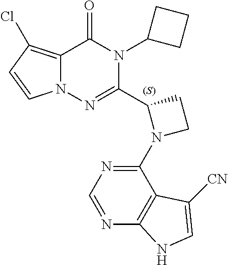 | 421.1 | ¹H NMR (400 MHz, CD₃OD) δ 8.04 (s, 1H), 7.87 (s, 1H), 7.32 (d, J = 2.8 Hz, 1H), 6.45 (d, J = 2.8 Hz, 1H), 4.79-4.74 (m, 2H), 4.44-4.38 (m, 1H), 4.17-4.12 (m, 1H), 3.82-3.76 (m, 1H), 3.04-2.95 (m, 1H), 2.638-2.558 (m, 2H), 0.628-0.494 (m, 4H). |
| 376 | 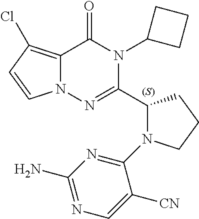 | 411.1 | ¹H NMR (400 MHz, DMSO-d₆) δ 8.24 (s, 1H), 7.53 (d, J = 2.8 Hz, 1H), 6.58 (d, J = 2.8 Hz, 1H), 4.10-4.02 (m, 2H), 4.00-3.88 (m, 2H), 2.40-2.30 (m, 1H), 2.23-2.21(m, 2H), 2.03-1.96 (m, 2H), 0.87-0.84 (m, 1H), 0.64-0.43 (m, 4H). |

### Example 56

### Compound 377

### (S)-2-(1-(2-amino-5-chloro-6-methylpyrimidin-4-yl)azetidin-2-yl)-5-chloro-3-phenylpyrrolo[2,1-f][1,2,4]triazin-4(3H)-one

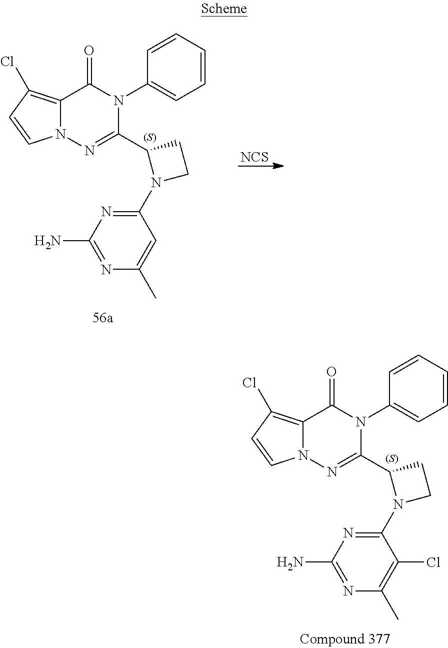

To a solution of 56a (prepared according to the procedures described in Example 1 using the corresponding reagents and intermediates) (50 mg, 0.12 mmol) in DCM (5 mL) was added NCS (20 mg, 0.15 mmol), the reaction was stirred at r.t. for 5 h, then concentrated and purified by p-TLC to give Compound 377 as a yellow solid. Yield: 30%. ¹H NMR (400 MHz, DMSO-d6) δ 7.71 (d, J=3.0 Hz, 1H), 7.65-7.50 (m, 4H), 7.41-7.34 (m, 1H), 6.64 (d, J=3.0 Hz, 1H), 6.17 (s, 2H), 4.78 (t, J=7.3 Hz, 1H), 4.20-4.15 (m, 1H), 4.00-3.94 (m, 1H), 2.45-2.38 (m, 1H), 2.13 (s, 3H), 1.98-1.87 (m, 1H). MS (m/z): 442.4 (M+1)⁺.

### Example 57

### Compound 378

### (S)-2-amino-4-(2-(5-chloro-4-oxo-3-phenyl-3,4-dihydropyrrolo[2,1-f][1,2,4]triazin-2-yl)azetidin-1-yl)-6-methoxypyrimidine-5-carbonitrile

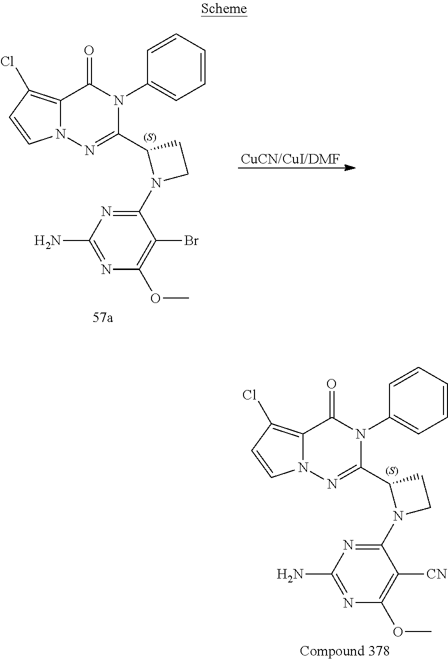

The mixture of 57a (prepared according to the procedures described in Example 56 using the corresponding reagents and intermediates) (23 mg, 0.046 mmol), CuCN (6 mg, 0.069 mmol) and CuI (1 mg, 0.005 mmol) in DMF (2 mL) was stirred at 120° C. under N₂ overnight. The reaction mixture was concentrated and purified flash column chromatography to give Compound 378 as a yellow solid. Yield: 29%. ¹H NMR (400 MHz, CD₃OD) δ 7.61-7.53 (m, 4H), 7.48 (d, J=3.0 Hz, 1H), 7.33-7.29 (m, 1H), 6.56 (d, J=3.2 Hz, 1H), 5.08 (brs, 1H), 4.23 (brs, 1H), 4.08-4.06 (m, 1H), 3.89 (s, 3H), 2.79-2.41 (m, 1H), 2.25-2.16 (m, 1H). MS (m/z): 449.1 (M+1)⁺.

### Example 58

### Compound 380

### (S)-4-(2-(5-chloro-4-oxo-3-phenyl-3,4-dihydropyrrolo[2,1-f][1,2,4]triazin-2-yl)-4-oxopyrrolidin-1-yl)-7H-pyrrolo[2,3-d]pyrimidine-5-carbonitrile

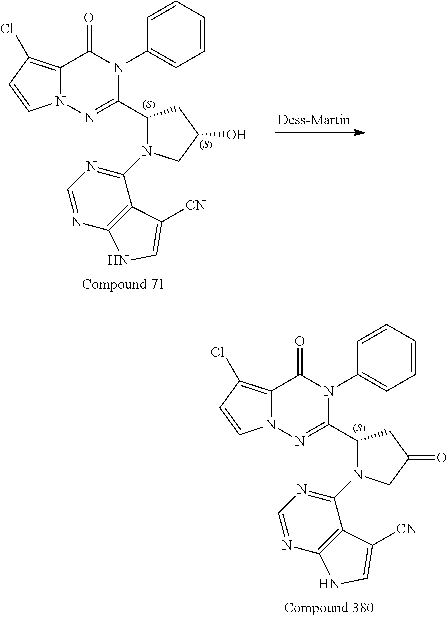

To a mixture of Compound 71(30 mg, 0.064 mmol) in dry DMF (25 mL) was added Dess-Martin reagent (54 mg, 0.128 mmol), the reaction was stirred at r.t. for 3 h, then filtered, the filtrate was purified by flash column chromatography to give Compound 380 as a yellow solid. Yield: 83%. ¹H NMR (400 MHz, CDCl₃) δ 8.38 (s, 1H), 7.78 (s, 1H), 7.67 (d, J=7.6 Hz, 1H), 7.56-7.46 (m, 3H), 7.18-7.16 (m, 1H), 7.02 (d, J=3.2 Hz, 1H), 6.35 (d, J=2.8 Hz, 1H), 5.51 (t, J=5.8 Hz, 1H), 4.66 (d, J=3.2 Hz, 2H), 2.69 (d, J=6.0 Hz, 2H). MS (m/z): 471.1 (M+1)⁺.

The following Compounds were prepared according to the procedure of Compound 380 using the corresponding reagents and intermediates under appropriate conditions that will be recognized by one skilled in the art:

| Compd. | | LC/MS | |
|---|---|---|---|
| No. | Structure | (M + 1)⁺ | NMR |
| 381 | 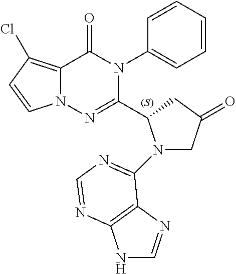 | 446.8 | ¹H NMR (400 MHz, CD₃OD) δ 8.31 (s, 1H), 8.05 (s, 1H), 8.01 (br, 1H), 7.68-7.63 (m, 1H), 7.61-7.55 (m, 2H), 7.45-7.43 (m, 1H), 7.22 (d, J = 2.8 Hz, 1H), 6.43 (d, J = 3.2 Hz, 1H), 4.50-4.43 (m, 1H), 3.73-3.69 (m, 2H), 1.87-1.84 (m, 2H). |
| 400 | 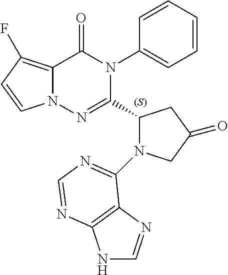 | 430.9 | ¹H NMR (400 MHz, DMSO-d₆) δ 8.34 (s, 1H), 8.24 (bs, 1H), 7.71-7.63 (m, 2H), 7.61-7.53 (m, 4H), 7.37 (d, J = 6.4 Hz, 1H), 6.40 (d, J = 2.8 Hz, 1H), 4.12-4.06 (m, 1H), 3.17 (s, 2H), 3.09 (d, J = 13.6 Hz, 1 H), 2.87-2.80 (m, 1H). |
| 401 | 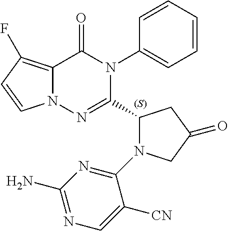 | 430.9 | ¹H NMR (400 MHz, DMSO-d₆) δ 8.31 (s, 1H), 7.89 (d, J = 8.0 Hz, 1H), 7.64-7.60 (m, 1H), 7.56-7.53 (m, 3H), 7.50-7.48 (m, 1H), 6.45 (d, J = 3.2 Hz, 1H), 5.33-5.31 (m, 0.2H), 5.12 (d, J = 8.8 Hz, 0.8H), 4.53 (d, J = 17.2 Hz, 0.5H), 4.23 (d, J = 17.2 Hz, 1H), 4.13-4.11 (m, 0.5H), 3.17 (d, J = 4.8 Hz, 2H), 2.99 (d, J = 18.8 Hz, 1H), 2.68-2.58 (m, 1H). |
| 402 | 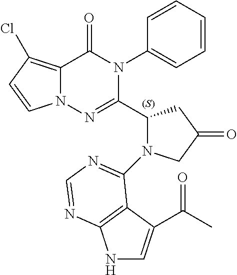 | 488.1 | ¹H NMR (400 MHz, DMSO-d₆) δ 12.52 (br, 1H), 8.26 (s, 1H), 8.25 (s, 1H), 7.64 (d, J = 7.4 Hz, 1H), 7.56-7.55 (m, 2H), 7.49-7.40 (m, 3H), 6.59 (dd, J = 2.9, 0.7 Hz, 1H), 5.28 (d, J = 8.9 Hz, 1H), 4.35-4.22 (m, 2H), 3.00 (d, J = 17.7 Hz, 1H), 2.82-2.75 (m, 1H), 2.50 (s, 3H). |
| 403 | 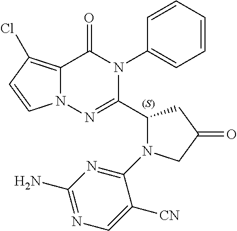 | 447.1 | ¹H NMR (400 MHz, DMSO-d6) δ 8.31 (d, J = 1.0 Hz, 1H), 7.90 (d, J = 7.6 Hz, 1H), 7.65-7.53 (m, 5H), 7.32-7.01 (br, 2H), 6.62 (dd, J = 3.0, 1.0 Hz, 1H), 5.10 (d, J = 9.8 Hz, 1H), 4.51 (d, J = 17.2 Hz, 1H), 4.23 (d, J = 17.3 Hz, 1H), 2.99 (d, J = 17.9 Hz, 1H), 2.64-2.57 (m, 1H). |
| 404 | 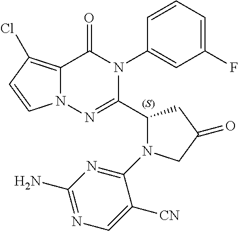 | 465.1 | ¹H NMR (400 MHz, DMSO-d₆) δ 8.32 (d, J = 1.6 Hz, 1H), 7.95-7.40 (m, 5H), 7.16 (br, 2H), 6.64-6.63 (m, 1H), 5.14 (d, J = 9.5 Hz, 0.5H), 5.04 (d, J = 8.5 Hz, 0.5H), 4.52 (dd, J = 17.2, 11.5 Hz, 1H), 4.24 (dd, J = 17.6, 6.6 Hz, 1H), 3.05-2.91 (m, 1H), 2.76-2.60 (m, 1H). |
| 445 | 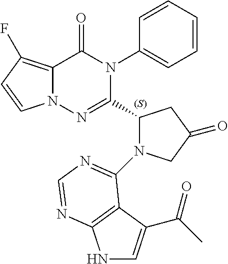 | 472.0 | ¹H NMR (400 MHz, DMSO-d₆ + D₂O) δ 8.20 (s, 2H), 7.57 (d, J = 7.6 Hz, 1H), 7.51 (brs, 2H), 7.45-7.41 (m, 1H), 7.38-7.35 (m, 1H), 7.32-7.30 (m, 1H), 6.39 (d, J = 3.2 Hz, 1H), 5.29 (d, J = 7.2 Hz, 1H), 4.29-4.17 (m, 2H), 2.77-2.70 (m, 1H), 2.52 (s, 3H). |
| 446 | 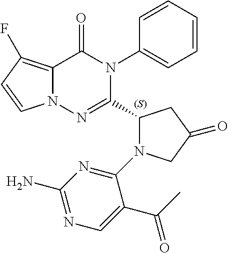 | 447.9 | ¹H NMR (400 MHz, DMSO-d₆) δ 8.51 (s, 1H), 7.78 (d, J = 8.0 Hz, 1H), 7.54-7.49 (m, 4H), 7.34-7.32 (m, 1H), 6.40 (d, J = 3.2 Hz, 1H), 5.31-5.28 (m, 1H), 5.15 (d, J = 8.0 Hz, 1H), 3.79-3.65 (m, 1H), 2.93 (d, J = 19.2 Hz, 1H), 2.66-2.54 (m, 1H), 2.42 (s, 3H). |

### Example 59

### Compound 189

### (S)-4-amino-6-(2-(5-methyl-4-oxo-3-phenyl-3,4-dihydropyrrolo[2,1-f][1,2,4]triazin-2-yl)pyrrolidin-1-yl)pyrimidine-5-carbonitrile

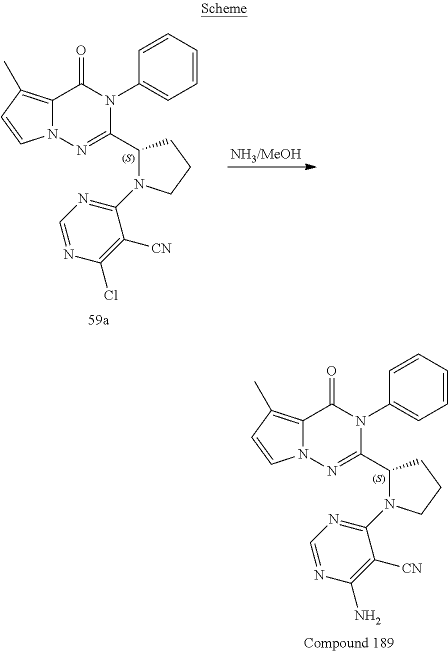

To a solution of 59a (prepared according to the procedures described in Example 1 using the corresponding reagents and intermediates) (49 mg, 0.11 mmol) in MeOH was added NH₃/MeOH (7 N, 5 mL), the mixture was stirred at reflux for 1 h, then concentrated and purified by flash column chromatography to give Compound 189 as a yellow solid. Yield: 44%. ¹H NMR (400 MHz, CDCl₃) δ 8.05 (s, 1H), 7.71-7.44 (m, 5H), 7.16 (d, J=2.5 Hz, 1H), 6.29 (d, J=2.1 Hz, 1H), 5.56 (s, 2H), 4.88-4.87 (m, 1H), 4.30-4.20 (m, 1H), 3.96-3.89 (m, 1H), 2.49 (s, 3H), 2.40-2.30 (m, 1H), 2.00-1.89 (m, 3H). MS (m/z): 412.7 (M+1)⁺.

### Example 60

### Compounds 382 and 383

### 5-chloro-2-((S)-1-(5-((S)-methylsulfinyl)-7H-pyrrolo[2,3-d]pyrimidin-4-yl)pyrrolidin-2-yl)-3-phenylpyrrolo[2,1-f][1,2,4]triazin-4(3H)-one and

### 5-chloro-2-((S)-1-(5-((R)-methylsulfinyl)-7H-pyrrolo[2,3-d]pyrimidin-4-yl)pyrrolidin-2-yl)-3-phenylpyrrolo[2,1-f][1,2,4]triazin-4(3H)-one

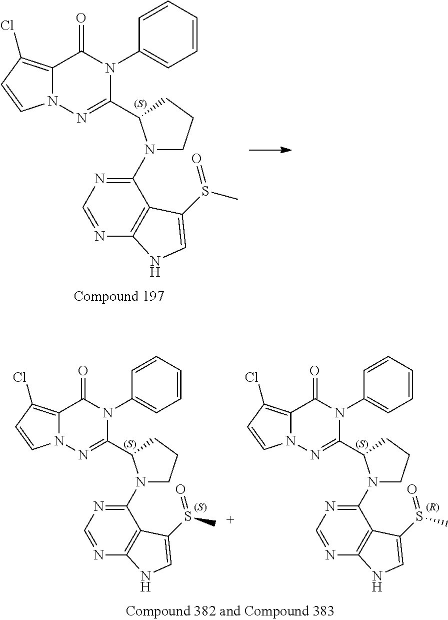

Compound 197 were resolved by chiral HPLC to produce the optically pure enantiomers Compound 382 and Compound 383. HPLC conditions: Gilson system, Column: CHIRALPAK Ia 20 mm I.D.×25 cm L; mobile phase: n-hexane/i-PrOH/DEA=7/3/0.01; flow rate, 10 mL/min; detector: UV 254 nm.

Compound 382 is the first eluent with at least 98% ee. ¹H NMR (400 MHz, DMSO-d6) δ 8.24 (s, 1H), 7.74 (d, J=8.2 Hz, 1H), 7.68-7.54 (m, 5H), 7.39 (d, J=3.0 Hz, 1H), 6.59 (d, J=3.0 Hz, 1H), 4.80-4.76 (m, 1H), 3.87-3.79 (m, 2H), 2.93 (s, 1H), 2.15-2.07 (m, 2H), 2.00-1.94 (m, 1H), 1.85-1.73 (m, 1H). MS (m/z): 494.1 (M+1)⁺.

Compound 383 is the second eluent with at least 98% ee. ¹H NMR (400 MHz, DMSO-d6) δ 8.23 (s, 1H), 7.85 (s, 1H), 7.77 (d, J=8.0 Hz, 1H), 7.64-7.53 (m, 4H), 7.49 (d, J=3.0 Hz, 1H), 6.58 (d, J=3.0 Hz, 1H), 4.68-4.65 (m, 1H), 4.25-4.18 (m, 1H), 3.69-3.63 (m, 1H), 2.88 (s, 3H), 2.29-2.18 (m, 2H), 1.97-1.88 (m, 2H). MS (m/z): 494.2 (M+1)⁺.

### Compounds 384 and 385

### (R)-2-amino-4-((1-(3-chloro-5-oxo-6-phenyl-5,6-dihydroimidazo[1,2-c]pyrimidin-7-yl)ethyl)amino)pyrimidine-5-carbonitrile and

### (S)-2-amino-4-((1-(3-chloro-5-oxo-6-phenyl-5,6-dihydroimidazo[1,2-c]pyrimidin-7-yl)ethyl)amino)pyrimidine-5-carbonitrile

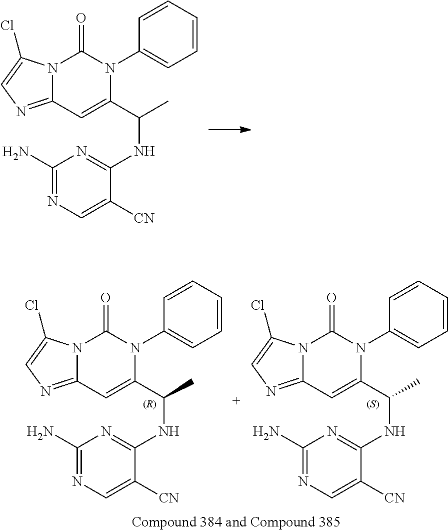

2-amino-4-((1-(3-chloro-5-oxo-6-phenyl-5,6-dihydroimidazo[1,2-c]pyrimidin-7-yl)ethyl)amino)pyrimidine-5-carbonitrile was resolved by chiral HPLC to produce the optically pure enantiomers Compound 384 and Compound 385. HPLC conditions: Gilson system, Column: CHIRALPAK Ia 20 mm I.D.×25 cm L; mobile phase: EtOH/DEA=100/0.1; flow rate, 8 mL/min; detector: UV 254 nm.

Compound 384 is the first eluent with at least 95% ee. MS (m/z): 407.0 (M+1)⁺.

Compound 385 is the second eluent with at least 90% ee. MS (m/z): 407.0 (M+1)⁺.

### Compounds 386 and 387

### 5-chloro-3-(3-fluorophenyl)-2-((S)-1-(5-((S)-methylsulfinyl)-7H-pyrrolo[2,3-d]pyrimidin-4-yl)pyrrolidin-2-yl)pyrrolo[2,1-f][1,2,4]triazin-4(3H)-one and

### 5-chloro-3-(3-fluorophenyl)-2-((S)-1-(5-((R)-methylsulfinyl)-7H-pyrrolo[2,3-d]pyrimidin-4-yl)pyrrolidin-2-yl)pyrrolo[2,1-f][1,2,4]triazin-4(3H)-one

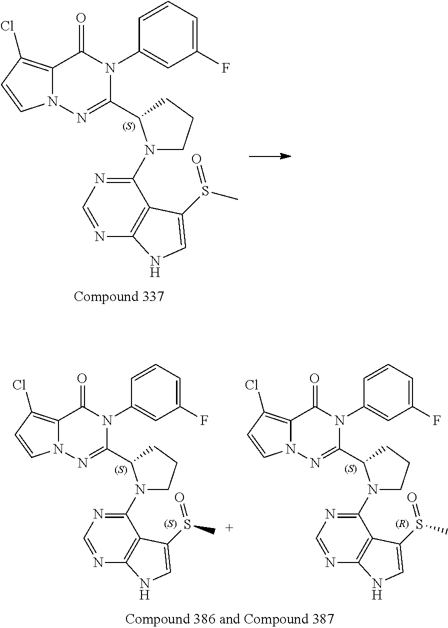

Compound 337 was resolved by p-TLC to produce the optically pure enantiomers Compound 386 and Compound 387 with at least 98% ee.

Under the HPLC analysis conditions below, the retention time of Compound 386 is 8.93 min, the retention time of Compound 387 is 8.61 min.

HPLC analysis conditions: Gilson system, Column: Daicel 4.6*250 mm IA; mobile phase: EtOH/DEA=100/0.1; flow rate, 0.5 mL/min; detector: UV 254 nm.

Compound 386: MS (m/z): 512.0 (M+1)⁺.

Compound 387: MS (m/z): 512.0 (M+1)⁺.

### Compounds 388 and 389

### 5-chloro-2-((S)-1-(5-((S)-methylsulfinyl)-7H-pyrrolo[2,3-d]pyrimidin-4-yl)pyrrolidin-2-yl)-3-(pyridin-2-yl)pyrrolo[2,1-f][1,2,4]triazin-4(3H)-one and

### 5-chloro-2-((S)-1-(5-((R)-methylsulfinyl)-7H-pyrrolo[2,3-d]pyrimidin-4-yl)pyrrolidin-2-yl)-3-(pyridin-2-yl)pyrrolo[2,1-f][1,2,4]triazin-4(3H)-one

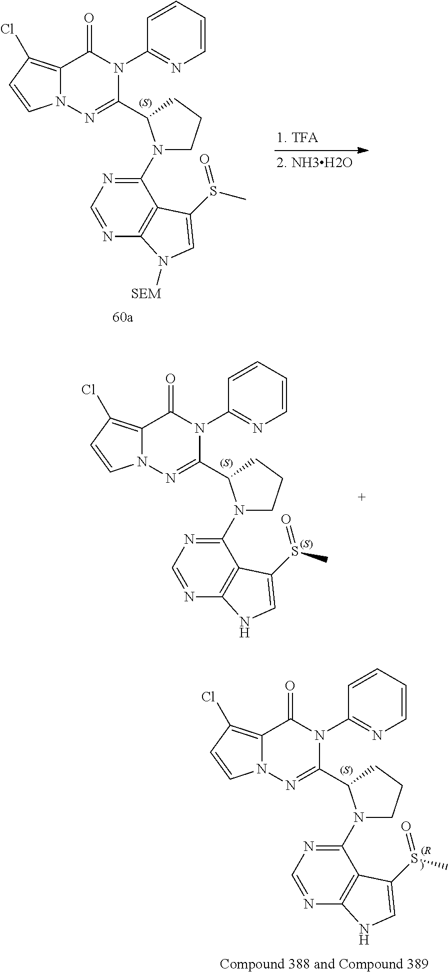

The mixture of 60a (prepared according to the procedures described in Example 41 using the corresponding reagents and intermediates) in TFA (2 mL) was stirred at r.t. for 1 h. The mixture was concentrated, the resulting residue was dissolved in MeOH (2 mL), and treated with NH₃.H₂O (25%), the reaction was stirred at r.t. for another 1 h. The mixture was concentrated and purified by flash column chromatography and p-TLC to give Compound 388 and Compound 389 as two yellow solids with at least 98% ee. Under the HPLC analysis conditions below, the retention time of Compound 388 is 8.91 min, the retention time of Compound 389 is 11.22 min.

HPLC analysis conditions: Gilson system, Column: Daicel 4.6*250 mm IA; mobile phase: Hexane: i-PrOH: Et₂NH=70:30:0.1; flow rate, 1 mL/min; detector: UV 254 nm.

Compound 388: ¹H NMR (400 MHz, DMSO-d₆) δ 8.70 (d, J=4.3 Hz, 1H), 8.11 (t, J=7.4 Hz, 1H), 8.06 (s, 1H), 7.83 (br, 1H), 7.71 (s, 1H), 7.64-7.59 (m, 1H), 7.51 (d, J=2.0 Hz, 1H), 6.63 (d, J=2.0 Hz, 1H), 4.73-4.54 (m, 1H), 3.90-3.85 (m, 2H), 2.87 (s, 3H), 2.15-2.10 (m, 2H), 2.04-1.97 (m, 1H), 1.82-1.75 (m, 1H). MS (m/z): 495.0 (M+1)⁺.

Compound 389: ¹H NMR (400 MHz, DMSO-d₆) δ 8.66 (s, 1H), 8.18 (s, 1H), 8.12-8.02 (m, 1H), 7.91-7.77 (m, 2H), 7.61-7.48 (m, 2H), 6.58 (d, J=2.9 Hz, 1H), 4.58-4.38 (m, 1H), 4.15-4.02 (m, 1H), 3.68-3.62 (m, 1H), 2.85 (s, 3H), 2.30-2.12 (m, 2H), 2.08-2.00 (m, 1H), 1.98-1.91 (m, 1H). MS (m/z): 495.1 (M+1)⁺.

### Compounds 390 and 391

### 5-chloro-2-((S)-1-(5-((S)-methylsulfinyl)-7H-pyrrolo[2,3-d]pyrimidin-4-yl)azetidin-2-yl)-3-(pyridin-2-yl)pyrrolo[2,1-f][1,2,4]triazin-4(3H)-one and

### 5-chloro-2-((S)-1-(5-((R)-methylsulfinyl)-7H-pyrrolo[2,3-d]pyrimidin-4-yl)azetidin-2-yl)-3-(pyridin-2-yl)pyrrolo[2,1-f][1,2,4]triazin-4(3H)-one

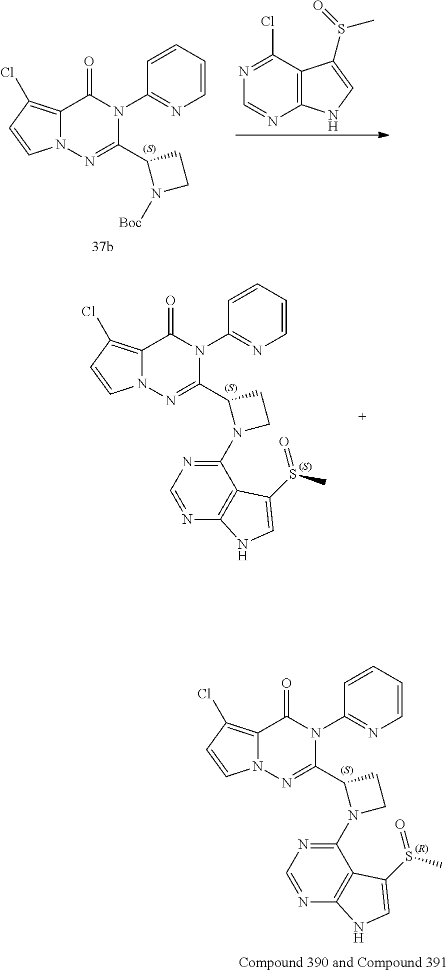

37b (40 mg, 0.1 mmol) was dissolved in MeOH (2 mL) and conc.HCl (2 mL), the mixture was concentrated at 50° C., the resulting residue was dissolved in n-BuOH (2 mL) and DIPEA (0.5 mL), then was added 4-chloro-5-(methylsulfinyl)-7H-pyrrolo[2,3-d]pyrimidine (21 mg, 0.1 mmol), the reaction as stirred at reflux for 3 h, then concentrated and purified by flash column chromatography to give Compound 390 and Compound 391 with at least 98% ee.

Under the HPLC analysis conditions below, the retention time of Compound 390 is 10.53 min, the retention time of Compound 391 is 11.64 min.

HPLC analysis conditions: Gilson system, Column: Daicel 4.6*250 mm IA; mobile phase: EtOH/DEA=100/0.1; flow rate, 0.5 mL/min; detector: UV 254 nm.

Compound 390: ¹H NMR (400 MHz, DMSO-d6) δ 8.71-8.70 (m, 1H), 8.17 (s, 1H), 8.11-8.07 (m, 1H), 7.78 (s, 1H), 7.72 (d, J=7.6 Hz, 1H), 7.64-7.60 (m, 2H), 6.67 (d, J=2.8 Hz, 1H), 5.21-5.18 (m, 1H), 4.34-4.29 (m, 1H), 3.94-3.88 (m, 1H), 2.88 (s, 3H), 2.56-2.55 (m, 1H), 1.90 (br, 1H). MS (m/z): 481.0 (M+1)⁺.

Compound 391: ¹H NMR (400 MHz, DMSO-d6) δ 8.70 (s, 1H), 8.16 (s, 1H), 8.11-8.07 (m, 1H), 7.87 (s, 1H), 7.73-7.69 (m, 2H), 7.62-7.59 (m, 1H), 6.66 (br, 1H), 5.18 (br, 1H), 4.59 (br, 1H), 3.78-3.76 (m, 1H), 2.91 (s, 3H), 2.54 (br, 1H), 1.83 (br, 1H). MS (m/z): 481.0 (M+1)⁺.

### Compounds 348 and 349

### (3S,5S)-5-(5-chloro-3-(3-fluorophenyl)-4-oxo-3,4-dihydropyrrolo[2,1-f][1,2,4]triazin-2-yl)-1-(5-((S)-methylsulfinyl)-7H-pyrrolo[2,3-d]pyrimidin-4-yl)pyrrolidine-3-carbonitrile and

### (3S,5S)-5-(5-chloro-3-(3-fluorophenyl)-4-oxo-3,4-dihydropyrrolo[2,1-f][1,2,4]triazin-2-yl)-1-(5-((R)-methylsulfinyl)-7H-pyrrolo[2,3-d]pyrimidin-4-yl)pyrrolidine-3-carbonitrile

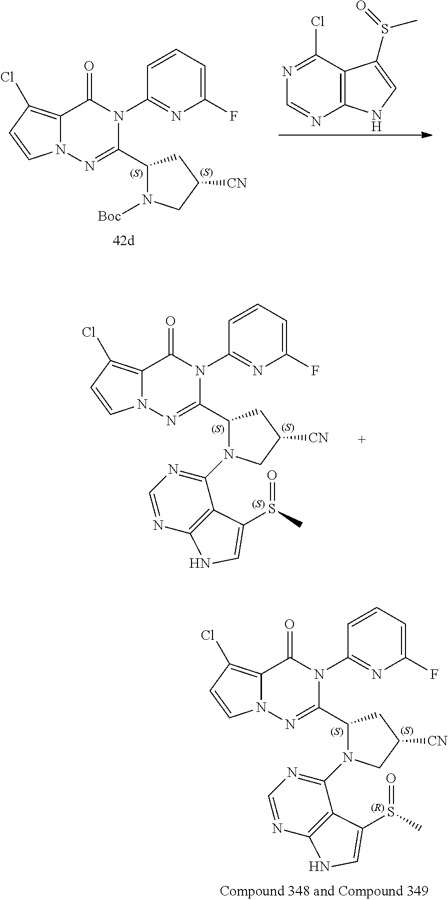

Compound 348 and Compound 349 with at least 98% ee were prepared similar to Compound 390 and Compound 391.

Under the HPLC analysis conditions below, the retention time of Compound 348 is 7.99 min, the retention time of Compound 349 is 7.83 min.

HPLC analysis conditions: Gilson system, Column: Daicel 4.6*250 mm IA; mobile phase: EtOH/DEA=100/0.1; flow rate, 0.5 mL/min; detector: UV 254 nm.

Compound 348: ¹H NMR (400 MHz, CD₃OD) δ 8.26 (s, 0.5H), 8.25 (s, 0.5H), 7.82 (s, 0.5H), 7.81 (s, 0.5H), 7.60-7.47 (m, 2H), 7.34-7.25 (m, 3H), 6.50 (d, J=3.2 Hz, 0.5H), 6.49 (d, J=3.2 Hz, 0.5H), 5.28-5.21 (m, 1H), 4.28-4.12 (m, 2H), 3.34-3.32 (m, 1H), 3.06 (s, 1.5H), 3.06 (s, 1.5H), 2.59-2.46 (m, 2H). MS (m/z): 537.1 (M+1)⁺.

Compound 349: ¹H NMR (400 MHz, CD₃OD) δ 8.13 (s, 0.5H), 8.12 (s, 0.5H), 7.92 (s, 0.5H), 7.91 (s, 0.5H), 7.52-7.46 (m, 1H), 7.39-7.33 (m, 1H), 7.29 (d, J=2.8 Hz, 0.5H), 7.287 (d, J=2.8 Hz, 0.5H), 7.23-7.20 (m, 1H), 7.15-7.05 (m, 1H), 6.43 (d, J=2.8 Hz, 0.5H), 6.42 (d, J=3.2 Hz, 0.5H), 5.40-5.23 (m, 1H), 4.41-4.35 (m, 1H), 4.15-4.09 (m, 1H), 3.28-3.24 (m, 1H), 3.05 (s, 3H), 2.60-2.43 (m, 2H). MS (m/z): 537.1 (M+1)⁺.

### Compounds 392 and 393

### 5-chloro-2-((2S,4S)-4-fluoro-1-(5-((S)-methylsulfinyl)-7H-pyrrolo[2,3-d]pyrimidin-4-yl)pyrrolidin-2-yl)-3-(3-fluorophenyl)pyrrolo[2,1-f][1,2,4]triazin-4(3H)-one and

### 5-chloro-2-((2S,4S)-4-fluoro-1-(5-((R)-methylsulfinyl)-7H-pyrrolo[2,3-d]pyrimidin-4-yl)pyrrolidin-2-yl)-3-(3-fluorophenyl)pyrrolo[2,1-f][1,2,4]triazin-4(3H)-one

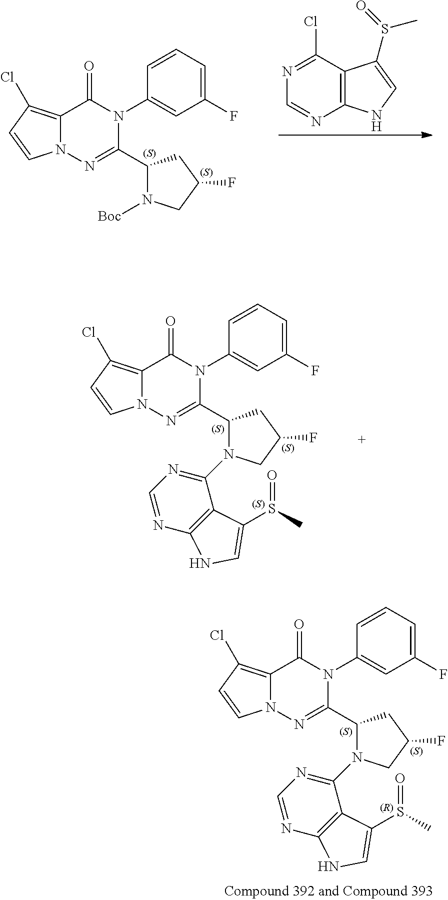

Compound 392 and Compound 393 were prepared similar to Compound 390 and Compound 391.

Under the HPLC analysis conditions below, the retention time of Compound 392 is 7.23 min, the retention time of Compound 393 is 9.20 min.

HPLC analysis conditions: Gilson system, Column: Daicel 4.6*250 mm IA; mobile phase: Hexane: i-PrOH: Et₂NH=70:30:0.1; flow rate, 1 mL/min; detector: UV 254 nm.

Compound 392: ¹H NMR (400 MHz, CD₃OD) δ 8.20 (d, J=0.8 Hz, 1H), 7.89 (s, 1H), 7.62-7.51 (m, 2H), 7.36-7.27 (m, 2H), 7.24 (dd, J=4.2, 3.0 Hz, 1H), 6.46 (dd, J=3.0, 1.5 Hz, 1H), 5.37-5.29 (m, 1H), 5.19-5.11 (m, 1H), 4.44-4.31 (m, 1H), 4.11-3.97 (m, 1H), 3.09 (s, 3H), 2.46-2.32 (m, 2H). MS (m/z): 530.1 (M+1)⁺.

Compound 393: ¹H NMR (400 MHz, CD₃OD) δ 8.30 (s, 1H), 7.96 (s, 1H), 7.68-7.51 (m, 2H), 7.42-7.26 (m, 2H), 7.25 (br, 1H), 6.45 (br, 1H), 5.46-5.25 (m, 1H), 5.24-5.11 (m, 1H), 4.93 (m, 1H), 4.05-3.85 (m, 1H), 3.09 (s, 3H), 2.62-2.24 (m, 2H). MS (m/z): 530.1 (M+1)⁺.

### Compounds 394 and 395

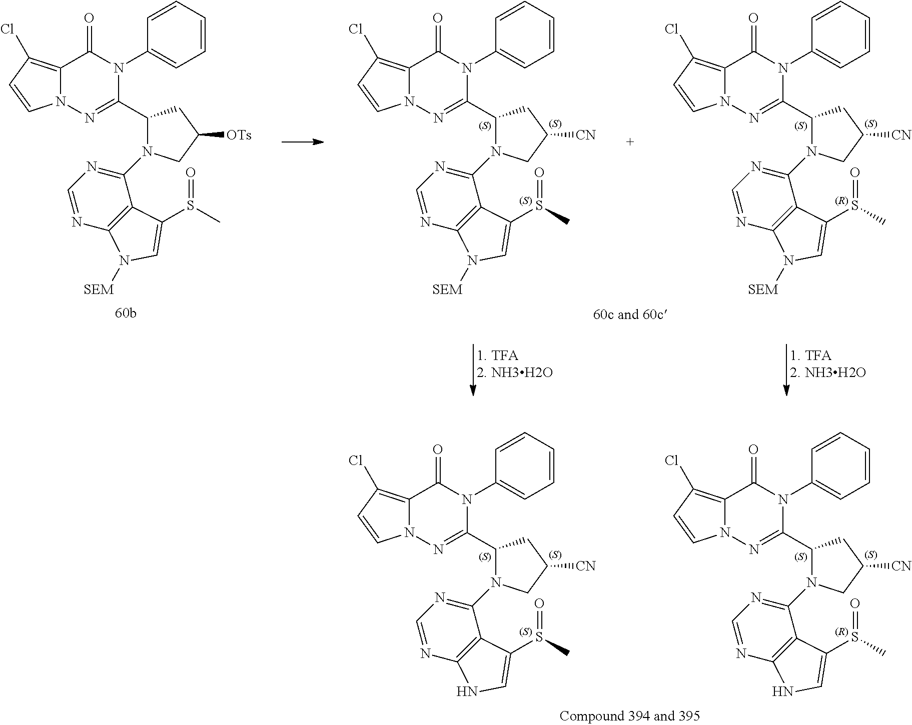

According to the procedures described in Example 48 using the corresponding reagents and intermediates, 60c and 60c′ were given after purification by flash column chromatography from the reaction of 60b and NaCN in DMSO.

The solution of 60c (30 mg, 0.046 mmol) in TFA (5 mL) was stirred at 0° C. for 1 h, then concentrated, the resulting residue was dissolved in MeOH (5 mL), and treated with NH₃.H₂O (2 mL), the mixture was stirred at r.t for 1 h, then concentrated and purified by p-TLC to give Compound 394 as a yellow solid. ¹H NMR (400 MHz, CD₃OD) δ 8.20 (s, 1H), 7.86 (s, 1H), 7.63-7.41 (m, 5H), 7.29 (d, J=3.0 Hz, 1H), 6.49 (d, J=3.0 Hz, 1H), 5.24 (t, J=7.6 Hz, 1H), 4.28-4.13 (m, 2H), 3.28-3.22 (m, 1H), 3.06 (s, 3H), 2.54-2.47 (m, 2H). MS (m/z): 519.1 (M+1)⁺.

Compound 395 was prepared according to the procedure of Compound 394. ¹H NMR (400 MHz, CD₃OD) δ 8.14 (s, 1H), 7.99 (s, 1H), 7.61-7.51 (m, 2H), 7.44-7.38 (m, 2H), 7.36 (d, J=3.0 Hz, 1H), 7.30-7.26 (m, 1H), 6.50 (d, J=3.0 Hz, 1H), 5.38-5.36 (m, 1H), 4.47-4.45 (m, 1H), 4.17-4.15 (m, 1H), 3.27-3.20 (m, 1H), 3.12 (s, 3H), 2.65-2.46 (m, 2H). MS (m/z): 519.1 (M+1)⁺.

Under the HPLC analysis conditions below, the retention time of Compound 394 is 8.22 min, the retention time of Compound 395 is 8.24 min.

HPLC analysis conditions: Gilson system, Column: Daicel 4.6*250 mm IA; mobile phase: EtOH/DEA=100/0.1; flow rate, 0.5 mL/min; detector: UV 254 nm.

### Compounds 396 and 397

### 5-fluoro-2-((S)-1-(5-((S)-methylsulfinyl)-7H-pyrrolo[2,3-d]pyrimidin-4-yl)pyrrolidin-2-yl)-3-phenylpyrrolo[2,1-f][1,2,4]triazin-4(3H)-one and

### 5-fluoro-2-((S)-1-(5-((R)-methylsulfinyl)-7H-pyrrolo[2,3-d]pyrimidin-4-yl)pyrrolidin-2-yl)-3-phenylpyrrolo[2,1-f][1,2,4]triazin-4(3H)-one

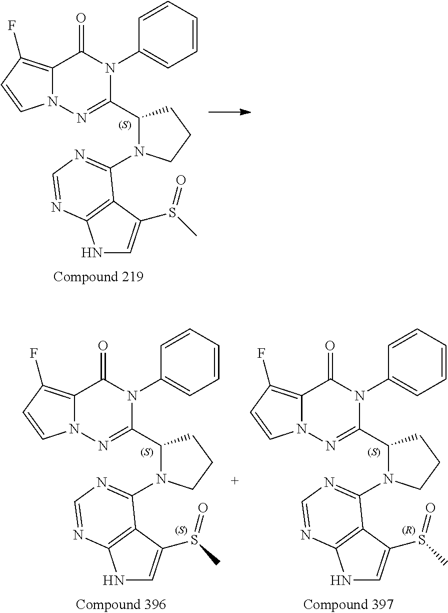

Compound 219 was resolved by p-TLC to produce the optically pure enantiomers Compound 396 and Compound 397 with at least 98% ee.

Under the HPLC analysis conditions below, the retention time of Compound 396 is 8.83 min, the retention time of Compound 397 is 8.50 min.

HPLC analysis conditions: Gilson system, Column: Daicel 4.6*250 mm IA; mobile phase: EtOH/DEA=100/0.1; flow rate, 0.5 mL/min; detector: UV 254 nm.

Compound 396: ¹H NMR (400 MHz, DMSO-d₆) δ 12.37 (brs, 1H), 8.25 (s, 1H), 7.73 (d, J=7.9 Hz, 1H), 7.67-7.54 (m, 5H), 7.26 (m, 1H), 6.413 (d, J=3.2 Hz, 1H), 4.79 (t, J=7.2 Hz, 1H), 3.84-3.80 (m, 2H), 2.93 (s, 3H), 2.11-2.05 (m, 2H), 2.01-1.96 (m, 1H), 1.81-1.76 (m, 1H). MS (m/z): 478.1 (M+1)⁺.

Compound 397: ¹H NMR (400 MHz, DMSO-d₆) δ 12.40 (brs, 1H), 8.26 (s, 1H), 7.87 (s, 1H), 7.78-7.75 (m, 1H), 7.64-7.52 (m, 4H), 7.38-7.37 (m, 1H), 6.40 (d, J=3.2 Hz, 1H), 4.68-4.66 (m, 1H), 4.17-4.15 (m, 1H), 3.69-3.67 (m, 1H), 2.88 (s, 3H), 2.33-2.19 (m, 2H), 2.01-1.89 (m, 2H). MS (m/z): 478.1 (M+1)⁺.

### Compounds 405 and 406

### (R)-3-(1-((5-acetyl-7H-pyrrolo[2,3-d]pyrimidin-4-yl)amino)ethyl)-8-fluoro-2-phenylpyrrolo[1,2-a]pyrazin-1(2H)-one and

### (S)-3-(1-((5-acetyl-7H-pyrrolo[2,3-d]pyrimidin-4-yl)amino)ethyl)-8-fluoro-2-phenylpyrrolo[1,2-a]pyrazin-1(2H)-one

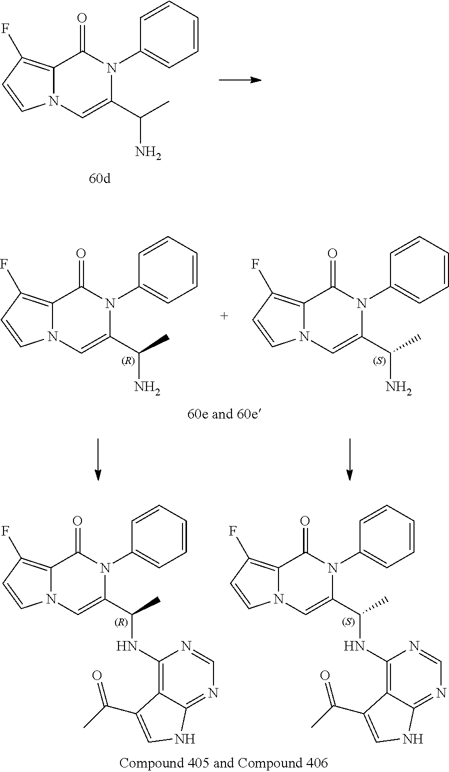

60d (prepared according to the procedures described in Example 6 using the corresponding reagents and intermediates) was resolved chiral HPLC to produce the optically pure enantiomers 60e and 60e′. HPLC conditions: Gilson system, Column: CHIRALPAK Ia 20 mm I.D.×25 cm L; mobile phase: Hexane/EtOH/Et₂NH=70/30/0.1; flow rate:10 mL/min; detector: UV 254 nm.

60e is the first eluent, 60e′ is the second eluent.

Compound 405 was prepared from 60e according to the procedures described in Example 6 using the corresponding reagents and intermediates. ¹H NMR (400 MHz, CD₃OD) δ 9.21 (d, J=7.0 Hz, 1H), 8.09 (d, J=0.9 Hz, 1H), 7.94 (s, 1H), 7.46-7.41 (m, 2H), 7.33 (d, J=7.9 Hz, 1H), 7.23-7.18 (m, 3H), 6.98 (t, J=7.7 Hz, 1H), 6.38-6.37 (m, 1H), 4.93-4.88 (m, 1H), 2.53 (s, 3H), 1.47 (d, J=6.7 Hz, 3H). MS (m/z): 431.1 (M+1)⁺.

Compound 406 was prepared from 60e′ according to the procedures described in Example 6 using the corresponding reagents and intermediates. ¹H NMR (400 MHz, CD₃OD) δ 9.21 (d, J=7.1 Hz, 1H), 8.09 (s, 1H), 7.94 (s, 1H), 7.46-7.41 (m, 2H), 7.33 (d, J=8.0 Hz, 1H), 7.23-7.18 (m, 3H), 6.97 (t, J=7.7 Hz, 1H), 6.398-6.38 (m, 1H), 4.93-4.88 (m, 1H), 2.53 (s, 3H), 1.47 (d, J=6.7 Hz, 3H). MS (m/z): 431.1 (M+1)⁺.

### Compound 407

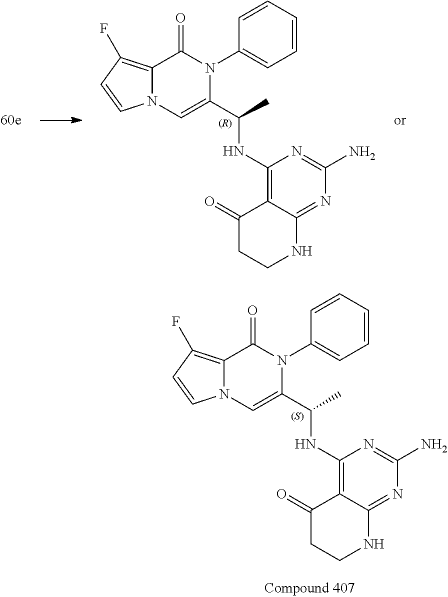

Compound 407 was prepared from 60e according to the procedures described in Example 1 using the corresponding reagents and intermediates. ¹H NMR (400 MHz, DMSO-d₆) δ 9.21 (d, J=7.6 Hz, 1H), 7.55-7.45 (m, 1H), 7.37-7.27 (m, 4H), 7.23-7.19 (m, 2H), 6.39-6.38 (m, 1H), 4.91-4.86 (m, 1H), 3.52-3.39 (m, 2H), 2.62-2.46 (m, 2H), 1.36 (d, J=6.8 Hz, 3H). MS (m/z): 434.1 (M+1)⁺.

### Compound 449

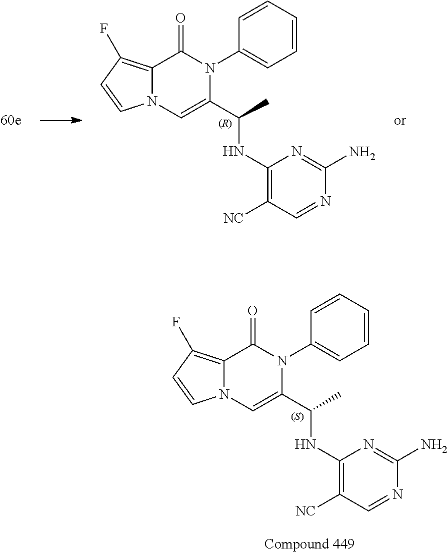

Compound 449 was prepared from 60e according to the procedures described in Example 6 using the corresponding reagents and intermediates. ¹H NMR (400 MHz, CD₃OD) δ 7.99 (brs, 1H), 7.45 (t, J=6.9 Hz, 1H), 7.39 (brs, 1H), 7.29-7.20 (m, 5H), 6.39-6.38 (m, 1H), 5.07-5.02 (m, 1H), 1.39 (d, J=6.6 Hz, 3H). MS (m/z): 390.1 (M+1)⁺.

### Compound 452

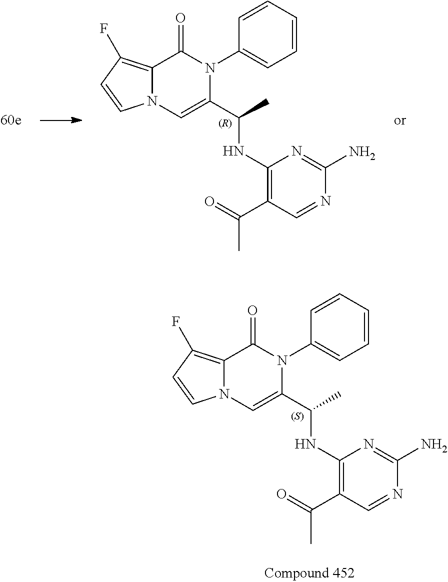

Compound 452 was prepared from 60e according to the procedures described in Example 6 using the corresponding reagents and intermediates. ¹H NMR (400 MHz, CD₃OD) δ 9.10 (d, J=7.5 Hz, 1H), 8.45 (s, 1H), 7.48-7.44 (m, 1H), 7.38-7.38 (m, 1H), 7.30-7.27 (m, 2H), 7.22-7.17 (m, 2H), 7.15-7.12 (m, 1H), 6.38 (d, J=3.1 Hz, 1H), 5.01-4.93 (m, 1H), 2.40 (s, 3H), 1.36 (d, J=6.8 Hz, 3H). MS (m/z): 407.1 (M+1)⁺.

### Compound 447 and 448

### (S)-7-(1-((5-acetyl-2-aminopyrimidin-4-yl)amino)ethyl)-3-chloro-6-phenylimidazo[1,2-c]pyrimidin-5(6H)-one and (R)-7-(1-((5-acetyl-2-aminopyrimidin-4-yl)amino)ethyl)-3-chloro-6-phenylimidazo[1,2-c]pyrimidin-5(6H)-one

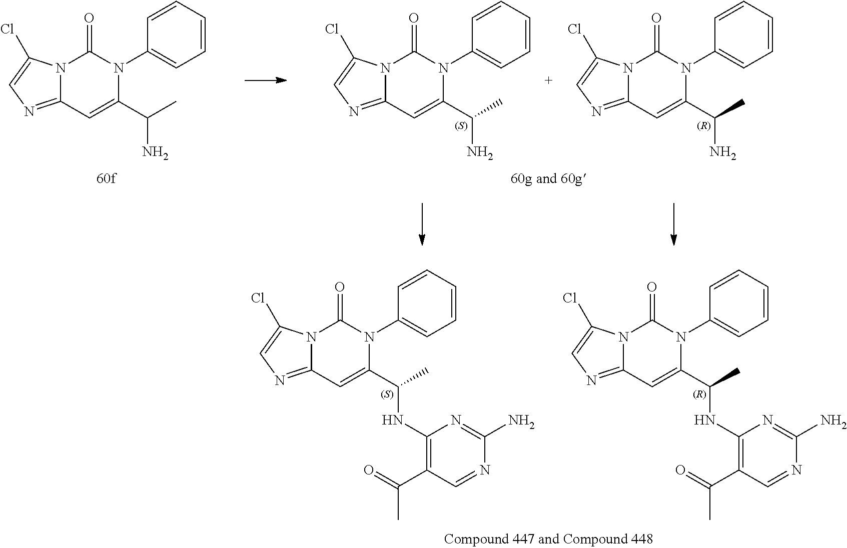

60f (prepared according to the procedures described in Example 19 using the corresponding reagents and intermediates) was resolved chiral HPLC to produce the optically pure enantiomers 60g and 60g′. HPLC conditions: Gilson system, Column: CHIRALPAK Ia 20 mm I.D.×25 cm L; mobile phase: EtOH/Et₂NH=100/0.1; flow rate: 8 mL/min; detector: UV 254 nm.

60g is the first eluent, 60g′ is the second eluent.

Compound 447 was prepared from 60g according to the procedures described in Example 38 using the corresponding reagents and intermediates. ¹H NMR (400 MHz, DMSO-d₆) δ 9.27 (d, J=7.6 Hz, 1H), 8.54 (s, 1H), 7.78-7.73 (m, 1H), 7.61-7.57 (m, 1H), 7.55-7.48 (m, 1H), 7.47-7.41 (m, 2H), 7.37 (s, 1H), 7.33-7.25 (m, 1H), 6.48 (s, 1H), 4.58-4.51 (m, 1H), 2.38 (s, 3H), 1.24 (d, J=6.8 Hz, 3H). MS (m/z): 424.2 (M+1)⁺.

Compound 448 was prepared from 60g′ according to the procedures described in Example 38 using the corresponding reagents and intermediates. 1H NMR (400 MHz, CD₃OD) δ 8.51 (s, 1H), 7.57-7.53 (m, 1H), 7.50-7.46 (m, 2H), 7.44-7.38 (m, 2H), 7.25 (s, 1H), 6.61 (s, 1H), 4.88-4.83 (m, 1H), 2.43 (s, 3H), 1.37 (d, J=6.8 Hz, 3H). MS (m/z): 424.2 (M+1)⁺.

### Compounds 450 and 451

### (S)-3-chloro-7-(1-((5-fluoro-7H-pyrrolo[2,3-d]pyrimidin-4-yl)amino)ethyl)-6-phenyl-imidazo[1,2-c]pyrimidin-5(6H)-one and (R)-3-chloro-7-(1-((5-fluoro-7H-pyrrolo[2,3-d]pyrimidin-4-yl)amino)ethyl)-6-phenylimidazo[1,2-c]pyrimidin-5(6H)-one

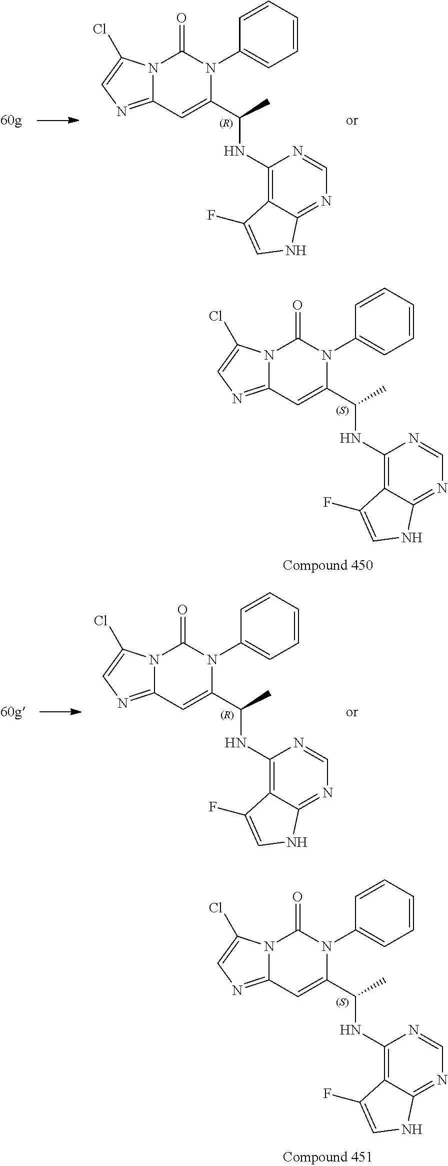

Compound 450 was prepared from 60g according to the procedures described in Example 1 using the corresponding reagents and intermediates. ¹H NMR (400 MHz, CD₃OD) δ 7.97 (s, 1H), 7.57 (d, J=8.0 Hz, 1H), 7.51-7.43 (m, 2H), 7.37-7.34 (m, 1H), 7.29-7.25 (m, 1H), 7.20 (d, J=1.2 Hz, 1H), 6.84 (d, J=2.8 Hz, 1H), 6.72 (s, 1H), 4.93-4.88 (m, 1H), 1.43 (d, J=6.8 Hz, 3H). MS (m/z): 424.1 (M+1)⁺.

Compound 451 was prepared from 60g′ according to the procedures described in Example 1 using the corresponding reagents and intermediates. ¹H NMR (400 MHz, CD₃OD) δ 8.00 (s, 1H), 7.61 (d, J=8.8 Hz, 1H), 7.54-7.46 (m, 2H), 7.40-7.37 (m, 1H), 7.31-7.28 (m, 1H), 7.23 (d, J=1.6 Hz, 1H), 6.87 (d, J=2.4 Hz, 1H), 6.75 (s, 1H), 4.96-4.41 (m, 1H), 1.65 (d, J=6.8 Hz, 3H). MS (m/z): 424.1 (M+1)⁺.

### Compounds 484 and 485

### (R)-2-(1-(5-acetyl-2-aminopyrimidin-4-yl)-3,3-dimethylazetidin-2-yl)-5-chloro-3-phenylpyrrolo[2,1-f][1,2,4]triazin-4(3H)-one and

### (S)-2-(1-(5-acetyl-2-aminopyrimidin-4-yl)-3,3-dimethylazetidin-2-yl)-5-chloro-3-phenylpyrrolo[2,1-f][1,2,4]triazin-4(3H)-one

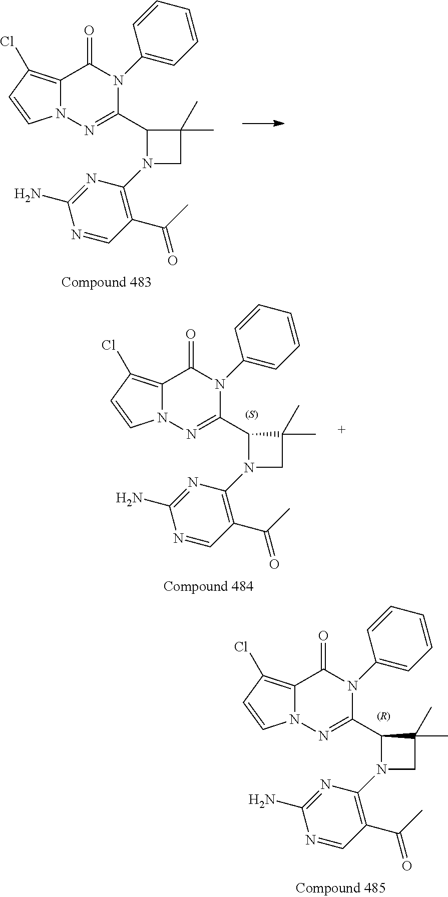

Compound 483 were resolved by chiral HPLC to produce the optically pure enantiomers Compound 484 and Compound 485. HPLC conditions: Gilson system, Column: CHIRALPAK Ia 20 mm I.D.×25 cm L; mobile phase: EtOH/DEA=100/0.1; flow rate, 8 mL/min; detector: UV 254 nm.

Compound 484 is the first eluent with at least 98% ee. MS (m/z): 464.2 (M+1)⁺.

Compound 485 is the second eluent with at least 98% ee. MS (m/z): 464.2 (M+1)⁺.

### Example 61

### Compound 486

### (S)-2-(1-(5-acetyl-2-aminopyrimidin-4-yl)azetidin-2-yl)-3-phenyl-5-(trifluoromethyl) pyrrolo[2,1-f][1,2,4]triazin-4(3H)-one

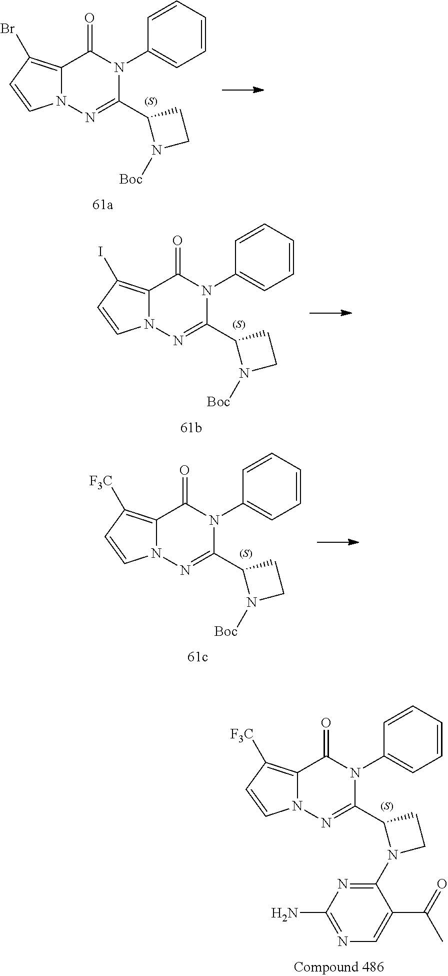

### Step 61-1 (S)-tert-butyl 2-(5-iodo-4-oxo-3-phenyl-3,4-dihydropyrrolo[2,1-f][1,2,4]triazin-2-yl)azetidine-1-carboxylate (61b)

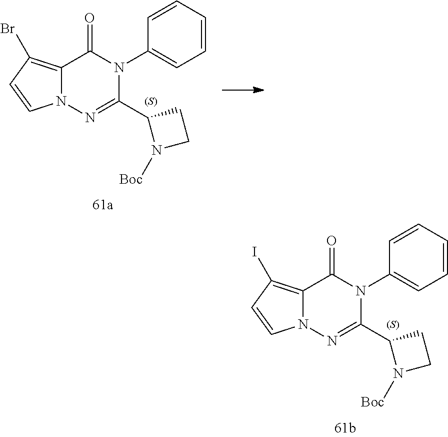

A solution of 61a (300 mg, 0.674 mmol)(prepared according to the procedure of Example 1), NaI (404 mg, 2.646 mmol), trans-1,2-bis(methylamino)cyclohexane (96 mg, 0.674 mmol) and CuI (64 mg, 0.337 mmol) in dioxane (8 ml) was stirred at reflux for 3 days. After cooling to the r.t., the reaction mixture was filtered through celite and washed with ethyl acetate, the resulting filtrate was concentrated and purified by chromatography to give 61b as a yellow solid. MS (m/z): 492.9 (M+H)⁺.

### Step 61-2 (5)-tert-butyl 2-(4-oxo-3-phenyl-5-(trifluoromethyl)-3,4-dihydropyrrolo[2,1-f][1,2,4]triazin-2-yl)azetidine-1-carboxylate (61c)

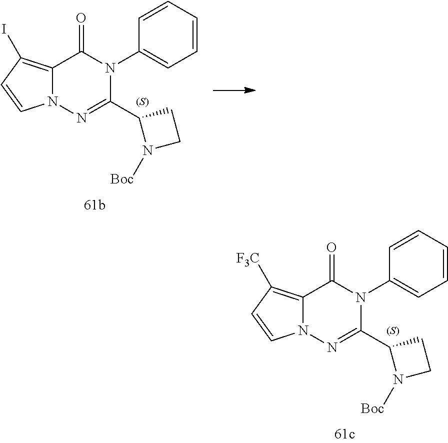

Under N₂ atmosphere 61b (200 mg, 0.4 mmol) and CuI (94 mg, 0.492 mmol) were dissolved in DMF (5 mL), to this mixture were added HMPA (0.35 mL, 2 mol) and methyl 2,2-difluoro-2-(fluorosulfonyl)acetate (0.25 mL, 2 mmol), the resulting mixture was stirred at 80° C. for 24 h, then poured into abundant ice-water and extracted with ethyl acetate. The organic layer was washed with water and brine, then concentrated and purified by chromatography to give 61c as a white solid. MS (m/z): 456.9 (M+Na)⁺.

### Step 61-3 (S)-2-(1-(5-acetyl-2-aminopyrimidin-4-yl)azetidin-2-yl)-3-phenyl-5-(trifluoromethyl)pyrrolo[2,1-f][1,2,4]triazin-4(3H)-one (Compound 486)

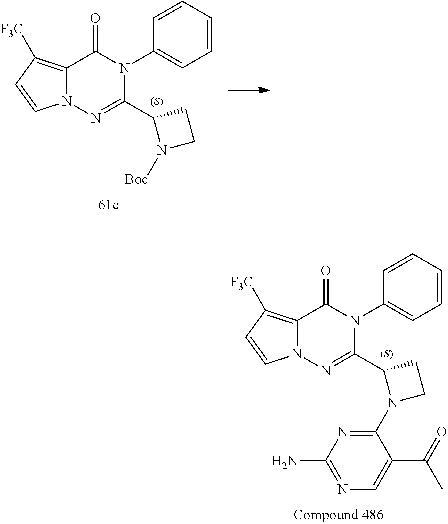

Compound 486 was prepared with 61c as the material according to the procedure of Example 1 from 1e to Compound 1. ¹H NMR (400 MHz, DMSO-d6) δ 8.47 (s, 1H), 7.770-7.55 (m, 5H), 7.46-7.43 (m, 1H), 6.95 (d, J=2.9, 1H), 6.82 (brs, 2H), 4.90 (brs, 1H), 4.20-4.14 (m, 1H), 3.49 (brs, 1H), 2.47-2.43 (m, 1H), 2.27 (brs, 3H), 1.92 (brs, 1H). MS (m/z): 470.1 (M+H)⁺.

The following Compounds were prepared according to the procedure of Compound 486 using the corresponding reagents and intermediates under appropriate conditions that will be recognized by one skilled in the art:

| Compd. | | LC/MS | |
|---|---|---|---|
| No. | Structure | (M + H)⁺ | NMR |
| 487 | 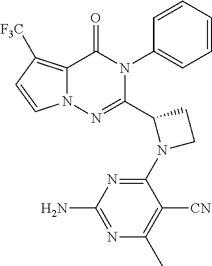 [SMILES: Cc1nc(N)nc(N2CC[C@H]2c2nn3ccc(C(F)(F)F)c3c(=O)n2-c2ccccc2)c1C#N] | 467.1 | ¹H NMR (400 MHz, DMSO-d6) δ 7.86 (d, J = 2.8, 1H), 7.64-7.56 (m, 4H), 7.47-7.46 (m, 1H), 6.98 (d, J = 2.9, 3H), 4.97 (brs, 1H), 4.08 (d, J = 13.2, 2H), 2.67-2.56 (m, 1H), 2.23 (s, 3H), 2.14- 2.04 (m, 1H). |
| 488 | 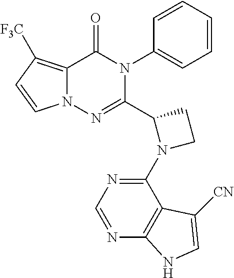 [SMILES: N#Cc1c[nH]c2ncnc(N3CC[C@H]3c3nn4ccc(C(F)(F)F)c4c(=O)n3-c3ccccc3)c12] | 477 | ¹H NMR (400 MHz, CD₃OD) δ 8.16 (s, 1H), 7.87 (s, 1H), 7.68-7.66 (m, 1H), 7.56-7.48 (m, 3H), 7.37 (d, J = 2.8 Hz, 1H), 7.33- 7.30 (m, 1H), 6.76 (d, J = 2.9 Hz, 1H), 5.28-5.24 (m, 1H), 4.60-4.53 (m, 1H), 4.24-4.18 (m, 1H), 2.59-2.51 (m, 1H), 2.22-2.14 (m, 1H). |
| 489 | 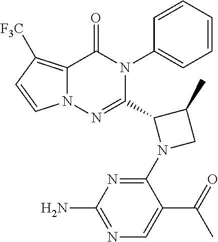 [SMILES: CC(=O)c1cnc(N)nc1N1C[C@H](C)[C@H]1c1nn2ccc(C(F)(F)F)c2c(=O)n1-c1ccccc1] | 484.2 | ¹H NMR (400 MHz, CD₃OD) δ 8.36 (s, 1H), 7.66-7.51 (m, 4H), 7.33-7.29 (m, 2H), 6.76 (d, J = 2.9 Hz, 1H), 4.91 (brs, 1H), 4.34 (brs, 1H), 3.29 (brs, 1H), 2.50 (brs, 1H), 2.19 (s, 3H), 0.62 (d, J = 6.8 Hz, 3H). |
| 490 | 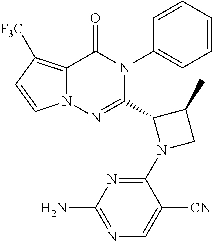 [SMILES: C[C@H]1CN(c2nc(N)ncc2C#N)[C@@H]1c1nn2ccc(C(F)(F)F)c2c(=O)n1-c1ccccc1] | 467.1 | ¹H NMR (400 MHz, CD₃OD) δ 8.01 (s, 1H), 7.55-7.48 (m, 4H), 7.43 (d, J = 2.6 Hz, 1H), 7.32-7.30 (m, 1H), 6.78 (d, J = 2.9 Hz, 1H), 4.76 (brs, 1H), 4.34 (brs, 1H), 3.60 (brs, 1H), 2.65 (brs, 1H), 0.66 (d, J = 6.7 Hz, 3H). |

The following compounds may be made using the procedures described in previously

| | | Cacl. |
|---|---|---|
| Compound | Structure | MS(M + 1) |
| 408 | 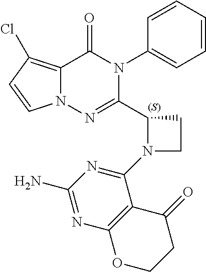 | 464.1 |
| 409 | 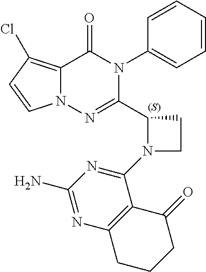 | 462.1 |
| 410 | 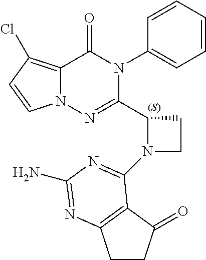 | 448.1 |
| 411 | 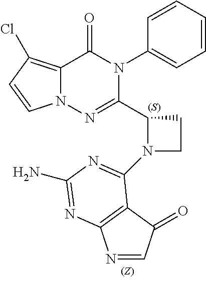 | 447.1 |
| 412 | 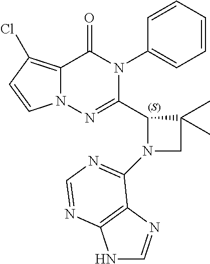 | 447.1 |
| 413 | 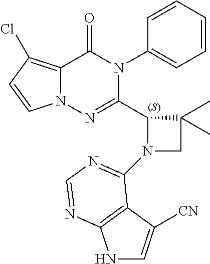 | 471.1 |
| 415 | 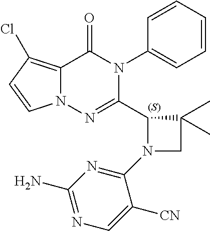 | 447.1 |
| 416 | 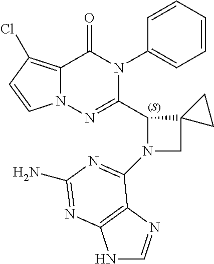 | 445.1 |
| 417 | 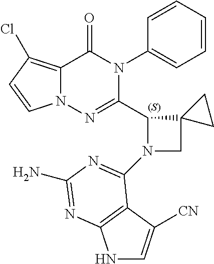 | 469.1 |
| 418 | 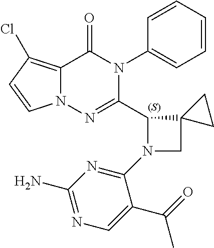 | 462.1 |
| 419 | 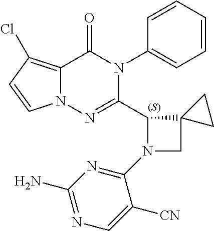 | 445.1 |
| 420 | 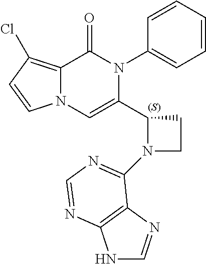 | 418.1 |
| 421 | 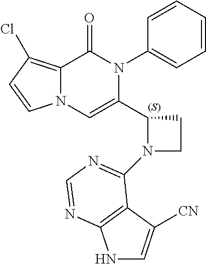 | 442.1 |
| 422 | 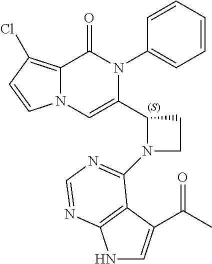 | 459.1 |
| 423 | 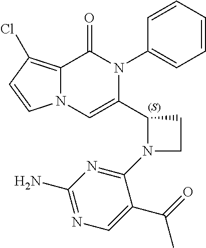 | 435.1 |
| 424 | 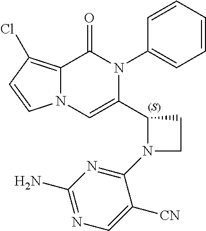 | 418.1 |
| 425 | 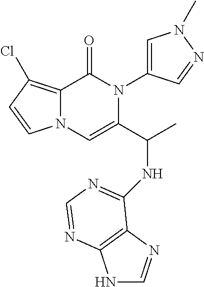 [SMILES: CC(Nc1ncnc2[nH]cnc12)c1cn2ccc(Cl)c2c(=O)n1-c1cnn(C)c1] | 410.1 |
| 426 | 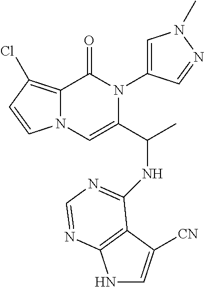 [SMILES: CC(Nc1ncnc2[nH]cc(C#N)c12)c1cn2ccc(Cl)c2c(=O)n1-c1cnn(C)c1] | 434.1 |
| 427 | 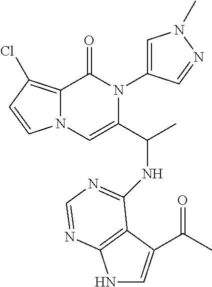 [SMILES: CC(=O)c1c[nH]c2ncnc(NC(C)c3cn4ccc(Cl)c4c(=O)n3-c3cnn(C)c3)c12] | 451.1 |
| 428 | 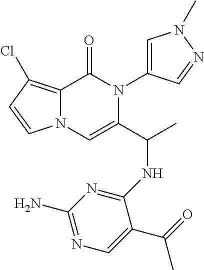 [SMILES: CC(=O)c1cnc(N)nc1NC(C)c1cn2ccc(Cl)c2c(=O)n1-c1cnn(C)c1] | 427.1 |
| 429 | 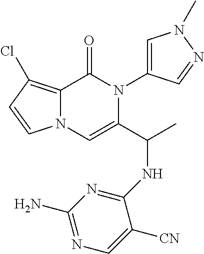 [SMILES: CC(Nc1nc(N)ncc1C#N)c1cn2ccc(Cl)c2c(=O)n1-c1cnn(C)c1] | 410.1 |
| 453 | 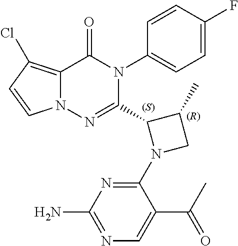 | 468.1 |
| 454 | 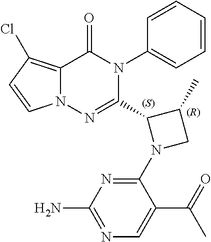 | 450.1 |
| 455 | 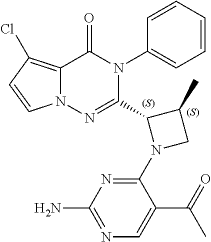 | 450.1 |
| 456 | 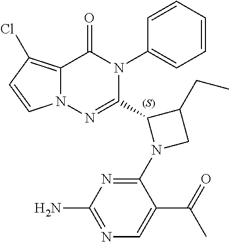 | 464.1 |
| 457 | 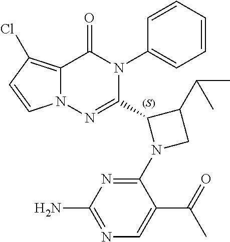 | 478.1 |
| 458 | 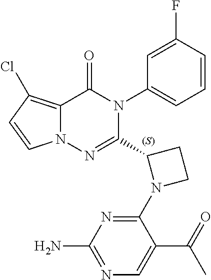 | 454.1 |
| 459 | 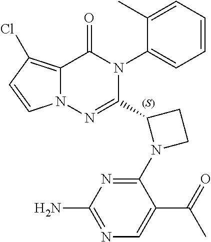 | 450.1 |
| 460 | 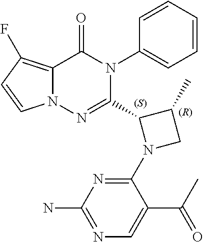 | 434.2 |
| 505 | 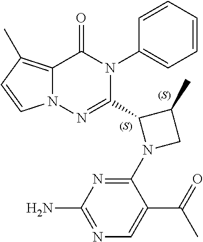 | 430.2 |
| 506 | 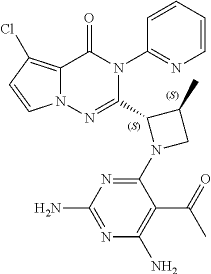 | 466.1 |
| 507 | 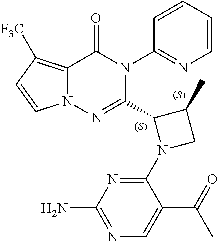 | 485.2 |
| 508 | 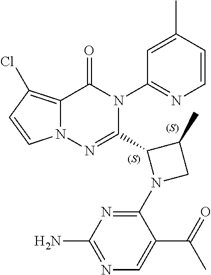 | 465.2 |
| 510 | 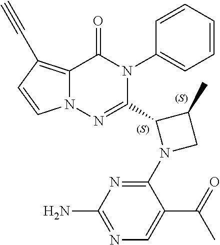 | 440.2 |
| 511 | 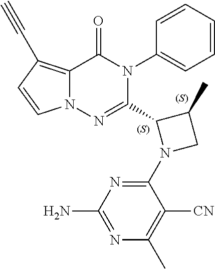 | 437.2 |
| 512 | 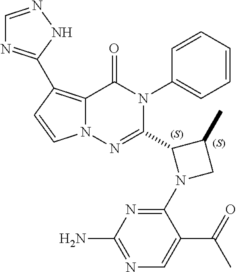 | 483.2 |
| 513 | 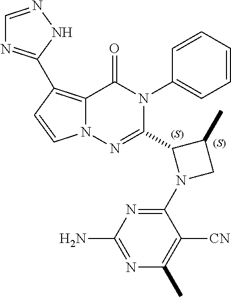 | 480.2 |
| 514 | 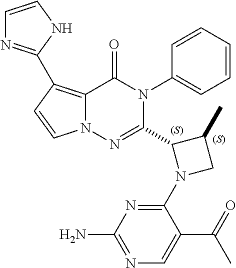 | 482.2 |
| 515 | 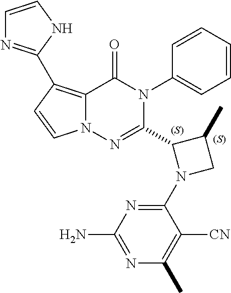 | 479.2 |
| 516 | 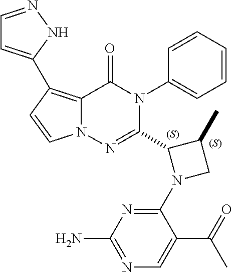 | 482.2 |
| 517 | 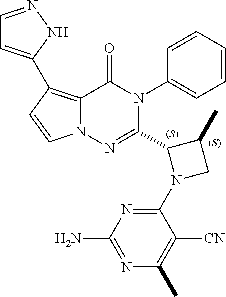 | 479.2 |
| 519 | 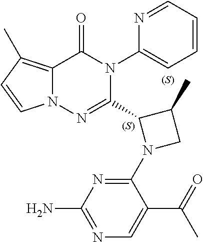 | 431.3 |
| 520 | 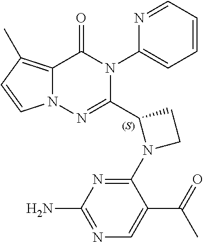 | 417.2 |
| 521 | 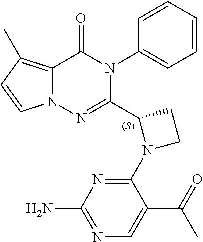 | 416.2 |

### Example 32

### Kinase Inhibition assays of p110α/p85α, p110β/p85α, p110δ/p85α and p110γ

PI₃K kinases including p110α/p85α, p110δ/p85α and p110γ are purchased from Invitrogen, and p110β/p85α is from Millipore.

Primary screening data and IC₅₀ values are measured using Transcreener™ KINASE Assay (Bellbrook, Catalog #3003-10K). The assay can be carried out according to the procedures suggested by the manufacturer. It is a universal, homogenous, high throughput screening (HTS) technology using a far-red, competitive fluorescence polarization immunoassay based on the detection of ADP to monitor the activity of enzymes that catalyze group transfer reactions. Briefly, the Transcreener KINASE Assay is designed as a simple two-part, endpoint assay.

In the first step, the 25 ul kinase reaction is performed by preparing a reaction mixture containing 5 ul test compound (2% DMSO final concentration), 10 ul kinase, buffer (50 mM HEPES, 100 mM NaCl, 1 mM EGTA, 0.03% CHAPS, 3 mM MgCl₂, and freshly supplemented 1 mM DTT), and 10 ul 30 uM PIP2/10 uM ATP. The plate is sealed and incubated for 80 min at room temperature. Next, 25 ul ADP detection mix is added per well. The plate is sealed again and incubated for 60 min at room temperature, and then measure fluorescence polarization by Tecan Infinite F500 Reader.

Data is analyzed and IC₅₀s are generated using the add-in software for Microsoft Excel, Xlfit™ (version 2.0). IH %=(ADP amount under 2% DMSO-ADP amount under test compound)/ADP amount under 2% DMSO.

In vitro activity data:

| | PI3Kα | | PI3Kβ | | PI3Kγ | | PI3Kδ | |
|---|---|---|---|---|---|---|---|---|
| Compd. | IH % @ | IC₅₀ | IH % @ | IC₅₀ | IH % @ | IC₅₀ | IH % @ | IC₅₀ |
| No. | 1 uM | (uM) | 1 uM | (uM) | 1 uM | (uM) | 1 uM | (uM) |
| 6 | −10.6 | | 28.0 | | 58.6 | | 91.5 | 0.093 |
| 7 | −18.6 | | 17.7 | | 16.6 | | 50.4 | |
| 8 | | | 33.3 | | 40.2 | | 76.2 | 0.272 |
| 25 | 80.9 | | 93.7 | 0.034 | 98.6 | 0.004 | >100 | 0.001 |
| 26 | 20.3 | | 88.7 | 0.091 | 93.3 | 0.012 | >100 | 0.002 |
| 27 | | | 82.6 | 0.100 | 92.7 | 0.051 | >100 | 0.003 |
| 28 | −0.3 | | 49.9 | | 92.2 | 0.032 | >100 | 0.014 |
| 29 | −1.5 | | 18.5 | | 72.5 | 0.271 | >100 | 0.084 |
| 30 | −20.5 | | 51.3 | | 74.1 | 0.094 | >100 | 0.009 |
| 31 | −17.7 | | 35.3 | | 81.5 | 0.153 | >100 | 0.016 |
| 32 | 54.5 | | 96.7 | 0.013 | 94.8 | 0.008 | >100 | 0.001 |
| 33 | | | −3.1 | | 63.7 | | 71.9 | 0.212 |
| 34 | −2.5 | | 12.4 | | 84.6 | 0.203 | 96.8 | 0.029 |
| 35 | −6.6 | | 24.7 | | 61.2 | | 94.1 | 0.057 |
| 36 | 6.4 | | 60.9 | | 90.8 | 0.035 | 99.1 | |
| 37 | | | 30.6 | | 83.3 | 0.089 | 81.2 | |
| 38 | −3.3 | | 54.8 | | 93.5 | 0.011 | >100 | 0.003 |
| 39 | | | 20.7 | | 16.5 | | 94.2 | |
| 40 | | | 19.8 | | 6.5 | | 74.0 | |
| 41 | | | 80.2 | 0.066 | >100 | 0.021 | 91.7 | 0.006 |
| 42 | 71.8 | | 79.8 | 0.186 | 91.2 | 0.005 | >100 | ~0.001 |
| 43 | 35.1 | | 66.0 | | 96.6 | 0.019 | >100 | 0.002 |
| 44 | 46.7 | | 74.3 | 0.302 | 95.2 | 0.005 | >100 | 0.001 |
| 45 | | | 71.9 | 0.795 | 80.9 | 0.172 | 100 | |
| 46 | | | 33.8 | | 68.1 | | >100 | 0.014 |
| 47 | | | 47.3 | | 84.7 | 0.152 | >100 | 0.026 |
| 48 | | | 69.7 | 0.501 | 86.1 | 0.058 | 98.4 | 0.004 |
| 49 | | | −4.8 | | 8.9 | | 8.4 | |
| 50 | | | 4.4 | | 89.3 | 0.149 | >100 | 0.029 |
| 51 | | | −7.4 | | 89.1 | 0.293 | 80.1 | 0.343 |
| 52 | | | 8.8 | | 89.3 | 0.107 | 87.2 | 0.110 |
| 53 | | | −11.0 | | 86.4 | 0.035 | 68.8 | |
| 54 | | | 26.0 | | 11.9 | | 90.1 | 0.207 |
| 55 | | | 23.8 | | 99.5 | 0.067 | 97.6 | 0.008 |
| 56 | | | 21.7 | | 83.9 | 0.287 | 91.1 | 0.156 |
| 57 | | | 37.1 | | 88.3 | 0.239 | 98.2 | 0.013 |
| 58 | | | 45.5 | | 97.6 | 0.073 | >100 | 0.005 |
| 59 | | | 34.7 | | 45.8 | | 73.6 | 0.392 |
| 60 | | | 3.2 | | 29.5 | | 69.0 | 0.325 |
| 61 | | | 7.9 | | 45.1 | | 73.9 | 0.309 |
| 62 | 7.1 | | 42.2 | | >100 | 0.039 | 95.4 | 0.039 |
| 63 | | | 93.7 | 0.061 | >100 | 0.081 | 97.7 | |
| 64 | 32.6 | | 78.8 | 0.251 | 89.9 | 0.041 | 98.9 | 0.003 |
| 65 | 52.6 | | 50.6 | | >100 | 0.078 | >100 | 0.014 |
| 66 | 75.6 | | 63.6 | | >100 | 0.014 | >100 | 0.012 |
| 67 | | | 71.3 | 0.188 | 61.0 | | 98.1 | 0.007 |
| 68 | | | 52.1 | | 73.8 | 0.078 | 98.5 | 0.028 |
| 69 | 13.0 | | 57.8 | | 68.8 | | 99.9 | 0.009 |
| 70 | 41.6 | | 92.1 | 0.220 | >100 | 0.025 | 99.1 | 0.003 |
| 71 | | | >100 | 0.031 | >100 | 0.009 | >100 | 0.001 |
| 72 | | | 13.5 | | 49.5 | | 91.6 | 0.088 |
| 73 | 33.6 | | 69.5 | 0.420 | 92.7 | 0.016 | >100 | 0.003 |
| 74 | | | >100 | 0.025 | >100 | 0.003 | >100 | 0.001 |
| 75 | | | 69.3 | 0.096 | 97.3 | 0.008 | 99.1 | 0.003 |
| 76 | | | 82.0 | 0.104 | 93.9 | 0.010 | 98.8 | 0.004 |
| 77 | | | 88.2 | 0.058 | 85.5 | 0.034 | 99.5 | |
| 78 | | | 92.4 | 0.026 | 91.2 | 0.018 | 98.2 | |
| 79 | | | 96.3 | 0.006 | 91.6 | 0.016 | 99.0 | |
| 80 | | | 58.9 | | 83.5 | 0.046 | >100 | 0.007 |
| 81 | | | 79.0 | 0.217 | 87.9 | 0.070 | >100 | 0.006 |
| 82 | | | 56.4 | | 78.6 | 0.194 | 98.7 | |
| 83 | | | 42.7 | | 78.6 | 0.309 | 97.3 | |
| 84 | −3.5 | | 59.3 | | 75.9 | 0.032 | >100 | 0.004 |
| 85 | 27.4 | | 74.7 | 0.311 | 87.8 | 0.030 | >100 | 0.001 |
| 86 | 17.8 | | 86.5 | 0.172 | 76.4 | 0.139 | 99.0 | 0.002 |
| 87 | | | 90.8 | 0.049 | >100 | 0.008 | | |
| 88 | | | 94.7 | 0.058 | 98.0 | 0.014 | 93.2 | |
| 89 | | | 96.1 | 0.017 | 94.8 | 0.016 | >100 | |
| 90 | | | 93.1 | 0.024 | 95.7 | 0.034 | >100 | |
| 91 | 48.3 | | 78.3 | 0.222 | 93.5 | 0.034 | >100 | 0.005 |
| 92 | 31.8 | | 65.2 | | 95.7 | 0.020 | >100 | 0.003 |
| 93 | | | 5.4 | | 53.7 | | 77.6 | 0.244 |
| 95 | | | 82.0 | 0.036 | >100 | 0.007 | 97.6 | 0.001 |
| 96 | | | 73.4 | 0.169 | 94.3 | 0.071 | 97.3 | 0.024 |
| 97 | | | 45.1 | | 84.6 | 0.144 | 55.1 | |
| 100 | | | 89.8 | 0.006 | >100 | 0.005 | >100 | 0.001 |
| 101 | 47.8 | | 81.6 | 0.138 | >100 | 0.016 | >100 | 0.003 |
| 102 | | | 92.3 | 0.061 | >100 | 0.014 | >100 | 0.001 |
| 103 | | | >100 | 0.046 | 98.2 | 0.019 | 99.7 | 0.001 |
| 104 | | | >100 | 0.017 | >100 | 0.003 | >100 | <0.0005 |
| 105 | | | 16.0 | | 90.4 | 0.080 | 90.0 | 0.015 |
| 107 | | | 34.5 | | 71.8 | 0.153 | 98.6 | 0.005 |
| 108 | | | 26.9 | | 90.0 | 0.199 | 75.9 | 0.097 |
| 109 | | | 61.0 | | 98.3 | 0.192 | 99.8 | 0.004 |
| 111 | | | 39.0 | | 67.2 | | 93.9 | 0.045 |
| 114 | | | 86.4 | 0.159 | 4.3 | | 93.7 | 0.027 |
| 115 | | | 80.2 | 0.143 | 91.7 | 0.003 | >100 | 0.002 |
| 116 | | | >100 | 0.128 | 96.9 | 0.045 | >100 | 0.005 |
| 117 | | | >100 | 0.038 | >100 | 0.043 | >100 | 0.005 |
| 118 | | | 19.1 | | 5.2 | | 77.5 | 0.471 |
| 119 | | | 47.8 | | 85.6 | 0.239 | 94.3 | |
| 120 | | | 74.7 | 0.237 | 85.9 | 0.295 | >100 | |
| 121 | | | 63.9 | | >100 | 0.105 | 92.7 | |
| 122 | | | 88.3 | 0.051 | >100 | 0.008 | >100 | 0.003 |
| 123 | | | 47.9 | | 67.9 | | 94.6 | 0.022 |
| 124 | | | 95.0 | 0.022 | >100 | 0.012 | 98.0 | 0.002 |
| 125 | | | 95.7 | 0.006 | 94.0 | 0.003 | >100 | 0.001 |
| 126 | | | 90.9 | 0.025 | >100 | 0.020 | >100 | 0.001 |
| 127 | 7.0 | | 71.3 | 0.307 | >100 | 0.057 | 99.2 | 0.005 |
| 128 | 40.3 | | 87.8 | 0.086 | 96.2 | 0.010 | 99.0 | 0.001 |
| 129 | | | 17.8 | | 33.3 | | 97.8 | 0.018 |
| 130 | | | 32.9 | | 20.8 | | 96.2 | 0.136 |
| 131 | | | 15.1 | | −9.7 | | 62.0 | |
| 132 | | | 74.5 | 0.338 | >100 | 0.070 | >100 | 0.009 |
| 133 | | | 11.5 | | 65.9 | | 88.1 | 0.172 |
| 134 | | | 59.2 | | >100 | 0.030 | >100 | 0.005 |
| 135 | | | 20.6 | | >100 | 0.012 | 74.5 | 0.051 |
| 136 | | | 27.6 | | 95.0 | 0.042 | 83.5 | 0.124 |
| 137 | | | 35.9 | | 89.3 | 0.013 | 96.8 | 0.036 |
| 138 | | | 42.3 | | 95.0 | 0.075 | >100 | 0.012 |
| 139 | | | 18.0 | | 46.5 | | 64.8 | |
| 140 | | | 15.0 | | 82.3 | 0.116 | >100 | 0.051 |
| 141 | | | 28.2 | | 92.3 | 0.151 | >100 | 0.005 |
| 142 | | | 13.5 | | 75.5 | 0.390 | 81.1 | 0.298 |
| 143 | | | 63.0 | | 82.3 | 0.095 | 88.8 | 0.070 |
| 144 | | | 62.5 | | 94.1 | 0.044 | >100 | 0.005 |
| 145 | | | 55.5 | | >100 | 0.009 | >100 | 0.002 |
| 146 | | | 77.9 | 0.120 | 97.3 | 0.009 | >100 | 0.001 |
| 147 | | | 65.3 | | 94.3 | 0.004 | >100 | 0.001 |
| 148 | | | 19.5 | | 83.0 | 0.173 | 86.7 | 0.044 |
| 149 | | | −35.9 | | 74.2 | 0.348 | 95.9 | 0.052 |
| 150 | | | 31.5 | | 92.6 | 0.092 | >100 | 0.003 |
| 151 | | | 11.4 | | 22.8 | | 52.4 | |
| 152 | | | 54.4 | | 79.3 | 0.287 | 99.2 | 0.005 |
| 153 | | | 56.5 | | 85.8 | 0.165 | >100 | 0.011 |
| 154 | | | 56.7 | | 93.7 | 0.040 | 97.6 | 0.003 |
| 155 | | | 56.0 | | 94.9 | 0.133 | 96.4 | 0.023 |
| 156 | | | 42.2 | | 64.0 | | 83.4 | 0.169 |
| 157 | | | 39.5 | | 79.9 | 0.280 | >100 | 0.021 |
| 158 | | | 71.1 | 0.473 | >100 | 0.046 | >100 | 0.006 |
| 159 | | | 32.8 | | 20.4 | | 85.0 | 0.127 |
| 160 | | | 11.4 | | 34.3 | | 80.2 | 0.140 |
| 161 | | | 15.3 | | −8.4 | | 45.7 | |
| 162 | | | 83.2 | 0.137 | 97.7 | 0.006 | >100 | 0.001 |
| 163 | | | −3.2 | | 0.6 | | 31.3 | |
| 164 | | | 22.9 | | 64.9 | | 62.3 | |
| 165 | | | 71.3 | 0.400 | >100 | 0.002 | >100 | 0.001 |
| 166 | | | >100 | 0.017 | >100 | 0.002 | 97.2 | 0.001 |
| 167 | | | 42.3 | | >100 | 0.021 | >100 | 0.005 |
| 168 | | | 98.8 | 0.047 | 95.1 | 0.015 | >100 | 0.001 |
| 169 | | | −21.1 | | 31.2 | | 88.0 | 0.004 |
| 170 | | | 4.6 | | 66.5 | | 96.1 | 0.005 |
| 171 | | | 25.2 | | 75.3 | 0.130 | 96.6 | 0.005 |
| 172 | | | 38.2 | | 79.8 | 0.297 | 99.6 | 0.002 |
| 173 | | | 25.7 | | 48.7 | | 96.3 | 0.004 |
| 174 | | | 97.7 | 0.023 | 94.0 | 0.031 | >100 | 0.001 |
| 175 | | | 90.9 | 0.078 | 87.6 | 0.105 | 99.5 | 0.001 |
| 176 | | | 16.8 | | 58.3 | | 97.1 | 0.005 |
| 177 | | | 17.0 | | 79.8 | 0.089 | 97.1 | 0.030 |
| 178 | | | 1.5 | | 7.6 | | 82.3 | 0.211 |
| 179 | | | 51.5 | | 97.9 | 0.015 | >100 | 0.002 |
| 180 | | | 92.8 | 0.041 | 98.7 | 0.002 | >100 | <0.00046 |
| 181 | | | 95.9 | 0.023 | >100 | 0.004 | >100 | <0.00046 |
| 182 | | | 93.3 | 0.062 | 94.9 | 0.007 | >100 | <0.00046 |
| 183 | | | 77.2 | 0.331 | >100 | 0.005 | >100 | <0.00046 |
| 184 | | | >100 | 0.038 | 98.4 | 0.008 | >100 | 0.0005 |
| 185 | | | 45.9 | | 99.0 | 0.005 | >100 | 0.006 |
| 186 | 28.4% | | >100% | 0.284 | >100% | 0.010 | >100% | 0.001 |
| 187 | 14.1% | | 84.4% | 0.088 | >100% | 0.033 | 99.0% | 0.001 |
| 188 | 14.7% | | 68.7% | 0.741 | >100% | 0.017 | 98.8% | 0.005 |

| | PI3Kα | | PI3Kβ | | PI3Kγ | | PI3Kδ | |
|---|---|---|---|---|---|---|---|---|
| | IH % | | IH % | | IH % | | IH % | |
| Compd. | @ 1 | IC50 | @ 1 | IC50 | @ 1 | IC50 | @ 1 | IC50 |
| No. | uM | (uM) | uM | (uM) | uM | (uM) | uM | (uM) |
| 189 | 54.0 | | 87.0 | 0.087 | 89.2 | 0.015 | 97.5 | 0.001 |
| 190 | | | 52.9 | | 84.1 | 0.067 | 92.0 | 0.003 |
| 191 | | | 56.8 | | >100 | 0.032 | >100 | 0.003 |
| 192 | | | 65.3 | | >100 | 0.018 | 98.5 | 0.004 |
| 193 | | | 31.5 | | 93.7 | 0.121 | >100 | 0.023 |
| 194 | | | 29.5 | | 75.1 | 0.150 | >100 | 0.023 |
| 195 | | | 31.2 | | 72.8 | 0.168 | >100 | 0.019 |
| 196 | | | 32.3 | | >100 | 0.065 | 97.1 | 0.069 |
| 197 | 28.4 | | >100 | 0.284 | >100 | 0.010 | >100 | 0.001 |
| 198 | 17.4 | | 82.4 | 0.323 | >100 | 0.010 | >100 | 0.001 |
| 199 | | | 17.1 | | 94.6 | 0.034 | 59.5 | 2.004 |
| 200 | | | 28.2 | | 93.4 | 0.190 | 90.9 | 0.196 |
| 201 | | | 25.3 | | >100 | 0.049 | >100 | 0.019 |
| 203 | | | 23.3 | | 65.3 | | 94.6 | 0.100 |
| 204 | | | 28.9 | | 84.2 | 0.250 | 85.1 | 0.109 |
| 205 | | | 21.6 | | 76.1 | 0.229 | 76.1 | 0.074 |
| 206 | | 0.372 | >100 | 0.181 | >100 | 0.001 | 94 | 0.007 |
| 207 | | | 80.4 | 0.298 | >100 | 0.007 | >100 | 0.001 |
| 208 | | | 81.7 | 0.089 | 92.2 | 0.003 | 97.4 | 0.004 |
| 209 | | | 53.5 | | 89.6 | 0.030 | 94.2 | 0.012 |
| 210 | | | 69.1 | 0.191 | 92.0 | 0.006 | 98.6 | 0.002 |
| 211 | | | 88.3 | 0.051 | 92.2 | 0.002 | 98.4 | 0.0005 |
| 212 | | >1 | 37.0 | | >100 | 0.027 | 95.3 | 0.012 |
| 213 | 65.2 | | 85.9 | 0.088 | >100 | 0.007 | >100 | 0.001 |
| 214 | | | 65.7 | 0.271 | >100 | 0.012 | 99.7 | 0.001 |
| 215 | | | 32.8 | | 88.1 | 0.135 | 98.5 | 0.052 |
| 216 | | | 65.1 | | 91.8 | 0.003 | 96.5 | 0.002 |
| 218 | | | 85.0 | 0.165 | 95.7 | 0.004 | 97.3 | 0.002 |
| 219 | | | 75.1 | 0.358 | 87.4 | 0.014 | 98.4 | 0.003 |
| 220 | | | 25.1 | | 54.8 | | 84.8 | 0.242 |
| 221 | | | 16.3 | | 88.6 | 0.024 | 68.0 | |
| 222 | | | 40.5 | | 88.1 | 0.021 | 43.0 | |
| 223 | | | 22.9 | | 71.6 | 0.182 | 81.0 | 0.059 |
| 224 | | | 22.7 | | >100 | 0.052 | 85.9 | 0.060 |
| 225 | | | 96.3 | 0.054 | >100 | 0.005 | >100 | 0.001 |
| 226 | | | 41.8 | | >100 | 0.030 | 98.7 | 0.009 |
| 227 | | | 59.6 | | >100 | 0.018 | >100 | 0.005 |
| 228 | | | 13.8 | | 59.9 | | 74.6 | 0.176 |
| 229 | | | 81.5 | 0.262 | 90.4 | 0.002 | 97.6 | 0.003 |
| 230 | | | 75.2 | 0.280 | 87.3 | 0.007 | >100 | 0.003 |
| 231 | | | 80.5 | 0.197 | 96.8 | 0.004 | 98.1 | 0.009 |
| 232 | | | 63.4 | | >100 | 0.014 | >100 | 0.006 |
| 233 | | | >100 | 0.026 | >100 | 0.013 | >100 | 0.004 |
| 234 | | | 83.4 | 0.05 | >100 | 0.012 | >100 | 0.002 |
| 235 | | | 69.3 | 0.211 | 96.2 | 0.012 | >100 | 0.004 |
| 236 | | | 79.8 | 0.081 | 94.9 | 0.004 | >100 | 0.002 |
| 237 | | | 37.6 | | 86.3 | 0.035 | >100 | 0.014 |
| 238 | | | 33.8 | | >100 | 0.018 | >100 | 0.014 |
| 239 | | | 59.8 | | >100 | 0.075 | 98.7 | 0.018 |
| 240 | | | 45.0 | | >100 | 0.036 | 98.1 | 0.034 |
| 241 | | | 31.9 | | 98.7 | 0.014 | 95.3 | 0.032 |
| 242 | | | 46.5 | | 98.9 | 0.019 | 96.8 | 0.01 |
| 244 | | | 58.4 | | 92.7 | 0.030 | 99.8 | 0.004 |
| 245 | | | 38.4 | | 77.5 | 0.337 | 78.0 | 0.341 |
| 246 | | | 2.5 | | 80.8 | 0.696 | 84.6 | 0.562 |
| 247 | | | 4.8 | | 73.0 | | 53.0 | |
| 248 | | | −10.7 | | 98.0 | 0.009 | 96.2 | 0.009 |
| 249 | | | 24.8 | | 98.0 | 0.029 | 99.3 | 0.008 |
| 250 | | | 33.4 | | 95.8 | 0.045 | 99.1 | 0.022 |
| 251 | | | 50.4 | | 56.3 | | 88.3 | 0.102 |
| 252 | | | 56.6 | | 68.9 | | 97.0 | 0.007 |
| 253 | | | 45.1 | | 69.0 | 0.553 | 92.8 | 0.052 |
| 255 | | | 72.6 | 0.304 | >100 | 0.073 | >100 | 0.004 |
| 256 | | | 68.1 | | >100 | 0.082 | 97.5 | 0.006 |
| 257 | | | 82.4 | 0.080 | >100 | 0.018 | >100 | 0.002 |
| 258 | | | 10.4 | | 73.0 | 0.467 | 94.7 | 0.076 |
| 259 | | | 41.5 | | 89.5 | 0.170 | 98.8 | 0.027 |
| 260 | | | 39.6 | | 90.9 | 0.163 | 98.0 | 0.04 |
| 261 | | | >100 | 0.031 | >100 | 0.003 | >100 | 0.001 |
| 262 | | | 88.8 | 0.018 | 93.4 | 0.011 | 97.1 | <0.001 |
| 263 | | | 74.5 | 0.118 | >100 | 0.017 | >100 | 0.004 |
| 264 | | | 92.8 | 0.069 | >100 | 0.003 | >100 | 0.001 |
| 265 | | | 68.6 | 0.300 | >100 | 0.011 | >100 | 0.001 |
| 266 | | | 49.9 | | >100 | 0.021 | >100 | 0.006 |
| 267 | | | 73.2 | 0.206 | >100 | 0.013 | 98.9 | 0.003 |
| 268 | 38.4 | | 80.3 | 0.17 | >100 | 0.013 | >100 | 0.003 |
| 269 | | | 38.1 | | >100 | 0.093 | 94.7 | 0.147 |
| 270 | | | 87.4 | 0.174 | 89.7 | 0.022 | >100 | 0.012 |
| 271 | | | 94.2 | 0.015 | 97.6 | 0.002 | >100 | 0.001 |
| 272 | | | 76.9 | 0.239 | >100 | 0.021 | 98.8 | 0.007 |
| 273 | | | 98.8 | 0.012 | 98.3 | 0.005 | >100 | 0.003 |
| 274 | | | 61.6 | | 86.8 | 0.101 | 97.8 | 0.003 |
| 275 | | | 97.5 | 0.012 | 97.8 | 0.001 | >100 | 0.0004 |
| 276 | | | 52.9 | | 95.7 | 0.006 | 99.1 | 0.001 |
| 277 | | | 81.4 | 0.247 | 97.0 | 0.011 | 99.5 | 0.001 |
| 278 | | | 76.2 | 0.189 | 97.7 | 0.003 | 98.4 | 0.002 |
| 279 | | | 43.0 | | 92.0 | 0.042 | >100 | 0.005 |
| 280 | | | −2.0 | | 87.2 | 0.256 | 47.0 | |
| 281 | | | 11.1 | | 62.2 | | 15.7 | |
| 282 | | | 19.9 | | 93.5 | 0.025 | 94.9 | 0.040 |
| 283 | | | 78.0 | 0.137 | >100 | 0.001 | >100 | 0.002 |
| 284 | | | 9.7 | | 51.2 | | 51.2 | |
| 285 | | | 79.0 | 0.257 | >100 | 0.037 | >100 | 0.004 |
| 286 | | | 25.2 | | 56.2 | | 88.8 | 0.029 |
| 287 | | | 73.9 | 0.463 | 96.9 | 0.068 | >100 | 0.005 |
| 288 | | | 94.5 | 0.093 | 95.8 | 0.021 | 99.7 | 0.004 |
| 290 | | | | 0.039 | | 0.004 | | 0.001 |
| 291 | | | 12.6 | | 91.1 | 0.143 | 80.4 | 0.300 |
| 292 | | | 45.1 | | 94.7 | 0.112 | >100 | 0.007 |
| 293 | | | 54.2 | | 94.7 | 0.103 | 98.9 | 0.014 |
| 294 | | | 70.6 | 0.475 | >100 | 0.026 | 99.3 | 0.003 |
| 296 | | | 6.8 | | 85.5 | 0.036 | 77.0 | 0.381 |
| 297 | | | 61.5 | | 92.7 | 0.015 | 96.0 | 0.006 |
| 298 | | | 17.8 | | 70.2 | 0.158 | 61.9 | |
| 299 | | 2.943 | 38.6 | 0.644 | 95.4 | 0.004 | 99.4 | 0.006 |
| 300 | | | 51.5 | | 82.7 | 0.148 | 99.1 | 0.029 |
| 301 | | | 79.3 | 0.223 | >100 | 0.013 | >100 | 0.004 |
| 302 | | | 57.8 | | 98.6 | 0.008 | 96.5 | 0.077 |
| 303 | | | 92.7 | 0.021 | 94.6 | 0.001 | 97.2 | 0.001 |
| 304 | | | 47.4 | | 93.6 | 0.016 | 98.2 | 0.042 |
| 305 | | | 91.6 | 0.125 | 97.6 | 0.007 | >100 | 0.021 |
| 306 | | | 92.8 | 0.016 | >100 | 0.029 | >100 | 0.011 |
| 307 | | | 80.7 | 0.213 | 95.9 | 0.032 | 98.9 | 0.005 |
| 308 | 9.7 | | 56.4 | | 96.7 | 0.037 | 99.7 | 0.021 |
| 309 | | | 35.5 | | 94.6 | 0.099 | >100 | 0.011 |
| 311 | 10.1 | | 79.4 | 0.379 | >100 | 0.034 | >100 | <0.0005 |
| 312 | | | 20.0 | | 93.7 | 0.067 | 97.1 | 0.023 |
| 313 | | | 52.6 | | 77.2 | 0.423 | 100.0 | 0.003 |
| 314 | | | 16.8 | | 54.1 | | 17.2 | |
| 320 | | | 5.5 | | 55.9 | | 29.1 | |
| 321 | | | 80.5 | 0.218 | >100 | 0.011 | 100.0 | 0.005 |
| 322 | | | 58.0 | | >100 | 0.027 | 99.5 | 0.005 |
| 323 | | | 6.1 | | >100 | 0.021 | 99.4 | 0.012 |
| 324 | | | 67.1 | 0.456 | >100 | 0.005 | 98.8 | 0.001 |
| 325 | | | | >1 | | 0.043 | | 0.524 |
| 326 | | | −23.5 | | 50.7 | | 7.1 | |
| 327 | | | 73.4 | 0.250 | 97.2 | 0.001 | 99.5 | 0.002 |
| 329 | | | −7.0/2. | | 91.6 | 0.227 | 50.3 | |
| 331 | | | 20.9 | | >100 | 0.147 | 71.9 | 0.121 |
| 334 | | | 11.2 | | 82.1 | 0.068 | 25.7 | |
| 335 | | | 17.0 | | 69.5 | | 48.4 | |
| 337 | | | 93.2 | 0.021 | >100 | 0.005 | 99.2 | 0.001 |
| 340 | | | 76.1 | 0.163 | 94.3 | 0.009 | 100.0 | 0.001 |
| 342 | | | 45.2 | | 77.1 | 0.272 | 92.1 | 0.038 |
| 344 | | | 57.3 | | 85.5 | 0.081 | 94.8 | 0.085 |
| 345 | | | 93.2 | 0.028 | 97.1 | 0.004 | >100 | 0.001 |
| 346 | | | 86.1 | 0.047 | 94.1 | 0.026 | >100 | 0.002 |
| 347 | | | 87.8 | 0.07 | 91.8 | 0.013 | 98.3 | 0.002 |
| 348 | | | 51.2 | | 75.6 | 0.312 | 96.9 | 0.039 |
| 349 | | | 29.5 | | 76.6 | 0.268 | 92.6 | 0.111 |
| 350 | | | >100 | 0.035 | >100 | 0.004 | >100 | 0.001 |
| 351 | | | 89.6 | 0.081 | 95.5 | 0.003 | >100 | 0.001 |
| 352 | | | 40.7 | | 97.2 | 0.011 | >100 | 0.034 |
| 353 | | | 14.6 | | 79.2 | 0.223 | 33.8 | |
| 357 | | | 5 | | 66.2 | | 37.9 | >0.3 |
| 358 | | | 62.0 | 0.269 | >100 | 0.066 | >100 | 0.017 |
| 359 | | | 94.8 | 0.044 | >100 | 0.003 | >100 | 0.001 |
| 360 | | | 95.3 | 0.012 | >100 | 0.005 | 99.2 | 0.001 |
| 361 | | | 79.2 | 0.103 | >100 | 0.027 | 97.9 | 0.025 |
| 362 | | | 4.1 | | 97.4 | 0.04 | 56.0 | |
| 363 | | | 68.3 | | 98.3 | 0.027 | 97.5 | 0.006 |
| 364 | | | 88.2 | 0.056 | >100 | 0.017 | 99.1 | 0.002 |
| 365 | | | 79.0 | 0.275 | 88.6 | 0.025 | 98.0 | 0.003 |
| 366 | | | 74.4 | 0.300 | 86.8 | 0.089 | 97.1 | 0.011 |
| 369 | | | 68.8 | 0.242 | 90.4 | 0.003 | >100 | 0.002 |
| 371 | | | 17.5 | | 74.4 | 0.317 | 89.8 | 0.070 |
| 372 | | | 42.6 | | 87.7 | 0.297 | 84.8 | 0.100 |
| 373 | | | 37.4 | >1 | | 0.361 | | 0.027 |
| 374 | | | 58.7 | 0.517 | | 0.155 | | 0.004 |
| 375 | | | 32.9 | | 65.7 | | 92.6 | 0.043 |
| 376 | | | 54.8 | | 93.2 | 0.026 | 99.1 | 0.006 |
| 377 | | | 39.8 | | 96.3 | 0.045 | 98.0 | 0.034 |
| 378 | | | 34.5 | | >100 | 0.179 | 87.2 | 0.220 |
| 379 | | | 14.5 | | | 0.035 | | 0.059 |
| 380 | | | 87.8 | 0.065 | >100 | 0.01 | 98.0 | 0.001 |
| 381 | | | | 0.199 | | 0.029 | | 0.003 |
| 382 | 14.1 | | 84.4 | 0.088 | >100 | 0.033 | 99.0 | 0.001 |
| 383 | 14.7 | | 68.7 | 0.741 | >100 | 0.017 | 98.8 | 0.004 |
| 384 | | | 24.7 | | 53.6 | | 91.0 | 0.240 |
| 385 | | | 83.3 | 0.075 | 95.9 | 0.010 | >100 | 0.004 |
| 386 | | | 76.8 | 0.322 | 95.0 | 0.021 | >100 | 0.003 |
| 387 | | | 39.6 | | >100 | 0.009 | 98.2 | 0.007 |
| 388 | | | 69.3 | | 80.7 | 0.173 | 96.2 | 0.002 |
| 389 | | | 32.7 | | 87.9 | 0.046 | 95.4 | 0.007 |
| 391 | | | 0.1 | | 90.5 | 0.129 | 91.4 | 0.185 |
| 392 | | | 67.2 | | 89.1 | 0.062 | 95.9 | 0.011 |
| 393 | | | 35.2 | | 90.7 | 0.009 | 94.7 | 0.009 |
| 394 | | | 71.3 | 0.256 | 93.1 | 0.038 | 99.2 | 0.021 |
| 395 | | | 22.4 | | 91.7 | 0.016 | 97.1 | 0.064 |
| 396 | | | 86.1 | 0.369 | 94.1 | 0.017 | >100 | 0.002 |
| 397 | | | 52.0 | 2.349 | 96.5 | 0.013 | >100 | 0.011 |
| 398 | | | 22.6 | | >100 | 0.018 | 99.1 | 0.025 |
| 399 | | | 3.5 | | 70.3 | | 35.1 | |
| 400 | | | 22.2 | | 70.4 | 0.081 | >100 | 0.012 |
| 401 | | | 46.7 | | 67.8 | 0.189 | >100 | 0.004 |
| 402 | | | 21.7 | | 65.8 | | 93.4 | 0.067 |
| 403 | | | 71.7 | 0.123 | 93.1 | 0.007 | 98.1 | 0.001 |
| 404 | | | 31.1 | | 95.6 | 0.010 | 93.7 | 0.003 |
| 405 | | | 86.5 | 0.332 | 92.3 | 0.002 | >100 | 0.003 |
| 406 | | | 7.7 | | 35.2 | | 90.0 | 0.073 |
| 407 | | | >100 | 0.068 | >100 | 0.002 | 94.9 | 0.001 |
| 430 | | | 28.1 | | 87.3 | 0.052 | 93.9 | 0.013 |
| 431 | | | 51.3 | | 95.9 | 0.008 | 96.2 | 0.007 |
| 432 | | | 43.2 | | 89.0 | 0.009 | 81.3 | 0.160 |
| 435 | | | 91.2 | 0.014 | 83.7 | 0.016 | 99.7 | 0.003 |
| 436 | | | 78.5 | 0.024 | 98.7 | 0.002 | >100 | 0.001 |
| 437 | | | 97.1 | 0.027 | 91.3 | 0.002 | 96.3 | 0.001 |
| 438 | | | 79.3 | 0.273 | 91.4 | 0.006 | 91.2 | 0.023 |
| 439 | | | 93.0 | 0.022 | 92.4 | 0.003 | 98.1 | 0.002 |
| 440 | | | 20.3 | | 95.8 | 0.017 | 95.2 | 0.011 |
| 441 | | | 56.2 | | 97.7 | 0.014 | 95.5 | 0.002 |
| 442 | | | 76.1 | | 94.6 | 0.001 | 94.2 | 0.012 |
| 445 | | | 22.3 | | 4.3 | | 77.5 | 0.240 |
| 446 | | | 20.1 | | 40.0 | | 85.8 | 0.319 |
| 447 | | | 95.8 | 0.022 | >100 | 0.004 | 99.5 | 0.0004 |
| 448 | | | 55.9 | | 82.3 | 0.105 | 98.8 | 0.017 |
| 449 | | | 87.2 | 0.045 | 97.8 | 0.004 | 96.1 | 0.001 |
| 450 | | | 76.9 | 0.042 | 98.6 | 0.017 | 99.3 | 0.0004 |
| 451 | | | 4.2 | | 40.5 | | 96.4 | 0.035 |
| 452 | | | 97.0 | 0.013 | 92.5 | 0.001 | >100 | 0.0003 |
| 461 | | | 80.5 | 0.054 | >100 | 0.007 | >100 | 0.0004 |
| 462 | | | 80.5 | 0.185 | >100 | 0.005 | >100 | 0.001 |
| 463 | | | 89.6 | 0.050 | >100 | 0.005 | >100 | 0.004 |
| 464 | | | 33.0 | | 80.6 | 0.138 | >100 | 0.005 |
| 465 | | | 45.0 | | 94.1 | 0.023 | >100 | 0.008 |
| 466 | | | 45.4 | | 93.9 | 0.048 | >100 | 0.020 |
| 467 | | | 77.1 | 0.374 | 94.3 | 0.005 | 89.7 | 0.001 |
| 468 | | | 74.0 | 0.311 | 96.6 | 0.016 | >100 | 0.02 |
| 469 | | | 47.1 | | 90.5 | 0.051 | >100 | 0.019 |
| 470 | | | 4.9 | | 36.6 | | 27.8 | |
| 471 | | | 47.1 | | 92.4 | 0.007 | >100 | 0.003 |
| 472 | | | 40.2 | | >100 | 0.01 | >100 | 0.038 |
| 473 | | 0.921 | 67.3 | 0.454 | 97.0 | 0.010 | >100 | 0.002 |
| 474 | | | 48.3 | | 98.2 | 0.009 | 99.3 | 0.007 |
| 475 | | | 80.3 | 0.083 | 93.3 | 0.013 | 94.9 | 0.002 |
| 476 | | | 78.1 | 0.079 | 89.5 | 0.024 | 95.9 | 0.001 |
| 477 | | | 31.7 | | 76.1 | 0.351 | >100 | 0.058 |
| 478 | | | 96.4 | 0.032 | >100 | 0.003 | >100 | 0.001 |
| 479 | | | 20.1 | | 94.7 | 0.120 | 91.6 | 0.010 |
| 480 | | 0.648 | 93.6 | 0.081 | >100 | 0.002 | >100 | 0.003 |
| 481 | | 0.558 | 92.0 | 0.078 | >100 | 0.003 | >100 | 0.001 |
| 482 | | | 91.9 | 0.266 | >100 | 0.007 | >100 | 0.001 |
| 484 | | | 88.3 | 0.022 | >100 | 0.006 | >100 | 0.0001 |
| 485 | | | −2.1 | | 45.2 | | >100 | 0.096 |
| 486 | | | 41.5 | | >100 | 0.013 | >100 | 0.022 |
| 487 | | | 64.7 | | >100 | 0.008 | >100 | 0.006 |
| 488 | | | 29.1 | | 87.3 | 0.063 | 95.9 | 0.018 |
| 489 | | | 63.6 | 0.296 | >100 | 0.008 | >100 | 0.003 |
| 490 | | | 33.4 | | >100 | 0.015 | 96.6 | 0.054 |
| 491 | | | 65.1 | 1.581 | 96. | 0.018 | >100 | 0.012 |
| 492 | | | 50.7 | | >100 | 0.012 | 97.73 | 0.03 |
| 493 | | | 73.6 | 0.25 | >100 | /0.008 | 99.7 | 0.006 |
| 494 | | | 55.70 | | 93.5 | | | |
| 495 | | | | | >100 | | 99.5 | |
| 496 | | | 18.3 | | 17.2 | | 34.2 | |
| 497 | | | 67.6 | | 91.1 | 0.011 | >100 | 0.006 |
| 498 | | | | | >100 | 0.013 | >100 | 0.021 |
| 499 | | | 86.1 | 0.084 | 96.0 | 0.005 | >100 | 0.002 |
| 500 | | | 71.5 | 0.674 | 98.7 | 0.007 | 95.4 | 0.006 |
| 501 | | | 24.0 | | >100 | 0.038 | 91.8 | 0.078 |
| 502 | | | 33.6 | | 66.4 | | 66.2 | |
| 503 | | | 6.2 | | 63.0 | | 49.2 | |
| 504 | | | 75.6 | 0.121 | >100 | 0.002 | >100 | 0.002 |
| 509 | | | | | >100 | | >100 | |
| 518 | | | | | >100 | | >100 | |

### Example 62

### Acumen Assay—Raw264.7 p-AKT Assay

### Reagents and Materials

| Reagent | Brand | Catalog No. |
|---|---|---|
| poly-D-lysine 96-well | Beckman | 356692 |
| black/clear plate | Dickinson | |
| DMEM | GIBCO | C11965 |
| FBS | GIBCO | 2013-04 |
| C5a | R&D | 2150-C5-025 |
| 4% Paraformaldehyde | DingGuo | DF021 |
| 10% Triton X-100 | Thermo Scientific | 28314 |
| BSA | Genview | DH016-4 |
| Rabbit anti-p-AKT(Ser473) | Cell Signal | #4060L |
| antibody | | |
| Goat anti-rabbit IgG Alexa 488 | Invitrogen | A11034 |
| Propidium Iodide (PI) | Sigma-Aldrich | P4170 |

Acumen® eX3 (A Multilaser Microplate Cytometer For Enhanced High Content Screening): TTP LabTech

### Acumen Protocol

3×10⁴ Raw264.7 macrophage cells were seeded into 96-well plates with DMEM+10% heat-inactivated FBS at 2,700 cells/well, 90 ul/well, overnight. After starvation for 3 hr at 37° C. under 5% CO₂, Raw264.7 cells were treated with 10 ul/well various concentrations of compound or 0.5% DMSO for 30 min, and then stimulated with 10 ul/well 10 nM C5a for 5 min.

1.) Cells were fixed 110 μL of 4% pre-warmed Paraformaldehyde (2% final), incubate for 45 min at room temperature.
2.) Remove paraformaldehyde solution. Add 100 μL of ice-cold 0.1% Triton X-100 in PBS and leave at 4° C. for 30 min.
3.) Wash once in 100 μL PBS.
4.) Incubate with 100 μL blocking buffer (1% BSA, in PBS) for 2 hours at room temperature.
5.) Wash once for 5 min with 100 ul PBS.
6.) Incubate with 40 μL 1:200 dilution of phospho AKT (Ser473) rabbit antibody in antibody dilution buffer (0.1% BSA, in PBS) overnight at 4° C.
7.) Wash for 3 times for 10 min with 100 ul PBS.
8.) Incubate for 90 min at room temperature with 50 μL of goat anti-rabbit Alex488 antibody at a 1:1,000 dilution in antibody dilution buffer (0.1% BSA, in PBS). Cover plate in foil to keep out of light.
9.) Wash for 3 times for 10 min with 100 μL PBS.
10.) Add 50 μL of 1.5 μM Propidium Iodide solution to each well to determine cell number at a 1:1,000 dilution in PBS (stock: 1.5 mM).
11.) Incubate at room temperature for 30 min.
12.) Seal the plate with a black cover-seal (supplied with plate).
13.) Load the plate into the Acumen Explorer and scan with the appropriate instrument settings.

| | PI3Kγ |
|---|---|
| | cell-C5a |
| Compd. | Raw264.7 |
| No. | IC₅₀ (uM) |
| 191 | 0.054 |
| 206 | 0.005 |
| 207 | 0.022 |
| 213 | 0.002 |
| 214 | 0.015 |
| 218 | 0.042 |
| 229 | 0.011 |
| 231 | 0.015 |
| 232 | 0.044 |
| 235 | 0.071 |
| 236 | 0.046 |
| 241 | 0.022 |
| 242 | 0.021 |
| 268 | 0.019 |
| 270 | 0.065 |
| 272 | 0.036 |
| 273 | 0.003 |
| 278 | 0.056 |
| 283 | 0.003 |
| 285 | 0.046 |
| 288 | 0.043 |
| 299 | 0.006 |
| 301 | 0.020 |
| 302 | 0.043 |
| 311 | 0.054 |
| 321 | 0.009 |
| 323 | 0.080 |
| 324 | 0.063 |
| 325 | 0.080 |
| 327 | 0.018 |
| 340 | 0.045 |
| 351 | 0.005 |
| 364 | 0.006 |
| 369 | 0.022 |
| 380 | 0.019 |
| 403 | 0.011 |
| 407 | 0.063 |
| 431 | 0.085 |
| 436 | 0.037 |
| 449 | 0.087 |
| 450 | 0.072 |
| 461 | 0.018 |
| 462 | 0.090 |
| 467 | 0.039 |
| 471 | 0.096 |
| 473 | 0.036 |
| 475 | 0.094 |
| 480 | 0.011 |
| 481 | 0.014 |
| 486 | 0.051 |
| 487 | 0.046 |
| 489 | 0.023 |
| 491 | 0.018 |
| 492 | 0.012 |
| 493 | 0.008 |
| 496 | 0.002 |

What claimed is:

1. A compound of formula I-1, I-2 or I-3: 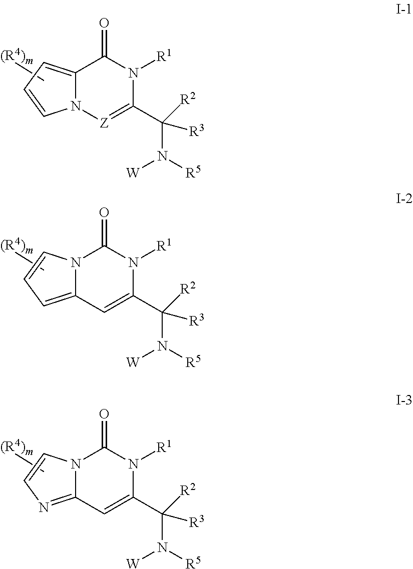
and/or its solvates, racemic mixture, enantiomers, diasteromers, tautomers, or mixtures of any ratio, or pharmaceutically acceptable salts thereof, wherein:
Z═N or CH;
R¹ is selected from optionally substituted C₁₋₆ alkyl, optionally substituted C₃₋₆ cycloalkyl, —(CR′R″)ₙ-heterocycle, —(CR′R″)ₙ-aryl, and —(CR′R″)ₙ-heteroaryl, wherein heterocycle, aryl and heteroaryl independently are 5-6 membered monocyclic ring, which are optionally substituted with one or more groups selected from hydrogen, halo, optionally substituted C₁₋₆ alkyl, optionally substituted C₁₋₆ alkoxyl, —CN, —CF₃, and —SO₂R′;
R² and R³ are each independently selected from hydrogen and optionally substituted C₁₋₄ alkyl;
R⁴ is selected from hydrogen, halo, —CN, optionally substituted C₁₋₆ alkyl, optionally substituted C₃₋₆ cycloalkyl, optionally substituted C₂₋₆ alkenyl, optionally substituted C₂₋₆ alkynyl, —C(O)NR′R″, and optionally substituted 5-6 membered monocyclic heteroaryl;
R⁵ is selected from hydrogen and optionally substituted C₁₋₄ alkyl;
or R³, R⁵ and the atoms they are attached to form an optionally substituted 4-6 membered mono- or bicyclic saturated or partially unsaturated heterocyclic ring;
R′ and R″ are each independently selected from hydrogen, halo, optionally substituted C₁₋₆ alkyl, optionally substituted C₃₋₆ cycloalkyl, and optionally substituted 4-6 membered monocyclic heterocycle;
or R′, R″ and the nitrogen or carbon atom they are both attached to form an optionally substituted 3-7 membered heterocycle;
each of m and n is 0, 1, 2, or 3;
each of p is 1 or 2;
W is a heteroaryl, which is optionally substituted with one or more groups selected from halo, —CN, —CF₃, —NO₂, —OR′, —NR′R″, —NR′COR″, —(CR′R″)ₙ—C(O)R′, —(CR′R″)ₙ—C(═N—OR′)—R″, —(CR′R″)ₙ—C(O)NR′R″, —(CR′R″)ₙ—S(O)ₚR′, —(CR′R″)ₙ—SR′, optionally substituted C₁₋₆ alkyl, optionally substituted C₂₋₆ alkenyl, optionally substituted C₂₋₆ alkynyl, optionally substituted C₁₋₆ alkoxy, optionally substituted 5-6 membered monocyclic heterocycle, and optionally substituted 5-6 membered monocyclic heteroaryl;
provided that for formula I-1, when Z═N, R³, R⁵ and the atoms they are attached to must form an optionally substituted 4-6 membered mono- or bicyclic saturated or partially unsaturated heterocyclic ring, with the provision that when R³, R⁵ and the atoms they are attached to form an optionally substituted 5 membered mono- or bicyclic saturated or partially unsaturated heterocyclic ring, R⁴ is not hydrogen, —CN, or aminomethyl.

2. A compound of formula I-1 according to claim 1, and/or its solvates, racemic mixture, enantiomers, diasteromers, tautomers, or mixtures of any ratio, or pharmaceutically acceptable salts thereof, wherein,
Z═N;
R¹ is selected from, optionally substituted C₁₋₆ alkyl, optionally substituted C₃₋₆ cycloalkyl, —(CR′R″)ₙ-heterocycle, —(CR′R″)ₙ-aryl, and —(CR′R″)ₙ-heteroaryl, wherein heterocycle, aryl and heteroaryl independently are 5-6 membered monocyclic ring, which are optionally substituted with one or more groups selected from halo, optionally substituted C₁₋₆ alkyl, optionally substituted C₁₋₆ alkoxyl, —CN, —CF₃, and —SO₂R′;
R² is selected from hydrogen and optionally substituted C₁₋₄ alkyl;
R³, R⁵ and the atoms they are attached to form an optionally substituted 4-6 membered mono- or bicyclic saturated or partially unsaturated heterocyclic ring;
R⁴ is selected from halo, C₁₋₆ alkyl, optionally substituted C₃₋₆ cycloalkyl, optionally substituted C₂₋₆ alkenyl, optionally substituted C₂₋₆ alkynyl, —C(O)NR′R″, and optionally substituted 5-6 membered monocyclic heteroaryl, wherein C₁-C₆ alkyl is optionally substituted with one or more groups selected from C₁-C₄ alkoxyl, —OH, and halo;
R′ and R″ are each independently selected from hydrogen, halo, optionally substituted C₁₋₆ alkyl, optionally substituted C₃₋₆ cycloalkyl, and optionally substituted 5-6 membered monocyclic heterocycle; or R′, R″ and the nitrogen or carbon atom they are both attached to form an optionally substituted 3-7 membered heterocycle;
each of m and n is 0, 1, 2, or 3;
each of p is 1 or 2;
W is a heteroaryl, which is optionally substituted with one or more groups selected from halo, —CN, —CF₃, —NO₂, —OR′, —NR′R″, —NR′COR″, —(CR′R″)ₙ—C(O)R′, —(CR′R″)ₙ—C(═N—OR′)—R″, —(CR′R″)ₙ—C(O)NR′R″, —(CR′R″)ₙ—S(O)ₚR′, —(CR′R″)ₙ—SR′, optionally substituted C₁₋₆ alkyl, optionally substituted C₂₋₆ alkenyl, optionally substituted C₂₋₆ alkynyl, optionally substituted C₁₋₆ alkoxy, optionally substituted 5-6 membered monocyclic heterocycle, and optionally substituted 5-6 membered monocyclic heteroaryl.

3. At least one compound of claim 2, and/or its solvates, racemic mixture, enantiomers, diasteromers, tautomers, or mixtures of any ratio, or pharmaceutically acceptable salts thereof, wherein,
R⁴ is selected from halo, C₁₋₆ alkyl, C₃-C₆ cycloalkyl, C₂-C₆ alkenyl, C₂-C₆ alkynyl, —C(O)NR′R″, wherein C₁-C₆ alkyl is optionally substituted with one or more groups selected from C₁-C₄ alkoxyl, —OH, and halo.

4. At least one compound of claim 3, and/or its solvates, racemic mixture, enantiomers, diasteromers, tautomers, or mixtures of any ratio, or pharmaceutically acceptable salts thereof, wherein R⁴ is selected from halo, —CF₃, and C₁₋₄ alkyl.

5. At least one compound of any one of claims 2-4, and/or its solvates, racemic mixture, enantiomers, diasteromers, tautomers, or mixtures of any ratio, or pharmaceutically acceptable salts thereof, wherein the said formula I-1 is

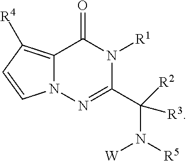

6. At least one compound of any one of claims 2 to 5, and/or its solvates, racemic mixture, enantiomers, diasteromers, tautomers, or mixtures of any ratio, or pharmaceutically acceptable salts thereof, wherein R³, R⁵ and the atoms they are attached to form an heterocyclic ring, which is optionally substituted

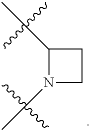 [SMILES: *C1CCN1*]

7. At least one compound of any one of claims 2 to 5, and/or its solvates, racemic mixture, enantiomers, diasteromers, tautomers, or mixtures of any ratio, or pharmaceutically acceptable salts thereof, wherein R³, R⁵ and the atoms they are attached to form an optionally substituted 5 membered saturated or partially unsaturated monocyclic heterocyclic ring.

8. At least one compound of claim 7, and/or its solvates, racemic mixture, enantiomers, diasteromers, tautomers, or mixtures of any ratio, or pharmaceutically acceptable salts thereof, wherein the said 5 membered saturated monocyclic heterocyclic ring is selected from 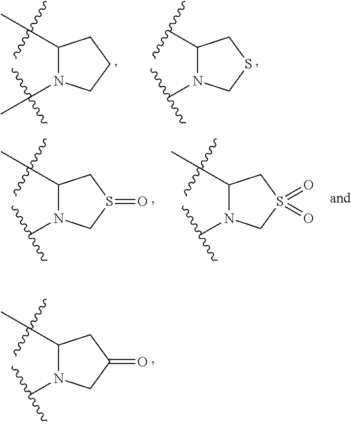 [SMILES: *C1CCCN1* ; *C1CSCN1* ; *C1CS(=O)CN1* ; *C1CS(=O)(=O)CN1* ; *C1CC(=O)CN1*]
each of which is optionally substituted.

9. At least one compound of any one of claims 2 to 5, and/or its solvates, racemic mixture, enantiomers, diasteromers, tautomers, or mixtures of any ratio, or pharmaceutically acceptable salts thereof, wherein R³, R⁵ and the atoms they are attached to form an optionally substituted 6 membered saturated or partially unsaturated mono or bicyclic heterocyclic ring.

10. At least one compound of claim 9, and/or its solvates, racemic mixture, enantiomers, diasteromers, tautomers, or mixtures of any ratio, or pharmaceutically acceptable salts thereof, wherein the said 6 membered mono or bicyclic saturated heterocyclic ring is 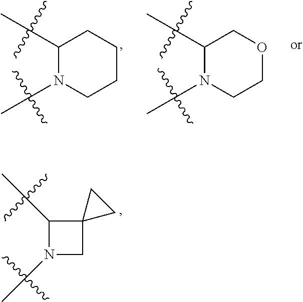 [SMILES: *C1CCCCN1* ; *C1COCCN1* ; *C1N(*)CC12CC2]
each of which is optionally substituted.

11. At least one compound of claim 1, and/or its solvates, racemic mixture, enantiomers, diasteromers, tautomers, or mixtures of any ratio, or pharmaceutically acceptable salts thereof, wherein,
Z═CH;
R² and R³ are each independently selected from hydrogen and optionally substituted C₁-C₄ alkyl;
R⁵ is selected from hydrogen and C₁-C₄ alkyl;
or R³, R⁵ and the atoms they are attached to form an optionally substituted 4-6 membered mono- or bicyclic saturated or partially unsaturated heterocyclic ring.

12. At least one compound of claim 11, and/or its solvates, racemic mixture, enantiomers, diasteromers, tautomers, or mixtures of any ratio, or pharmaceutically acceptable salts thereof, wherein,
R⁴ is selected from hydrogen, halo, optionally substituted C₁-C₆ alkyl, and optionally substituted 5-6 membered monocyclic heteroaryl.

13. At least one compound of claim 12, and/or its solvates, racemic mixture, enantiomers, diasteromers, tautomers, or mixtures of any ratio, or pharmaceutically acceptable salts thereof, wherein R⁴ is selected from hydrogen, halo, C₁-C₄ alkyl and 5-6 membered monocyclic heteroaryl, wherein 5-6 membered monocyclic heteroaryl is optionally substituted with C₁₋₄ alkyl.

14. At least one compound of any one of claims 11-13, and/or its solvates, racemic mixture, enantiomers, diasteromers, tautomers, or mixtures of any ratio, or pharmaceutically acceptable salts thereof, wherein the said formula I-1, I-2 and I-3 are II-1, II-2 and II-3 respectively,

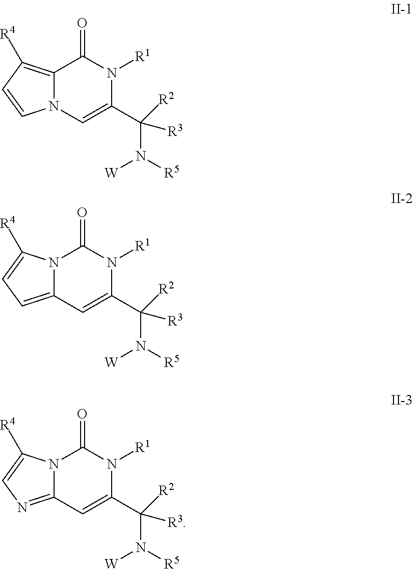

15. At least one compound of any one of claims 11-14, and/or its solvates, racemic mixture, enantiomers, diasteromers, tautomers, or mixtures of any ratio, or pharmaceutically acceptable salts thereof, wherein R³, R⁵ and the atoms they are attached to form an optionally substituted 4-6 membered saturated or partially unsaturated mono- or bicyclic heterocyclic ring.

16. At least one compound of claim 15, and/or its solvates, racemic mixture, enantiomers, diasteromers, tautomers, or mixtures of any ratio, or pharmaceutically acceptable salts thereof, wherein R³, R⁵ and the atoms they are attached to form an optionally substituted heterocycle selected from:

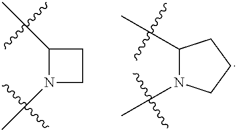 [SMILES: *C1CCN1* ; *C1CCCN1*]

17. At least one compound of any one of claims 1-16, and/or its solvates, racemic mixture, enantiomers, diasteromers, tautomers, or mixtures of any ratio, or pharmaceutically acceptable salts thereof, wherein the said heterocyclic ring, which is formed by R³, R⁵ and the atoms they are attached to, can be optionally substituted with one or more groups selected from halo, —OH, —CN, oxo, —SO₂R^{a}, —OR^{a}, and optionally substituted C₁₋₆ alkyl;
wherein R^{a} is C₁₋₆ alkyl, which is optional substituted with C₁-C₄ alkoxy.

18. At least one compound of any one of claims 1-17, and/or its solvates, racemic mixture, enantiomers, diasteromers, tautomers, or mixtures of any ratio, or pharmaceutically acceptable salts thereof, wherein R² is hydrogen.

19. At least one compound of any one of claims 11-14, and/or its solvates, racemic mixture, enantiomers, diasteromers, tautomers, or mixtures of any ratio, or pharmaceutically acceptable salts thereof, wherein R² and R³ are each independently H, methyl or ethyl.

20. At least one compound of claim 19, and/or its solvates, racemic mixture, enantiomers, diasteromers, tautomers, or mixtures of any ratio, or pharmaceutically acceptable salts thereof, wherein R⁵═H.

21. At least one compound of any one of claims 1-20, and/or its solvates, racemic mixture, enantiomers, diasteromers, tautomers, or mixtures of any ratio, or pharmaceutically acceptable salts thereof, wherein R¹ is selected from, C₁-C₆ alkyl, C₃-C₆cycloalkyl, —(CR′R″)ₙ-morpholinyl, —(CR′R″)ₙ-phenyl, —(CR′R″)ₙ-pyridinyl, or —(CR′R″)ₙ-pyrimidinyl, in which each of alkyl, morpholinyl, phenyl, pyridinyl and pyrimidinyl independently are optionally substituted with one or more groups selected from halo, C₁-C₄ alkyl, C₁-C₄ alkoxyl, —CN, —CF₃, and —SO₂R′.

22. At least one compound of claim 21, and/or its solvates, racemic mixture, enantiomers, diasteromers, tautomers, or mixtures of any ratio, or pharmaceutically acceptable salts thereof, wherein R¹ is (CR′R″)ₙ-phenyl, n is 0 and said phenyl can be optionally substituted with one or more groups selected from halo, —CN, C₁-C₄ alkoxyl, and —SO₂R′.

23. At least one compound of claim 22, and/or its solvates, racemic mixture, enantiomers, diasteromers, tautomers, or mixtures of any ratio, or pharmaceutically acceptable salts thereof, wherein said phenyl is phenyl optionally substituted with one or more halo.

24. At least one compound of any one of claims 1-4, 6-13 and 15-23, and/or its solvates, racemic mixture, enantiomers, diasteromers, tautomers, or mixtures of any ratio, or pharmaceutically acceptable salts thereof, wherein m=0, 1 or 2.

25. At least one compound of any one of claims 1-24, and/or its solvates, racemic mixture, enantiomers, diasteromers, tautomers, or mixtures of any ratio, or pharmaceutically acceptable salts thereof, wherein W is selected from IV-1 to IV-22,

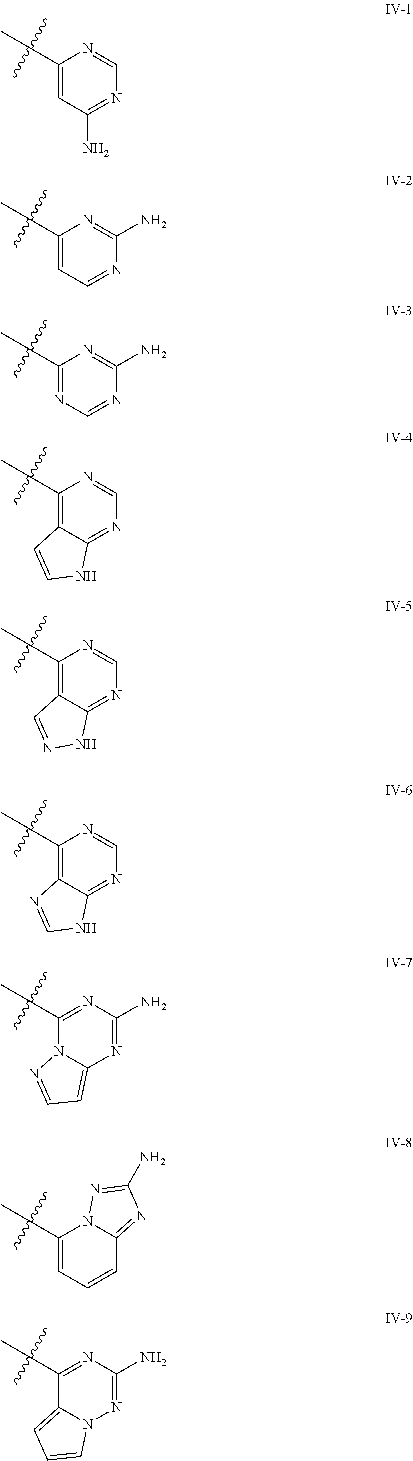

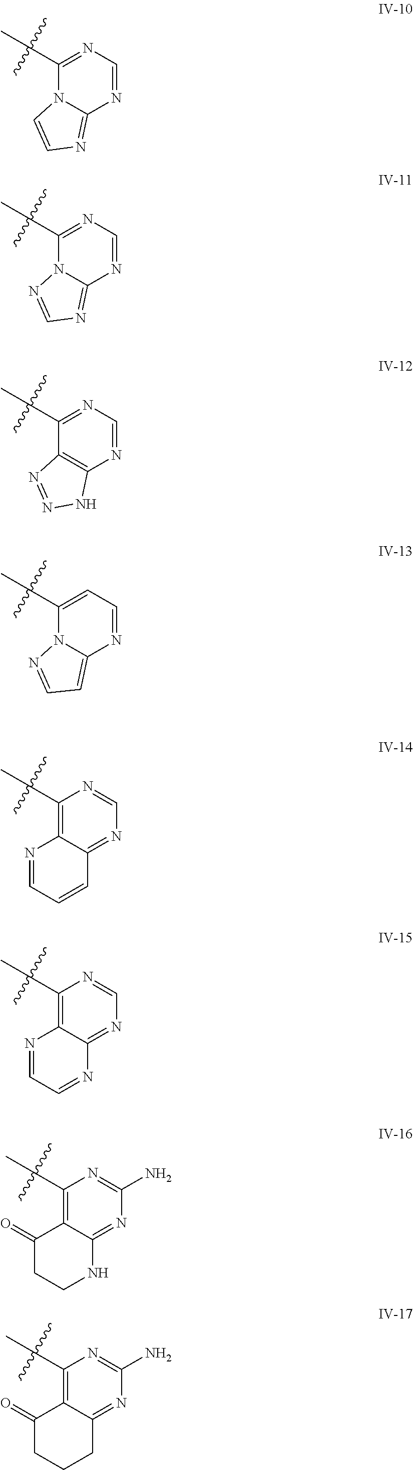

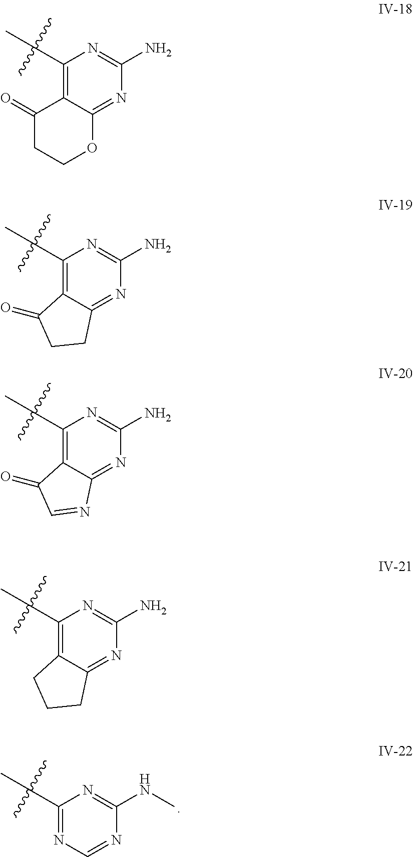

26. At least one compound of claim 25, and/or its solvates, racemic mixture, enantiomers, diasteromers, tautomers, or mixtures of any ratio, or pharmaceutically acceptable salts thereof, wherein W is optionally substituted with one or more groups selected from halo, —CN, —CF₃, —NO₂, —OR′, —NR′R″, —C(O)NR′R″, —NR′COR″, —C(O)R′, —C(═N—OR′)—R″, —S(O)ₚR′, —SR′, C₁₋₆ alkyl, C₂₋₆ alkenyl, C₂₋₆alkynyl, C₁₋₆alkoxy, 5-6 membered monocyclic heterocycle and 5-6 membered monocyclic heteroaryl; wherein alkyl, alkenyl, alkynyl, heterocycle and heteroaryl is optionally substituted with one or more groups selected from —OH, —CN, C₁₋₄ alkoxy, C₁₋₄ alkyl, and —NR′R″;
R′ and R″ are each independently selected from hydrogen, C₁₋₆ alkyl, C₃₋₆ cycloalkyl or 4-6 membered heterocycle, wherein alkyl is optionally substituted with one or more groups selected from —OH, halo and C₁₋₄alkoxy.

27. At least one compound of claim 26, and/or its solvates, racemic mixture, enantiomers, diasteromers, tautomers, or mixtures of any ratio, or pharmaceutically acceptable salts thereof, wherein W is IV-2, which is substituted with one or more groups selected from —CN, —NH₂, C₁-C₆ alkyl and —C(O)R′; R′ is C₁-C₆ alkyl optionally substituted with one or more halo, or R′ is C₃₋₆ cyclcoalkyl optionally substituted with one or more halo.

28. At least one compound of claim 26, and/or its solvates, racemic mixture, enantiomers, diasteromers, tautomers, or mixtures of any ratio, or pharmaceutically acceptable salts thereof, wherein W is IV-4, which is substituted with one or more groups selected from —CN, halo and —C(O)R′.

29. At least one compound of any one of claims 1 to 28, and/or its solvates, racemic mixture, enantiomers, diasteromers, tautomers, or mixtures of any ratio, or pharmaceutically acceptable salts thereof, wherein R′ and R″ are each independently selected from hydrogen, C₁₋₆ alkyl, and optionally substituted C₃₋₆ cycloalkyl.

30. At least one compound selected from compounds 1 to 521 and/or at least one its solvates, racemic mixture, enantiomers, diasteromers, tautomers, or mixtures of any ratio, or pharmaceutically acceptable salt thereof.

31. A composition comprising at least one compound of any one of claims 1-30, and/or at least one pharmaceutically acceptable salt thereof and at least one pharmaceutically acceptable carrier.

32. A method of inhibiting the activity of a PI₃K kinase comprising contacting the kinase with an effective amount of at least one compound of any one of claims 1-30, and/or its solvates, racemic mixture, enantiomers, diasteromers, tautomers, or mixtures of any ratio, or pharmaceutically acceptable salts thereof.

33. A method of treating a disease responsive to inhibition of PI₃K, comprising administrating to a subject in need thereof a therapeutically effective amount of at least one compound of any one of claims 1-30, and/or its solvates, racemic mixture, enantiomers, diasteromers, tautomers, or mixtures of any ratio, or pharmaceutically acceptable salts thereof.

34. A method of claim 33, wherein the disease responsive to inhibition of PI₃K is immune-based disease or cancer.

35. The method of claim 34, wherein said immune-based disease is rheumatoid arthritis, COPD, multiple sclerosis, asthma, glomerulonephritis, lupus, or inflammation related to any of the aforementioned; wherein said cancer is lymphoma or acute myeloid leukemia, multiple myelomia or chronic lymphocytic leukemia.

36. The method of any one of claims 33-35, wherein the said compound and/or its solvates, racemic mixture, enantiomers, diasteromers, tautomers, or mixtures of any ratio, or pharmaceutically acceptable salts thereof is administered in combination with another kinase inhibitor that inhibits a kinase activity other than a PI₃K kinase.

37. The compound of any one of claims 1-30, and/or its solvates, racemic mixture, enantiomers, diasteromers, tautomers, or mixtures of any ratio, or pharmaceutically acceptable salts thereof, for use in the treatment of a disease responsive to inhibition of PI₃K.

38. The compound of claim 37, wherein the disease responsive to inhibition of PI₃K is immune-based disease or cancer.

39. The compound of claim 38, wherein said immune-based disease is rheumatoid arthritis, COPD, multiple sclerosis, asthma, glomerulonephritis, lupus, or inflammation related to any of the aforementioned; wherein said cancer is lymphoma or acute myeloid leukemia, multiple myelomia or chronic lymphocytic leukemia.
